(19) United States
(12) Patent Application Publication
(10) Pub. No.: US 2026/0131011 A1
(43) Pub. Date: May 14, 2026

(54) Anti-Viral Therapeutic Peptides, Conjugates, and Methods of Use Thereof

(71) Applicant: Decoy Therapeutics Inc., Cambridge, MA (US)

(72) Inventors: Chinh H. DUONG, New Haven, CT (US); Barbara L. HIBNER, Winchester, MA (US); Janna N. HALLORAN, Port Monmouth, NJ (US); Linhai JIANG, Forest Hills, NY (US)

(21) Appl. No.: 19/195,255

(22) Filed: Apr. 30, 2025

Related U.S. Application Data
(60) Provisional application No. 63/640,306, filed on Apr. 30, 2024.
(60) Provisional application No. 63/776,437, filed on Mar. 24, 2025.

(51) Int. Cl. A61K 47/54; A61K 31/14; A61K 47/64; C07K 14/005

(52) U.S. Cl. CPC A61K 47/554; A61K 31/14; A61K 47/64; C07K 14/005; C07K 2319/00; C12N 2770/20022; C12N 2770/20033

(57) ABSTRACT

The application provides a plurality of new therapeutic peptides, which are designed based on the heptad repeat region of a viral spike fusion protein (such as SARS-Cov2, MERS-CoV, or HCov-OC43) or a heptad repeat region in a paramyxovirus (such as Measles, Nipah, or HPIV3)), new therapeutic conjugates that comprising these therapeutic peptides, and methods of using the therapeutic peptide conjugates for the treatment of a condition or disease associated with a viral infection.

### RELATED APPLICATIONS

This application claims the benefit of U.S. Provisional Application No. 63/776,437, filed on Mar. 24, 2025 and U.S. Provisional Application No. 63/640,306, filed on Apr. 30, 2024. The entire teachings of the above applications are incorporated herein by reference.

### SEQUENCE LISTING

The sequence listing submitted via EFS, in compliance with 37 CFR § 1.52(e)(5), is incorporated herein by reference. The sequence listing XML file submitted via EFS contains the file “43143005US2 Seq List.xml”, created on Aug. 22, 2025, which is 1,447,948 bytes in size.

### BACKGROUND OF THE INVENTION

The emergence of novel coronaviruses, including SARS-CoV-2, MERS, and HCoV-OC43, has significantly impacted global health, presenting unprecedented challenges to societies worldwide. The coronavirus family is characterized by enveloped, positive-sense, single-stranded RNA viruses that primarily infect mammals, with certain strains capable of crossing species barriers to infect humans. The most notable representatives of this viral family in recent years are the Severe Acute Respiratory Syndrome Coronavirus (SARS-CoV-2), the Middle East Respiratory Syndrome Coronavirus (MERS-CoV), and the human coronavirus OC43 (HCoV-OC43).

The coronavirus disease 2019 (COVID-19) pandemic, caused by SARS-CoV-2, has led to widespread illness, significant loss of life, and socio-economic disruptions. The virus primarily targets the respiratory system, leading to a spectrum of symptoms ranging from mild respiratory distress to severe pneumonia and acute respiratory distress syndrome (ARDS). Its high transmission rate, facilitated by respiratory droplets and asymptomatic carriers, has posed formidable challenges for containment efforts and public health measures.

MERS-CoV, identified in 2012, is another member of the coronavirus family that causes severe respiratory illness. While its incidence is lower than that of SARS-CoV-2, MERS-CoV has a higher mortality rate. HCoV-OC43, a strain of the common cold coronavirus, typically induces mild respiratory symptoms in healthy individuals. However, it can lead to severe complications in vulnerable populations, such as the elderly and those with underlying health conditions.

Paramyxoviridae is a family of negative-strand RNA viruses. These viruses are found in vertebrates and cause diseases such as measles, mumps, and respiratory tract infections.

The Paramyxoviridae viral family includes established human pathogens such as measles virus, mumps virus, and the human parainfluenza viruses (HPIVs); highly lethal zoonotic pathogens such as Nipah virus; and a number of recently identified agents, such as Sosuga virus. Paramyxoviruses are members of the order Mononegavirales and have nonsegmented, negative-sense RNA genomes encapsidated into a ribonucleoprotein (RNP) complex within an enveloped virion. All paramyxovirus genomes encode for a nucleocapsid (N) protein, a phospho-(P) protein, a matrix (M) protein, a fusion (F) glycoprotein, an attachment hemagglutinin (H)/hemagglutinin-neuraminidase (HN)/glyco-(G) protein, and an RNA-dependent RNA polymerase, or large (L) protein. In addition, some paramyxoviruses encode a small hydrophobic protein and RNA editing of the P protein gene leads to expression of additional nonstructural proteins that play key roles in the antiviral response.

The high human-to-human transmission rate of paramyxoviruses such as measles virus and the high case fatality rate associated with other family members such as Nipah virus are of concern. Effective live-attenuated vaccines and a subunit vaccine have been developed for a small subset of paramyxoviruses, and these have been used to limit geographical distribution and eradicate important human and veterinary diseases. However, no effective approved antiviral treatments or vaccines exist to mitigate the majority of paramyxoviral diseases and we are woefully unprepared should a respiratory paramyxovirus emerge and spread in humans with no preexisting immunity.

For example, measles is a highly contagious viral infection that can cause serious health complications, especially in children younger than 5 years old. Measles is caused by a single-stranded, enveloped RNA virus with 1 serotype, which is classified as a member of the genus Morbillivirus in the Paramyxoviridae family. Despite measles being declared eliminated from the United States in 2000, measles is still common in many parts of the world and is brought into the United States from other countries.

The search for effective antiviral therapeutics against coronaviruses and paramyxoviruses faces obstacles, including the rapid mutation rates of these RNA viruses and the need for broad-spectrum drugs that can combat various strains. The development of antiviral drugs like remdesivir and monoclonal antibodies has shown promise in mitigating the severity of illness, but their efficacy varies, and challenges in accessibility and affordability persist.

Ongoing research continues to unravel the complexities of coronaviruses and paramyxoviruses and their variants, aiming to enhance our understanding of viral biology, host-pathogen interactions, and immune responses. As the scientific community grapples with the evolving nature of these viruses, it becomes increasingly vital to develop innovative therapeutics to safeguard human health in the face of emerging infectious threats.

### SUMMARY OF THE INVENTION

The present invention is based on the discovery of a new therapeutic polypeptide modified from a C-terminus heptad repeat (HRC) region of a coronaviral spike fusion protein, which possesses unexpected therapeutic effects. The new therapeutic polypeptide comprises a HRC peptide and a terminal extension amino acid sequence, wherein the terminal extension amino acid sequence is designed to provide, inter alia, tailorable flexibility, helicity, or rigidity, thereby effectively facilitating the binding of HRC peptide to the target viral HRN region and inhibiting the viral entry into the host cell. Furthermore, the terminal extension amino acid sequence is also designed to comprise a reactive group that allows convenient and precise reaction of the polypeptide with other moiety to produce therapeutic peptide conjugates, for example through click chemistry, automated flow chemistry, and/or solid phase peptide synthesis.

The coronavirus includes SARS-CoV, SARS-CoV-2, MERS-CoV, HCoV-OC43, HCOV-229E, HCoV-L63, HCoV-HKU1, and any variants thereof. The HRC peptide is as defined herein, and preferably comprises an amino acid sequence having at least 50% identity to a fragment of a wild type HRC region of a coronaviral spike fusion protein.

Also provided is a new therapeutic peptide conjugate that comprises the polypeptide, a moiety preferably capable of integrating cell membranes, and an optional targeting peptide such as a receptor binding domain (RBD) binding peptide or an ACE2 targeting peptide. Preferably, the polypeptide, the moiety, and the optional targeting peptide are linked to each other via a multivalent moiety.

Also provided is a method of using the polypeptide or the peptide conjugate for the treatment of a condition or disease associated with a coronaviral infection in a subject in need.

The present invention also provides a method of using the polypeptide or the peptide conjugate for the treatment of a condition or disease associated with a paramyxovirus, thereby preventing or reducing subsequent infections. In embodiments, the paramyxovirus is selected from the measles virus, the mumps virus, the human parainfluenza virus (HPIV) or the Nipah virus. Preferably, the paramyxovirus is the measles virus. Preferably, the paramyxovirus is the mumps virus. Preferably, the paramyxovirus is the human parainfluenza virus (HPIV). Preferably, the paramyxovirus is the Nipah virus.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A through FIG. 1E show AlphaFold predicted structures of viral HR1 and HR2 six helical bundles for the HR1 and HR2 sequences from Tables 7 and 8, respectively, and an overlay of the bundles is shown.

FIG. 2 shows the MMGBSA binding energy (kcal/mole) for each conjugated ligand with HR1 proteins of measles, Nipah and HPIV3.

### DETAILED DESCRIPTION OF THE INVENTION

### Polypeptide of the Invention

Human-infecting coronaviruses encompass a range of strains, including human coronavirus OC43 (HCoV-OC43), human coronavirus HKU1 (HCoV-HKU1), human coronavirus 229E (HCoV-229E), human coronavirus NL63 (HCoV-NL63), severe acute respiratory syndrome coronavirus (SARS-CoV), Middle East respiratory syndrome-related coronavirus (MERS-CoV), and severe acute respiratory syndrome coronavirus 2 (SARS-CoV-2). The dynamic nature of coronaviruses is evident through their continual evolution, giving rise to new variants that swiftly outpace established therapies and pose ongoing risks to human health.

Coronaviruses target human cells via the spike protein binding domain (RBD) attaching to the human angiotensin converting enzyme 2 (hACE2) receptor on host cells. The coronavirus spike(S) glycoprotein is a class I viral fusion protein on the outer envelope of the virion that plays a critical role in viral infection by recognizing host cell receptors and mediating fusion of the viral and cellular membranes. Coronavirus entry into host cells is mediated by the transmembrane spike(S) glycoprotein that forms homotrimers protruding from the viral surface. S comprises two functional subunits responsible for binding to the host cell receptor (S1 subunit) and fusion of the viral and cellular membranes (S2 subunit).

S1 serves the function of receptor-binding and contains a signal peptide (SP) at the N terminus, an N-terminal domain (NTD), and receptor-binding domain (RBD). S2 functions in membrane fusion to facilitate cell entry, and it contains a fusion peptide (FP) domain, internal fusion peptide (IFP), two heptad-repeat domains (HR1 and HR2, or also called HRN and HRC), transmembrane domain, and a C-terminal domain.

After binding, the spike protein is activated by the host cell transmembrane protease/serine subfamily member 2 (TMPRSS2) and consequently the virus undergoes fusion with the endosomal membrane for entry into the cell. The membrane fusion domain of the spike protein in coronaviruses is highly conserved so targeting membrane fusion may result in durable long-lasting therapeutics. The mechanisms of the viral entry of SARS-CoV-2 as an example have been illustrated in Jackson et al, “Mechanisms of SARS-CoV-2 entry into cells.” Nat Rev Mol Cell Biol 23, 3-20 (2022). https://doi.org/10.1038/s41580-021-00418-x.

Paramyxoviruses are members of the order Mononegavirales and have nonsegmented, negative-sense RNA genomes encapsulated into a ribonucleoprotein (RNP) complex within an enveloped virion. All paramyxovirus genomes encode for a nucleocapsid (N) protein, a phospho-(P) protein, a matrix (M) protein, a fusion (F) glycoprotein, an attachment hemagglutinin (H)/hemagglutinin-neuraminidase (HN)/glyco-(G) protein, and an RNA-dependent RNA polymerase, or large (L) protein. In addition, some paramyxoviruses encode a small hydrophobic protein and RNA editing of the P protein gene leads to the expression of additional nonstructural proteins that play key roles in the antiviral response. The ectodomain of paramyxovirus F proteins contains two conserved heptad repeat regions, the first (the N-terminal heptad repeat [“HRN” also referred to as “HR1”]) adjacent to the fusion peptide and the second (the C-terminal heptad repeat [“HRC” also referred to as HR2]) immediately preceding the transmembrane domain.

As used herein HRN/HR1 and HRC/HR2 are used interchangeably to refer to the heptad repeat region of coronavirus or paramyxovirus depending on the context in which it is being used. For example, when discussing a coronavirus, e.g., SARS-CoV-2, the HRN/HR1 and HRC/HR2 is from the coronavirus. Likewise, when discussing a paramyxovirus, e.g., measles, the HRN/HR1 and HRC/HR2 is from the paramyxovirus.

The polypeptide of the invention is modified from a C-terminus heptad repeat (HRC) region of a coronaviral spike fusion protein and comprises 20 to 80 amino acid residues in length. In some embodiments, the polypeptide has about 21 amino acid residues in length. In some embodiments, the polypeptide has about 25 amino acid residues in length. In some embodiments, the polypeptide has about 30, 31, 32, 33, 34, 35, 36, 37, 38, 39, 40, 41, 42, 43, 44, 45, 46, 47, 48, 49, or 50 amino acid residues in length. In some embodiments, the polypeptide has about 36 amino acid residues in length. In some embodiments, the polypeptide has about 38 or 39 amino acid residues in length. The polypeptide comprises an HRC peptide and at least one terminal extension amino acid sequence. In some embodiments wherein the coronavirus is SARS-CoV-2, the term “HRC region” refers to the 1163-1213 residues of the SARS-CoV-2 S protein.

In some embodiments wherein the virus is a paramyxovirus, the term “HRC region” refers to the sequences in Table 4 below.

The term “HRC peptide”, “HRC sequence”, or “HRC peptide sequence” as used herein refers to a peptide sequence that is derived or modified from, or is designed to mirror certain bioactivity of, the HRC region of a coronaviral spike fusion protein (S2), which therefore includes the amino acid sequence of wild type HRC region of S2, a variant thereof, a fragment thereof, a mutated form thereof, an analog thereof, and a synthetic peptide that mimics the bioactivity of any one of the above. The HRC region of S2 is involved in the formation of the six-helix bundle structure that drives viral fusion. The HRC peptide as described herein can bind to the N-terminal heptad repeat (HRN) region of the fusion protein S2, blocking the formation of the six-helix bundle and preventing viral fusion and entry into host cells. The conserved nature of the fusion regions (HRC/HRN) makes them an ideal target to develop peptide inhibitors. Examples for HRC peptide analogs can be found in Yan et al., Journal of Structural Biology 155 (2006) 162-175, https://doi.org/10.1016/j.jsb.2006.03.024; K. Yang, et al. Structure-based design of a SARS-CoV-2 Omicron-specific inhibitor, Proc. Natl. Acad. Sci. U.S.A. 120 (13) e2300360120, https://doi.org/10.1073/pnas.2300360120 (2023); Xing L, et al. A Five-Helix-Based SARS-CoV-2 Fusion Inhibitor Targeting Heptad Repeat 2 Domain against SARS-CoV-2 and Its Variants of Concern. Viruses. 2022; 14 (3): 597. https://doi.org/10.3390/v14030597.

It has also been surprisingly discovered that the HRC peptide of the invention can bind to the N-terminal heptad repeat (HRN) region of the F glycoprotein of paramyxoviruses, thereby affecting paramyxovirus fusion and entry into host cells.

The term “terminal extension amino acid sequence”, or “terminal extension sequence”, or the acronym “TE”, as used herein refers to an amino acid sequence that is located at the N-terminus, or the C-terminus, or both terminuses of the polypeptide; and has 1 to 30 amino acid residues in length. One function of TE sequence is to modify the flexibility, helicity, or rigidity of the polypeptide to optimize the binding specificity and affinity, stability, and manufacturability of the polypeptide. Chen et al., Advanced Drug Delivery Reviews, Volume 65, Issue 10, 15 Oct. 2013, 1357-1369, https://doi.org/10.1016/j.addr.2012.09.039. Each terminal extension (TE) sequence is independently linked to the HRC peptide sequence directly or through optional additional domains. Therefore, the polypeptide of the invention can be preferably represented by:

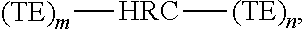

wherein m is 1, n is 0; m is 0, n is 1; or m and n are both 1; HRC represents HRC peptide sequence, TE represents terminal extension sequence, and each TE can be identical to or different from each other. Each domain, TE or HRC, can be independently designed to increase the binding affinity, vary flexibility, helicity, or rigidity, thereby maximizing the therapeutic effects of the polypeptide.

For example, the coronavirus is SARS-CoV-2; and the HRC sequence is derived from the HRC region of a SARS-CoV-2 variant, and for example has about 2%, about 4%, about 6%, about 8%, about 10%, about 20%, about 30%, about 40%, about 50%, about 60%, about 70%, about 75%, about 80%, about 85%, about 90%, about 92%, about 94%, about 96%, about 98%, about 99% identity to SEQ ID NO. 1:

(SEQ ID NO. 1)
DISGINASVVNIQKEIDRLNEVAKNLNESLIDLQEL.

As another example, the coronavirus is MERS-CoV; and the HRC sequence is derived from the HRC region of a MERS-CoV variant, and for example has about 2%, about 4%, about 6%, about 8%, about 10%, about 20%, about 30%, about 40%, about 50%, about 60%, about 70%, about 75%, about 80%, about 85%, about 90%, about 92%, about 94%, about 96%, about 98%, about 99% identity to SEQ ID NO. 2:

(SEQ ID NO. 2)
SLTQINTTLLDLTYEMLSLQQVVKALNESYIDLKEL.

As another example, the coronavirus is HCoV-OC43; and the HRC sequence is derived from the HRC region of a HCoV-OC43 variant, and for example has about 2%, about 4%, about 6%, about 8%, about 10%, about 20%, about 30%, about 40%, about 50%, about 60%, about 70%, about 75%, about 80%, about 85%, about 90%, about 92%, about 94%, about 96%, about 98%, about 99% identity to SEQ ID NO. 3:

(SEQ ID NO. 3)
SLDYINVTFLDLQDEMNRLQEAIKVLNQSYINLDEI.

For example, the paramyxoviridae is measles; and the HRC sequence is derived from the HRC region of measles, and for example has about 2%, about 4%, about 6%, about 8%, about 10%, about 20%, about 30%, about 40%, about 50%, about 60%, about 70%, about 75%, about 80%, about 85%, about 90%, about 92%, about 94%, about 96%, about 98%, about 99% identity to SEQ ID NO. 7:

(SEQ ID NO. 7)
ISLERLDVGTNLGNAIAKLEDAKELLESSDQILRSM.

As another example, the paramyxoviridae is Nipah; and the HRC sequence is derived from the HRC region of Nipah, and for example has about 2%, about 4%, about 6%, about 8%, about 10%, about 20%, about 30%, about 40%, about 50%, about 60%, about 70%, about 75%, about 80%, about 85%, about 90%, about 92%, about 94%, about 96%, about 98%, about 99% identity to SEQ ID NO. 8:

(SEQ ID NO. 8)
VFTDKVDISSQISSMNQSLQQSKDYIKEAQRLLDTV.

As another example, the paramyxoviridae is HPIV3; and the HRC sequence is derived from the HRC region of HPIV3, and for example has about 2%, about 4%, about 6%, about 8%, about 10%, about 20%, about 30%, about 40%, about 50%, about 60%, about 70%, about 75%, about 80%, about 85%, about 90%, about 92%, about 94%, about 96%, about 98%, about 99% identity to SEQ ID NO. 9:

(SEQ ID NO. 9)
VALDPIDISIELNKAKSDLEESKEWIRRSNQKLDSI.

As another example, the paramyxoviridae is HPIV3 variant; and the HRC sequence is derived from the HRC region of a HPIV3 variant (HPIV3_3001), and for example has about 2%, about 4%, about 6%, about 8%, about 10%, about 20%, about 30%, about 40%, about 50%, about 60%, about 70%, about 75%, about 80%, about 85%, about 90%, about 92%, about 94%, about 96%, about 98%, about 99% identity to SEQ ID NO. 10:

(SEQ ID NO. 10)
VALDPIDISIVLNKIKSDLEESKEWIRRSNKILDSI

As another example, the paramyxoviridae is a second HPIV3 variant; and the HRC sequence is derived from the HRC region of the second HPIV3 variant (HPIV3_3002), and for example has about 2%, about 4%, about 6%, about 8%, about 10%, about 20%, about 30%, about 40%, about 50%, about 60%, about 70%, about 75%, about 80%, about 85%, about 90%, about 92%, about 94%, about 96%, about 98%, about 99% identity to SEQ ID NO. 11:

(SEQ ID NO. 11)
VALDPIDISIVLNKIKSQLEESKWEIRRSNKILDSI.

As another example, the paramyxoviridae is a third HPIV3 variant; and the HRC sequence is derived from the HRC region of the third HPIV3 variant (HPIV3_3003), and for example has about 2%, about 4%, about 6%, about 8%, about 10%, about 20%, about 30%, about 40%, about 50%, about 60%, about 70%, about 75%, about 80%, about 85%, about 90%, about 92%, about 94%, about 96%, about 98%, about 99% identity to SEQ ID NO. 12:

(SEQ ID NO. 12)
VALDPIDFSIVLNKIKSQLEESKWEIRRSNKILDSI.

### Terminal Extension Sequence

In some embodiments, the terminal extension (TE) sequence comprises a cysteine (Cys or C) at the terminus. In some embodiments, the terminal extension (TE) sequence comprises a lysine (lys or K) at the terminus.

In some embodiments, the terminal extension (TE) sequence has 1 to 25 amino acid residues in length. In some embodiments, the TE sequence has 1 to 24 amino acid residues in length. In some embodiments, the TE sequence has 1 to 23 amino acid residues in length. In some embodiments, the TE sequence has 1 to 22 amino acid residues in length. In some embodiments, the TE sequence has 1 to 21 amino acid residues in length. In some embodiments, the TE sequence has 1 to 20 amino acid residues in length. In some embodiments, the TE sequence has 1, 2, 3, 4, 5, 6, 7, 8, 9, 10, 11, 12, 13, 14, 15, 16, 17, 18, 19, or 20 amino acid residues in length. In some embodiments, the TE sequence has 1, 2, 3, 4, 5, 6, 7, 8, 9, or 10 amino acid residues in length. Preferably, the TE sequence has about 6 amino acid residues in length.

In some embodiments, the TE sequence comprises one or more amino acids selected from threonine (Thr or T), lysine (lys or K), glutamate (Glu or E), serine (Ser or S), glycine (Gly or G), valine (Val or V), glutamine (Gln or Q), leucine (Leu or L), alanine (Ala or A), phenylalanine (Phe or F), arginine (Arg or R), and aspartate (Asp or D).

In some embodiments, the TE sequence comprises a Cys at the terminus, and in addition to the Cys at the terminus of the TE sequence, the TE sequence further comprises one or more amino acids selected from threonine (Thr or T), lysine (lys or K), glutamate (Glu or E), serine (Ser or S), glycine (Gly or G), valine (Val or V), glutamine (Gln or Q), leucine (Leu or L), alanine (Ala or A), phenylalanine (Phe or F), arginine (Arg or R), and aspartate (Asp or D).

In some embodiments, the TE sequence comprises a Lys at the terminus, and in addition to the Lys at the terminus of the TE sequence, the TE sequence further comprises one or more amino acids selected from threonine (Thr or T), lysine (lys or K), glutamate (Glu or E), serine (Ser or S), glycine (Gly or G), valine (Val or V), glutamine (Gln or Q), leucine (Leu or L), alanine (Ala or A), phenylalanine (Phe or F), arginine (Arg or R), and aspartate (Asp or D).

In some embodiments, the TE sequence comprises one or more amino acids selected from G, S, E, L, A, V, and K, and C. In some embodiments, the TE sequence comprises C at the terminus, and further comprises one or more amino acids selected from G, S, E, L, A, V, K, and C. In some embodiments, the TE sequence comprises C at the terminus, and further comprises one or more amino acids selected from G, S, E, L, A, V, and K. In some embodiments, the TE sequence comprises K at the terminus, and further comprises one or more amino acids selected from G, S, E, L, A, V, K, and C.

In some embodiments, the TE sequence comprises two or more amino acids selected from G, S, and C. In some embodiments, the TE sequence comprises C at the terminus and further comprises G, S, or both.

In some embodiments, the TE sequence is selected from:

C,
(GS)ₙ-GC,
(GSG)ₙ-C,
(G)ₙ-C,
(S)ₘ-(GGS)ₙ-C,
(S)ₘ-(GGGS)ₙ-C,
(S)ₘ-(GGGGS)ₙ-C,
(A)ₘ-(EAAAK)ₙ-(A)ₘ-C,
(EAAAK)ₙ-(GS)ₘ-GC,
(SA)ₙ-C,
(EL)ₙ-(GS)ₙ-GC,
and
(GSA)ₙ-(AG)ₘ-(SG)ₘ-(EF)ₘ-C,

wherein each n is independently selected from 1, 2, 3, 4, 5, and 6, and each m is independently selected from 0, 1, 2, 3, 4, 5, and 6. In some cases, n is 1, 2, 3, 4 or 5. In some cases, n is 1, 2, 3, or 4. In some cases, n is 1, 2, or 3. In some cases, m is 0, 1, 2, 3, 4 or 5. In some cases, m is 0, 1, 2, 3, or 4. In some cases, m is 0, 1, 2, or 3.

In some embodiments, the TE sequence comprises an amino acid sequence having at least 70% identity to an amino acid sequence selected from THESE AMINO ACID SEQUENCES. In some embodiments, the TE sequence is an amino acid sequence having at least 70% identity to an amino acid sequence selected from:

GSGSGC,
ELGSGSGC,
GGGGSC,
GGGGGGGGC,
GGGGGGC,
EAAAKC,
EAAAKEAAAKC,
AEAAAKAC,
GSGC,
GC,
GSAGSAAGSGEFC,
KESGSVSSEQLAQFRSLDC,
and
EGKSSGSGSESKSTC.

In some embodiments, the terminal extension amino acid sequence has at least 50%, at least 60%, at least 70%, at least 80%, at least 90%, at least 95%, at least 97%, or at least 99% identity to GSGSGC.

In some embodiments, the TE amino acid sequence is located at the C-terminus of the polypeptide.

In additional embodiments, the TE sequence is located at the N-terminus of the polypeptide and has at least 70% identity to an amino acid sequence selected from:

CGSGSG,
CGSGSGLE,
CGGGGGGGG,
CGGGGGG,
CKAAAE,
CKAAAEKAAAE,
CAKAAAEA,
CGSG,
CG,
CFEGSGAASGASG,
CDLSRFQALQESSVSGSEK,
and
CTSKSESGSGSSKGE.

### HRC Sequence

In the polypeptide of the invention, which is preferably represented by (TE)ₘ-HRC-(TE)ₙ, the HRC sequence has 12 to 60 amino acid residues in length. In some embodiments, the HRC sequence has about 18 to about 50 amino acid residues in length. In some embodiments, the HRC sequence has about 20 to 48 amino acid residues in length. In some embodiments, the HRC sequence has about 21, 22, 23, 24, 25, 26, 27, 28, 29, 30, 31, 32, 33, 34, 35, 36, 37, 38, 39, 40, 41, 42, 43, 44, 45, 46, 47, or 48 amino acid residues in length. In some embodiments, the HRC sequence has about 21 amino acid residues in length. In some embodiments, the HRC sequence has about 24 amino acid residues in length. In some embodiments, the HRC sequence has about 25 amino acid residues in length. In some embodiments, the HRC sequence has about 28 amino acid residues in length. In some embodiments, the HRC sequence has about 39 amino acid residues in length. In some embodiments, the HRC sequence has about 40 amino acid residues in length. In some embodiments, the HRC sequence has about 47 amino acid residues in length. In some embodiments, the HRC sequence has about 48 amino acid residues in length. Preferably, the HRC sequence has about 36 amino acid residues in length. Computational tools can be applied to evaluate the effects of mutations and modified chain lengths on the potency of the polypeptide. Dehouck et al., “BeAtMuSiC: Prediction of changes in protein-protein binding affinity on mutations”, Nucleic Acids Res. 2013 July; 41 (Web Server issue): W333-9. doi: 10.1093/nar/gkt450.

The HRC sequence comprises an amino acid sequence that comprises a modified fusion core sequence. The modified fusion core sequence is a fragment derived from the S2 subunit of the coronavirus. It functions as the shortest fusion inhibitor with the minimal necessary amino acids, for binding to the N-terminal heptad repeat (HRN) region of the fusion protein S2, blocking the formation of the six-helix bundle, and preventing viral fusion and entry into host cells. The HRC sequence further optionally comprises extension amino acid sequence in addition to the modified fusion core sequence. The HRC sequence can be represented by:

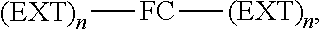

Each n is independently 0, 1, or 2. Each EXT represents an extension amino acid sequence, and each EXT can be identical to or different from each other. FC represents the modified fusion core sequence, namely the shortest fusion inhibitor.

In some embodiments, each n is 0, and the HRC sequence is an FC as described herein.

In some embodiments, one of the two n is 0 and the other is 1, the HRC sequence can be represented as EXT-FC or FC-EXT.

In some embodiments, both n is 1, the HRC sequence can be represented as EXT-FC-EXT.

In some embodiments, each EXT sequence and FC sequence are derived from the HRC region of the SARS-CoV-2 S protein.

As used herein, the term “derived from” has a more generic meaning in the sense that it allows for modifications which may render said composition of matter different from its natural occurrence while maintaining, at least to a substantial degree, function. Maintenance of a substantial degree of function preferably refers to maintenance of at least 10%, at least 20%, at least 30%, at least 40%, at least 50%, at least 60%, at least 70%, at least 80%, at least 90% function, or full maintenance (100%) of function or activity.

In some embodiments, each EXT sequence and FC sequence independently comprise one or more mutations relative to an amino acid sequence in the HRC region of a coronavirus.

As used herein, the term “amino acid” is used to refer to any organic molecule that contains at least one amino group and at least one carboxyl group. Typically, at least one amino group is at the a position relative to a carboxyl group. The amino acids may be naturally occurring. Naturally occurring amino acids include, for example, the twenty most common levorotatory (L) amino acids normally found in mammalian proteins, i.e., alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val). Other naturally occurring amino acids include, for example, amino acids that are synthesized in metabolic processes not associated with protein synthesis. For example, the amino acids ornithine (Orn) and citrulline (Cit) are synthesized in mammalian metabolism during the production of urea. Another example of a naturally occurring amino acid includes hydroxyproline (Hyp).

Mutations in the polypeptide of this invention may include substitutions, deletions, including internal deletions, additions, including additions yielding fusion proteins, or conservative substitutions of amino acid residues within and/or adjacent to the amino acid sequence, but that result in a “silent” change, in that the change produces a functionally-equivalent polypeptide sequence. Conservative amino acid substitutions may be made on the basis of similarity in polarity, charge, solubility, hydrophobicity, hydrophilicity, and/or the amphipathic nature of the residues involved. For example, non-polar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. In addition, proline can influence chain orientation. Non-conservative substitutions will entail exchanging a member of one of these classes for a member of another class. Furthermore, if desired, non-classical amino acids or chemical amino acid analogs (i.e., non-natural amino acides) can be introduced as a substitution or addition into the peptide sequence. Non-classical amino acids include, but are not limited to, the D-isomers of the common amino acids, gamma amino acid, a, a-disubstituted amino acids, N-alkylamino acids, C-a-methyl amino acids, P-amino acids, and P-methyl amino acids, α-amino isobutyric acid (Aib, also called 2-aminoisobutyric acid or 2-amino isobutyric acid), 4-aminobutyric acid, Abu, 2-amino butyric acid, γ-Abu, ε-Ahx, 6-amino hexanoic acid, 3-amino propionic acid, diaminobutyric acid (Dab), diaminopropionic acid (Dap), ornithine (Orn), homoarginine (hArg), norleucine, homoisoleucine, norvaline, hydroxyproline, sarcosine, citrulline (Cit), cysteic acid, t-butylglycine, t-butylalanine, phenylglycine, cyclohexylalanine, β-alanine, fluoro-amino acids, designer amino acids such as β-methyl amino acids, Ca-methyl amino acids, Nα-methyl amino acids, and amino acid analogs in general.

In some embodiments, non-classical amino acids include, but are not limited to, 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), Naphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), Tetrahydro-isoquinoline-3-carboxylic Acid (Tic), methylated. Preferably, non-classical amino acids include Aib, Orn, Dab, and Dap.

In some embodiments, one or more of the amino acid residues of the peptide have been chemically modified (e.g., by alkylation, acylation, ester formation, or amide formation). Nonlimiting examples of such amino acid residues include for example 2-aminoadipic acid, 3-aminoadipic acid, 8-alanine, 8-aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, alloisoleucine, N-methylglycine, N-methylisoleucine, 6-N-methyllysine (Mlz), (6-N,6-N)dimethyllysine (Mly), N(α)-methyl-L-lysine (nme-K), N-methylvaline, norvaline, nor-leucine, ornithine, and statine halogenated amino acids.

Preferable examples of non-classical amino acids used for this invention include Aib, Orn, Dap, Dab, Mlz, Mly, nme-K, hArg, and Cit. In some cases, the peptide of the invention comprises one or more amino acid residues selected from Aib, Orn, Dap, Dab, Mlz, Mly, nme-K, hArg, and Cit.

In some cases, the peptide of the invention comprises one or more hArg to preserve resistance to trypsin and trypsin-like proteases while extending the side chain to enhance fit into the binding pocket of HRN (HR1). In some cases, the peptide comprises a substitution of wild-type arginine (R) or ornithine (Orn) with homoarginine (hArg) to maintain trypsin and trypsin-like protease resistance while increasing the side chain length to enhance binding interactions with the HR1 (HRN) pocket.

In some cases, the peptide of the invention comprises a substitution of alanine (A) with α-aminoisobutyric acid (Aib) to enhance resistance to DPPIV-mediated degradation, thereby improving peptide stability.

In some cases, lysine (K) residues in the peptides are substituted with modified lysines such as N-methyl lysine (nme-K), ornithine (Orn), 2,4-diaminobutyric acid (Dab), 2,3-diaminopropionic acid (Dap), methyllysine (Mly), or methylornithine (Mlz) to confer resistance to trypsin and trypsin-like proteases.

In some cases, arginine (R) residues are substituted with citrulline (Cit), ornithine (Orn), or histidine (H) to preserve peptide integrity by increasing resistance to proteolytic cleavage by trypsin and related enzymes.

In some cases, peptides are engineered with N-terminal extensions to improve potency. In some cases, peptides are engineered with C-terminal truncations to enhance manufacturability and simplify peptide conjugation. In some cases, peptides are engineered with both N-terminal extensions to improve potency and C-terminal truncations to enhance manufacturability and simplify peptide conjugation.

In some cases, the peptide is modified by substituting valine (Val) with isoleucine (Ile) at specific positions to improve manufacturability without sacrificing functional activity.

In some cases, specific point mutations such as R1185H or D1184E are introduced into the peptide sequences to improve protease resistance and/or binding properties.

In some embodiments, mutations introduced to the polypeptide are designed to enhance the binding affinity of the polypeptide to the coronavirus spike protein. In preferred embodiments, mutations introduced to the polypeptide are designed to enhance the binding affinity of the polypeptide to the SARS-CoV-2 spike protein. The mutation may be introduced to improve the binding energy. Kandeel et al., Biomol Ther 29 (3), 282-289 (2021). doi.org/10.4062/biomolther.2020.201.

In some embodiments, the polypeptide of the invention also can be engineered to enhance the manufacturability, preferably through introducing specific mutations. In some embodiments, the polypeptide of the invention also can be engineered to optimize the formulation process for administration (such as intranasal administration), preferably through introducing specific mutations.

In some embodiments, the polypeptide of the invention is modified to improve its resistance to one or more proteases found in the human body. Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, cathepsins (e.g. cathepsin B, cathepsin C, cathepsin D, cathepsin E, cathepsin K, cathepsin L, cathepsin S, etc.), kallikreins, hK1, hK10, hK15, KLK7, granzyme B, plasmin, collagenase, Type IV collagenase, stromelysin, factor XA, chymotrypsin-like protease, trypsin, trypsin-like protease, elastase-like protease, subtilisin-like protease, actinidain, bromelain, calpain, caspases (e.g. caspase-3), Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, meprin, urokinase plasminogen activator (uPA), enterokinase, prostate-specific antigen (PSA, hK3), interleukin-1β converting enzyme, thrombin, FAP (FAP-α), dipeptidyl peptidase, and dipeptidyl peptidase IV (DPPIV/CD26). In some embodiments, mutations are introduced to the polypeptide to improve the resistance to one or more proteases. For example, the polypeptide can include the amino acid residue Aib to improve its resistance to DPPIV. For example, the polypeptide of the invention can include a point mutation from alanine (A) to Aib to improve its resistance to DPPIV. As another example, the polypeptide of the invention can include the amino acid residue Dab to improve its resistance to trypsin or a trypsin-like protease. Preferably, the polypeptide of the invention can include a point mutation from lysine (K) to Dab to improve its resistance to trypsin or a trypsin-like protease. The polypeptide of the invention can include the amino acid residue Dap to improve its resistance to trypsin or a trypsin-like protease. Preferably, the polypeptide of the invention can include a point mutation from lysine (K) to Dap to improve its resistance to trypsin or a trypsin-like protease. The polypeptide of the invention can include the amino acid residue Orn to improve its resistance to trypsin or a trypsin-like protease. Preferably, the polypeptide of the invention can include a point mutation from lysine (K) to Orn to improve its resistance to trypsin or a trypsin-like protease. As an additional example, the polypeptide of the invention can include a point mutation from arginine (R) to histidine (H) or a non-classical amino acid as described herein to improve its resistance to trypsin or a trypsin-like protease. In some embodiments, the polypeptide of the invention includes mutations from A to Aib, K to Dab, K to Orn, K to Dap, R to H, or any combination thereof.

In some cases, the peptides of the invention comprise one or more mutations or modifications that are suggested by various computational and rational design tools. For example, in some instances, the peptides are modified based on computational predictions, which identifies amino acid substitutions that optimize side chain interactions. Such modifications may enhance binding affinity, improve protease resistance, or increase overall structural stability.

In some cases, the peptides are engineered using machine learning-based platforms, such as copilot-assisted sequence design tools. These AI-driven systems can suggest beneficial mutations by predicting sequence variations that improve folding, binding, stability, or manufacturability based on learned patterns from large protein structure databases. The resulting peptide variants may exhibit enhanced biological properties and improved manufacturability.

In some cases, the peptides of the invention are designed as pan-inhibitors, meaning they are capable of inhibiting multiple related targets within a viral family or class of proteins. Such peptides may block a broad range of viruses or biological pathways, thereby providing robust and versatile therapeutic potential.

In some cases, the peptides are engineered as selective inhibitors, targeting a specific virus or protein with high specificity. These selective inhibitors are designed to minimize off-target effects while maximizing potency against the intended pathogen.

In some cases, the peptides function as pan-paramyxovirus inhibitors, capable of inhibiting a wide array of paramyxoviruses, including measles virus, mumps virus, parainfluenza (HPIV3) viruses, and Nipah virus. These peptides may disrupt conserved regions of viral fusion machinery, providing broad-spectrum antiviral activity across the paramyxovirus family.

In some cases, the peptides are developed as measles-selective inhibitors, designed specifically to block fusion or entry of the measles virus by targeting unique features of the measles fusion protein.

In some cases, the peptides are tailored as mumps virus-selective inhibitors, focusing on the inhibition of mumps virus fusion or replication processes without substantially affecting other related viruses.

In some cases, the peptides are designed as parainfluenza virus-selective inhibitors (HPIV3-selective inhibitors), specifically interfering with the entry or fusion machinery of parainfluenza viruses, thereby preventing infection while minimizing impacts on other paramyxoviruses.

In some cases, the peptides are engineered as Nipah virus-selective inhibitors, targeting critical regions of the Nipah virus fusion protein to block its ability to mediate membrane fusion and viral entry.

In some cases, the peptides are designed as pan-coronavirus inhibitors, capable of inhibiting multiple coronaviruses, such as SARS-CoV, MERS-CoV, and SARS-CoV-2, by targeting conserved regions of the viral spike protein or essential viral enzymes. Such peptides may provide broad-spectrum protection against known and emerging coronaviruses.

In some cases, the peptides are optimized as SARS-CoV-2-selective inhibitors, designed to interfere with the viral spike protein's ability to bind to host receptors or mediate membrane fusion, thereby preventing infection by SARS-CoV-2 specifically.

In some cases, the peptides are developed as MERS-CoV-selective inhibitors, targeting unique structural features of the MERS coronavirus spike protein to inhibit viral entry and prevent infection.

When the coronavirus is a SARS-CoV-2 variant, the FC is an amino acid sequence that has up to 15 amino acid mutations relative to IQKEIDRLNEVAKNLNESLID (SEQ ID NO. 13), or an amino acid sequence that has up to 15 amino acid mutations relative to the inversion of DILSENLNKAVENLRDIEKQI (SEQ ID NO. 14). In some cases, the FC is an amino acid sequence that has up to 14 amino acid mutations relative to IQKEIDRLNEVAKNLNESLID (SEQ ID NO. 13), or an amino acid sequence that has up to 14 amino acid mutations relative to the inversion of DILSENLNKAVENLRDIEKQI (SEQ ID NO. 14). In some cases, the FC is an amino acid sequence that has up to 13 amino acid mutations relative to IQKEIDRLNEVAKNLNESLID (SEQ ID NO. 13), or an amino acid sequence that has up to 13 amino acid mutations relative to the inversion of DILSENLNKAVENLRDIEKQI (SEQ ID NO. 14). In some cases, the FC is an amino acid sequence that has up to 12 amino acid mutations relative to IQKEIDRLNEVAKNLNESLID (SEQ ID NO. 13), or an amino acid sequence that has up to 12 amino acid mutations relative to the inversion of DILSENLNKAVENLRDIEKQI (SEQ ID NO. 14). In some cases, the FC is an amino acid sequence that has up to 11 amino acid mutations relative to IQKEIDRLNEVAKNLNESLID (SEQ ID NO. 13), or an amino acid sequence that has up to 11 amino acid mutations relative to the inversion of DILSENLNKAVENLRDIEKQI (SEQ ID NO. 14). In some cases, the FC is an amino acid sequence that has up to 11 amino acid mutations relative to IQKEIDRLNEVAKNLNESLID (SEQ ID NO. 13).

In some embodiments, FC is an amino acid sequence that has 1, 2, 3, 4, 5, 6, 7, 8, 9, 10, 11, 12, 13, 14, 15, 16, 17, 18, 19, or 20 amino acid mutations relative to IQKEIDRLNEVAKNLNESLID (SEQ ID NO. 13). In some embodiments, FC is an amino acid sequence that has 1, 2, 3, 4, 5, 6, 7, 8, 9, 10, 11, 12, 13, 14, 15, 16, 17, 18, or 19 amino acid mutations relative to IQKEIDRLNEVAKNLNESLID (SEQ ID NO. 13). In some embodiments, FC is an amino acid sequence that has 1, 2, 3, 4, 5, 6, 7, 8, 9, 10, 11, 12, 13, 14, 15, 16, 17, or 18 amino acid mutations relative to IQKEIDRLNEVAKNLNESLID (SEQ ID NO. 13). In some embodiments, FC is an amino acid sequence that has 1, 2, 3, 4, 5, 6, 7, 8, 9, 10, 11, 12, 13, 14, 15, 16, or 17 amino acid mutations relative to IQKEIDRLNEVAKNLNESLID (SEQ ID NO. 13). In some embodiments, FC is an amino acid sequence that has 1, 2, 3, 4, 5, 6, 7, 8, 9, 10, 11, 12, 13, 14, 15, or 16 amino acid mutations relative to IQKEIDRLNEVAKNLNESLID (SEQ ID NO. 13). In some embodiments, FC is an amino acid sequence that has 1, 2, 3, 4, 5, 6, 7, 8, 9, 10, 11, 12, 13, 14, or 15 amino acid mutations relative to IQKEIDRLNEVAKNLNESLID (SEQ ID NO. 13). In some embodiments, FC is an amino acid sequence that has 1, 2, 3, 4, 5, 6, 7, 8, 9, 10, 11, 12, 13, or 14 amino acid mutations relative to IQKEIDRLNEVAKNLNESLID (SEQ ID NO. 13). In some embodiments, FC is an amino acid sequence that has 1, 2, 3, 4, 5, 6, 7, 8, 9, 10, 11, 12, or 13 amino acid mutations relative to IQKEIDRLNEVAKNLNESLID (SEQ ID NO. 13). In some embodiments, FC is an amino acid sequence that has 1, 2, 3, 4, 5, 6, 7, 8, 9, 10, 11, or 12 amino acid mutations relative to IQKEIDRLNEVAKNLNESLID (SEQ ID NO. 13). In some embodiments, FC is an amino acid sequence that has 1, 2, 3, 4, 5, 6, 7, 8, 9, 10, or 11 amino acid mutations relative to IQKEIDRLNEVAKNLNESLID (SEQ ID NO. 13). In some embodiments, FC is an amino acid sequence that has 1, 2, 3, 4, 5, 6, 7, 8, 9, or 10 amino acid mutations relative to IQKEIDRLNEVAKNLNESLID (SEQ ID NO. 13). In some embodiments, the amino acid mutations are independently located at the positions selected from Xaa1-Xaa21, wherein the positions of Xaa1-Xaa21 are represented by: Xaa1-Xaa2-Xaa3-Xaa4-Xaa5-Xaa6-Xaa7-Xaa8-Xaa9-Xaa10-Xaa11-Xaa12-Xaa13-Xaa14-Xaa15-Xaa16-Xaa17-Xaa18-Xaa19-Xaa20-Xaa21, corresponding to IQKEIDRLNEVAKNLNESLID (SEQ ID NO. 13). For example, Xaa1 represents the position of the first amino acid residue at N-terminus, i.e., the position of I and Xaa2 represents the position of the second amino acid residue at N-terminus, i.e., the position of Q.

In some cases, Xaa1 is mutated, i.e., Xaa1 is an amino acid rather than I. In some cases, Xaa1 is mutated and Xaa1 is A or L, preferably A.

In some cases, Xaa2 is mutated, i.e., Xaa2 is an amino acid rather than Q. In some cases, Xaa2 is mutated and Xaa2 is T, K or E.

In some cases, Xaa3 is mutated, i.e., Xaa3 is an amino acid rather than K. In some cases, Xaa3 is mutated, and Xaa3 is Y, D, L, Orn, Dab, Dap, Mlz, Mly, or nme-K.

In some cases, Xaa4 is mutated, i.e., Xaa4 is an amino acid rather than E. In some cases, Xaa4 is mutated, and Xaa4 is P, A, K, T, or Y; preferably P or K.

In some cases, Xaa5 is mutated, i.e., Xaa5 is an amino acid rather than I. In some cases, Xaa5 is mutated, and Xaa5 is A, M, K, G, or E; preferably A or M.

In some cases, Xaa6 is mutated, i.e., Xaa6 is an amino acid rather than D. In some cases, Xaa6 is mutated, and Xaa6 is selected from A, E, K, L, M, or N; preferably A, E, K, L, or N.

In some cases, Xaa7 is mutated, i.e., Xaa7 is an amino acid rather than R. In some cases, Xaa7 is mutated, Xaa7 is selected from P, E, H, K, S, A, L, Orn, Cit; preferably, P, E, S, H, K, Orn, or Cit.

In some cases, Xaa8 is mutated, i.e., Xaa8 is an amino acid rather than L. In some cases, Xaa8 is mutated, and Xaa8 is A or I.

In some cases, Xaa9 is mutated, i.e., Xaa9 is an amino acid rather than N. In some cases, Xaa9 is mutated, and Xaa9 is selected from E, D, G or Q; preferably E, D, or G.

In some cases, Xaa10 is mutated, i.e., Xaa10 is an amino acid rather than E. In some cases, Xaa10 is mutated, and Xaa10 is K or Q; preferably K.

In some cases, Xaa11 is mutated, i.e., Xaa11 is an amino acid rather than V. In some cases, Xaa11 is mutated, and Xaa11 is selected from A, K, L, or I.

In some cases, Xaa12 is mutated, i.e., Xaa12 is an amino acid rather than A. In some cases, Xaa12 is mutated, and Xaa12 is I or V; preferably I.

In some cases, Xaa13 is mutated, i.e., Xaa13 is an amino acid rather than K. In some cases, Xaa13 is mutated, and Xaa13 is D, E, Orn, Dab, Dap, Mlz, Mly, or nme-K.

In some cases, Xaa14 is mutated, i.e., Xaa14 is an amino acid rather than N. In some cases, Xaa14 is mutated, and Xaa14 is selected from P, A, E, V, S, and K.

In some cases, Xaa15 is mutated, i.e., Xaa15 is an amino acid rather than L. In some cases, Xaa15 is mutated, and Xaa15 is A.

In some cases, Xaa16 is mutated, i.e., Xaa16 is an amino acid rather than N. In some cases, Xaa16 is mutated, and Xaa16 is E, G, P, Q.

In some cases, Xaa17 is mutated, i.e., Xaa17 is an amino acid rather than E. In some cases, Xaa17 is mutated, and Xaa17 is K or Q.

In some cases, Xaa18 is mutated, i.e., Xaa18 is an amino acid rather than S. In some cases, Xaa18 is mutated, and Xaa18 is P or K.

In some cases, Xaa19 is mutated, i.e., Xaa19 is an amino acid rather than L. In some cases, Xaa19 is mutated, and Xaa19 is Y or I.

In some cases, Xaa20 is mutated, i.e., Xaa20 is an amino acid rather than I. In some cases, Xaa20 is mutated, and Xaa20 is A or E.

In some cases, Xaa21 is mutated, i.e., Xaa21 is an amino acid rather than D. In some cases, Xaa21 is mutated, and Xaa21 is A, E, K, N, or G; preferably A, E, K, or G.

In some embodiments, FC is an amino acid sequence that has 1, 2, 3, 4, 5, 6, or 7 amino acid mutations relative to IQKEIDRLNEVAKNLNESLID (SEQ ID NO. 13), and the amino acid mutations are independently located at the positions selected from Xaa1, Xaa4, Xaa5, Xaa7, Xaa14, Xaa15, and Xaa18 as represented by:

Xaa1-QK-Xaa4-Xaa5-D-Xaa7-LNEVAK-
Xaa14-Xaa15-NESLID.

When any of Xaa1, Xaa4, Xaa5, Xaa7, Xaa14, Xaa15, and Xaa18 are independently mutated, the mutation at each position is as defined above.

In some embodiments, FC is an amino acid sequence that has 1, 2, 3, 4, 5, 6, 7 or 8 amino acid mutations relative to IQKEIDRLNEVAKNLNESLID (SEQ ID NO. 13), and the amino acid mutations are independently located at the positions selected from Xaa3, Xaa6, Xaa7, Xaa12, Xaa13, Xaa16, and Xaa21 as represented by:

IQ-Xaa3-EI-Xaa6-Xaa7-LNEV-Xaa12-
Xaa13-NL-Xaa16-ESLI-Xaa21.

When any of Xaa3, Xaa6, Xaa7, Xaa12, Xaa13, Xaa16, and Xaa21 is independently mutated, the mutation at each position is as defined above.

In some embodiments, FC is an amino acid sequence that has at least one amino acid mutations relative to IQKEIDRLNEVAKNLNESLID (SEQ ID NO. 13). In some embodiments, FC is an amino acid sequence that has at least one amino acid mutations relative to IQKEIDRLNEVAKNLNESLID (SEQ ID NO. 13), and the amino acid mutation is A to I at Xaa12, N to G at Xaa16, or D to E at Xaa21; preferably the amino acid mutation is A to I at Xaa12.

In some embodiments, FC is an amino acid sequence that has at least two amino acid mutations relative to IQKEIDRLNEVAKNLNESLID (SEQ ID NO. 13). In some embodiments, FC is an amino acid sequence that has at least two amino acid mutations relative to IQKEIDRLNEVAKNLNESLID (SEQ ID NO. 13), and the amino acid mutations are N to G at Xaa16 and D to E, at Xaa21.

In some embodiments, FC is an amino acid sequence that has at least three amino acid mutations relative to IQKEIDRLNEVAKNLNESLID (SEQ ID NO. 13). In some embodiments, FC is an amino acid sequence that has at least three amino acid mutations relative to IQKEIDRLNEVAKNLNESLID (SEQ ID NO. 13), and the amino acid mutations are A to I at Xaa12, N to G at Xaa16, and D to E, at Xaa21.

In some embodiments, FC has at least 70% identity to Xaa1-Xaa2-Xaa3-Xaa4-Xaa5-Xaa6-Xaa7-Xaa8-Xaa9-Xaa10-Xaa11-Xaa12-Xaa13-Xaa14-Xaa15-Xaa16-Xaa17-Xaa18-Xaa19-Xaa20-Xaa21. In some embodiments, FC is an amino acid sequence represented by Xaa1-Xaa2-Xaa3-Xaa4-Xaa5-Xaa6-Xaa7-Xaa8-Xaa9-Xaa10-Xaa11-Xaa12-Xaa13-Xaa14-Xaa15-Xaa16-Xaa17-Xaa18-Xaa19-Xaa20-Xaa21.
Xaa1 is I, A or L, preferably I.
Xaa2 is Q, T, K or E.
Xaa3 is K, Y, D, L, Orn, Dab, Dap, Mlz, Mly, or nme-K.
Xaa4 is E, P, A, K, T, or Y; preferably E, P or K.
Xaa5 is I, A, M, K, G, or E; preferably I, A or M.
Xaa6 is selected from D, A, E, K, L, M, and N; preferably D, A, E, K, L, and N.
Xaa7 is selected from R, P, E, H, K, S, A, L, Orn, and Cit; preferably, R, P, E, S, H, K, Orn, and Cit.
Xaa8 is L, A, or I.
Xaa9 is selected from N, E, D, G or Q; preferably N, E, D, or G.
Xaa10 is E, K, or Q; preferably E or K.
Xaa11 is selected from V, A, K, L, and I.
Xaa12 is A, I or V; preferably A or I.
Xaa13 is selected from K, D, E, Orn, Dab, Dap, Mlz, Mly, or nme-K.
Xaa14 is selected from N, P, A, E, V, S, and K; preferably Xaa14 is N.
Xaa15 is L or A; preferably L.
Xaa16 is N, E, G, P, or Q; preferably N or G.
Xaa17 is E, K, or Q; preferably E.
Xaa18 is S, P, or K; preferably S.
Xaa19 is L, I, or Y; preferably L.
Xaa20 is selected from I, A, and E; preferably, A.
Xaa21 is selected from D, A, E, K, N, and G; preferably D, A, E, K, and G; more preferably, D and E.

In some embodiments, FC is an amino acid sequence having at least 30%, at least 40%, at least 50%, at least 60%, at least 70%, at least 80%, at least 90%, at least 95%, at least 97%, or at least 99% identity to any one of These amino acid sequences in Table 1.

TABLE 1
Exemplary Fusion Core (FC) Sequences

| FC Sequence | SEQ ID NO. |
|---|---|
| IQKEIDRLNEVAKNLNESLID | 15 |
| IQKPIDRLNEVAKNLNESLID | 16 |
| IQKPIDPLNEVAKPLNEPLID | 17 |
| AQKEADRANEAAKNANESLID | 18 |
| IQKEIDRLNEVIKNLNESLID | 19 |
| IQKEIARLNEVAKALNESLID | 20 |
| IQKEIARLNEVAKALNESLAA | 21 |
| IQKEIARLNEVIKALNESLID | 22 |
| IQKEIERLNKVAKNLNESLID | 23 |
| IQKKIEELNKKAEELNKKLID | 24 |
| LQDEMNRLQEAIKVLNQSYIN | 25 |
| LTYEMLSLQQVVKALNESYID | 26 |
| IKKEIEHLNEIAKSLNESLID | 27 |
| IKKEIEHLNEIAKNLNESLID | 28 |
| IEYEIKKLEEVAKKLEESLID | 29 |
| IEYEIKKLEEVAKKLEESYID | 30 |
| DILSENLNKAVENLRDIEKQI | 31 |
| EAAAKEAAAKEAAAKEAAAKE | 32 |
| IQKEIDRLNEVAKNLNESLIE | 33 |
| IQKEIDRLNEVAKNLNESLIK | 34 |
| IQKEIDRLNEVAKNLNESLIG | 35 |
| IQKEIDRLNEVAKNLGESLIG | 36 |
| IQKEIDRLNEVAKNLPESLIG | 37 |
| IQKEIDRLDEVAKNLNESLID | 38 |
| IQKEIDRLGEVAKNLNESLID | 39 |
| IQKEIDRLNEVAKNLGESLIE | 40 |
| IQKEIDRLDEVAKNLPESLIE | 41 |
| IQDEIEHLNELIDNLNESLID | 42 |
| IQLEIEHLNELIENLGESLIE | 43 |
| IQLEIEHLNEVIENLGESLIE | 44 |
| IQLEIEHLNELIENLGESLIE | 45 |
| IQLEIDHLNEVIENLGESLIE | 46 |
| IQKEIDRLNEVIKNLGESLIE | 47 |

TABLE 1a
Exemplary Fusion Core (FC) Sequences

| FC Sequence | SEQ ID NO. |
|---|---|
| IQOrnEIDOrnLNEVAOrnnNLNESLID | 48 |
| IQDabEIDOrnLNEVADabNLNESLID | 49 |
| IQDapEIDOrnLNEVADapNLNESLID | 50 |
| IQMlzEIDOrnLNEVAMlzNLNESLID | 51 |
| IQMlyEIDOrnLNEVAMlyNLNESLID | 52 |
| IQnme-KEIDOrnLNEVAOrnnNLNESLID | 53 |
| IQnme-KEIDOrnLNEVADabNLNESLID | 54 |
| IQnme-KEIDOrnLNEVADapNLNESLID | 55 |
| IQnme-KEIDOrnLNEVAMlzNLNESLID | 56 |
| IQnme-KEIDOrnLNEVAMlyNLNESLID | 57 |
| IQOrnEIDCitLNEVAOrnNLNESLID | 58 |
| IQDabEIDCitLNEVADabNLNESLID | 59 |
| IQDapEIDCitLNEVADapNLNESLID | 60 |
| IQMIZEIDCitLNEVAMlzNLNESLID | 61 |
| IQMlyEIDCitLNEVAMlyNLNESLID | 62 |
| IQnme-KEIDCitLNEVAOrnNLNESLID | 63 |
| IQnme-KEIDCitLNEVADabNLNESLID | 64 |
| IQnme-KEIDCitLNEVADapNLNESLID | 65 |
| IQnme-KEIDCitLNEVAMlzNLNESLID | 66 |
| IQnme-KEIDCitLNEVAMlyNLNESLID | 67 |
| IQOrnEIDHLNEVIOrnnNLNESLID | 68 |
| IQDabEIDHLNEVIDabNLNESLID | 69 |
| IQDapEIDHLNEVIDapNLNESLID | 70 |
| IQOrnEIEHLNEVIOrnNLNESLID | 71 |
| IQDabEIEHLNEVIDabNLNESLID | 72 |
| IQDapEIEHLNEVIDapNLNESLID | 73 |
| IQMlzEIEHLNEVIMlzNLNESLID | 74 |
| IQMlyEIEHLNEVIMlyNLNESLID | 75 |
| IQnme-KEIEHLNEVIOrnNLNESLID | 76 |
| IQnme-KEIEHLNEVIDabNLNESLID | 77 |
| IQnme-KEIEHLNEVIDapNLNESLID | 78 |
| IQnme-KEIEHLNEVIMlzNLNESLID | 79 |
| IQnme-KEIEHLNEVIMlyNLNESLID | 80 |
| IQOrnEIDOrnLNEVIOrnNLNESLID | 81 |
| IQDabEIDOrnLNEVIDabNLNESLID | 82 |
| IQDapEIDOrnLNEVIDapNLNESLID | 83 |
| IQMlzEIDOrnLNEVIMlzNLNESLID | 84 |
| IQMlyEIDOrnLNEVIMlyNLNESLID | 85 |
| IQnme-KEIDOrnLNEVIOrnNLNESLID | 86 |
| IQnme-KEIDOrnLNEVIDabNLNESLID | 87 |
| IQnme-KEIDOrnLNEVIDapNLNESLID | 88 |
| IQnme-KEIDOrnLNEVIMlzNLNESLID | 89 |
| IQnme-KEIDOrnLNEVIMlyNLNESLID | 90 |
| IQOrnEIDCitLNEVIOrnNLNESLID | 91 |
| IQDabEIDCitLNEVIDabNLNESLID | 92 |
| IQDapEIDCitLNEVIDapNLNESLID | 93 |
| IQMlzEIDCitLNEVIMlzNLNESLID | 94 |
| IQMlyEIDCitLNEVIMlyNLNESLID | 95 |
| IQnme-KEIDCitLNEVIOrnNLNESLID | 96 |
| IQnme-KEIDCitLNEVIDabNLNESLID | 97 |
| IQnme-KEIDCitLNEVIDapNLNESLID | 98 |
| IQnme-KEIDCitLNEVIMlzNLNESLID | 99 |
| IQnme-KEIDCitLNEVIMlyNLNESLID | 100 |
| IQOrnEIDHLNEVAOrnNLGESLIE | 101 |
| IQDabEIDHLNEVADabNLGESLIE | 102 |
| IQDapEIDHLNEVADapNLGESLIE | 103 |
| IQOrnEIEHLNEVAOrnNLGESLIE | 104 |
| IQDabEIEHLNEVADabNLGESLIE | 105 |
| IQDapEIEHLNEVADapNLGESLIE | 106 |
| IQMlzEIEHLNEVAMlzNLGESLIE | 107 |
| IQMlyEIEHLNEVAMlyNLGESLIE | 108 |
| IQnme-KEIEHLNEVAOrnNLGESLIE | 109 |
| IQnme-KEIEHLNEVADabNLGESLIE | 110 |
| IQnme-KEIEHLNEVADapNLGESLIE | 111 |
| IQnme-KEIEHLNEVAMlzNLGESLIE | 112 |
| IQnme-KEIEHLNEVAMlyNLGESLIE | 113 |
| IQOrnEIDOrnLNEVAOrnNLGESLIE | 114 |
| IQDabEIDOrnLNEVADabNLGESLIE | 115 |
| IQDapEIDOrnLNEVADapNLGESLIE | 116 |
| IQMlzEIDOrnLNEVAMlzNLGESLIE | 117 |
| IQMlyEIDOrnLNEVAMlyNLGESLIE | 118 |
| IQnme-KEIDOrnLNEVAOrnNLGESLIE | 119 |
| IQnme-KEIDOrnnLNEVADabNLGESLIE | 120 |
| IQnme-KEIDOrnLNEVADapNLGESLIE | 121 |
| IQnme-KEIDOrnnLNEVAMlzNLGESLIE | 122 |
| IQnme-KEIDOrnLNEVAMlyNLGESLIE | 123 |
| IQOrnEIDCitLNEVAOrnNLGESLIE | 124 |
| IQDabEIDCitLNEVADabNLGESLIE | 125 |
| IQDapEIDCitLNEVADapNLGESLIE | 126 |
| IQMIZEIDCitLNEVAMlzNLGESLIE | 127 |
| IQMlyEIDCitLNEVAMlyNLGESLIE | 128 |
| IQnme-KEIDCitLNEVAOrnNLGESLIE | 129 |
| IQnme-KEIDCitLNEVADabNLGESLIE | 130 |
| IQnme-KEIDCitLNEVADapNLGESLIE | 131 |
| IQnme-KEIDCitLNEVAMlzNLGESLIE | 132 |
| IQnme-KEIDCitLNEVAMlyNLGESLIE | 133 |
| IQOrnEIDHLNEVIOrnNLGESLIE | 134 |
| IQDabEIDHLNEVIDabNLGESLIE | 135 |
| IQDapEIDHLNEVIDapNLGESLIE | 136 |
| IQOrnEIEHLNEVIOrnNLGESLIE | 137 |
| IQDabEIEHLNEVIDabNLGESLIE | 138 |
| IQDapEIEHLNEVIDapNLGESLIE | 139 |
| IQMlzEIEHLNEVIMlzNLGESLIE | 140 |
| IQMlyEIEHLNEVIMlyNLGESLIE | 141 |
| IQnme-KEIEHLNEVIOrnNLGESLIE | 142 |
| IQnme-KEIEHLNEVIDabNLGESLIE | 143 |
| IQnme-KEIEHLNEVIDapNLGESLIE | 144 |
| IQnme-KEIEHLNEVIMlzNLGESLIE | 145 |
| IQnme-KEIEHLNEVIMlyNLGESLIE | 146 |
| IQOrnEIDOrnLNEVIOrnNLGESLIE | 147 |
| IQDabEIDOrnLNEVIDabNLGESLIE | 148 |
| IQDapEIDOrnLNEVIDapNLGESLIE | 149 |
| IQMlzEIDOrnLNEVIMlzNLGESLIE | 150 |
| IQMlyEIDOrnLNEVIMlyNLGESLIE | 151 |
| IQnme-KEIDOrnnLNEVIOrnNLGESLIE | 152 |
| IQnme-KEIDOrnLNEVIDabNLGESLIE | 153 |
| IQnme-KEIDOrnLNEVIDapNLGESLIE | 154 |
| IQnme-KEIDOrnLNEVIMlzNLGESLIE | 155 |
| IQnme-KEIDOrnLNEVIMlyNLGESLIE | 156 |
| IQOrnEIDCitLNEVIOrnNLGESLIE | 157 |
| IQDabEIDCitLNEVIDabNLGESLIE | 158 |
| IQDapEIDCitLNEVIDapNLGESLIE | 159 |
| IQMIZEIDCitLNEVIMlzNLGESLIE | 160 |
| IQMlyEIDCitLNEVIMlyNLGESLIE | 161 |
| IQnme-KEIDCitLNEVIOrnNLGESLIE | 162 |
| IQnme-KEIDCitLNEVIDabNLGESLIE | 163 |
| IQnme-KEIDCitLNEVIDapNLGESLIE | 164 |
| IQnme-KEIDCitLNEVIMlzNLGESLIE | 165 |
| IQnme-KEIDCitLNEVIMlyNLGESLIE | 166 |
| IDRLNEVAKNLNESLIDLQELGKYE | 167 |
| IQKEIDRLNEVAKNL | 168 |
| IQKEIDRLNEVAKNLNESLIDLQEL | 169 |
| IQKEIDRLNEVAKNLNES | 170 |
| IQKEIDRLNEVAKNLNES | 171 |
| IQKEIDRLNEVAKNLNESLIDL | 172 |
| IQKEIDRLNEV | 173 |
| IEYEIDRLNEVAKNLNESLID | 174 |
| IQKEIDHLNEVAKNLNESLID | 175 |
| IQSEIDSLNELIDNLNESLID | 176 |
| IQDEIDHLNELIDNLNESLID | 177 |
| IQLEIEHLNEVIENLGESLIE | 178 |
| IQKEIDRLNEVIKNLGESLIE | 179 |
| IQKEIDHLNEVIKNLGESLIE | 180 |
| IQKEIEHLNEVIKNLGESLIE | 181 |
| IQLEIEHLNEVAENLNESLID | 182 |
| IQKEIEHLNEVAKNLNESLID | 183 |
| IQLEIDRLNEVAKNLNESLID | 184 |
| IQKEIDRLNEVAENLNESLID | 185 |
| IQKEIDRLNEVAKNLNESLID | 186 |
| IQLEIEHLNEVIENLGESLIELQ | 187 |
| IQLEIEHLNELIENLGESLIE | 188 |
| IQLEIDHLNELIENLGESLIE | 189 |
| IQLEINHLNELIENLGESLIE | 190 |
| IQLEILHLNELIENLGESLIE | 191 |
| IQLEISHLNELIENLGESLIE | 192 |
| IQLEIEHLNELIENLGESLID | 193 |
| IQLEIEHLNELIENLNESLID | 194 |
| IQQEIDRLNEVAKNLNESLID | 195 |
| IQEEIDRLNEVAKNLNESLID | 196 |
| IQSEIDRLNEVAKNLNESLID | 197 |
| IQNEIDRLNEVAKNLNESLID | 198 |
| IQKEIDNLNEVAKNLNESLID | 199 |
| IQKEIDQLNEVAKNLNESLID | 200 |
| IQKEIDELNEVAKNLNESLID | 201 |
| IQKEIDRLNEIAKNLNESLID | 202 |
| IQKEIDRLNEMAKNLNESLID | 203 |
| IQKEIDRLNELAKNLNESLID | 204 |
| IQKEIDRLNEAAKNLNESLID | 205 |
| IQKEIDRLNETAKNLNESLID | 206 |
| IQKEIDRLNEVSKNLNESLID | 207 |
| IQKEIDRLNEVVKNLNESLID | 208 |
| IQKEIDRLNEVCKNLNESLID | 209 |
| IQKEIDRLNEVTKNLNESLID | 210 |
| IQKEIDRLNEVGKNLNESLID | 211 |
| IQKEIDRLNELIKNLNESLID | 212 |
| IQKEIDRLNEIIKNLNESLID | 213 |
| IQKEIDRLNEISKNLNESLID | 214 |
| IQKEIDRLNEAIKNLNESLID | 215 |
| IQKEIDRLNETIKNLNESLID | 216 |
| IQKEIDRLNEVAQNLNESLID | 217 |
| IQKEIDRLNEVASNLNESLID | 218 |
| IQKEIDRLNEVANNLNESLID | 219 |
| IQKEIDRLNEVAKNLGESLID | 220 |
| IQKEIDRLNEVIKNLNESLID | 221 |

TABLE 1b
Exemplary Fusion Core (FC) Sequences

| FC Sequences | SEQ ID NO. |
|---|---|
| IQEEIEIILTVAIELNSTIIY | 222 |
| ISEEISILLTVAEELESALIY | 223 |
| IETEIQIILTVAIELNSTIIY | 224 |
| IEEEIQIILQVAIELNSTIIY | 225 |
| IQEYIETLLTVATELNSALIF | 226 |
| IQEEIEIILQVAIELNSTIIY | 227 |
| IQEYIETLLQVATELNSALIF | 228 |
| IQTEIEIILTVAIELNSTIIY | 229 |
| IQTEIDIILQVAIELNSTIIY | 230 |
| IQTEIDIILTVAIELNSTIIY | 231 |
| IQTEIEIILQVAIELNSTIIY | 232 |
| IQEYIDQLLIVATELNSALIY | 233 |
| IQEYIDTLLQVATELNSALIF | 234 |
| IEEHIQTLLTVATELNSALIF | 235 |
| IEDEIQTLLQVAIELNSALIF | 236 |
| IEEHIQQLLQVAIELNSALIF | 237 |
| IEEYIQTLLTVATELNSALIF | 238 |
| IQEYIDQLLIVATELNSALIF | 239 |
| IQEYIDQLLQVAIELNSALIF | 240 |
| IQLEIEHLNEVIENLGESLIE | 241 |
| IQLEIEHLNELIENLGESLIE | 242 |
| IQEYIDHLLTVASELESALIY | 243 |
| IQOrnEIEHLNEVIOrnNLNESLID | 244 |
| IQOrnEIDOrnLNEVIOrnNLNESLID | 245 |
| IQOrnEIEHLNEVAOrnNLGESLIE | 246 |
| IQOrnEIDOrnLNEVAOrnNLGESLIE | 247 |
| IQOrnEIEHLNEVIOrnNLGESLIE | 248 |
| IQOrnEIDOrnLNEVIOrnNLGESLIE | 249 |
| IQKEIDRLNEVAKNLNESLID | 250 |
| IQKEIDRLNEVAKNLNESLIDLQEL | 251 |
| IQOrnEIDhArgLNEVIOrnNLNESLID | 252 |
| IQOrnEIDOrnLNEIIOrnNLNESLID | 253 |

When one or more extension amino acid sequence (EXT) is present, each EXT has independently 1 to 25 amino acid residues in length. In some embodiments, each EXT has independently 2 to 24 amino acid residues in length. In some embodiments, each EXT has independently 3, 4, 5, 6, 7, 8, 9, 10, 11, 12, 13, 14, 15, 16, 17, 18, 19, 20, 21, 22, or 23 amino acid residues in length. In some embodiments, each EXT has independently 3, 4, 5, 6, 11, 12, 13, 14, 15, 16, 17, 18, 19, 20, 21, 22, or 23 amino acid residues in length.

In some embodiments, one EXT is present at the C-terminus of the HRC sequence.

In some embodiments, one EXT is present at the C-terminus of the HRC sequence, and the EXT has 3, 4, 5, or 6 amino acid residues in length, preferably 4 or 5 amino acid residues in length.

In some embodiments, one EXT is present at the C-terminus of the HRC sequence, and the EXT has at least 70% identity to an amino acid sequence derived from the HRC region of SARS-CoV-2 S protein.

In some embodiments, one EXT is present at the N-terminus of the HRC sequence.

In some embodiments, one EXT is present at the N-terminus of the HRC sequence and the EXT has 3, about 11, about 14, about 17, 18, about 22, or 23 amino acid residues in length, preferably about 11 amino acid residues in length.

In some embodiments, one EXT is present at each terminus of the HRC sequence.

In some embodiments, one EXT is present at each terminus of the HRC sequence; the EXT at the C-terminus of the HRC sequence has 3, 4, 5, or 6 amino acid residues in length, preferably 4 or 5 amino acid residues in length; and the EXT at the N-terminus of the HRC sequence has 3, about 11, about 14, about 17, 18, about 22, or 23 amino acid residues in length, preferably about 11 amino acid residues in length.

In some embodiments, each EXT is an amino acid sequence having at least 50%, at least 60%, at least 70%, at least 80%, at least 90%, at least 95%, at least 97%, or at least 99% identity to any one of:

LQEL,
LQELG,
DISGINASVVN,
DIAAINASVAN,
DISGINASVVE,
DISGINASVVD,
DISGINASVEN,
SIDQINATFVD,
PDVDLGDISGINASVVN,
PDVDLGGDISGINASVVN,
KNHTSPDVDLGDISGINASVVN,
KNHTSPDVDLGGDISGINASVVN,
DLGDISGINASVVN,
VVN,
DLGDISGINLTILN,
DLGDISGINLSVVN,
DLGDISGINLSILN,
LGDISGINLSILN,
LGDISGINLTILN,
LGDISGINLSVVN,
DISGINAibSVVN,
LG,
LGDISGINAibSVVN.

In some embodiments, one EXT is present at the C-terminus of the HRC sequence, and the EXT has at least 50%, at least 60%, at least 70%, at least 80%, at least 90%, at least 95%, at least 97%, or at least 99% identity to LQEL, or LQELG, preferably LQEL.

In some embodiments, one EXT is present at the C-terminus of the HRC sequence, and the EXT is LQEL or LQELG, preferably LQEL.

In some embodiments, one EXT is present at the N-terminus of the HRC sequence, and the EXT has at least 50%, at least 60%, at least 70%, at least 80%, at least 90%, at least 95%, at least 97%, or at least 99% identity to any one of EXT amino acid sequences as described above.

In some embodiments, one EXT is present at the N-terminus of the HRC sequence, and the EXT is an amino acid sequence selected from EXT amino acid sequences as described above.

In some embodiments, the HRC sequence is represented by EXT-FC or FC-EXT.

In some embodiments, the HRC sequence is represented by EXT-FC or FC-EXT, wherein FC is an amino acid sequence having at least 70% identity to any one of the amino acid sequences presented in Table 1 or Table 1a, the EXT has at least 70% identity to any one of these amino acid sequences.

In some embodiments, the HRC sequence is represented by EXT-FC, wherein FC is an amino acid sequence having at least 70% identity to any one of the amino acid sequences presented in Table 1 or Table 1a, the EXT has at least 70% identity to any one of these amino acid sequences.

In some preferred embodiments, the HRC sequence is represented by FC-EXT, wherein FC is an amino acid sequence having at least 70% identity to any one of the amino acid sequences presented in Table 1 or Table 1a, the EXT has at least 70% identity to LQEL or LQELG.

In some preferred embodiments, the HRC sequence is represented by EXT-FC-EXT, wherein FC is an amino acid sequence having at least 70% identity to any one of the amino acid sequences presented in Table 1 or Table 1a, the EXT at C-terminus has at least 70% identity to LQEL or LQELG, and the EXT at N-terminus has at least 70% identity to any one of:

DISGINASVVN,
DIAAINASVAN,
DISGINASVVE,
DISGINASVVD,
DISGINASVEN,
SIDQINATFVD,
PDVDLGDISGINASVVN,
PDVDLGGDISGINASVVN,
KNHTSPDVDLGDISGINASVVN,
KNHTSPDVDLGGDISGINASVVN,
DLGDISGINASVVN,
VVN,
DLGDISGINLTILN,
DLGDISGINLSVVN,
DLGDISGINLSILN,
LGDISGINLSILN,
LGDISGINLTILN,
LGDISGINLSVVN,
DISGINAibSVVN,
LG,
LGDISGINAibSVVN.

In some preferred embodiments, the HRC sequence is represented by EXT-FC-EXT, wherein FC is an amino acid sequence having at least 70% identity any one of the amino acid sequences presented in Table 1 or Table 1a, the EXT at C-terminus is an amino acid sequence selected from LQEL or LQELG, and the EXT at N-terminus is an amino acid sequence selected from these amino acid sequences:

DISGINASVVN,
DIAAINASVAN,
DISGINASVVE,
DISGINASVVD,
DISGINASVEN,
SIDQINATFVD,
PDVDLGDISGINASVVN,
PDVDLGGDISGINASVVN,
KNHTSPDVDLGDISGINASVVN,
KNHTSPDVDLGGDISGINASVVN,
DLGDISGINASVVN,
VVN,
DLGDISGINLTILN,
DLGDISGINLSVVN,
DLGDISGINLSILN,
LGDISGINLSILN,
LGDISGINLTILN,
LGDISGINLSVVN,
DISGINAibSVVN,
LG,
LGDISGINAibSVVN.

In additional embodiments, the HRC sequence comprises an amino acid sequence having at least 70% identity to an inverted sequence of any one of HRC sequence as described above. In some cases, the HRC sequence has at least 70% identity to an inverted sequence of any one of HRC sequence as described above. In some cases, the HRC sequence is an inverted sequence of any one of HRC sequence as described above. As used herein, the term “reverted sequence” means an amino acid sequence that is the complete reverse of the original sequence, or that contains a reversed portion of the original sequence with the other portion remaining the same. It means the order of the amino acids, in whole or in the reversed portion, has been reversed. For example, a reverted sequence of DISGINASVVNIQKEIDRLNEVAKNLNESLIDLQEL (SEQ ID NO. 1) includes the complete reverse: LEQLDILSENLNKAVENLRDIEKQINVVSANIGSID (SEQ ID NO. 272), as well as a partial reverse, i.e., DISGINASVVNDILSENLNKAVENLRDIEKQILQEL (SEQ ID NO. 1041), wherein only the fusion core FC sequence is reversed. In preferred embodiments, a “reverted sequence” refers to a complete reverse of the original sequence.

In some embodiments, the HRC sequence is a truncated sequence with respect to SEQ ID NO. 1. The term “truncation” refers to the shortening of an amino acid sequence by removing one or more amino acids from one or both ends of the amino acid sequence, resulting in a truncated sequence. The truncated sequence may or may not further include point mutation with respect to the original amino acid sequence. For example, IQKEIDRLNEVAKNLNESLIDLQEL is a truncated sequence with respect to SEQ ID NO. 1. VVNIQKEIDRLNEVAKNLNESLID is another truncated sequence with respect to SEQ ID NO. 1.

In some cases, the HRC sequence is a truncated sequence with respect to SEQ ID NO. 1, and the truncated sequence is up to 16 amino acids shorter than SEQ ID NO. 1. In some cases, the HRC sequence is a truncated sequence with respect to SEQ ID NO. 1, and the truncated sequence is up to 15 amino acids shorter than SEQ ID NO. 1. In some cases, the HRC sequence is a truncated sequence with respect to SEQ ID NO. 1, and the truncated sequence is up to 14 amino acids shorter than SEQ ID NO. 1. In some cases, the HRC sequence is a truncated sequence with respect to SEQ ID NO. 1, and the truncated sequence is up to 13 amino acids shorter than SEQ ID NO. 1. In some cases, the HRC sequence is a truncated sequence with respect to SEQ ID NO. 1, and the truncated sequence is up to 12 amino acids shorter than SEQ ID NO. 1. In some cases, the HRC sequence is a truncated sequence with respect to SEQ ID NO. 1, and the truncated sequence is up to 11 amino acids shorter than SEQ ID NO. 1.

In some embodiments, the HRC sequence is an extension sequence with respect to SEQ ID NO. 1. As used herein, the term “extension” refers to the addition of one or more amino acids to an original amino acid sequence, resulting in a longer amino acid sequence compared to the original, i.e., an extension sequence. The extension sequence may or may not further include point mutations with respect to the original amino acid sequence.

For example, PDVDLGDISGINASVVNIQKEIDRLNEVAKNLNESLIDLQEL (SEQ ID NO. 255) is an extension sequence with respect to SEQ ID NO. 1.

In some cases, the HRC sequence is an extension sequence with respect to SEQ ID NO. 1, and the extension sequence is up to 16 amino acids longer than SEQ ID NO. 1. In some cases, the HRC sequence is an extension sequence with respect to SEQ ID NO. 1, and the extension sequence is up to 15 amino acids longer than SEQ ID NO. 1. In some cases, the HRC sequence is an extension sequence with respect to SEQ ID NO. 1, and the extension sequence is up to 14 amino acids longer than SEQ ID NO. 1. In some cases, the HRC sequence is an extension sequence with respect to SEQ ID NO. 1, and the extension sequence is up to 13 amino acids longer than SEQ ID NO. 1. In some cases, the HRC sequence is an extension sequence with respect to SEQ ID NO. 1, and the extension sequence is up to 12 amino acids longer than SEQ ID NO. 1. In some cases, the HRC sequence is an extension sequence with respect to SEQ ID NO. 1, and the extension sequence is up to 11 amino acids longer than SEQ ID NO. 1.

In some embodiments, the HRC sequence comprises an amino acid sequence that contains point mutations with respect to SEQ ID NO. 1. In some cases, the HRC sequence has up to 20 amino acid mutations relative to DISGINASVVNIQKEIDRLNEVAKNLNESLIDLQEL SEQ ID NO. 1. In some cases, the HRC sequence has up to 15 amino acid mutations relative to SEQ ID NO. 1. In some cases, the HRC sequence has up to 12 amino acid mutations relative to SEQ ID NO. 1. In some cases, the HRC sequence has up to 10 amino acid mutations relative to SEQ ID NO. 1. In some cases, the HRC sequence has 1, 2, 3, 4, 5, 6, 7, 8, or 9 amino acid mutations relative to SEQ ID NO. 1.

In some embodiments, the HRC sequence comprises an amino acid that has at least 50%, at least 60%, at least 70%, at least 80%, at least 90%, at least 95%, at least 97%, or at least 99% identity to any one of These amino acid sequences in Table 2. In some embodiments, the HRC sequence is an amino acid sequence having at least 50%, at least 60%, at least 70%, at least 80%, at least 90%, at least 95%, at least 97%, or at least 99% identity to any one of These amino acid sequences in Table 2. In some embodiments, the HRC sequence is an amino acid sequence selected from These amino acid sequences in Table 2.

TABLE 2
Exemplary HRC Sequences

| HRC Sequence | SEQ ID NO. |
|---|---|
| DISGINASVVNIQKEIDRLNEVAKNLNESLIDLQEL | 254 |
| PDVDLGDISGINASVVNIQKEIDRLNEVAKNLNESLIDLQEL | 255 |
| IQKEIDRLNEVAKNLNESLIDLQEL | 256 |
| DISGINASVVNIQKPIDRLNEVAKNLNESLIDLQEL | 257 |
| DISGINASVVNIQKPIDPLNEVAKPLNEPLIDLQEL | 258 |
| DISGINASVVNAQKEADRANEAAKNANESLIDLQEL | 259 |
| DISGINASVVNIQKEIDRLNEVIKNLNESLIDLQEL | 260 |
| DISGINASVVNIQKEIARLNEVAKALNESLIDLQEL | 261 |
| DIAAINASVANIQKEIARLNEVAKALNESLAALQAL | 262 |
| DISGINASVVNIQKEIARLNEVIKALNESLIDLQEL | 263 |
| DISGINASVVNIQKEIERLNKVAKNLNESLIDLQEL | 264 |
| DISGINASVVEIQKKIEELNKKAEELNKKLIDLQEL | 265 |
| DISGINASVVDIKKEIEHLNEIAKSLNESLIDLQEL | 266 |
| DISGINASVVDIKKEIEHLNEIAKNLNESLIDLQEL | 267 |
| SLDQINVTFLDLEYEMKKLEEAIKKLEESYIDLKEL | 268 |
| DISQINASVVNIEYEIKKLEEVAKKLEESLIDLQEL | 269 |
| SIDQINATFVDIEYEIKKLEEVAKKLEESYIDLQEL | 270 |
| KNHTSPDVDLGDISGINASVVNIQKEIDRLNEVAKNLNESLIDLQEL | 271 |
| LEQLDILSENLNKAVENLRDIEKQINVVSANIGSID | 272 |
| EAAAKEAAAKEAAAKEAAAKEAAAKEAAAKEAAAKEAAAK | 273 |
| PDVDLGGDISGINASVVNIQKEIDRLNEVAKNLNESLIDLQEL | 274 |
| KNHTSPDVDLGGDISGINASVVNIQKEIDRLNEVAKNLNESLIDLQEL | 275 |
| DLGDISGINASVVNIQKEIDRLNEVAKNLNESLIDLQELG | 276 |
| VVNIQKEIDRLNEVAKNLNESLID | 277 |
| DISGINASVVNIQKEIDRLNEVAKNLNESLIELQEL | 278 |
| DISGINASVVNIQKEIDRLNEVAKNLNESLIKLQEL | 279 |
| DISGINASVVNIQKEIDRLNEVAKNLNESLIGLQEL | 280 |
| DISGINASVVNIQKEIDRLNEVAKNLGESLIGLQEL | 281 |
| DISGINASVVNIQKEIDRLNEVAKNLPESLIGLQEL | 282 |
| DISGINASVVNIQKEIDRLDEVAKNLNESLIDLQEL | 283 |
| DISGINASVVNIQKEIDRLGEVAKNLNESLIDLQEL | 284 |
| DISGINASVENIQKEIDRLNEVAKNLNESLIDLQEL | 285 |
| DISGINASVVNIQKEIDRLNEVAKNLGESLIELQEL | 286 |
| DISGINASVVNIQKEIDRLDEVAKNLPESLIELQEL | 287 |
| IEEIQKKIEEIQKKIEEIQKKIEEIQKK | 288 |
| EKLDIYSENLNKAVENLTGLEQKLLQQVDLTAYSYILDE | 289 |
| IPNFGSLTQINTTLLDLTYEMLSLQQVVKALNESYIDLKE | 290 |
| DLGDISGINLTILNIQDEIEHLNELIDNLNESLIDLQEL | 291 |
| DLGDISGINLTILNIQLEIEHLNELIENLGESLIELQEL | 292 |
| DLGDISGINLSVVNIQLEIEHLNEVIENLGESLIELQEL | 293 |
| DLGDISGINLSILNIQLEIEHLNEVIENLGESLIELQEL | 294 |
| LGDISGINLSILNIQLEIEHLNEVIENLGESLIELQEL | 295 |
| LGDISGINLTILNIQLEIEHLNELIENLGESLIELQEL | 296 |
| LGDISGINLSVVNIQLEIEHLNEVIENLGESLIELQEL | 297 |
| LGDISGINLSVVNIQLEIDHLNEVIENLGESLIELQEL | 298 |
| DLGDISGINLSVVNIQLEIDHLNEVIENLGESLIELQEL | 299 |
| DLGDISGINLSVVNIQLEIEHLNELIENLGESLIELQEL | 300 |

TABLE 2a
Additional exemplary HRC Sequences

| HRC Sequence | SEQ ID NO. |
|---|---|
| DISGINASVVNIQKEIDRLNEVIKNLGESLIELQEL | 301 |
| LGDISGINASVVNIQKEIDRLNEVIKNLGESLIELQEL | 302 |
| LGDISGINAibSVVNIQOrnEIDHLNEVAOrnNLNESLIDLQEL | 303 |
| LGDISGINAibSVVNIQDabEIDHLNEVADabNLNESLIDLQEL | 304 |
| LGDISGINAibSVVNIQDapEIDHLNEVADapNLNESLIDLQEL | 305 |
| LGDISGINAibSVVNIQOrnEIEHLNEVAOrnNLNESLIDLQEL | 306 |
| LGDISGINAibSVVNIQDabEIEHLNEVADabNLNESLIDLQEL | 307 |
| LGDISGINAibSVVNIQDapEIEHLNEVADapNLNESLIDLQEL | 308 |
| LGDISGINAibSVVNIQMlzEIEHLNEVAMlzNLNESLIDLQEL | 309 |
| LGDISGINAibSVVNIQMlyEIEHLNEVAMlyNLNESLIDLQEL | 310 |
| LGDISGINAibSVVNIQnme-KEIEHLNEVAOrnNLNESLIDLQEL | 311 |
| LGDISGINAibSVVNIQnme-KEIEHLNEVADabNLNESLIDLQEL | 312 |
| LGDISGINAibSVVNIQnme-KEIEHLNEVADapNLNESLIDLQEL | 313 |
| LGDISGINAibSVVNIQnme-KEIEHLNEVAMlzNLNESLIDLQEL | 314 |
| LGDISGINAibSVVNIQnme-KEIEHLNEVAMlyNLNESLIDLQEL | 315 |
| LGDISGINAibSVVNIQOrnEIDOrnLNEVAOrnNLNESLIDLQEL | 316 |
| LGDISGINAibSVVNIQDabEIDOrnLNEVADabNLNESLIDLQEL | 317 |
| LGDISGINAibSVVNIQDapEIDOrnLNEVADapNLNESLIDLQEL | 318 |
| LGDISGINAibSVVNIQMlzEIDOrnLNEVAMlzNLNESLIDLQEL | 319 |
| LGDISGINAibSVVNIQMlyEIDOrnLNEVAMlyNLNESLIDLQEL | 320 |
| LGDISGINAibSVVNIQnme-KEIDOrnLNEVAOrnnNLNESLIDLQEL | 321 |
| LGDISGINAibSVVNIQnme-KEIDOrnLNEVADabNLNESLIDLQEL | 322 |
| LGDISGINAibSVVNIQnme-KEIDOrnLNEVADapNLNESLIDLQEL | 323 |
| LGDISGINAibSVVNIQnme-KEIDOrnLNEVAMlzNLNESLIDLQEL | 324 |
| LGDISGINAibSVVNIQnme-KEIDOrnLNEVAMlyNLNESLIDLQEL | 325 |
| LGDISGINAibSVVNIQOrnEIDCitLNEVAOrnNLNESLIDLQEL | 326 |
| LGDISGINAibSVVNIQDabEIDCitLNEVADabNLNESLIDLQEL | 327 |
| LGDISGINAibSVVNIQDapEIDCitLNEVADapNLNESLIDLQEL | 328 |
| LGDISGINAibSVVNIQMIZEIDCitLNEVAMlzNLNESLIDLQEL | 329 |
| LGDISGINAibSVVNIQMlyEIDCitLNEVAMlyNLNESLIDLQEL | 330 |
| LGDISGINAibSVVNIQnme-KEIDCitLNEVAOrnNLNESLIDLQEL | 331 |
| LGDISGINAibSVVNIQnme-KEIDCitLNEVADabNLNESLIDLQEL | 332 |
| LGDISGINAibSVVNIQnme-KEIDCitLNEVADapNLNESLIDLQEL | 333 |
| LGDISGINAibSVVNIQnme-KEIDCitLNEVAMlzNLNESLIDLQEL | 334 |
| LGDISGINAibSVVNIQnme-KEIDCitLNEVAMlyNLNESLIDLQEL | 335 |
| LGDISGINAibSVVNIQOrnEIDHLNEVIOrnNLNESLIDLQEL | 336 |
| LGDISGINAibSVVNIQDabEIDHLNEVIDabNLNESLIDLQEL | 337 |
| LGDISGINAibSVVNIQDapEIDHLNEVIDapNLNESLIDLQEL | 338 |
| LGDISGINAibSVVNIQOrnEIEHLNEVIOrnNLNESLIDLQEL | 339 |
| LGDISGINAibSVVNIQDabEIEHLNEVIDabNLNESLIDLQEL | 340 |
| LGDISGINAibSVVNIQDapEIEHLNEVIDapNLNESLIDLQEL | 341 |
| LGDISGINAibSVVNIQMlzEIEHLNEVIMlzNLNESLIDLQEL | 342 |
| LGDISGINAibSVVNIQMlyEIEHLNEVIMlyNLNESLIDLQEL | 343 |
| LGDISGINAibSVVNIQnme-KEIEHLNEVIOrnnNLNESLIDLQEL | 344 |
| LGDISGINAibSVVNIQnme-KEIEHLNEVIDabNLNESLIDLQEL | 345 |
| LGDISGINAibSVVNIQnme-KEIEHLNEVIDapNLNESLIDLQEL | 346 |
| LGDISGINAibSVVNIQnme-KEIEHLNEVIMlzNLNESLIDLQEL | 347 |
| LGDISGINAibSVVNIQnme-KEIEHLNEVIMlyNLNESLIDLQEL | 348 |
| LGDISGINAibSVVNIQOrnEIDOrnLNEVIOrnNLNESLIDLQEL | 349 |
| LGDISGINAibSVVNIQDabEIDOrnLNEVIDabNLNESLIDLQEL | 350 |
| LGDISGINAibSVVNIQDapEIDOrnLNEVIDapNLNESLIDLQEL | 351 |
| LGDISGINAibSVVNIQMlzEIDOrnLNEVIMlzNLNESLIDLQEL | 352 |
| LGDISGINAibSVVNIQMlyEIDOrnLNEVIMlyNLNESLIDLQEL | 353 |
| LGDISGINAibSVVNIQnme-KEIDOrnLNEVIOrnNLNESLIDLQEL | 354 |
| LGDISGINAibSVVNIQnme-KEIDOrnLNEVIDabNLNESLIDLQEL | 355 |
| LGDISGINAibSVVNIQnme-KEIDOrnLNEVIDapNLNESLIDLQEL | 356 |
| LGDISGINAibSVVNIQnme-KEIDOrnLNEVIMlzNLNESLIDLQEL | 357 |
| LGDISGINAibSVVNIQnme-KEIDOrnLNEVIMlyNLNESLIDLQEL | 358 |
| LGDISGINAibSVVNIQOrnEIDCitLNEVIOrnNLNESLIDLQEL | 359 |
| LGDISGINAibSVVNIQDabEIDCitLNEVIDabNLNESLIDLQEL | 360 |
| LGDISGINAibSVVNIQDapEIDCitLNEVIDapNLNESLIDLQEL | 361 |
| LGDISGINAibSVVNIQMIZEIDCitLNEVIMlzNLNESLIDLQEL | 362 |
| LGDISGINAibSVVNIQMlyEIDCitLNEVIMlyNLNESLIDLQEL | 363 |
| LGDISGINAibSVVNIQnme-KEIDCitLNEVIOrnNLNESLIDLQEL | 364 |
| LGDISGINAibSVVNIQnme-KEIDCitLNEVIDabNLNESLIDLQEL | 365 |
| LGDISGINAibSVVNIQnme-KEIDCitLNEVIDapNLNESLIDLQEL | 366 |
| LGDISGINAibSVVNIQnme-KEIDCitLNEVIMlzNLNESLIDLQEL | 367 |
| LGDISGINAibSVVNIQnme-KEIDCitLNEVIMlyNLNESLIDLQEL | 368 |
| LGDISGINAibSVVNIQOrnEIDHLNEVAOrnNLGESLIELQEL | 369 |
| LGDISGINAibSVVNIQDabEIDHLNEVADabNLGESLIELQEL | 370 |
| LGDISGINAibSVVNIQDapEIDHLNEVADapNLGESLIELQEL | 371 |
| LGDISGINAibSVVNIQOrnEIEHLNEVAOrnNLGESLIELQEL | 372 |
| LGDISGINAibSVVNIQDabEIEHLNEVADabNLGESLIELQEL | 373 |
| LGDISGINAibSVVNIQDapEIEHLNEVADapNLGESLIELQEL | 374 |
| LGDISGINAibSVVNIQMlzEIEHLNEVAMlzNLGESLIELQEL | 375 |
| LGDISGINAibSVVNIQMlyEIEHLNEVAMlyNLGESLIELQEL | 376 |
| LGDISGINAibSVVNIQnme-KEIEHLNEVAOrnNLGESLIELQEL | 377 |
| LGDISGINAibSVVNIQnme-KEIEHLNEVADabNLGESLIELQEL | 378 |
| LGDISGINAibSVVNIQnme-KEIEHLNEVADapNLGESLIELQEL | 379 |
| LGDISGINAibSVVNIQnme-KEIEHLNEVAMlzNLGESLIELQEL | 380 |
| LGDISGINAibSVVNIQnme-KEIEHLNEVAMlyNLGESLIELQEL | 381 |
| LGDISGINAibSVVNIQOrnEIDOrnLNEVAOrnNLGESLIELQEL | 382 |
| LGDISGINAibSVVNIQDabEIDOrnLNEVADabNLGESLIELQEL | 383 |
| LGDISGINAibSVVNIQDapEIDOrnLNEVADapNLGESLIELQEL | 384 |
| LGDISGINAibSVVNIQMlzEIDOrnLNEVAMlzNLGESLIELQEL | 385 |
| LGDISGINAibSVVNIQMlyEIDOrnLNEVAMlyNLGESLIELQEL | 386 |
| LGDISGINAibSVVNIQnme-KEIDOrnLNEVAOrnNLGESLIELQEL | 387 |
| LGDISGINAibSVVNIQnme-KEIDOrnLNEVADabNLGESLIELQEL | 388 |
| LGDISGINAibSVVNIQnme-KEIDOrnLNEVADapNLGESLIELQEL | 389 |
| LGDISGINAibSVVNIQnme-KEIDOrnLNEVAMlzNLGESLIELQEL | 390 |
| LGDISGINAibSVVNIQnme-KEIDOrnLNEVAMlyNLGESLIELQEL | 391 |
| LGDISGINAibSVVNIQOrnEIDCitLNEVAOrnNLGESLIELQEL | 392 |
| LGDISGINAibSVVNIQDabEIDCitLNEVADabNLGESLIELQEL | 393 |
| LGDISGINAibSVVNIQDapEIDCitLNEVADapNLGESLIELQEL | 394 |
| LGDISGINAibSVVNIQMIZEIDCitLNEVAMlzNLGESLIELQEL | 395 |
| LGDISGINAibSVVNIQMlyEIDCitLNEVAMlyNLGESLIELQEL | 396 |
| LGDISGINAibSVVNIQnme-KEIDCitLNEVAOrnNLGESLIELQEL | 397 |
| LGDISGINAibSVVNIQnme-KEIDCitLNEVADabNLGESLIELQEL | 398 |
| LGDISGINAibSVVNIQnme-KEIDCitLNEVADapNLGESLIELQEL | 399 |
| LGDISGINAibSVVNIQnme-KEIDCitLNEVAMlzNLGESLIELQEL | 400 |
| LGDISGINAibSVVNIQnme-KEIDCitLNEVAMlyNLGESLIELQEL | 401 |
| LGDISGINAibSVVNIQOrnEIDHLNEVIOrnNLGESLIELQEL | 402 |
| LGDISGINAibSVVNIQDabEIDHLNEVIDabNLGESLIELQEL | 403 |
| LGDISGINAibSVVNIQDapEIDHLNEVIDapNLGESLIELQEL | 404 |
| LGDISGINAibSVVNIQOrnEIEHLNEVIOrnNLGESLIELQEL | 405 |
| LGDISGINAibSVVNIQDabEIEHLNEVIDabNLGESLIELQEL | 406 |
| LGDISGINAibSVVNIQDapEIEHLNEVIDapNLGESLIELQEL | 407 |
| LGDISGINAibSVVNIQMlzEIEHLNEVIMlzNLGESLIELQEL | 408 |
| LGDISGINAibSVVNIQMlyEIEHLNEVIMlyNLGESLIELQEL | 409 |
| LGDISGINAibSVVNIQnme-KEIEHLNEVIOrnNLGESLIELQEL | 410 |
| LGDISGINAibSVVNIQnme-KEIEHLNEVIDabNLGESLIELQEL | 411 |
| LGDISGINAibSVVNIQnme-KEIEHLNEVIDapNLGESLIELQEL | 412 |
| LGDISGINAibSVVNIQnme-KEIEHLNEVIMlzNLGESLIELQEL | 413 |
| LGDISGINAibSVVNIQnme-KEIEHLNEVIMlyNLGESLIELQEL | 414 |
| LGDISGINAibSVVNIQOrnEIDOrnLNEVIOrnNLGESLIELQEL | 415 |
| LGDISGINAibSVVNIQDabEIDOrnLNEVIDabNLGESLIELQEL | 416 |
| LGDISGINAibSVVNIQDapEIDOrnLNEVIDapNLGESLIELQEL | 417 |
| LGDISGINAibSVVNIQMlzEIDOrnLNEVIMlzNLGESLIELQEL | 418 |
| LGDISGINAibSVVNIQMlyEIDOrnLNEVIMlyNLGESLIELQEL | 419 |
| LGDISGINAibSVVNIQnme-KEIDOrnLNEVIOrnNLGESLIELQEL | 420 |
| LGDISGINAibSVVNIQnme-KEIDOrnLNEVIDabNLGESLIELQEL | 421 |
| LGDISGINAibSVVNIQnme-KEIDOrnLNEVIDapNLGESLIELQEL | 422 |
| LGDISGINAibSVVNIQnme-KEIDOrnLNEVIMlzNLGESLIELQEL | 423 |
| LGDISGINAibSVVNIQnme-KEIDOrnLNEVIMlyNLGESLIELQEL | 424 |
| LGDISGINAibSVVNIQOrnEIDCitLNEVIOrnNLGESLIELQEL | 425 |
| LGDISGINAibSVVNIQDabEIDCitLNEVIDabNLGESLIELQEL | 426 |
| LGDISGINAibSVVNIQDapEIDCitLNEVIDapNLGESLIELQEL | 427 |
| LGDISGINAibSVVNIQMIZEIDCitLNEVIMlzNLGESLIELQEL | 428 |
| LGDISGINAibSVVNIQMlyEIDCitLNEVIMlyNLGESLIELQEL | 429 |
| LGDISGINAibSVVNIQnme-KEIDCitLNEVIOrnNLGESLIELQEL | 430 |
| LGDISGINAibSVVNIQnme-KEIDCitLNEVIDabNLGESLIELQEL | 431 |
| LGDISGINAibSVVNIQnme-KEIDCitLNEVIDapNLGESLIELQEL | 432 |
| LGDISGINAibSVVNIQnme-KEIDCitLNEVIMlzNLGESLIELQEL | 433 |
| LGDISGINAibSVVNIQnme-KEIDCitLNEVIMlyNLGESLIELQEL | 434 |
| VDLGDISGINASVVNIQKEIDRLNEVAKNLNESLIDLQEL | 435 |
| SVVNIQKEIDRLNEVAKNLNESLIDLQELGKYEQYIK | 436 |
| GINASVVNIQKEIDRLNEVAKNL | 437 |
| GINASVVNIQKEIDRLNEVAKNLNESLIDL | 438 |
| GINASVVNIQKEIDRLNEVAKNLNESLIDLQELGKYE | 439 |
| ISYVEITVPNIQKEIDRLNEVAKNLNES | 440 |
| DISYVEITVPNIQKEIDRLNEVAKNLN | 441 |
| ISYVEITVPNIQKEIDRLNEVAKNLNESLIDLQ | 442 |
| DISYVEITVPNIQKEIDRLNEVAKNLNESLIDLQ | 443 |
| PDVDLGDISYVEITVPNIQKEIDRLNEVAKNLNESLIDLQ | 444 |
| PDVDLGDISYVEITVPNIQKEIDRLNEVAK | 445 |
| PDVDLGDISGINASVVNIQKEIDRLNEVAKNL | 446 |
| DISGINASVVNIQKEIDRLNEVAENLNESLIDLQEL | 447 |
| DISGINASVVNIEYEIDRLNEVAKNLNESLIDLQEL | 448 |
| DISGINASVVNIQKEIDHLNEVAKNLNESLIDLQEL | 449 |
| PDLGDISGINESVVNIQSEIDSLNELIDNLNESLIDLQEL | 450 |
| PDLGDISGINESVVNIQSEIDSLNELIDNLNESLIDLQEL | 451 |
| DLGDISGINESVVNIQSEIDSLNELIDNLNESLIDLQEL | 452 |
| DLGDISGINESVVNIQSEIDSLNELIDNLNESLIDLQEL | 453 |
| DLGDISGINQSVVNIQSEIDSLNELIDNLNESLIDLQEL | 454 |
| DLGDISGINLSVVNIQSEIDSLNELIDNLNESLIDLQEL | 455 |
| DLGDISGINTSVVNIQSEIDSLNELIDNLNESLIDLQEL | 456 |
| PDLGDISGINESLLNIQSEIDSLNELIDNLNESLIDLQEL | 457 |
| PDLGDISGINESILNIQSEIDSLNELIDNLNESLIDLQEL | 458 |
| PDLGDISGINATILNIQSEIDSLNELIDNLNESLIDLQEL | 459 |
| DLGDISGINATILNIQDEIDHLNELIDNLNESLIDLQEL | 460 |
| DLGDISGINLTILNIQDEIDHLNELIDNLNESLIDLQEL | 461 |
| DLGDISGINLTILNIQLEIEHLNEVIENLGESLIELQEL | 462 |
| DISGINASVVNIQKEIDRLNEVIKNLGESLIELQEL | 463 |
| DISGINASVVNIQKEIDHLNEVIKNLGESLIELQEL | 464 |
| DISGINASVVNIQKEIEHLNEVIKNLGESLIELQEL | 465 |
| DLGDISGINASVVNIQKEIEHLNEVIKNLGESLIELQEL | 466 |
| DLGDISGINASVVNIQKEIEHLNEVIKNLGESLIELQEL | 467 |
| DLGDISGINLSVVNIQLEIEHLNEVAENLNESLIDLQEL | 468 |
| DLGDISGINLSVVNIQLEIEHLNEVAENLNESLIDLQEL | 469 |
| LGDISGINASVVNIQKEIEHLNEVAKNLNESLIDLQEL | 470 |
| LGDISGINASVVNIQLEIDRLNEVAKNLNESLIDLQEL | 471 |
| LGDISGINLSVVNIQKEIDRLNEVAKNLNESLIDLQEL | 472 |
| LGDISGINASVLNIQKEIDRLNEVAKNLNESLIDLQEL | 473 |
| LGDISGINASIVNIQKEIDRLNEVAKNLNESLIDLQEL | 474 |
| LGDISGINASLVNIQKEIDRLNEVAKNLNESLIDLQEL | 475 |
| LGDISGINASILNIQKEIDRLNEVAKNLNESLIDLQEL | 476 |
| LGDISGINASVVNIQKEIDRLNEVAENLNESLIDLQEL | 477 |
| LGDISGINASVVNIQKEIDRLNEVAKNLNESLIDLQEL | 478 |
| DLGDISGINLSVVNIQLEIEHLNEVIENLGESLIELQELGS | 479 |
| DLGDISGINLTILDIQLEIEHLNELIENLGESLIELQEL | 480 |
| DLGDISGINLTILNIQLEIDHLNELIENLGESLIELQEL | 481 |
| DLGDISGINLTILNIQLEINHLNELIENLGESLIELQEL | 482 |
| DLGDISGINLTILNIQLEILHLNELIENLGESLIELQEL | 483 |
| DLGDISGINLTILNIQLEISHLNELIENLGESLIELQEL | 484 |
| DLGDISGINLTILNIQLEIEHLNELIENLGESLIDLQEL | 485 |
| DLGDISGINLTILNIQLEIEHLNELIENLNESLIDLQEL | 486 |
| DLGDISGINSSVVNIQKEIDRLNEVAKNLNESLIDLQEL | 487 |
| DLGDISGINGSVVNIQKEIDRLNEVAKNLNESLIDLQEL | 488 |
| DLGDISGINTSVVNIQKEIDRLNEVAKNLNESLIDLQEL | 489 |
| DLGDISGINASIVNIQKEIDRLNEVAKNLNESLIDLQEL | 490 |
| DLGDISGINASLVNIQKEIDRLNEVAKNLNESLIDLQEL | 491 |
| DLGDISGINASMVNIQKEIDRLNEVAKNLNESLIDLQEL | 492 |
| DLGDISGINASVINIQKEIDRLNEVAKNLNESLIDLQEL | 493 |
| DLGDISGINASVLNIQKEIDRLNEVAKNLNESLIDLQEL | 494 |
| DLGDISGINASVMNIQKEIDRLNEVAKNLNESLIDLQEL | 495 |
| DLGDISGINASIINIQKEIDRLNEVAKNLNESLIDLQEL | 496 |
| DLGDISGINASLLNIQKEIDRLNEVAKNLNESLIDLQEL | 497 |
| DLGDISGINASMMNIQKEIDRLNEVAKNLNESLIDLQEL | 498 |
| DLGDISGINASVVNIQQEIDRLNEVAKNLNESLIDLQEL | 499 |
| DLGDISGINASVVNIQEEIDRLNEVAKNLNESLIDLQEL | 500 |
| DLGDISGINASVVNIQSEIDRLNEVAKNLNESLIDLQEL | 501 |
| DLGDISGINASVVNIQNEIDRLNEVAKNLNESLIDLQEL | 502 |
| DLGDISGINASVVNIQKEIDNLNEVAKNLNESLIDLQEL | 503 |
| DLGDISGINASVVNIQKEIDQLNEVAKNLNESLIDLQEL | 504 |
| DLGDISGINASVVNIQKEIDELNEVAKNLNESLIDLQEL | 505 |
| DLGDISGINASVVNIQKEIDRLNEIAKNLNESLIDLQEL | 506 |
| DLGDISGINASVVNIQKEIDRLNEMAKNLNESLIDLQEL | 507 |
| DLGDISGINASVVNIQKEIDRLNELAKNLNESLIDLQEL | 508 |
| DLGDISGINASVVNIQKEIDRLNEAAKNLNESLIDLQEL | 509 |
| DLGDISGINASVVNIQKEIDRLNETAKNLNESLIDLQEL | 510 |
| DLGDISGINASVVNIQKEIDRLNEVSKNLNESLIDLQEL | 511 |
| DLGDISGINASVVNIQKEIDRLNEVVKNLNESLIDLQEL | 512 |
| DLGDISGINASVVNIQKEIDRLNEVCKNLNESLIDLQEL | 513 |
| DLGDISGINASVVNIQKEIDRLNEVTKNLNESLIDLQEL | 514 |
| DLGDISGINASVVNIQKEIDRLNEVGKNLNESLIDLQEL | 515 |
| DLGDISGINASVVNIQKEIDRLNELIKNLNESLIDLQEL | 516 |
| DLGDISGINASVVNIQKEIDRLNEIIKNLNESLIDLQEL | 517 |
| DLGDISGINASVVNIQKEIDRLNEISKNLNESLIDLQEL | 518 |
| DLGDISGINASVVNIQKEIDRLNEAIKNLNESLIDLQEL | 519 |
| DLGDISGINASVVNIQKEIDRLNETIKNLNESLIDLQEL | 520 |
| DLGDISGINASVVNIQKEIDRLNEVAQNLNESLIDLQEL | 521 |
| DLGDISGINASVVNIQKEIDRLNEVAENLNESLIDLQEL | 522 |
| DLGDISGINASVVNIQKEIDRLNEVASNLNESLIDLQEL | 523 |
| DLGDISGINASVVNIQKEIDRLNEVANNLNESLIDLQEL | 524 |
| DLGDISGINASVVNIQKEIDRLNEVAKNLGESLIDLQEL | 525 |
| DLGDISGINASIVNIQKEIDRLNEVIKNLNESLIDLQEL | 526 |
| CGSGSGLEQLDILSENLNKAVENLRDIEKQINVVSANIGS | 527 |
| EEEELTTLNEELSHLATLLEETVAELQALIDNIA | 528 |
| QVEELLQQLQEQIASTQAELGKLQDVRAALEAQLAA | 529 |
| DISSIAAAFVDIQEYIDHLLTVASELESALIYLIPL | 530 |
| DISSIAAAFVDISEYIEHLLTVASELNSALIFLIPI | 531 |
| DISSIAAAFVDISEYIEHLLTVASELESALIFLIPI | 532 |
| DYSWIAAATVDIETEIQILLTVASELNSALIYLIPL | 533 |
| DYSWIAAAFVDIETEIQILLQVATELNSALIFLIPI | 534 |
| DYSWIAAAFVDIETEIQILLQVATELESALIFLIPI | 535 |
| DYSWIAAAFVDIEEEIQILLQVATELNSALIFLIPL | 536 |
| DYSWIAAAFVDIEEEIQILLQVATELESALIFLIPI | 537 |
| DISSIAAAFVDIQTEIDIILTVANELNSALINLIPL | 538 |
| DYSWIAAAFVDIETEIQILLQVATELNSALIFLIPL | 539 |
| SLLAEIQQLQQEEAAALGKLQEAVGKLNELVKELLE | 540 |
| QALQQLLQQAQEQTAQLLSKIQEALGKLEEAIKELL | 541 |
| DALQQIQQLQAQQAEALGKLAAAVGELAANLAELAA | 542 |
| AALAEIEKLKAEEAAALAKLQEAVGKLAEALAELAA | 543 |
| GLLAQIKELQAQEAAALAKLAESVGKLAELLKELKE | 544 |
| LAEIAALQAQAAEALSKLAASLGKLEEYLKKLQEIL | 545 |
| AAAEQIAALQQQIAKLQAELGKLQDERAKLEAELAA | 546 |
| SAATLAQIEALQAEIGKLQAEVNQLQAQLAQLQELL | 547 |
| LLAEIEALKAEEASALAKLASSLGKLAEYLAKLKEL | 548 |
| AAAVAAQVAQLQAETAAALGKLQEAVAALQAALAAL | 549 |
| AAAAAELAAAASKLQDSLSSLASALSTAQELAKLAE | 550 |
| SAAAAEAAAWQAKLQDALAKTASAKAEAEKLKKELE | 551 |
| SAAAAEAAAYQAKLQASLSETASASSEASKLASLLS | 552 |
| DALLAAAQAQIDAALGKLQELLSEAEALAKKAAELE | 553 |
| AAAAAAAKAEELAAEQASVLGQLQDIVAATAALLAA | 554 |
| AALAAIAASQAEAAALLAKLQEAVGKTAALQAEAEA | 555 |
| GLEELQKLQEERAKAESELGKLQDVQAQLQAQLQAL | 556 |
| SLLQQLLEQAAQLQATAAALGELAAAVAALQEALAA | 557 |
| AAATVAAAQAAFAAALAKAQAAAAAAQAALAAQLAQ | 558 |
| ADVAATLAALAAQQASLAAQLAKLQKEAAELAALLA | 559 |

TABLE 2b
Additional exemplary HRC Sequences

| HRC sequences | SEQ ID NO. |
|---|---|
| SISWIPAAFVDIQEEIEIILTVAIELNSTIIYLIPL | 560 |
| DISSIAAAFVDISEEISILLTVAEELESALIYLIPL | 561 |
| SISWIPAAFVDIETEIQUILTVAIELNSTIIYLIPL | 562 |
| SISWIPAAFVDIEEEIQIILQVAIELNSTIIYLIPL | 563 |
| DISWIAAAYVDIQEYIETLLTVATELNSALIFLIPI | 564 |
| SISWIPAAFVDIQEEIEIILQVAIELNSTIIYLIPL | 565 |
| DISSIAAATVDIQEYIETLLQVATELNSALIFLIPI | 566 |
| SISWIPAAFVDIQTEIEIILTVAIELNSTIIYLIPL | 567 |
| SISWIPAAFVDIQTEIDIILQVAIELNSTHIYLIPL | 568 |
| SISWIPAAFVDIQTEIDIILTVAIELNSTIIYLIPL | 569 |
| SISWIPAAFVDIQTEIEIILQVAIELNSTIIYLIPL | 570 |
| DISSIIAATVDIQEYIDQLLIVATELNSALIYLIPI | 571 |
| DISSIIAAFVDIQEYIDTLLQVATELNSALIFLIPI | 572 |
| DISSIIAAFVDIEEHIQTLLTVATELNSALIFLIPI | 573 |
| SISWISASFVDIQTEIEIILQVAIELNSTIIYLIPL | 574 |
| DISSIAAAFVDISEEISILLTVAEELESALIYLIPI | 575 |
| DISWIAAAYVDIEEHIQTLLTVATELNSALIFLIPI | 576 |
| DVSSIEASYVDIEDEIQTLLQVAIELNSALIFLIPI | 577 |
| DISSIEASYVDIEEHIQQLLQVAIELNSALIFLIPI | 578 |
| DISWIAAAFVDIEEYIQTLLTVATELNSALIFLIPI | 579 |
| DISSIIAAFVDIQEYIDQLLIVATELNSALIFLIPI | 580 |
| DISSIEASFVDIQEYIDQLLQVAIELNSALIFLIPI | 581 |
| DISGINLSILNIQLEIEHLNEVIENLGESLIELQEL | 582 |
| DISGINLTILNIQLEIEHLNELIENLGESLIELQEL | 583 |
| DISGINLSVVNIQLEIEHLNELIENLGESLIELQEL | 584 |
| DISSIAAAFVDIQEYIDHLLTVASELESALIYLIPL | 585 |
| DISGINAibSVVNIQOrnEIEHLNEVIOrnNLNESLIDLQEL | 586 |
| DISGINAibSVVNIQOrnEIDOrnLNEVIOrnNLNESLIDLQEL | 587 |
| DISGINAibSVVNIQOrnEIEHLNEVAOrnNLGESLIELQEL | 588 |
| DISGINAibSVVNIQOrnEIDOrnLNEVAOrnNLGESLIELQEL | 589 |
| DISGINAibSVVNIQOrnEIEHLNEVIOrnNLGESLIELQEL | 590 |
| DISGINAibSVVNIQOrnEIDOrnLNEVIOrnNLGESLIELQEL | 591 |
| LGDISGINAibSVVNIQOrnEIDOrnLNEVIOrnNLNESLIDLQEL | 592 |
| DISGINAibSVVNIQOrnEIDOrnLNEVIOrnNLNESLIDLQEL | 593 |
| DISGINAibSVVNIQOrnEIDOrnLNEVIOrnNLNESLIDLQEL | 594 |
| DISGINASVVNIQOrnEIDOrnLNEVIOrnNLNESLIDLQEL | 595 |
| DISGINAibSVVNIQOrnEIDhArgLNEVIOrnNLNESLIDLQEL | 596 |
| DISGINASVVNIQOrnEIDhArgLNEVIOrnNLNESLIDLQEL | 597 |
| DISGINAibSIVNIQOrnEIDOrnLNEIIOrnNLNESLIDLQEL | 598 |

The polypeptide of the invention is preferably represented by (TE)ₘ-HRC-(TE)ₙ, each TE and HRC are as described above. Preferably, m is 0 and nis 1, and the polypeptide of the invention is represented by HRC-TE.

In some embodiments, the coronavirus is a SARS-CoV-2 variant, the polypeptide of the invention is HRC-TE, wherein the HRC sequence is an amino acid sequence that comprises up to 20, up to 15, up to 12, or preferably up to 9 amino acid mutations relative to SEQ ID NO. 1; and the TE sequence is any amino acid sequence selected from SEQ ID NO. T-T.

In additional embodiments, the coronavirus is a SARS-CoV-2 variant, the polypeptide of the invention is TE-HRC, wherein the HRC sequence is an inverted sequence as defined herein with respect to SEQ ID NO. 1; and the TE sequence is any amino acid sequence selected from SEQ ID NO. TR-TR.

In some embodiments, the coronavirus is a MERS-CoV variant. The HRC sequence comprises an amino acid that has at least 50%, at least 60%, at least 70%, at least 80%, at least 90%, at least 95%, at least 97%, or at least 99% identity to any one of SEQ ID NO. 2. In some cases, the HRC sequence is an amino acid sequence having at least 50%, at least 60%, at least 70%, at least 80%, at least 90%, at least 95%, at least 97%, or at least 99% identity to any one of SEQ ID NO. 2. In some cases, the HRC sequence is SEQ ID No. 2. The TE sequence is any TE sequence as described herein.

In additional embodiments, the coronavirus is a MERS-CoV variant, the polypeptide of the invention is TE-HRC, wherein the HRC sequence is an inverted sequence as defined herein with respect to SEQ ID NO. 2; and the TE sequence is any TE sequence as described herein.

In some embodiments, the coronavirus is a HCoV-OC43 variant. The HRC sequence comprises an amino acid that has at least 50%, at least 60%, at least 70%, at least 80%, at least 90%, at least 95%, at least 97%, or at least 99% identity to any one of SEQ ID NO. 3. In some cases, the HRC sequence is an amino acid sequence having at least 50%, at least 60%, at least 70%, at least 80%, at least 90%, at least 95%, at least 97%, or at least 99% identity to any one of SEQ ID NO. 3. In some cases, the HRC sequence is SEQ ID NO. 3. The TE sequence is any TE sequence as described herein.

In additional embodiments, the coronavirus is a HCoV-OC43 variant, the polypeptide of the invention is TE-HRC, wherein the HRC sequence is an inverted sequence as defined herein with respect to SEQ ID NO. 3; and the TE sequence is any amino acid sequence selected from any TE sequence as described herein.

In some embodiments, the paramyxovirus is measles. The HRC sequence comprises an amino acid that has at least 50%, at least 60%, at least 70%, at least 80%, at least 90%, at least 95%, at least 97%, or at least 99% identity to SEQ ID NO. 7. In some cases, the HRC sequence is an amino acid sequence having at least 50%, at least 60%, at least 70%, at least 80%, at least 90%, at least 95%, at least 97%, or at least 99% identity to SEQ ID NO. 7. In some cases, the HRC sequence is SEQ ID No. 7. The TE sequence is any TE sequence as described herein.

In additional embodiments, the paramyxovirus is measles, the polypeptide of the invention is TE-HRC, wherein the HRC sequence is an inverted sequence as defined herein with respect to SEQ ID NO. 7; and the TE sequence is any TE sequence as described herein.

In some embodiments, the paramyxovirus is Nipah. The HRC sequence comprises an amino acid that has at least 50%, at least 60%, at least 70%, at least 80%, at least 90%, at least 95%, at least 97%, or at least 99% identity to SEQ ID NO. 8. In some cases, the HRC sequence is an amino acid sequence having at least 50%, at least 60%, at least 70%, at least 80%, at least 90%, at least 95%, at least 97%, or at least 99% identity to SEQ ID NO. 8. In some cases, the HRC sequence is SEQ ID No. 8. The TE sequence is any TE sequence as described herein.

In additional embodiments, the paramyxovirus is Nipah, the polypeptide of the invention is TE-HRC, wherein the HRC sequence is an inverted sequence as defined herein with respect to SEQ ID NO. 8; and the TE sequence is any TE sequence as described herein.

In some embodiments, the paramyxovirus is HPIV3 (native). The HRC sequence comprises an amino acid that has at least 50%, at least 60%, at least 70%, at least 80%, at least 90%, at least 95%, at least 97%, or at least 99% identity to SEQ ID NO. 9. In some cases, the HRC sequence is an amino acid sequence having at least 50%, at least 60%, at least 70%, at least 80%, at least 90%, at least 95%, at least 97%, or at least 99% identity to SEQ ID NO. 9. In some cases, the HRC sequence is SEQ ID NO. 9. The TE sequence is any TE sequence as described herein.

In additional embodiments, the paramyxovirus is HPIV3 (native), the polypeptide of the invention is TE-HRC, wherein the HRC sequence is an inverted sequence as defined herein with respect to SEQ ID NO. 9; and the TE sequence is any TE sequence as described herein.

In some embodiments, the paramyxovirus is a HPIV3 variant (HPIV3_3001). The HRC sequence comprises an amino acid that has at least 50%, at least 60%, at least 70%, at least 80%, at least 90%, at least 95%, at least 97%, or at least 99% identity to SEQ ID NO. 10. In some cases, the HRC sequence is an amino acid sequence having at least 50%, at least 60%, at least 70%, at least 80%, at least 90%, at least 95%, at least 97%, or at least 99% identity to SEQ ID NO. 10. In some cases, the HRC sequence is SEQ ID NO. 10. The TE sequence is any TE sequence as described herein.

In additional embodiments, the paramyxovirus is a HPIV3 variant (HPIV3_3001), the polypeptide of the invention is TE-HRC, wherein the HRC sequence is an inverted sequence as defined herein with respect to SEQ ID NO. 10; and the TE sequence is any TE sequence as described herein.

In some embodiments, the paramyxovirus is a HPIV3 variant (HPIV3_3002). The HRC sequence comprises an amino acid that has at least 50%, at least 60%, at least 70%, at least 80%, at least 90%, at least 95%, at least 97%, or at least 99% identity to SEQ ID NO. 11. In some cases, the HRC sequence is an amino acid sequence having at least 50%, at least 60%, at least 70%, at least 80%, at least 90%, at least 95%, at least 97%, or at least 99% identity to SEQ ID NO. 11. In some cases, the HRC sequence is SEQ ID NO. 11. The TE sequence is any TE sequence as described herein.

In additional embodiments, the paramyxovirus is a HPIV3 variant (HPIV3_3002), the polypeptide of the invention is TE-HRC, wherein the HRC sequence is an inverted sequence as defined herein with respect to SEQ ID NO. 11; and the TE sequence is any TE sequence as described herein.

In some embodiments, the paramyxovirus is a HPIV3 variant (HPIV3_3003). The HRC sequence comprises an amino acid that has at least 50%, at least 60%, at least 70%, at least 80%, at least 90%, at least 95%, at least 97%, or at least 99% identity to SEQ ID NO. 12. In some cases, the HRC sequence is an amino acid sequence having at least 50%, at least 60%, at least 70%, at least 80%, at least 90%, at least 95%, at least 97%, or at least 99% identity to SEQ ID NO. 12. In some cases, the HRC sequence is SEQ ID NO. 12. The TE sequence is any TE sequence as described herein.

In additional embodiments, the paramyxovirus is a HPIV3 variant (HPIV3_3003), the polypeptide of the invention is TE-HRC, wherein the HRC sequence is an inverted sequence as defined herein with respect to SEQ ID NO. 12; and the TE sequence is any TE sequence as described herein.

In some embodiments, the polypeptide of the invention comprises an amino acid that has at least 50%, at least 60%, at least 70%, at least 80%, at least 90%, at least 95%, at least 97%, or at least 99% identity to any one of these amino acid sequences in Table 3 or Table 3a. In some embodiments, the polypeptide of the invention is an amino acid sequence having at least 50%, at least 60%, at least 70%, at least 80%, at least 90%, at least 95%, at least 97%, or at least 99% identity to any one of These amino acid sequences in Table 3. In some embodiments, the polypeptide of the invention is an amino acid sequence selected from these amino acid sequences in Table 3 or Table 3a.

TABLE 3
Exemplary polypeptide sequences of the invention

| | SEQ ID |
|---|---|
| Polypeptide Sequence | NO. |
| PDVDLGDISGINASVVNIQKEIDRLNEVAKNLNESLIDLQELGSGSGC | 599 |
| IQKEIDRLNEVAKNLNESLIDLQELGSGSGC | 600 |
| DISGINASVVNIQKPIDRLNEVAKNLNESLIDLQELGSGSGC | 601 |
| DISGINASVVNIQKPIDPLNEVAKPLNEPLIDLQELGSGSGC | 602 |
| DISGINASVVNAQKEADRANEAAKNANESLIDLQELGSGSGC | 603 |
| DISGINASVVNIQKEIDRLNEVIKNLNESLIDLQELGSGSGC | 604 |
| DISGINASVVNIQKEIARLNEVAKALNESLIDLQELGSGSGC | 605 |
| DIAAINASVANIQKEIARLNEVAKALNESLAALQALGSGSGC | 606 |
| DISGINASVVNIQKEIARLNEVIKALNESLIDLQELGSGSGC | 607 |
| DISGINASVVNIQKEIERLNKVAKNLNESLIDLQELGSGSGC | 608 |
| DISGINASVVEIQKKIEELNKKAEELNKKLIDLQELGSGSGC | 609 |
| SLDYINVTFLDLQDEMNRLQEAIKVLNQSYINLDEIGSGSGC | 610 |
| SLTQINTTLLDLTYEMLSLQQVVKALNESYIDLKELGSGSGC | 611 |
| DISGINASVVDIKKEIEHLNEIAKSLNESLIDLQELGSGSGC | 612 |
| DISGINASVVDIKKEIEHLNEIAKNLNESLIDLQELGSGSGC | 613 |
| SLDQINVTFLDLEYEMKKLEEAIKKLEESYIDLKELGSGSGC | 614 |
| DISQINASVVNIEYEIKKLEEVAKKLEESLIDLQELGSGSGC | 615 |
| SIDQINATFVDIEYEIKKLEEVAKKLEESYIDLQELGSGSGC | 616 |
| KNHTSPDVDLGDISGINASVVNIQKEIDRLNEVAKNLNESLIDLQELGSGSGC | 617 |
| DISGINASVVNIQKEIDRLNEVAKNLNESLIDLQELGGGGSC | 618 |
| DISGINASVVNIQKEIDRLNEVAKNLNESLIDLQELGGGGGGGGC | 619 |
| DISGINASVVNIQKEIDRLNEVAKNLNESLIDLQELGGGGGGC | 620 |
| DISGINASVVNIQKEIDRLNEVAKNLNESLIDLQELEAAAKC | 621 |
| DISGINASVVNIQKEIDRLNEVAKNLNESLIDLQELEAAAKEAAAKC | 622 |
| DISGINASVVNIQKEIDRLNEVAKNLNESLIDLQELAEAAAKAC | 623 |
| DISGINASVVNIQKEIDRLNEVAKNLNESLIDLQELGSGC | 624 |
| DISGINASVVNIQKEIDRLNEVAKNLNESLIDLQELC | 625 |
| DISGINASVVNIQKEIDRLNEVAKNLNESLIDLQELGSAGSAAGSGEFC | 626 |
| DISGINASVVNIQKEIDRLNEVAKNLNESLIDLQELKESGSVSSEQLAQFRSLDC | 627 |
| DISGINASVVNIQKEIDRLNEVAKNLNESLIDLQELEGKSSGSGSESKSTC | 628 |
| LEQLDILSENLNKAVENLRDIEKQINVVSANIGSIDGSGSGC | 629 |
| EAAAKEAAAKEAAAKEAAAKEAAAKEAAAKEAAAKEAAAKC | 630 |
| EAAAKEAAAKEAAAKEAAAKEAAAKEAAAKEAAAKEAAAKGSGSGC | 631 |
| PDVDLGGDISGINASVVNIQKEIDRLNEVAKNLNESLIDLQELGSGSGC | 632 |
| KNHTSPDVDLGGDISGINASVVNIQKEIDRLNEVAKNLNESLIDLQELGSGSGC | 633 |
| DLGDISGINASVVNIQKEIDRLNEVAKNLNESLIDLQELGGSGSGC | 634 |
| VVNIQKEIDRLNEVAKNLNESLIDGSGSGC | 635 |
| DISGINASVVNIQKEIDRLNEVAKNLNESLIELQELGSGSGC | 636 |
| DISGINASVVNIQKEIDRLNEVAKNLNESLIKLQELGSGSGC | 637 |
| DISGINASVVNIQKEIDRLNEVAKNLNESLIGLQELGSGSGC | 638 |
| DISGINASVVNIQKEIDRLNEVAKNLGESLIGLQELGSGSGC | 639 |
| DISGINASVVNIQKEIDRLNEVAKNLPESLIGLQELGSGSGC | 640 |
| DISGINASVVNIQKEIDRLDEVAKNLNESLIDLQELGSGSGC | 641 |
| DISGINASVVNIQKEIDRLGEVAKNLNESLIDLQELGSGSGC | 642 |
| DISGINASVENIQKEIDRLNEVAKNLNESLIDLQELGSGSGC | 643 |
| DISGINASVVNIQKEIDRLNEVAKNLGESLIELQELGSGSGC | 644 |
| DISGINASVVNIQKEIDRLDEVAKNLPESLIELQELGSGSGC | 645 |
| CGSGSGDISGINASVVNIQKEIDRLNEVAKNLNESLIDLQEL | 646 |
| IEEIQKKIEEIQKKIEEIQKKIEEIQKKGSGSGC | 647 |
| EKLDIYSENLNKAVENLIGLEQKLLQQVDLTAYSYILDEGSGSGC | 648 |
| IPNFGSLTQINTTLLDLTYEMLSLQQVVKALNESYIDLKEGSGSGC | 649 |
| DLGDISGINLTILNIQDEIEHLNELIDNLNESLIDLQELGSGC | 650 |
| DLGDISGINLTILNIQLEIEHLNELIENLGESLIELQELGSGC | 651 |
| DLGDISGINLSVVNIQLEIEHLNEVIENLGESLIELQELGSGC | 652 |
| DLGDISGINLSILNIQLEIEHLNEVIENLGESLIELQELGSGC | 653 |
| LGDISGINLSILNIQLEIEHLNEVIENLGESLIELQELGSGC | 654 |
| LGDISGINLTILNIQLEIEHLNELIENLGESLIELQELGSGC | 655 |
| DLGDISGINLSILNIQLEIEHLNEVIENLGESLIELQELGSGSGC | 656 |
| LGDISGINLSILNIQLEIEHLNEVIENLGESLIELQELGSGSGC | 657 |
| LGDISGINLSVVNIQLEIEHLNEVIENLGESLIELQELGSGSGC | 658 |
| LGDISGINLSVVNIQLEIDHLNEVIENLGESLIELQELGSGSGC | 659 |
| DLGDISGINLSVVNIQLEIDHLNEVIENLGESLIELQELGSGSGC | 660 |
| DLGDISGINLSVVNIQLEIEHLNELIENLGESLIELQELGSGSGC | 661 |

TABLE 3a
Exemplary polypeptide sequences of the invention

| Polypeptide Sequence | SEQ ID NO. |
|---|---|
| DISGINASVVNIQKEIDRLNEVIKNLGESLIELQELGC | 662 |
| LGDISGINASVVNIQKEIDRLNEVIKNLGESLIELQELGC | 663 |
| LGDISGINAibSVVNIQOrnEIDHLNEVAOrnNLNESLIDLQELGC | 664 |
| LGDISGINAibSVVNIQDabEIDHLNEVADabNLNESLIDLQELGC | 665 |
| LGDISGINAibSVVNIQDapEIDHLNEVADapNLNESLIDLQELGC | 666 |
| LGDISGINAibSVVNIQOrnEIEHLNEVAOrnNLNESLIDLQELGC | 667 |
| LGDISGINAibSVVNIQDabEIEHLNEVADabNLNESLIDLQELGC | 668 |
| LGDISGINAibSVVNIQDapEIEHLNEVADapNLNESLIDLQELGC | 669 |
| LGDISGINAibSVVNIQMlzEIEHLNEVAMlzNLNESLIDLQELGC | 670 |
| LGDISGINAibSVVNIQMlyEIEHLNEVAMlyNLNESLIDLQELGC | 671 |
| LGDISGINAibSVVNIQnme-KEIEHLNEVAOrnNLNESLIDLQELGC | 672 |
| LGDISGINAibSVVNIQnme-KEIEHLNEVADabNLNESLIDLQELGC | 673 |
| LGDISGINAibSVVNIQnme-KEIEHLNEVADapNLNESLIDLQELGC | 674 |
| LGDISGINAibSVVNIQnme-KEIEHLNEVAMlzNLNESLIDLQELGC | 675 |
| LGDISGINAibSVVNIQnme-KEIEHLNEVAMlyNLNESLIDLQELGC | 676 |
| LGDISGINAibSVVNIQOrnEIDOrnLNEVAOrnNLNESLIDLQELGC | 677 |
| LGDISGINAibSVVNIQDabEIDOrnLNEVADabNLNESLIDLQELGC | 678 |
| LGDISGINAibSVVNIQDapEIDOrnLNEVADapNLNESLIDLQELGC | 679 |
| LGDISGINAibSVVNIQMlzEIDOrnLNEVAMlzNLNESLIDLQELGC | 680 |
| LGDISGINAibSVVNIQMlyEIDOrnLNEVAMlyNLNESLIDLQELGC | 681 |
| LGDISGINAibSVVNIQnme-KEIDOrnLNEVAOrnNLNESLIDLQELGC | 682 |
| LGDISGINAibSVVNIQnme-KEIDOrnLNEVADabNLNESLIDLQELGC | 683 |
| LGDISGINAibSVVNIQnme-KEIDOrnLNEVADapNLNESLIDLQELGC | 684 |
| LGDISGINAibSVVNIQnme-KEIDOrnLNEVAMlzNLNESLIDLQELGC | 685 |
| LGDISGINAibSVVNIQnme-KEIDOrnLNEVAMlyNLNESLIDLQELGC | 686 |
| LGDISGINAibSVVNIQOrnEIDCitLNEVAOrnNLNESLIDLQELGC | 687 |
| LGDISGINAibSVVNIQDabEIDCitLNEVADabNLNESLIDLQELGC | 688 |
| LGDISGINAibSVVNIQDapEIDCitLNEVADapNLNESLIDLQELGC | 689 |
| LGDISGINAibSVVNIQMlzEIDCitLNEVAMlzNLNESLIDLQELGC | 690 |
| LGDISGINAibSVVNIQMlyEIDCitLNEVAMlyNLNESLIDLQELGC | 691 |
| LGDISGINAibSVVNIQnme-KEIDCitLNEVAOrnNLNESLIDLQELGC | 692 |
| LGDISGINAibSVVNIQnme-KEIDCitLNEVADabNLNESLIDLQELGC | 693 |
| LGDISGINAibSVVNIQnme-KEIDCitLNEVADapNLNESLIDLQELGC | 694 |
| LGDISGINAibSVVNIQnme-KEIDCitLNEVAMlzNLNESLIDLQELGC | 695 |
| LGDISGINAibSVVNIQnme-KEIDCitLNEVAMlyNLNESLIDLQELGC | 696 |
| LGDISGINAibSVVNIQOrnEIDHLNEVIOrnNLNESLIDLQELGC | 697 |
| LGDISGINAibSVVNIQDabEIDHLNEVIDabNLNESLIDLQELGC | 698 |
| LGDISGINAibSVVNIQDapEIDHLNEVIDapNLNESLIDLQELGC | 699 |
| LGDISGINAibSVVNIQOrnEIEHLNEVIOrnNLNESLIDLQELGC | 700 |
| LGDISGINAibSVVNIQDabEIEHLNEVIDabNLNESLIDLQELGC | 701 |
| LGDISGINAibSVVNIQDapEIEHLNEVIDapNLNESLIDLQELGC | 702 |
| LGDISGINAibSVVNIQMlzEIEHLNEVIMlzNLNESLIDLQELGC | 703 |
| LGDISGINAibSVVNIQMlyEIEHLNEVIMlyNLNESLIDLQELGC | 704 |
| LGDISGINAibSVVNIQnme-KEIEHLNEVIOrnNLNESLIDLQELGC | 705 |
| LGDISGINAibSVVNIQnme-KEIEHLNEVIDabNLNESLIDLQELGC | 706 |
| LGDISGINAibSVVNIQnme-KEIEHLNEVIDapNLNESLIDLQELGC | 707 |
| LGDISGINAibSVVNIQnme-KEIEHLNEVIMlzNLNESLIDLQELGC | 708 |
| LGDISGINAibSVVNIQnme-KEIEHLNEVIMlyNLNESLIDLQELGC | 709 |
| LGDISGINAibSVVNIQOrnEIDOrnLNEVIOrnNLNESLIDLQELGC | 710 |
| LGDISGINAibSVVNIQDabEIDOrnLNEVIDabNLNESLIDLQELGC | 711 |
| LGDISGINAibSVVNIQDapEIDOrnLNEVIDapNLNESLIDLQELGC | 712 |
| LGDISGINAibSVVNIQMlzEIDOrnLNEVIMlzNLNESLIDLQELGC | 713 |
| LGDISGINAibSVVNIQMlyEIDOrnLNEVIMlyNLNESLIDLQELGC | 714 |
| LGDISGINAibSVVNIQnme-KEIDOrnLNEVIOrnNLNESLIDLQELGC | 715 |
| LGDISGINAibSVVNIQnme-KEIDOrnLNEVIDabNLNESLIDLQELGC | 716 |
| LGDISGINAibSVVNIQnme-KEIDOrnLNEVIDapNLNESLIDLQELGC | 717 |
| LGDISGINAibSVVNIQnme-KEIDOrnLNEVIMlzNLNESLIDLQELGC | 718 |
| LGDISGINAibSVVNIQnme-KEIDOrnLNEVIMlyNLNESLIDLQELGC | 719 |
| LGDISGINAibSVVNIQOrnEIDCitLNEVIOrnNLNESLIDLQELGC | 720 |
| LGDISGINAibSVVNIQDabEIDCitLNEVIDabNLNESLIDLQELGC | 721 |
| LGDISGINAibSVVNIQDapEIDCitLNEVIDapNLNESLIDLQELGC | 722 |
| LGDISGINAibSVVNIQMlzEIDCitLNEVIMlzNLNESLIDLQELGC | 723 |
| LGDISGINAibSVVNIQMlyEIDCitLNEVIMlyNLNESLIDLQELGC | 724 |
| LGDISGINAibSVVNIQnme-KEIDCitLNEVIOrnNLNESLIDLQELGC | 725 |
| LGDISGINAibSVVNIQnme-KEIDCitLNEVIDabNLNESLIDLQELGC | 726 |
| LGDISGINAibSVVNIQnme-KEIDCitLNEVIDapNLNESLIDLQELGC | 727 |
| LGDISGINAibSVVNIQnme-KEIDCitLNEVIMlzNLNESLIDLQELGC | 728 |
| LGDISGINAibSVVNIQnme-KEIDCitLNEVIMlyNLNESLIDLQELGC | 729 |
| LGDISGINAibSVVNIQOrnEIDHLNEVAOrnNLGESLIELQELGC | 730 |
| LGDISGINAibSVVNIQDabEIDHLNEVADabNLGESLIELQELGC | 731 |
| LGDISGINAibSVVNIQDapEIDHLNEVADapNLGESLIELQELGC | 732 |
| LGDISGINAibSVVNIQOrnEIEHLNEVAOrnNLGESLIELQELGC | 733 |
| LGDISGINAibSVVNIQDabEIEHLNEVADabNLGESLIELQELGC | 734 |
| LGDISGINAibSVVNIQDapEIEHLNEVADapNLGESLIELQELGC | 735 |
| LGDISGINAibSVVNIQMlZEIEHLNEVAMlzNLGESLIELQELGC | 736 |
| LGDISGINAibSVVNIQMlyEIEHLNEVAMlyNLGESLIELQELGC | 737 |
| LGDISGINAibSVVNIQnme-KEIEHLNEVAOrnNLGESLIELQELGC | 738 |
| LGDISGINAibSVVNIQnme-KEIEHLNEVADabNLGESLIELQELGC | 739 |
| LGDISGINAibSVVNIQnme-KEIEHLNEVADapNLGESLIELQELGC | 740 |
| LGDISGINAibSVVNIQnme-KEIEHLNEVAMlzNLGESLIELQELGC | 741 |
| LGDISGINAibSVVNIQnme-KEIEHLNEVAMlyNLGESLIELQELGC | 742 |
| LGDISGINAibSVVNIQOrnEIDOrnLNEVAOrnNLGESLIELQELGC | 743 |
| LGDISGINAibSVVNIQDabEIDOrnLNEVADabNLGESLIELQELGC | 744 |
| LGDISGINAibSVVNIQDapEIDOrnLNEVADapNLGESLIELQELGC | 745 |
| LGDISGINAibSVVNIQMlzEIDOrnLNEVAMlzNLGESLIELQELGC | 746 |
| LGDISGINAibSVVNIQMlyEIDOrnLNEVAMlyNLGESLIELQELGC | 747 |
| LGDISGINAibSVVNIQnme-KEIDOrnLNEVAOrnNLGESLIELQELGC | 748 |
| LGDISGINAibSVVNIQnme-KEIDOrnLNEVADabNLGESLIELQELGC | 749 |
| LGDISGINAibSVVNIQnme-KEIDOrnLNEVADapNLGESLIELQELGC | 750 |
| LGDISGINAibSVVNIQnme-KEIDOrnLNEVAMlzNLGESLIELQELGC | 751 |
| LGDISGINAibSVVNIQnme-KEIDOrnLNEVAMlyNLGESLIELQELGC | 752 |
| LGDISGINAibSVVNIQOrnEIDCitLNEVAOrnNLGESLIELQELGC | 753 |
| LGDISGINAibSVVNIQDabEIDCitLNEVADabNLGESLIELQELGC | 754 |
| LGDISGINAibSVVNIQDapEIDCitLNEVADapNLGESLIELQELGC | 755 |
| LGDISGINAibSVVNIQMlZEIDCitLNEVAMlzNLGESLIELQELGC | 756 |
| LGDISGINAibSVVNIQMlyEIDCitLNEVAMlyNLGESLIELQELGC | 757 |
| LGDISGINAibSVVNIQnme-KEIDCitLNEVAOrnNLGESLIELQELGC | 758 |
| LGDISGINAibSVVNIQnme-KEIDCitLNEVADabNLGESLIELQELGC | 759 |
| LGDISGINAibSVVNIQnme-KEIDCitLNEVADapNLGESLIELQELGC | 760 |
| LGDISGINAibSVVNIQnme-KEIDCitLNEVAMlzNLGESLIELQELGC | 761 |
| LGDISGINAibSVVNIQnme-KEIDCitLNEVAMlyNLGESLIELQELGC | 762 |
| LGDISGINAibSVVNIQOrnEIDHLNEVIOrnNLGESLIELQELGC | 763 |
| LGDISGINAibSVVNIQDabEIDHLNEVIDabNLGESLIELQELGC | 764 |
| LGDISGINAibSVVNIQDapEIDHLNEVIDapNLGESLIELQELGC | 765 |
| LGDISGINAibSVVNIQOrnEIEHLNEVIOrnNLGESLIELQELGC | 766 |
| LGDISGINAibSVVNIQDabEIEHLNEVIDabNLGESLIELQELGC | 767 |
| LGDISGINAibSVVNIQDapEIEHLNEVIDapNLGESLIELQELGC | 768 |
| LGDISGINAibSVVNIQMlzEIEHLNEVIMlzNLGESLIELQELGC | 769 |
| LGDISGINAibSVVNIQMlyEIEHLNEVIMlyNLGESLIELQELGC | 770 |
| LGDISGINAibSVVNIQnme-KEIEHLNEVIOrnNLGESLIELQELGC | 771 |
| LGDISGINAibSVVNIQnme-KEIEHLNEVIDabNLGESLIELQELGC | 772 |
| LGDISGINAibSVVNIQnme-KEIEHLNEVIDapNLGESLIELQELGC | 773 |
| LGDISGINAibSVVNIQnme-KEIEHLNEVIMlzNLGESLIELQELGC | 774 |
| LGDISGINAibSVVNIQnme-KEIEHLNEVIMlyNLGESLIELQELGC | 775 |
| LGDISGINAibSVVNIQOrnEIDOrnLNEVIOrnNLGESLIELQELGC | 776 |
| LGDISGINAibSVVNIQDabEIDOrnLNEVIDabNLGESLIELQELGC | 777 |
| LGDISGINAibSVVNIQDapEIDOrnLNEVIDapNLGESLIELQELGC | 778 |
| LGDISGINAibSVVNIQMlzEIDOrnLNEVIMlzNLGESLIELQELGC | 779 |
| LGDISGINAibSVVNIQMlyEIDOrnLNEVIMlyNLGESLIELQELGC | 780 |
| LGDISGINAibSVVNIQnme-KEIDOrnLNEVIOrnNLGESLIELQELGC | 781 |
| LGDISGINAibSVVNIQnme-KEIDOrnLNEVIDabNLGESLIELQELGC | 782 |
| LGDISGINAibSVVNIQnme-KEIDOrnLNEVIDapNLGESLIELQELGC | 783 |
| LGDISGINAibSVVNIQnme-KEIDOrnLNEVIMlzNLGESLIELQELGC | 784 |
| LGDISGINAibSVVNIQnme-KEIDOrnLNEVIMlyNLGESLIELQELGC | 785 |
| LGDISGINAibSVVNIQOrnEIDCitLNEVIOrnNLGESLIELQELGC | 786 |
| LGDISGINAibSVVNIQDabEIDCitLNEVIDabNLGESLIELQELGC | 787 |
| LGDISGINAibSVVNIQDapEIDCitLNEVIDapNLGESLIELQELGC | 788 |
| LGDISGINAibSVVNIQMlzEIDCitLNEVIMlzNLGESLIELQELGC | 789 |
| LGDISGINAibSVVNIQMlyEIDCitLNEVIMlyNLGESLIELQELGC | 790 |
| LGDISGINAibSVVNIQnme-KEIDCitLNEVIOrnNLGESLIELQELGC | 791 |
| LGDISGINAibSVVNIQnme-KEIDCitLNEVIDabNLGESLIELQELGC | 792 |
| LGDISGINAibSVVNIQnme-KEIDCitLNEVIDapNLGESLIELQELGC | 793 |
| LGDISGINAibSVVNIQnme-KEIDCitLNEVIMlzNLGESLIELQELGC | 794 |
| LGDISGINAibSVVNIQnme-KEIDCitLNEVIMlyNLGESLIELQELGC | 795 |
| VDLGDISGINASVVNIQKEIDRLNEVAKNLNESLIDLQEL | 796 |
| SVVNIQKEIDRLNEVAKNLNESLIDLQELGKYEQYIK | 797 |
| GINASVVNIQKEIDRLNEVAKNLGSGSGC | 798 |
| GINASVVNIQKEIDRLNEVAKNLNESLIDLGSGSGC | 799 |
| GINASVVNIQKEIDRLNEVAKNLNESLIDLQELGKYEGSGSGC | 800 |
| ISYVEITVPNIQKEIDRLNEVAKNLNES | 801 |
| DISYVEITVPNIQKEIDRLNEVAKNLNES | 802 |
| ISYVEITVPNIQKEIDRLNEVAKNLNESLIDLQEL | 803 |
| DISYVEITVPNIQKEIDRLNEVAKNLNESLIDLQEL | 804 |
| PDVDLGDISYVEITVPNIQKEIDRLNEVAKNLNESLIDLQEL | 805 |
| PDVDLGDISYVEITVPNIQKEIDRLNEVAKNL | 806 |
| DISGINASVVNIQKEIDRLNEVAKNLNESLIDLQELG | 807 |
| PDVDLGDISGINASVVNIQKEIDRLNEVAKNL | 808 |
| DISGINASVVNIQKEIDRLNEVAENLNESLIDLQELGSGSGC | 809 |
| DISGINASVVNIEYEIDRLNEVAKNLNESLIDLQELGSGSGC | 810 |
| DISGINASVVNIQKEIDHLNEVAKNLNESLIDLQELGSGSGC | 811 |
| PDLGDISGINESVVNIQSEIDSLNELIDNLNESLIDLQELGSGSGC | 812 |
| PDLGDISGINESVVNIQSEIDSLNELIDNLNESLIDLQELGC | 813 |
| DLGDISGINESVVNIQSEIDSLNELIDNLNESLIDLQELGSGSGC | 814 |
| DLGDISGINESVVNIQSEIDSLNELIDNLNESLIDLQELGC | 815 |
| DLGDISGINQSVVNIQSEIDSLNELIDNLNESLIDLQELGC | 816 |
| DLGDISGINLSVVNIQSEIDSLNELIDNLNESLIDLQELGC | 817 |
| DLGDISGINTSVVNIQSEIDSLNELIDNLNESLIDLQELGC | 818 |
| PDLGDISGINESLLNIQSEIDSLNELIDNLNESLIDLQELGC | 819 |
| PDLGDISGINESILNIQSEIDSLNELIDNLNESLIDLQELGC | 820 |
| PDLGDISGINATILNIQSEIDSLNELIDNLNESLIDLQELGC | 821 |
| DLGDISGINATILNIQDEIDHLNELIDNLNESLIDLQELGC | 822 |
| DLGDISGINLTILNIQDEIDHLNELIDNLNESLIDLQELGSGC | 823 |
| DLGDISGINLTILNIQLEIEHLNEVIENLGESLIELQELGSGC | 824 |
| DISGINASVVNIQKEIDRLNEVIKNLGESLIELQELGSGSGC | 825 |
| DISGINASVVNIQKEIDHLNEVIKNLGESLIELQELGSGSGC | 826 |
| DISGINASVVNIQKEIEHLNEVIKNLGESLIELQELGSGSGC | 827 |
| DLGDISGINASVVNIQKEIEHLNEVIKNLGESLIELQELGSGSGC | 828 |
| DLGDISGINASVVNIQKEIEHLNEVIKNLGESLIELQELGSGC | 829 |
| DLGDISGINLSVVNIQLEIEHLNEVAENLNESLIDLQELGSGC | 830 |
| DLGDISGINLSVVNIQLEIEHLNEVAENLNESLIDLQELGC | 831 |
| LGDISGINASVVNIQKEIEHLNEVAKNLNESLIDLQELGSGSGC | 832 |
| LGDISGINASVVNIQLEIDRLNEVAKNLNESLIDLQELGSGSGC | 833 |
| LGDISGINLSVVNIQKEIDRLNEVAKNLNESLIDLQELGSGSGC | 834 |
| LGDISGINASVLNIQKEIDRLNEVAKNLNESLIDLQELGSGSGC | 835 |
| LGDISGINASIVNIQKEIDRLNEVAKNLNESLIDLQELGSGSGC | 836 |
| LGDISGINASLVNIQKEIDRLNEVAKNLNESLIDLQELGSGSGC | 837 |
| LGDISGINASILNIQKEIDRLNEVAKNLNESLIDLQELGSGSGC | 838 |
| LGDISGINASVVNIQKEIDRLNEVAENLNESLIDLQELGSGSGC | 839 |
| LGDISGINASVVNIQKEIDRLNEVAKNLNESLIDLQELGSGC | 840 |
| DLGDISGINLSVVNIQLEIEHLNEVIENLGESLIELQELGSGSGC | 841 |
| DLGDISGINLTILDIQLEIEHLNELIENLGESLIELQELGSGC | 842 |
| DLGDISGINLTILNIQLEIDHLNELIENLGESLIELQELGSGC | 843 |
| DLGDISGINLTILNIQLEINHLNELIENLGESLIELQELGSGC | 844 |
| DLGDISGINLTILNIQLEILHLNELIENLGESLIELQELGSGC | 845 |
| DLGDISGINLTILNIQLEISHLNELIENLGESLIELQELGSGC | 846 |
| DLGDISGINLTILNIQLEIEHLNELIENLGESLIDLQELGSGC | 847 |
| DLGDISGINLTILNIQLEIEHLNELIENLNESLIDLQELGSGC | 848 |
| DLGDISGINSSVVNIQKEIDRLNEVAKNLNESLIDLQELGC | 849 |
| DLGDISGINGSVVNIQKEIDRLNEVAKNLNESLIDLQELGC | 850 |
| DLGDISGINTSVVNIQKEIDRLNEVAKNLNESLIDLQELGC | 851 |
| DLGDISGINASIVNIQKEIDRLNEVAKNLNESLIDLQELGC | 852 |
| DLGDISGINASLVNIQKEIDRLNEVAKNLNESLIDLQELGC | 853 |
| DLGDISGINASMVNIQKEIDRLNEVAKNLNESLIDLQELGC | 854 |
| DLGDISGINASVINIQKEIDRLNEVAKNLNESLIDLQELGC | 855 |
| DLGDISGINASVLNIQKEIDRLNEVAKNLNESLIDLQELGC | 856 |
| DLGDISGINASVMNIQKEIDRLNEVAKNLNESLIDLQELGC | 857 |
| DLGDISGINASIINIQKEIDRLNEVAKNLNESLIDLQELGC | 858 |
| DLGDISGINASLLNIQKEIDRLNEVAKNLNESLIDLQELGC | 859 |
| DLGDISGINASMMNIQKEIDRLNEVAKNLNESLIDLQELGC | 860 |
| DLGDISGINASVVNIQQEIDRLNEVAKNLNESLIDLQELGC | 861 |
| DLGDISGINASVVNIQEEIDRLNEVAKNLNESLIDLQELGC | 862 |
| DLGDISGINASVVNIQSEIDRLNEVAKNLNESLIDLQELGC | 863 |
| DLGDISGINASVVNIQNEIDRLNEVAKNLNESLIDLQELGC | 864 |
| DLGDISGINASVVNIQKEIDNLNEVAKNLNESLIDLQELGC | 865 |
| DLGDISGINASVVNIQKEIDQLNEVAKNLNESLIDLQELGC | 866 |
| DLGDISGINASVVNIQKEIDELNEVAKNLNESLIDLQELGC | 867 |
| DLGDISGINASVVNIQKEIDRLNEIAKNLNESLIDLQELGC | 868 |
| DLGDISGINASVVNIQKEIDRLNEMAKNLNESLIDLQELGC | 869 |
| DLGDISGINASVVNIQKEIDRLNELAKNLNESLIDLQELGC | 870 |
| DLGDISGINASVVNIQKEIDRLNEAAKNLNESLIDLQELGC | 871 |
| DLGDISGINASVVNIQKEIDRLNETAKNLNESLIDLQELGC | 872 |
| DLGDISGINASVVNIQKEIDRLNEVSKNLNESLIDLQELGC | 873 |
| DLGDISGINASVVNIQKEIDRLNEVVKNLNESLIDLQELGC | 874 |
| DLGDISGINASVVNIQKEIDRLNEVCKNLNESLIDLQELGC | 875 |
| DLGDISGINASVVNIQKEIDRLNEVTKNLNESLIDLQELGC | 876 |
| DLGDISGINASVVNIQKEIDRLNEVGKNLNESLIDLQELGC | 877 |
| DLGDISGINASVVNIQKEIDRLNELIKNLNESLIDLQELGC | 878 |
| DLGDISGINASVVNIQKEIDRLNEIIKNLNESLIDLQELGC | 879 |
| DLGDISGINASVVNIQKEIDRLNEISKNLNESLIDLQELGC | 880 |
| DLGDISGINASVVNIQKEIDRLNEAIKNLNESLIDLQELGC | 881 |
| DLGDISGINASVVNIQKEIDRLNETIKNLNESLIDLQELGC | 882 |
| DLGDISGINASVVNIQKEIDRLNEVAQNLNESLIDLQELGC | 883 |
| DLGDISGINASVVNIQKEIDRLNEVAENLNESLIDLQELGC | 884 |
| DLGDISGINASVVNIQKEIDRLNEVASNLNESLIDLQELGC | 885 |
| DLGDISGINASVVNIQKEIDRLNEVANNLNESLIDLQELGC | 886 |
| DLGDISGINASVVNIQKEIDRLNEVAKNLGESLIDLQELGC | 887 |
| DLGDISGINASIVNIQKEIDRLNEVIKNLNESLIDLQELGC | 888 |
| CGSGSGLEQLDILSENLNKAVENLRDIEKQINVVSANIGSID | 889 |
| EEEELTTLNEELSHLATLLEETVAELQALIDNIAST | 890 |
| QVEELLQQLQEQIASTQAELGKLQDVRAALEAQLAALC | 891 |
| DISSIAAAFVDIQEYIDHLLTVASELESALIYLIPLGC | 892 |
| DISSIAAAFVDISEYIEHLLTVASELNSALIFLIPIGC | 893 |
| DISSIAAAFVDISEYIEHLLTVASELESALIFLIPIGC | 894 |
| DYSWIAAATVDIETEIQILLTVASELNSALIYLIPLGC | 895 |
| DYSWIAAAFVDIETEIQILLQVATELNSALIFLIPIGC | 896 |
| DYSWIAAAFVDIETEIQILLQVATELESALIFLIPIGC | 897 |
| DYSWIAAAFVDIEEEIQILLQVATELNSALIFLIPLGC | 898 |
| DYSWIAAAFVDIEEEIQILLQVATELESALIFLIPIGC | 899 |
| DISSIAAAFVDIQTEIDIILTVANELNSALINLIPLGC | 900 |
| DYSWIAAAFVDIETEIQILLQVATELNSALIFLIPLGC | 901 |
| SLLAEIQQLQQEEAAALGKLQEAVGKLNELVKELLELC | 902 |
| QALQQLLQQAQEQTAQLLSKIQEALGKLEEAIKELLEC | 903 |
| DALQQIQQLQAQQAEALGKLAAAVGELAANLAELAAIC | 904 |
| AALAEIEKLKAEEAAALAKLQEAVGKLAEALAELAAIC | 905 |
| GLLAQIKELQAQEAAALAKLAESVGKLAELLKELKEIC | 906 |
| LAEIAALQAQAAEALSKLAASLGKLEEYLKKLQEILEC | 907 |
| AAAEQIAALQQQIAKLQAELGKLQDERAKLEAELAALC | 908 |
| SAATLAQIEALQAEIGKLQAEVNQLQAQLAQLQELLQC | 909 |
| LLAEIEALKAEEASALAKLASSLGKLAEYLAKLKELLC | 910 |
| AAAVAAQVAQLQAETAAALGKLQEAVAALQAALAALQC | 911 |
| AAAAAELAAAASKLQDSLSSLASALSTAQELAKLAEEC | 912 |
| SAAAAEAAAWQAKLQDALAKTASAKAEAEKLKKELEEC | 913 |
| SAAAAEAAAYQAKLQASLSETASASSEASKLASLLSEC | 914 |
| DALLAAAQAQIDAALGKLQELLSEAEALAKKAAELEAC | 915 |
| AAAAAAAKAEELAAEQASVLGQLQDIVAATAALLAALC | 916 |
| AALAAIAASQAEAAALLAKLQEAVGKTAALQAEAEAIC | 917 |
| GLEELQKLQEERAKAESELGKLQDVQAQLQAQLQALQC | 918 |
| SLLQQLLEQAAQLQATAAALGELAAAVAALQEALAALC | 919 |
| AAATVAAAQAAFAAALAKAQAAAAAAQAALAAQLAQLC | 920 |
| ADVAATLAALAAQQASLAAQLAKLQKEAAELAALLAAC | 921 |

TABLE 3b
Exemplary polypeptide sequences of the invention

| Polypeptide Sequence | SEQ ID NO. |
|---|---|
| SISWIPAAFVDIQEEIEIILTVAIELNSTIIYLIPL | 922 |
| DISSIAAAFVDISEEISILLTVAEELESALIYLIPL | 923 |
| SISWIP AAFVDIETEIQIILTVAIELNSTIIYLIPL | 924 |
| SISWIPAAFVDIEEEIQIILQVAIELNSTIIYLIPL | 925 |
| DISWIAAAYVDIQEYIETLLTVATELNSALIFLIPI | 926 |
| SISWIPAAFVDIQEEIEIILQVAIELNSTIIYLIPL | 927 |
| DISSIAAATVDIQEYIETLLQVATELNSALIFLIPI | 928 |
| SISWIPAAFVDIQTEIEIILTVAIELNSTIIYLIPL | 929 |
| SISWIPAAFVDIQTEIDIILQVAIELNSTIIYLIPL | 930 |
| SISWIPAAFVDIQTEIDIILTVAIELNSTIIYLIPL | 931 |
| SISWIPAAFVDIQTEIEIILQVAIELNSTIIYLIPL | 932 |
| DISSIIAATVDIQEYIDQLLIVATELNSALIYLIPI | 933 |
| DISSIIAAFVDIQEYIDTLLQVATELNSALIFLIPI | 934 |
| DISSIIAAFVDIEEHIQTLLTVATELNSALIFLIPI | 935 |
| SISWISASFVDIQTEIEIILQVAIELNSTIIYLIPL | 936 |
| DISSIAAAFVDISEEISILLTVAEELESALIYLIPI | 937 |
| DISWIAAAYVDIEEHIQTLLTVATELNSALIFLIPI | 938 |
| DVSSIEASYVDIEDEIQTLLQVAIELNSALIFLIPI | 939 |
| DISSIEASYVDIEEHIQQLLQVAIELNSALIFLIPI | 940 |
| DISWIAAAFVDIEEYIQTLLTVATELNSALIFLIPI | 941 |
| DISSIIAAFVDIQEYIDQLLIVATELNSALIFLIPI | 942 |
| DISSIEASFVDIQEYIDQLLQVAIELNSALIFLIPI | 943 |
| DISGINLSILNIQLEIEHLNEVIENLGESLIELQELC | 944 |
| DISGINLTILNIQLEIEHLNELIENLGESLIELQELC | 945 |
| DISGINLSVVNIQLEIEHLNELIENLGESLIELQELC | 946 |
| DISSIAAAFVDIQEYIDHLLTVASELESALIYLIPLC | 947 |
| DISGINAibSVVNIQOrnEIEHLNEVIOrnNLNESLIDLQELC | 948 |
| DISGINAibSVVNIQOrnEIDOrnLNEVIOrnNLNESLIDLQELC | 949 |
| DISGINAibSVVNIQOrnEIEHLNEVAOrnNLGESLIELQELC | 950 |
| DISGINAibSVVNIQOrnEIDOrnLNEVAOrnNLGESLIELQELC | 951 |
| DISGINAibSVVNIQOrnEIEHLNEVIOrnNLGESLIELQELC | 952 |
| DISGINAibSVVNIQOrnEIDOrnLNEVIOrnNLGESLIELQELC | 953 |
| LGDISGINAibSVVNIQOrnEIDOrnLNEVIOrnNLNESLIDLQEL | 954 |
| DISGINASVVNIQKEIDRLNEVAKNLNESLIDLQELGSGSG | 955 |
| DISGINASVVNIQKEIDRLNEVAKNLNESLIDLQELEAAAK | 956 |
| DISGINASVVNIQKEIDRLNEVAKNLNESLIDLQELEAAAKEAAAK | 957 |
| DISGINAibSVVNIQOrnEIDOrnLNEVIOrnNLNESLIDLQEL | 958 |
| DISGINAibSVVNIQOrnEIDOrnLNEVIOrnNLNESLIDLQELGSGSG | 959 |
| DISGINASVVNIQOrnEIDOrnLNEVIOrnNLNESLIDLQEL | 960 |
| DISGINAibSVVNIQOrnEIDhArgLNEVIOrnNLNESLIDLQEL | 961 |
| DISGINASVVNIQOrnEIDhArgLNEVIOrnNLNESLIDLQEL | 962 |
| DISGINAibSIVNIQOrnEIDOrnLNEIIOrnNLNESLIDLQEL | 963 |

Furthermore, exemplary Terminal Extension (TE) sequences, Extension (EXT) sequences are also provided in the tables below.

TABLE 4
Exemplary Terminal Extension (TE) Sequences
(preferably located at the C-terminus)

| Terminal Extension Sequences | SEQ ID NO. |
|---|---|
| GSGSGC | 964 |
| GGGGSC | 965 |
| GGGGGGGGC | 966 |
| GGGGGGC | 967 |
| EAAAK | 968 |
| EAAAKC | 969 |
| EAAAKEAAAKC | 970 |
| AEAAAKAC | 971 |
| GSGC | 972 |
| GSAGSAAGSGEFC | 973 |
| KESGSVSSEQLAQFRSLDC | 974 |
| EGKSSGSGSESKSTC | 975 |
| ELGSGSGC | 976 |

TABLE 4a
Exemplary Terminal Extension (TE) Sequences
(preferably located at the N-terminus)

| Terminal Extension Sequences | SEQ ID NO. |
|---|---|
| CGSGSG, | 977 |
| CGSGSGLE, | 978 |
| CGGGGGGGG, | 979 |
| CGGGGGG, | 980 |
| KAAAE | 981 |
| CKAAAE, | 982 |
| CKAAAEKAAAE, | 983 |
| CAKAAAEA, | 984 |
| CGSG, | 985 |
| CFEGSGAASGASG, | 986 |
| CDLSRFQALQESSVSGSEK | 987 |
| CTSKSESGSGSSKGE | 988 |
| CSGGGG | 989 |

TABLE 5
Exemplary N-terminal Extension (EXT) Sequences

| N-terminal Extension (EXT) Sequences | SEQ ID NO. |
|---|---|
| DISGINASVVN | 990 |
| PDVDLGDISGINASVVN | 991 |
| DIAAINASVAN | 992 |
| DISGINASVVE | 993 |
| SLDYINVTFLD | 994 |
| SLTQINTTLLD | 995 |
| DISGINASVVD | 996 |
| SLDQINVTFLD | 997 |
| DISQINASVVN | 998 |
| SIDQINATFVD | 999 |
| KNHTSPDVDLGDISGINASVVN | 1000 |
| EAAAKEAAAKEAAAK | 1001 |
| PDVDLGGDISGINASVVN | 1002 |
| KNHTSPDVDLGGDISGINASVVN | 1003 |
| DLGDISGINASVVN | 1004 |
| VVN | 1005 |
| DISGINASVEN | 1006 |
| DLGDISGINLTILN | 1007 |
| DLGDISGINLSVVN | 1008 |
| LGDISGINLTILN | 1009 |
| DLGDISGINLSILN | 1010 |
| LGDISGINLSILN | 1011 |
| LGDISGINLSVVN | 1012 |
| DISSIAAATVD | 1013 |
| SISWIPAAFVD | 1014 |
| DISSIIAATVD | 1015 |
| SISWISASFVD | 1016 |
| DISWIAAAYVD | 1017 |
| DVSSIEASYVD | 1018 |
| DISSIEASYVD | 1019 |
| DISWIAAAFVD | 1020 |
| DISSIIAAFVD | 1021 |
| DISSIEASFVD | 1022 |
| DISGINLSILN | 1023 |
| DISGINLTILN | 1024 |
| DISGINLSVVN | 1025 |
| DISSIAAAFVD | 1026 |
| DISGINAibSVVNIQ | 1027 |
| LGDISGINAibSVVN | 1028 |
| DISGINAibSVVN | 1029 |
| DISGINAibSIVN | 1030 |

TABLE 6
Exemplary C-terminal Extension (EXT) Sequences

| C-terminal Extension | SEQ ID |
|---|---|
| (EXT) Sequences | NO. |
| LQEL | 1031 |
| LQAL | 1032 |
| LDEI | 1033 |
| LKEL | 1034 |
| AAAK | 1035 |
| LQELG | 1036 |
| IQKK | 1037 |
| ILDE | 1038 |
| DLKE | 1039 |
| LIPL | 1040 |

### HRN and HRC Sequences of Paramyxovirus

Table 7 and Table 8 provide the HRN (HR1) protein receptor sequences and native HRC (HR2) ligand sequences for Measles, Nipah and human parainfluenza virus (HPIV3), as well as three variants of the HPIV3 ligand.

TABLE 7
HR 1(Protein receptor) sequences

| | |
|---|---|
| Measles | MLNSQAIDNLRASLETTNQAIEAIRQA |
| | GQGMILAVQGVQDYINNELIPSMNQL |
| | SCDLIGQ |
| | (SEQ ID NO. 4) |
| Nipah | MKNADNINKLKSSIESTNEAVVKLQE |
| | TAEKTVYVLTALQDYINTNLVPTIDK |
| | ISCKQTEL |
| | (SEQ ID NO. 5) |
| HPIV3 | KQARSDIEKLKEAIRDTNKAVQSVQS |
| | SIGNLIVAIKSVQDYVNKEIVPSIAR |
| | LGCEAAGL |
| | (SEQ ID NO. 6) |

TABLE 8
HR2(Ligand) sequences

| | |
|---|---|
| Measles native | ISLERLDVGTNLGNAIAKLEDA |
| | KELLESSDQILRSM |
| | (SEQ ID NO. 7) |
| Nipah_native | VFTDKVDISSQISSMNQSLQQS |
| | KDYIKEAQRLLDTV |
| | (SEQ ID NO. 8) |
| HPIV3_3001 | VALDPIDISIVLNKIKSDLEES |
| | KEWIRRSNKILDSI |
| | (SEQ ID NO. 10) |
| HPIV3_3002 | VALDPIDISIVLNKIKSQLEES |
| | KWEIRRSNKILDSI |
| | (SEQ ID NO. 11) |
| HPIV3_3003 | VALDPIDFSIVLNKIKSQLEES |
| | KWEIRRSNKILDSI |
| | (SEQ ID NO. 12) |
| HPIV3_3004 | VALDPIDISIELNKAKSDLEES |
| (native) | KEWIRRSNQKLDSI |
| | (SEQ ID NO. 9) |

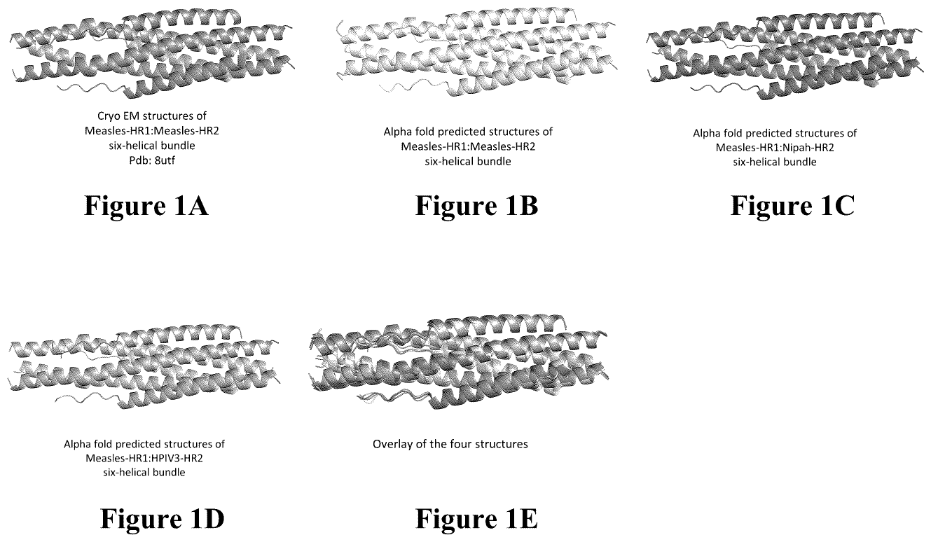

FIG. 1A through FIG. 1E show alpha fold predicted structures of viral HR1 and HR2 six helical bundles and an overlay of the bundles shown.

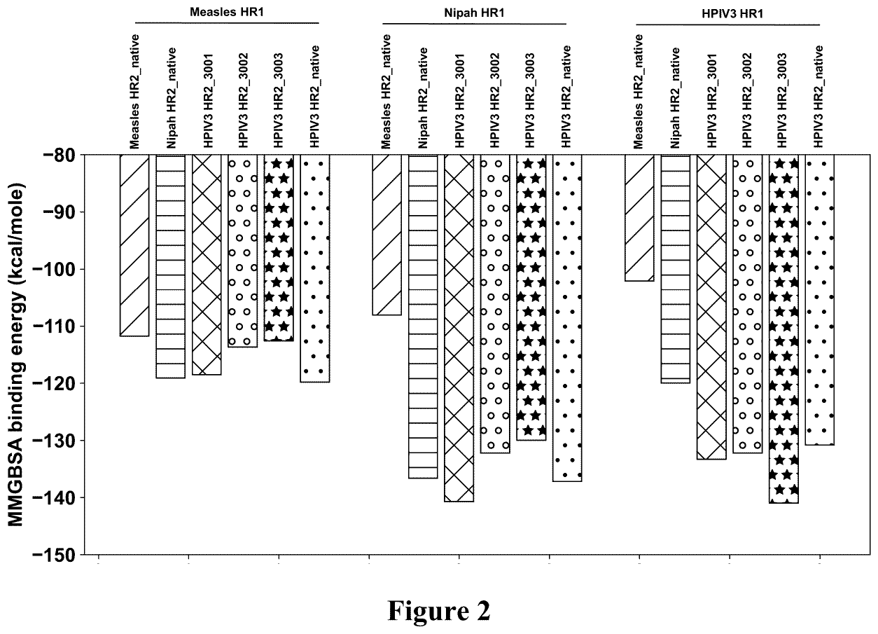

FIG. 2 shows the ligand binding (EC50 (uM)) to certain viral proteins. The ligands are conjugated to cholesterol as disclosed in PCT/US24/31869, filed on May 31, 2024.

FIG. 3 shows the MMGBSA binding energy (kcal/mole) for each conjugated ligand with HR1 proteins of measles, Nipah and HPIV3.

While this invention has been particularly shown and described with references to preferred embodiments thereof, it will be understood by those skilled in the art that various changes in form and details may be made therein without departing from the scope of the invention encompassed by the appended claims.

### Conjugate

This invention also provides a peptide conjugate that comprises the polypeptide of the invention as described above, and other moieties directly or indirectly conjugated to the polypeptide. Moieties as used in this invention include but are not limited to small molecules (e.g., drug molecules, image agents including fluorophores or radiolabels), proteins (e.g., antibodies, enzymes), polymers (e.g., PEG, polysaccharides), lipids (e.g., liposome, fatty acids), peptides, metal chelators, carbohydrates, optional linkers.

In some embodiments, the moieties include polyethylene glycol (PEG). PEG moieties can improve the pharmacokinetic properties of the polypeptide of the invention. For example, PEG can increase solubility, extend circulation time by reducing renal clearance, and decrease immunogenicity, leading to a longer duration of action and improved bioavailability.

In some embodiments, the moieties include lipids. Lipid conjugation can enhance the membrane anchoring and permeability of the polypeptide of the invention, facilitating their function on the cell surface and entry into cells.

In some embodiments, the moieties include a multivalent core moiety that is conjugated with the polypeptide of the invention and other moieties such as a lipid directly or via linkers, and that functions as a nexus for the peptide inhibitor and other moieties. In some cases, it allows multiple identical or distinctive polypeptides of the invention in one peptide conjugate to enhance the inhibition effects or impart therapeutic versatilities to the peptide conjugate.

In preferred embodiments, the moieties include a membrane-anchoring group.

In some cases, the peptide conjugate can be designed to serve as a pan-inhibitor, capable of targeting multiple related viruses or proteins across a viral family or protein class. For example, the conjugate may incorporate peptides that collectively inhibit a broad spectrum of paramyxoviruses, coronaviruses, or other viral groups by engaging conserved regions critical for viral entry or replication (preferably paramyxoviruses, or coronaviruses).

In some cases, the peptide conjugate is engineered to function as a selective inhibitor, wherein the conjugated peptides are specifically tailored to block a particular virus or biological target with high specificity. Such selective conjugates may enhance antiviral potency, improve pharmacokinetic properties, extend serum half-life, promote multivalent binding to target sites, or enable targeted delivery to specific tissues or cell types.

In some cases, the peptide conjugate comprises peptides that act as pan-paramyxovirus inhibitors, blocking a range of viruses including measles virus, mumps virus, parainfluenza (HPIV3) viruses, and Nipah virus by targeting conserved fusion machinery or other essential viral components.

In some cases, the peptide conjugate comprises peptides that act as pan-coronavirus inhibitors, capable of targeting SARS-CoV, MERS-CoV, SARS-CoV-2, and related emerging coronaviruses by interfering with conserved regions of the spike protein, preferably the HR1/HR2 domains.

In some cases, the peptide conjugate comprises selective inhibitors targeting the same virus, such as peptides that are individually selective for measles virus, mumps virus, parainfluenza virus, Nipah virus, MERS-CoV, or SARS-CoV-2, respectively. The use of highly selective peptides may reduce off-target effects and maximize therapeutic precision.

In some cases, while the individual peptides of the invention are selective for specific viruses, a peptide conjugate comprising two or more different selective peptides can function collectively as a pan-inhibitor. For example, a conjugate combining a measles virus-selective peptide with a mumps virus-selective peptide and a parainfluenza virus-selective peptide may exhibit broad-spectrum activity against the paramyxovirus family as a whole.

In some cases, the peptide conjugate comprises one or more (e.g., two, three, or four) polypeptides having at least a 70%, 80%, 90%, or 95% identity to those selected from SEQ ID NO. 1-3, 7-12, and those in Tables 3, 3a and 3b.

In some cases, the peptide conjugate comprises one or more (e.g., two, three, or four) polypeptides having at least a 96%, 97%, 98%, or 99% identity to those selected from SEQ ID NO. 1-3, 7-12, and those in Tables 3, 3a and 3b.

In some cases, the peptide conjugate comprises one or more (e.g., two, three, or four) polypeptides selected from SEQ ID NO. 1-3, 7-12, and those in Tables 3, 3a and 3b.

In some cases, the peptide conjugate comprises one or more (e.g., two, three, or four) polypeptides selected from those in Tables 3, 3a and 3b.

### Membrane-Anchoring Group

A membrane-anchoring group refers to a chemical group or component that is incorporated into the peptide conjugate and acts as an anchoring agent to anchor the peptide conjugate to cellular membrane, to facilitate targeting the fusion process of the coronavirus and inhibit viral entry.

In some embodiments, a membrane-anchoring group possesses amphipathic properties, allowing it to interact with the lipid bilayer of cell membranes. In some embodiments, a membrane-anchoring group is a naturally occurring component of the lipid bilayer of cell membranes, or a component mimicking the properties of a component of the lipid bilayer. In some embodiments, the membrane-anchoring group is hydrophobic.

In some embodiments, a membrane-anchoring group is an amino acid sequence, such as those cell membrane anchor molecules described in U.S. patent application Ser. No. 16/964,796, or those short peptide motifs as described in Bioconjugate Chem. 2014, 25, 12, 2134-2143, or those cell-penetrating peptides (CPPs) with the ability to translocate across cell membranes as described in Patel et al., “Cell-penetrating peptide sequence and modification dependent uptake and subcellular distribution of green florescent protein in different cell lines”, Sci Rep 9, 6298 (2019). https://doi.org/10.1038/s41598-019-42456-8.

Examples of member-anchoring group that can be used for this invention include but are not limited to: fatty acid or long alkyl chain or 3β-cholesterylamine or 3β-cholesterylthiol or other cholesteryl analogues and derivatives, 3β-cholesterylamine type molecule enables endosome recycling of conjugate for long cell surface anchoring half-life. It can be either in monomer or dimer or trimer or oligomer format within the conjugate. The antibody binding molecule can also be either in monomer or dimer or trimer or oligomer format within the conjugate. Examples of 3β-cholesterylamine, 3β-cholesterylamine containing moiety and their derivatives that can be used for the conjugate can be found in U.S. patent application Ser. No. 15/945,741.

In some embodiments, the membrane-anchoring group can be a lipid-based moiety including fats, waxes, steroids, cholesterol, fat-soluble vitamins, monoglycerides, diglycerides, triglycerides, phospholipids, sphingolipids, glycolipids, cationic or anionic lipids, derivatized lipids. Preferably membrane-anchoring moiety is a membrane integrating lipid including cholesterol, sphingolipid, sphingomyelin, glycolipid, glycerophospholipid (such as phosphatidylcholine, phosphatidylethanolamine and phosphatidylserine), ergosterol, 7-dihydrocholosterol and stigmasterol.

In some embodiments, the membrane-anchoring group can be cholesterol. Cholesterols can include, cholesterol, 3β-amino-5-cholestene, 3β-thiol-5-cholestene, 3β-carboxymethoxyl-5-cholestene, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate. In any embodiments wherein Membrane-anchoring moiety is cholesterol, B is preferably linked directly or indirectly to a cholesterol hydroxyl group, such as 3-OH.

In some embodiments, the membrane-anchoring group can be a phospholipid. Phospholipids that can be used in this application include, without limitation, egg phosphatidylcholine (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the I position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include dipalmitoylphosphatidylcholine (DPPC), a major constituent of naturally occurring lung surfactant. Other examples include dimyristoylphosphatidycholine (DMPC) and dimyristoylphosphatidylglycerol (DMPG) dipalmitoylphosphatideholine (DPPQ) and dipalmitoylphosphatidylglycerol (DPPG) distearoylphosphatidylcholine (DSPQ) and distearoylphosphatidylglycerol (DSPG), dioleylphosphatidyl-ethanolarnine (DOPE) and mixed phospholipids like palmitoylstearoylphosphatidyl-choline (PSPC) and palmitoylstearolphosphatidylglycerol (PSPG), and single acylated phospholipids like mono-oleoyl-phosphatidylethanolamine (MOPE).

In some embodiments, the membrane-anchoring group can be a sphingolipid, including sphingosine, sphingomyelins, cerebroside, sulfatides, globosides, gangliosides, galactocerebroside, glucocerebroside, GM2 ganglioside, GM1 ganglioside, and glycoplipids including ceramide trihexoside.

In some embodiments, the membrane-anchoring group can be a tocopherol. The tocopherols can include tocopherols, esters of tocopherols including tocopherol hemi-succinates, salts of tocopherols including tocopherol hydrogen sulfates and tocopherol sulfates.

In some preferable embodiments, the membrane-anchoring group is cholesterol.

### Multivalent Core

In some embodiments, the peptide conjugate comprises a multivalent core moiety B. The multivalent core moiety B is a multimeric core which provides a framework that covalently links the polypeptide of the invention to the membrane-anchoring moiety. The peptide conjugate of the invention therefore can be represented by:

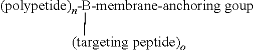

wherein the polypeptide represents the polypeptide of the invention; the targeting peptide is a peptide that targets a cell membrane receptor, for example the targeting peptide is a receptor binding domain (RBD) binding peptide or an ACE2 targeting peptide; n is 1, 2, 3, 4, 5, or 6; preferably 1, 2, or 3; o is 0, 1, 2, 3, or 4, preferable 0 or 1.

In some embodiments, the peptide conjugate of the invention is synthesized using click chemistry. Click chemistry handles are chemical moieties that provide a reactive group that can partake in a click chemistry reaction. Click chemistry reactions and suitable chemical groups for click chemistry reactions are well known to those of skill in the art, and include, but are not limited to terminal alkynes, azides, strained alkynes, dienes, dieneophiles, alkoxyamines, carbonyls, phosphines, hydrazides, thiols, and alkenes. For example, in some embodiments, an azide and an alkyne are used in a click chemistry reaction.

In some embodiments where copper-catalyzed azide-alkyne cycloaddition (CuAAC) is the click-chemistry employed for functionalizing materials as disclosed herein, the “click-chemistry compatible” compounds include a terminal alkyne and/or terminal azide functional group.

An exemplary click-chemistry reaction is CuAAC, although skilled artisans will appreciate that other click-chemistry compatible reactions that would be appreciated as equivalent to CuAAC may be employed without departing from the scope of the inventive concepts described herein. For instance, in various embodiments click-chemistry compatible reactions may include CuAAC, strain-promoted azide-alkyne cycloaddition (SPAAC), strain-promoted alkyne-nitrone cycloaddition (SPANC), strained alkene reactions such as alkene-azide cycloaddition, etc. Click-chemistry compatible reactions may also be considered to include alkene-tetrazine inverse-demand Diers-Alder reactions, alkene-tetrazole photoclick reactions, Michael additions of thiols, nucleophilic substitution of thiols with amines, and certain Diels-Alder reactions, etc. such as disclosed by Becer, et al. “Click chemistry beyond metal-catalyzed cycloaddition.” Angew. Chem. Int. Ed. 2009, 48: p. 4900-4908, and equivalents thereof as would be understood by a person having ordinary skill in the art upon reading the present disclosures.

Accordingly, click-chemistry compatible groups, compounds, etc. should be understood to include one or more suitable chemical moieties conveying capability to participate in any combination of the foregoing exemplary click chemistries, in various embodiments.

When the conjugate of this invention is synthesized using click chemistry, the polypeptide preferably comprises a cysteine (Cys or C) at the C-terminus.

In some embodiments, B comprises optionally cysteine residue, one or more X, and optionally Y, and/or optionally Z, wherein X, Y and Z are defined herein.

The optional cysteine residue, one or more X, optional Y, and optional Z can be in any order, wherein the component of B listed first is bound to the polypeptide of the invention and the component listed last is bound to the Membrane-anchoring moiety. For example, wherein B comprises, in order, one or more cysteine residue, one or more X, and Z, the polypeptide is bound to the one or more cysteine and Z is bound to the Membrane-anchoring moiety.

In some embodiments, B comprises one or more X.

In some embodiments, B comprises cysteine and X.

In some embodiments, B comprises one or more X and Z. In some embodiments, B comprises Z, and one or more X.

In some embodiments, B comprises one or more cysteine, one or more X and Z. In some embodiments, B comprises Z, one or more cysteine, and one or more X.

In some embodiments, B comprises Y, and one or more X. In some embodiments, B comprises Y, one or more X, and Z.

In some embodiments, B comprises Y, one or more cysteine, and one or more X. In some embodiments, B comprises Y, one or more cysteine, one or more X, and Z.

In some embodiments, the cysteine at the C-terminus of the polypeptide binds to B. In some embodiments, the cysteine at the C-terminus of the polypeptide is attached to one or more X via a thioether bond. In some embodiments, the cysteine at the C-terminus of the polypeptide is linked to the cysteine in B when present and the one or more cysteines in B is attached to the one or more X via a thioether bond. In some embodiments, the cysteine at the C-terminus of the polypeptide, or the one or more cysteines in B when present, have the following structure:

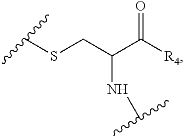

wherein R₄ is OH or NH₂, the —S— bond is covalently linked with X, the —NH— bond is covalently linked with a polypeptide (or the rest of the polypeptide when the cysteine is the cysteine at the C-terminus of the polypeptide) directly or indirectly via one or more Y.

In some embodiments, X comprises one or more sulfur aryl linkage, nitrogen aryl linkage or other linkages such as triazoles, amides, sulfur-sp3 carbon bonds, or a hydrophilic linker. In some embodiments, hydrophilic linker is selected from such as polyethyleneglycol (PEG), polyethyleneimine, polyacetal polymer, poly(l-hydroxymethylethylene hydroxymethyl-formal) (PHF) or a carbohydrate. In some embodiments, the hydrophilic linker can be polymeric and comprise 1, 2, 3, 4, 5, 6, 7, 8, 9, 10 or more monomers. In some embodiments, the hydrophilic linker is polyethyleneglycol (PEG). In some embodiments, the hydrophilic linker is polyethyleneglycol with 4 monomers (PEG4). In one embodiment, X comprises one or more sulfur aryl linkage. In one embodiment, X comprises one or more nitrogen aryl linkage. In one embodiment, X comprises one or more sulfur-sp3 carbon bonds.

In some embodiments, X is represented by —R_{A}—X₁—R_{B}—, wherein the left of R_{A} is covalently linked to cysteine via a thioether bond and the right of R_{B} is covalently linked to Membrane-anchoring moiety directly or indirectly via one or more Z; R_{A} is selected from a substituted or unsubstituted alkyl group, a substituted or unsubstituted aryl group, a substituted or unsubstituted heteroaryl group, a substituted or unsubstituted cycloalkyl group, or a substituted or unsubstituted cycloheteroalkyl group, such as a —(CH₂)₁₋₆—, a substituted or unsubstituted phenyl, a substituted or unsubstituted 5- to 8-membered cycloalkyl, a substituted or unsubstituted 5- or 6-membered cycloheteroalkyl; X₁ is —C(O)O—, —OC(O)—, —SC(O)—, —C(O)NH—, —NHC(O)—, —(O)CS—, —SONH—, —NHCONH—, —S(O)—, or —S(O)²—; R_{B} is a substituted or unsubstituted alkyl group, a —(CH₂O)₁₋₈—, —(CH₂CH₂O)₁₋₈—, —(OCH₂)₁₋₈—, or —(OCH₂CH₂)₁₋₈—.

In some embodiments, the one or more X is attached via a thioether bond directly with the membrane-anchoring moiety. In some embodiments, the one or more X is attached to Z, when present, which is then attached to the membrane-anchoring moiety.

In some embodiments, B further comprises Y.

In some embodiments, Y comprises one or more amino acids. The amino acids may be naturally occurring or synthetic. Y may comprise 1 or more amino acids, for example, 1 to 12, 1 to 6 or 1 to 4. The one or more amino acids can be added to the linker in stepwise fashion. For example, a first amino acid is added to the component of B and then, prior to the addition of a second amino acid, a polypeptide is attached to the first amino acid. After attachment of the polypeptide to the first amino acid, a subsequent amino acid is attached to the previous amino acid and allows for the attachment of a further polypeptide and so on. In some embodiments, the amino acid of the linker is one or more diamino acids, such as lysine, arginine, ornithine, diaminopimelic acid (DAP). In some embodiments, the amino acid of the linker is one or more lysine.

In some embodiments, Z, when present, comes between X and the membrane-anchoring moiety and binds B to the membrane-anchoring moiety.

In some embodiments, Z, when present, comprises a moiety having a structure according to formula (I):

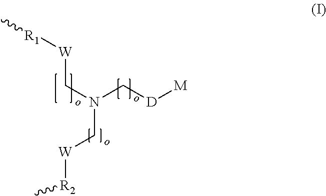

wherein each of R₁ and R₂ is independently selected from the group consisting of:
(i) absent
(ii) a structure according to formula (II):

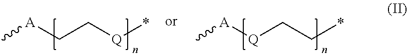

and
(iii) a structure according to formula (III):

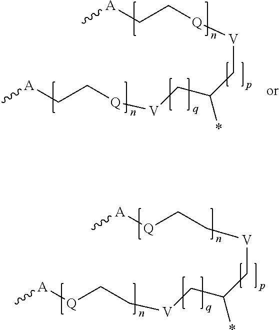

wherein [symbol] represents a bond covalently linked to X;
W is in each instance independently selected from —C(O)O—, —OC(O)—, —O—, C(O)—, (CH₂)ₘ—, and —NHC(O)—, most preferably W is —C(O)NH—;
V is in each instance independently selected from —(CH₂)ₘ—, —(CH₂)ₘ—C(O)—, —C(O)—(CH₂)ₘ—, —(CH₂)ₘ—C(O)—O—, —O—C(O)—(CH₂)ₘ—, —(CH₂)ₘ—C(O)—O—, —O—C(O)—(CH₂)ₘ—, —C(O)O—, and —OC(O)—; most preferably
V is —CH₂CH₂—C(O)NH—;
D is in each instance either —O— or —S—;
A is in each instance independently selected from —C(O)CH₂—, —CH₂C(O)—, —CH₂—, —C(O)—, —CH₂C(O)—, and —C(O)CH₂—; most preferably A is —CH₂—;
Q is in each instance independently selected from —CH₂—, —O—, —CH₂O—, and —OCH₂—; most preferably Z is —O—;
R3 is in each case independently selected from any of said polypeptides, which may be the same or different;
m is in each instance independently selected from an integer of between 0 and 5, i.e., 0, 1, 2, 3, 4, or 5; preferably between 0 and 3, preferably m is the same in each instance;
n is in each instance independently selected from an integer of between 0 and 40, i.e., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; preferably between 3 and 10, preferably n is the same in each instance;
is in each case independently selected from an integer of between 0 and 5, i.e., 0, 1, 2, 3, 4, or 5; preferably 1 or 2, preferably o is the same in each instance;
p is in each instance independently selected from an integer of between 0 and 5, i.e., 0, 1, 2, 3, 4, or 5; preferably between 0 and 3, preferably p is the same in each instance;
q is in each instance independently selected from an integer of between 0 and 5, i.e., 0, 1, 2, 3, 4, or 5; preferably between 0 and 3; preferably q is the same in each instance and/or preferably q<p;
M represents the Membrane-anchoring moiety; and
wherein * marks, where the structures (II-III) are linked to structure (I).

In some embodiments, Z, when present, comprises a moiety having a structure according to formula (IV):

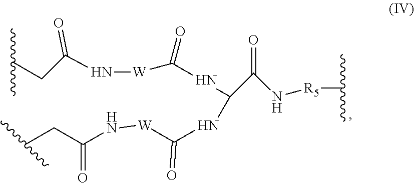

wherein R₅ is selected from hydrophilic linker such as polyethyleneglycol (PEG), polyethyleneimine, polyacetal polymer, poly(l-hydroxymethylethylene hydroxymethyl-formal) (PHF) or a carbohydrate; and
W is in each instance independently selected from direct bond, hydrophilic linker is selected from such as polyethyleneglycol (PEG), polyethyleneimine, polyacetal polymer, poly(l-hydroxymethylethylene hydroxymethyl-formal) (PHF) or a carbohydrate.

In some embodiments, the hydrophilic linker of R₅ can be polymeric and comprise 1, 2, 3, 4, 5, 6, 7, 8, 9, 10 or more monomers. In some embodiments, the hydrophilic linker of R₅ is polyethyleneglycol (PEG). In some embodiments, the hydrophilic linker of R₅ is polyethyleneglycol with 4 monomers (PEG4).

In some embodiments, the hydrophilic linker of W can be at each instance independently polymeric and comprise 1, 2, 3, 4, 5, 6, 7, 8, 9, 10 or more monomers. In some embodiments, the hydrophilic linker is polyethyleneglycol (PEG). In some embodiments, the hydrophilic linker of W can be at each instance independently polyethyleneglycol with 4 monomers (PEG4).

In some embodiments, the peptide conjugates are synthesized using automated flow chemistry, preferably using an automated flow peptide synthesis (AFPS) system. An automated flow peptide synthesis (AFPS), as disclosed in U.S. Pat. No. 10,683,325 B2 is a solid phase peptide synthesis system equipped with feedback control and can afford a high degree of control over individual coupling reactions for making peptides and/or minimize side reactions.

In any embodiments where the peptide conjugates are synthesized using automated flow chemistry, B is a multivalent moiety that is designed to allow the rapid synthesis of the peptide conjugates via automated flow chemistry and can be tailored to covalently link the Peptide, the Membrane-anchoring moiety, and other optional modules to improve anti-viral activities. B preferably comprises at least one diaminoaliphatic acids (such as diamino acids, lysine, arginine, ornithine, diaminopimelic acid, diaminopropanoic (Dap), diaminobutyric Acid (Dab)), optionally spacers, and optionally function groups such as amides, esters, and ethers. In some embodiments, B comprises 1, 2, 3, 4, 5, 6 or more diamino acids each independently selected from lysine, arginine, ornithine, and DAP. In some embodiments, B comprises 1, 2, 3, 4, 5, 6, or more lysines.

In some embodiments, B comprises one diamino acid selected from lysine, arginine, ornithine, and DAP; preferably one lysine. In some embodiments, B comprises two diamino acids each independently selected from lysine, arginine, ornithine, and DAP; preferably two lysines. In some embodiments, B comprises three diamino acids each independently selected from lysine, arginine, ornithine, and DAP; preferably three lysines. In some embodiments, B comprises four diamino acids each independently selected from lysine, arginine, ornithine, and DAP; preferably four lysines. In some embodiments, B comprises five diamino acids each independently selected from lysine, arginine, ornithine, and DAP; preferably five lysines. In some embodiments, B comprises six diamino acids each independently selected from lysine, arginine, ornithine, and DAP; preferably six lysines. When two or more diamino acids are present in B, the two or more diamino acids can be covalently linked to each other via amide bonds or via amino acid linkers such as GS linkers or polymeric linkers such as PEG linkers. In one preferable embodiment, B comprises two lysines covalently linked to each other via an amide bond. In additional preferable embodiments, B comprises one lysine. In yet additional embodiments, B comprises three lysines, covalently linked to each other via two amide bonds. In another embodiment, B comprises four lysines, covalently linked to each other via three amide bonds.

In some embodiments, when the diamino acid is lysine, B is represented by the Formula (B1):

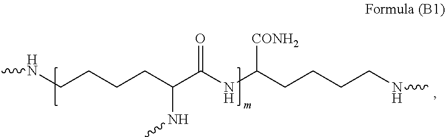

wherein [symbol] represents covalent bonds linking to the moieties of the compound that comprise one or more fusion peptide inhibitors, a membrane anchoring moiety, optionally one or more spike binding peptide, and optionally one or more targeting peptide; m is an integer that can be 0, 1, 2, 3, 4, or more. Preferably, [symbol] represents a covalent bond to —CO— group. Preferably, m is 0, 1, or 2.

In some embodiments, the peptide conjugate is represented by Formula (V):

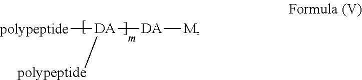

wherein polypeptide and Membrane-anchoring moiety (M) are as defined above, including all and preferable embodiments; m is 0, 1, 2, 3, 4, preferably, m is 0, 1, and 2; and each DA is independently selected from a diaminoaliphatic acid, preferably a diamino acid. In some embodiments, each Linker can be independently absent.

In some embodiments, each DA is independently selected from lysine, arginine, ornithine, and DAP. Preferably, each DA is lysine.

In some embodiments, B comprises one or more lysines, one or more spacers, and one or more additional function groups such as amides, esters, or ethers. The term “spacer”, as used herein, refers to a hydrophilic and biocompatible molecule or a chemical group that is inserted between two lysines, a lysine and a fusion peptide inhibitor, a lysine and a membrane anchoring moiety, a lysine and a spike binding peptide, or a lysine and a targeting peptide, to increase the distance between them. The spacer is used to avoid steric hindrance, reduce aggregation, improve solubility and the accessibility of the compound to the target. A common type of spacer used in the compounds is polyethylene glycol (PEG), which is a hydrophilic and biocompatible polymer that can increase the solubility and stability of the compounds in vivo. When a hydrophilic spacer PEG is present in B, a variety of PEG derivatives can be used for synthesizing the compound, such as, without limitation, amine-PEG-carboxyl acid, amine-PEG-maleimide, amine-PEG-biotin, amine-PEG-azido, azido-PEG-carboxyl acid, amine-PEG-NHS ester, maleimide-PEG-NHS ester, and biotin-PEG-NHS ester. An amine-PEG-carboxyl acid is preferably used for synthesizing the compound, preferably via automated flow chemistry, such as H₂N-PEG₁₋₄₀-COOH, H₂N-PEG₁₋₄₀-CH₂COOH, or H₂N-PEG₁₋₄₀-CH₂CH₂COOH. Other hydrophilic spacers can be used, such as polyethyleneamine, polyacetal polymer, poly(l-hydroxymethylethylene hydroxymethyl-formal) (PHF) or a carbohydrate. The length of the hydrophilic spacer can correspond to the span of the protein gap to facilitate the orientation of the HRC peptide to bind the HRN domain. B and the spacer, Linker and the spacer, or Peptide and the spacer can be joined to each other by the residue of a chemical reaction (such as an automated flow chemistry reaction).

Therefore, in some embodiments, B can be for example represented by the Formula (B2):

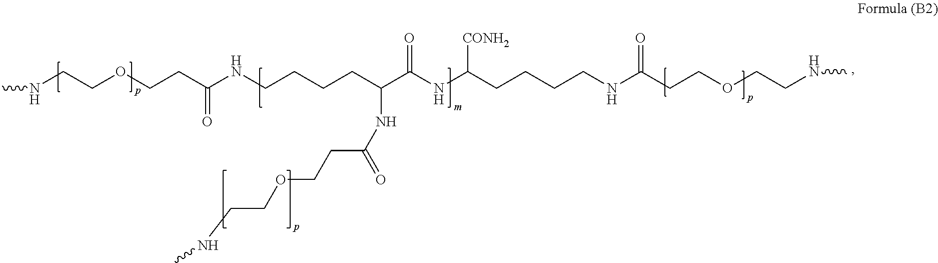

wherein [symbol] and m are as defined above, including all and preferable embodiments. p is an integer selected from 0 to 40.

When a spacer PEG is present in B, in some embodiments, the compound can be therefore represented by Formula (VI):

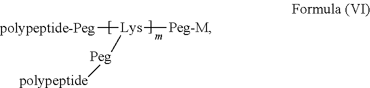

wherein polypeptide, Membrane-anchoring group (M) are as defined above, including all and preferable embodiments; m is 0, 1, 2, 3, 4, preferably, m is 0, 1, and 2; and each Peg is independently selected from a PEG spacer.

With reference to Formula (VI), Membrane-anchoring moiety is cholesterol and B is Formula (B2), resulting in the structure of the peptide conjugate as shown by Formula (VII):

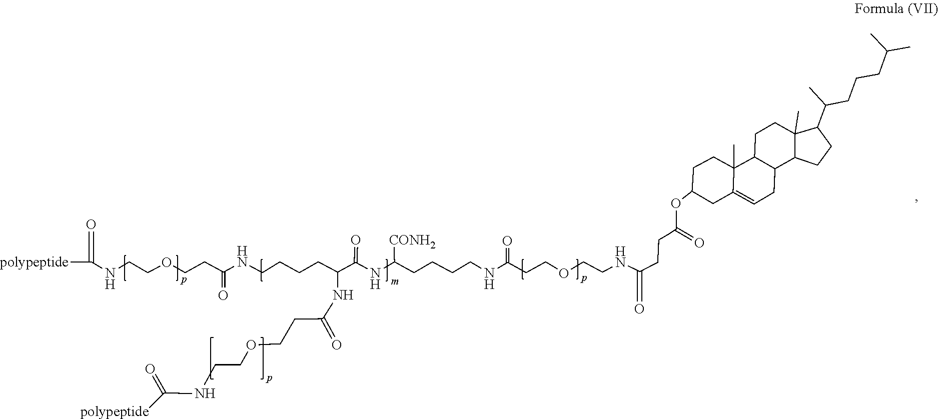

wherein polypeptide, m, and p are as defined above, including all and preferable embodiments.

### Linker

In some embodiments, the peptide conjugate further optionally comprises one or more linkers. In some cases, linkers are present between polypeptide and multivalent core B. In some cases, linkers are present between membrane-anchoring group M and multivalent core B. In some cases, linkers are present between polypeptide and multivalent core B as well as between membrane-anchoring group M and multivalent core B. In some cases, targeting peptide is present, and linker can also be present between a targeting peptide and B. In some cases, more than one polypeptide is present in the peptide conjugate, linkers are present between certain polypeptides and B whereas absent between the other polypeptides and B. Each linker is independent and can be identical to or different from each other.

Linker may comprise a non-amino acid subunit. In some embodiments, examples of the non-amino acid subunit of the linker are —(OCH₂CH₂)ₘ— where m is from 1 to 15, for example 2 to 10, 2 to 6 or 4. Introduction of a (poly)ethyleneglycol group assists solubility in aqueous media. In some embodiments, examples of the non-amino acid portion of the linker are —CH₂C(O)— and —CH₂C(O)NHCH₂CH₂(OCH₂CH₂)₄C(O)—.

For example, it can be advantageous to use a linker with 3 subunits. A first optional subunit which comprises a flexible peptide, such as -(G)ₘ- or -(GS)ₘG-, where m is an integer of 1, 2, 3, 4, 5 or more, such as 2. A second subunit can be a residue of a chemical reaction (such as an automated flow chemistry), such as a peptide bond, ester, or ether involving the N-terminus, C-terminus or side chain of the Peptide or first subunit. The residue can be non-cleavable, such as that formed with carbodiimide or sulfhydryl maleimide. A third optional subunit can be a hydrophilic spacer, a PEG spacer, such as polyethyleneglycol, polyethyleneamine, polyacetal polymer, poly(l-hydroxymethylethylene hydroxymethyl-formal) (PHF) or a carbohydrate. The hydrophilic spacers can generally be polymeric and comprise 1, 2, 3, 4, 5, 6, 7, 8, 9, 10 or more monomers. Polyethyleneglycol with 4 monomers (PEG4) is satisfactory. When the Peptide that a linker is connected to is a HRC peptide, the length of the hydrophilic spacer can correspond to the span of the protein gap to facilitate the orientation of the HRC peptide to bind the HRN domain.

Each linker can be independently designed to modify the activities of the peptide conjugate, such as flexible linkers to increase flexibility or rigid linkers to maintain a fixed distance. Chen et al. “Fusion Protein Linkers: Property, Design And Functionality”, Adv Drug Deliv Rev. 2013 65 (10): 1357-69. Preferably, when the Peptide is a HRC peptide, the Linker is selected from a flexible linker to facilitate the orientation of the HRC peptide to bind the HRN domain.

Suitable peptide linkers include polypeptides of between about 1 amino acid and about 40 amino acid residues in length, or between about 3 amino acids and about 25 amino acid residues in length. Peptide linkers with a degree of flexibility can be used. The use of small amino acids, such as glycine (G or Gly), serine (S or Ser), and alanine (A or Ala), are of use in creating a flexible peptide. A variety of different linkers are commercially available and are considered suitable for use.

Examples of each linker include glycine polymers (G)n, glycine-serine polymers (including, for example, (GS)ₙ, (GSGGS)ₙ, (GGSGGS)ₙ, (GGGS)ₙ, where n is an integer of at least one), glycine-alanine (G-A) polymers, alanine-serine (A-S) polymers. Exemplary Linker can comprise amino acid sequences including, but not limited to, GGSG, GGSGG, GSGSG, GSGGG, GGGSG, GSSSG, and the like.

Each linker is independently optional. Each linker is independently a bivalent moiety that covalently binds to a Peptide and to B. Each linker independently comprises ester, amide, disulfide, thiol, peptide, or polymeric moiety (such as polyethylene glycol (PEG)).

Each linker can independently have 1, 2, 3, 4, 5 or more subunits or segments. In some embodiments, each linker independently comprises a subunit with one or more amino acids. The amino acids may be naturally occurring or synthetic. Thus, the linker may comprise (Gly)ₙ₊₁, (GlySerGly)ₙ or (Gly-Pro)ₙ where n is 1 or greater, for example, 1 to 12, 1 to 6 or 1 to 4. GlySerGly is one example of a sequence of amino acids which may form the linker or part of the linker.

B can be covalently connected to a convenient position on the membrane-anchoring moiety. In some embodiments, connection is via a hydroxy group of the Membrane-anchoring moiety. For example, when the Membrane-anchoring moiety is cholesterol, B can be connected to the cholesterol by a group —C(O)— or —C₁₋₄ alkylene C(O)—, such as —CH₂C(O)—.

Nonlimiting examples of the peptide conjugate structure are presented in Table 4:

TABLE 9
Exemplary peptide conjugates

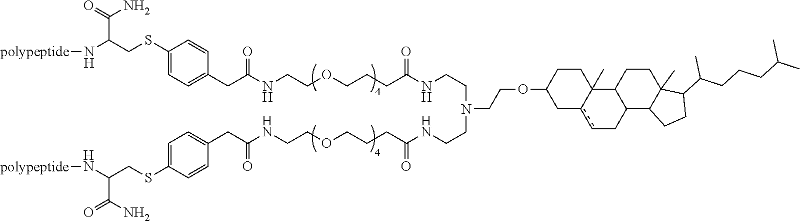

Peptide Conjugate 1

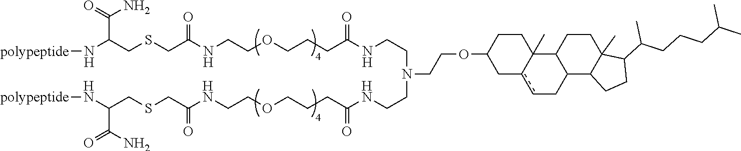

Peptide Conjugate 2

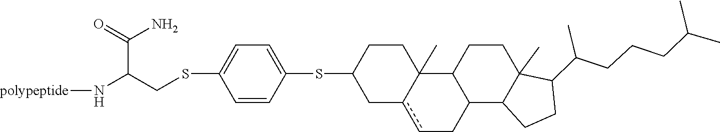

Peptide Conjugate 3

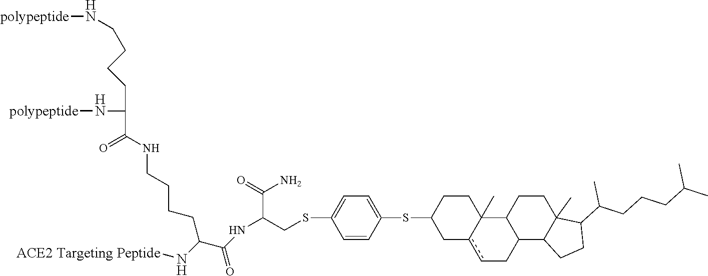

Peptide Conjugate 4

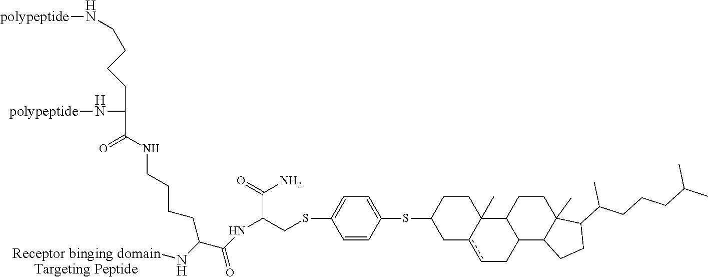

Peptide Conjugate 5

| | |
|---|---|
| 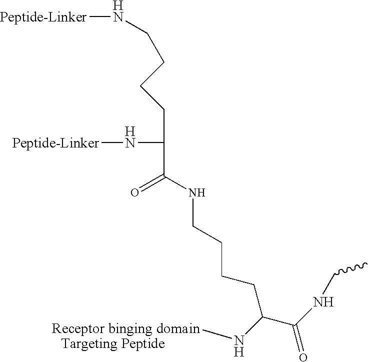 | 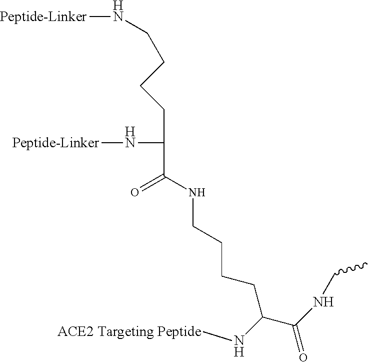 |

Peptide Conjugate 6 (left) and Peptide Conjugate 7 (right),
The NH group with [symbol] is bound to cholesterol via a linker.

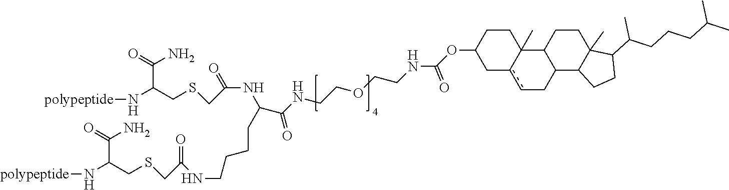

Peptide Conjugate 8

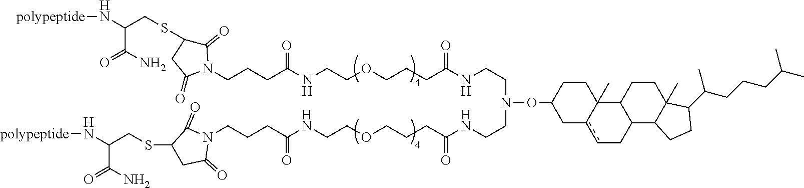

Peptide Conjugate 9

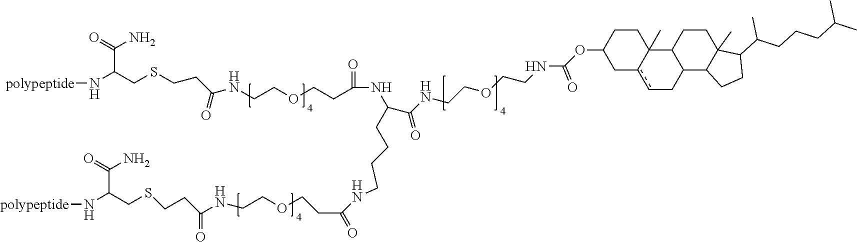

Peptide Conjugate 10

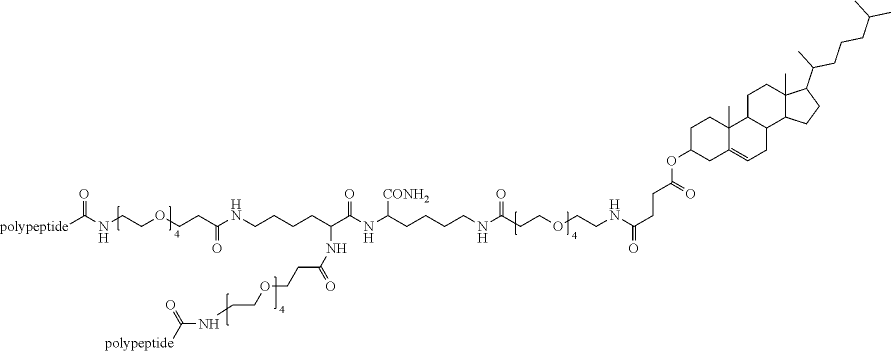

Peptide Conjugate 11

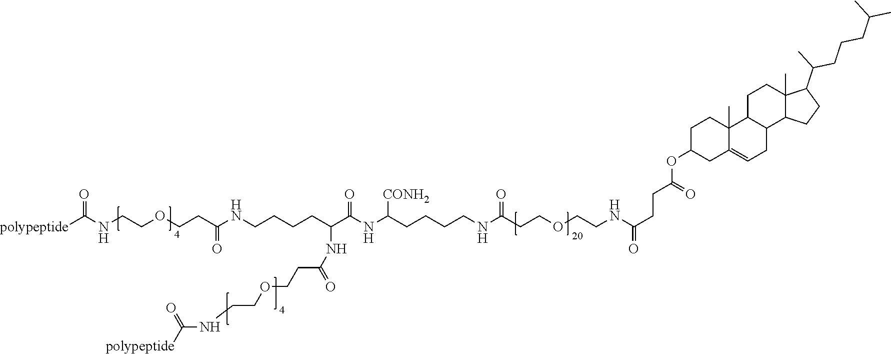

Peptide Conjugate 12

The polypeptide in any one of Peptide Conjugates 1-12 is a polypeptide of the invention as described herein.

Peptide Conjugates 1˜4 and 8-10 are examples that comprise a cysteine group:

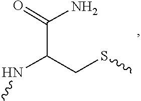

and are preferably synthesized using click chemistry. When the polypeptide is directly linked to the cysteine as shown in Peptide Conjugates 8-10, the cysteine group preferably corresponds to the cysteine at the C-terminus of the polypeptide, and therefore the polypeptide and the cysteine group together in any one of the Peptide Conjugates 8-10 correspond to any polypeptide as described herein that comprises a cysteine at C-terminus.

When the polypeptide is directly linked to the lysine as shown in Peptide Conjugates 6, 7, 11, and 12, the lysine group preferably corresponds to the lysine at the C-terminus of the polypeptide, and therefore the polypeptide and the lysine group together in any one of the Peptide Conjugates 8-10 correspond to any polypeptide as described herein that comprises a lysine at C-terminus.

Preferably, with reference to the core structures in Table 4, each polypeptide is the same for the same peptide conjugate.

Preferably, the conjugate of the invention is any one of Peptide Conjugate 8-10, and each polypeptide and cysteine group together is independently any one of These amino acid sequences in Table 3.

### Pharmaceutical Composition

In some embodiments, the compositions of the invention comprise a polypeptide as described herein and a pharmaceutically acceptable carrier. In some embodiments, the compositions of the invention comprise a peptide conjugate as described herein and a pharmaceutically acceptable carrier. For example, the composition can be administered systemically or locally. The composition can be administered for oral, intravenous, intramuscular, rectal, cutaneous, subcutaneous, topical, transdermal, sublingual, nasal, inhalation, or vaginal delivery, for example. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice (see, e.g., Remington: The Science and Practice of Pharmacy, 22^{nd} edition, 2013, ed. L. V. Allen, Pharmaceutical Press, Philadelphia, and Encyclopedia of Pharmaceutical Technology, 4.sup.th Edition, ed. J. Swarbrick, 2013, CRC Press, New York).

The polypeptides or peptide conjugates may be formulated in a variety of ways that are known in the art. For example, one or more polypeptides or peptide conjugates of the invention and any additional biologically active agent, if present, as defined herein may be formulated together or separately.

Each polypeptide or peptide conjugate of the invention, alone or in combination with one or more active agents as described herein, can be formulated for controlled release (e.g., sustained or measured) administration, as described in U.S. Patent Application Publication Nos. 2003/0152637 and 2005/0025765, each incorporated herein by reference. For example, a polypeptide or peptide conjugate of the invention, alone or in combination with one or more of the biologically active agents as described herein, can be incorporated into a capsule or tablet that is administered to the patient.

Controlled release formulations known in the art include specially coated pellets, polymer formulations or matrices for surgical insertion or as sustained release microparticles or nanoparticles, e.g., microspheres or microcapsules, for implantation, insertion, infusion or injection, wherein the slow release of the active medicament is brought about through sustained or controlled diffusion out of the matrix and/or selective breakdown of the coating of the preparation or selective breakdown of a polymer matrix. Other formulations or vehicles for controlled, sustained or immediate delivery of an agent to a preferred localized site in a patient include, e.g., lipid nanoparticles (LNP), suspensions, emulsions, gels, liposomes, and any other suitable art known delivery vehicle or formulation acceptable for subcutaneous or intramuscular administration.

Suitable biocompatible polymers can be utilized as the controlled release material. The polymeric material may comprise biocompatible, biodegradable polymers, and, in preferred embodiments, is preferably a copolymer of lactic and glycolic acid. Preferred controlled release materials which are useful in the formulations of the invention include the polyanhydrides, polyesters, co-polymers of lactic acid and glycolic acid (preferably wherein the weight ratio of lactic acid to glycolic acid is no more than 4:1 i.e., 80% or less lactic acid to 20% or more glycolic acid by weight) and polyorthoesters containing a catalyst or degradation enhancing polypeptide or peptide conjugate, for example, containing at least 1% by weight anhydride catalyst such as maleic anhydride. Examples of polyesters include polylactic acid, polyglycolic acid and polylactic acid-polyglycolic acid copolymers. Other useful polymers include protein polymers such as collagen, gelatin, fibrin and fibrinogen and polysaccharides such as hyaluronic acid.

In additional embodiments, the controlled release material, which in effect acts as a carrier for a polypeptide or peptide conjugate of the invention can further include a bioadhesive polymer such as pectins (polygalacturonic acid), mucopolysaccharides (hyaluronic acid, mucin) or non-toxic lectins or the polymer itself may be bioadhesive, e.g., polyanhydride or polysaccharides such as chitosan. In some embodiments where the biodegradable polymer comprises a gel, one such useful polymer is a thermally gelling polymer, e.g., polyethylene oxide, polypropylene oxide (PEO-PPO) block copolymer such as PLURONIC™ F127 from BASF Wyandotte.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, taste masking agents (such as hydroxypropyl methylcellulose, hydroxypropyl cellulose) and the like.

One or more polypeptides or peptide conjugates of the invention may be mixed together in a tablet, capsule, or other vehicle, or may be partitioned. In one example, a polypeptide or peptide conjugate of the invention is contained on the inside of the tablet, and the biologically active agent is on the outside of the tablet, such that a substantial portion of the biologically active agent is released prior to the release of the polypeptide or peptide conjugate of the invention.

Formulations for oral use may also be provided as chewable tablets, or as hard gelatin capsules wherein the active ingredient(s) are mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment. Formulations to the mouth may also be provided as a mouthwash, an oral spray, oral rinse solution, or oral ointment, or oral gel.

Dissolution or diffusion controlled release can be achieved by appropriate coating of a tablet, capsule, pellet, or granulate formulation of polypeptide or peptide conjugates, or by incorporating the polypeptide or peptide conjugate into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

Liquid forms in which the polypeptides or peptide conjugates and compositions of the present invention can be incorporated for administration orally include aqueous solutions, organic solution, mixed aqueous and organic solution, suitably flavored syrups, aqueous or oil suspensions, and flavored emulsions with edible oils such as cottonseed oil, sesame oil, coconut oil, or peanut oil, as well as elixirs and similar pharmaceutical vehicles.

Formulations suitable for parenteral administration (e.g., by injection), include aqueous or non-aqueous, isotonic, pyrogen-free, sterile liquids (e.g., solutions, suspensions), in which the polypeptide or peptide conjugate is dissolved, suspended, or otherwise provided (e.g., in a liposome or other microparticulate). Such liquids may additionally contain other pharmaceutically acceptable ingredients, such as anti-oxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the polypeptide or peptide conjugate in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

The composition of the invention can comprise a liquid vehicle which is suitable for nasal administration. The vehicle is preferably an aqueous solution. More preferably, the vehicle is an aqueous solution which includes a viscosity enhancing agent and, optionally one or more additional excipients which, for example, improve formulation stability and/or comfort upon administration. In some preferred embodiments, the vehicle solution is a mixed aqueous and organic solution, such as a mixture of aqueous and propylene glycol.

A variety of viscosity enhancing agents are known in the art. Viscosity enhancing agents include hydrophilic polymers, such as polysaccharides, polysaccharide derivatives, proteins and synthetic polymers. Examples include, but are not limited to, acacia, tragacanth, alginic acid, carrageenan, locust bean gum, guar gum, gelatin, hyaluronic acid, polyacrylate, polyacrylate/alkylacrylate copolymers, polyvinyl alcohol, polyvinylpyrrolidone, starch, propylene glycol alginate, maltodextrin, and cellulose ether derivatives, such as methylcellulose, hydroxyethylcellulose, hydroxypropylmethylcellulose, hydroxypropylcellulose, and carboxymethylcellulose. Where possible, salt forms of any of the foregoing are preferred. Preferred viscosity enhancing agents include hyaluronic acid, including sodium hyaluronate; carboxymethylcellulose, including sodium carboxymethylcellulose and calcium carboxymethylcellulose; methylcellulose, hydroxyethylcellulose, hydroxypropylmethylcellulose, and hydroxypropylcellulose. A composition for nasal transmucosal delivery may contain one or more different mucoadhesives in any combination. In some preferred embodiments, a mucoadhesive is selected from CMC-Na, HPMC, polyacrylamide, Carbopol, PVP, or any combination thereof. In preferred embodiments, a mucoadhesive is CMC-Na. In other preferred embodiments, a mucoadhesive is a mixture of HPMC, polyacrylamide, and Carbopol. In yet other preferred embodiments, a mucoadhesive is a mixture of HPMC, polyacrylamide, and PVP.

The composition optionally includes one or more additional excipients which, for example, increase the ease of administration, the comfort of the subject, or the stability of the composition. Suitable additional excipients include, but are not limited to, tonicity modifiers, such as sodium chloride and dextrose; antioxidants, such as butylated hydroxyanisole; buffers, such as sodium bicarbonate, sodium citrate and sodium phosphate; preservatives, such as benzalkonium chloride, ethanol, propylene glycol, benzoyl alcohol, phenethyl alcohol, chlorobutanol or methylparaben; pH adjusters, such as hydrochloric acid, sulfuric acid and sodium hydroxide; surfactants, such as Polysorbate 80, Polysorbate 20, and polyoxyl 400 stearate; chelating agents, such as disodium EDTA; antioxidants; co-solvents, such as ethanol, PEG 400, and propylene glycol; penetration enhancers, such as oleic acid; and humectants, such as glycerin (see S. Thorat, Sch. J. App. Med. Sci. 2016, 4(8D):2976-2985; D. Marx et al., IntechOpen, DOI: 10.5772/59468. Available from: intechopen.com/books/drug-discovery-and-development-from-molecules-to-medicine/intranasal-drug-administration-an-attractive-delivery-route-for-some-drugs).

In one embodiment, the vehicle consists of sodium hyaluronate, aloe vera, allantoin, sodium chloride, sodium bicarbonate, glycerin, propylene glycol, propylene glycol, benzalkonium chloride and USP grade purified water. A suitable vehicle is sold by NEILMED™ under the tradename NASOGEL™.

The amount of active agent in the composition can vary, for example, from about 0.5% by weight to about 25% by weight.

The pH of the formulation is tolerable in the nasal cavity and preferably in the range of about 5.0 to about 8.0. Buffers that can be used in the formulation include, but are not limited to phosphate, TRIS, [tris(hydroxymethyl)methylamino] propanesulfonic acid, 2-(bis(2-hydroxyethyl)amino)acetic acid, and N-[tris(hydroxymethyl)methyl]glycine, and Alkaline Buffer (Seachem).

A pharmaceutical composition suitable for nasal or pulmonary administration comprising a water soluble solvent selected from the group consisting of propylene glycol, glycerin, polyethylene glycol, and combinations thereof. The composition can further comprise one or more of a polysaccharide gum, a non-ionic surfactant, and a preservative. An exemplary polysaccharide gum is sclerotium gum. Exemplary surfactants are poloxamers, including, but not limited, to poloxamer 188. The preservative for example, can be benzalkonium chloride.

The composition can be a dry powder and delivered by a dry powder inhaler, suspended in a propellant or in an aqueous suspension or solution and delivered via a nebulizer.

For example, a solution or suspension of the active agent and a pulmonary excipient, such as lactose, can be spray dried to form particles having a fine particle fraction sufficient to deliver to the lungs or upper respiratory system. Alternatively, an aqueous solution or suspension can be sonicated, thereby aerosolizing the solution/suspension to a droplet size that can be inhaled, e.g., via a nebulizer.

Excipients include carbohydrates including monosaccharides, disaccharides and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins and maltodextrins. Other excipients suitable for use with the present invention, including amino acids, are known in the art such as those disclosed in WO 95/31479, WO 96/32096, and WO 96/32149. Mixtures of carbohydrates and amino acids are further held to be within the scope of the present invention. The inclusion of both inorganic (e.g., sodium chloride, etc.), organic acids and their salts (e.g., carboxylic acids and their salts such as sodium citrate, sodium ascorbate, magnesium gluconate, sodium gluconate, tromethamine hydrochloride, etc.) and buffers is also contemplated.

The compositions may be used in the form of dry powders or in the form of stabilized dispersions comprising a non-aqueous phase. Accordingly, the dispersions or powders of the present invention may be used in conjunction with metered dose inhalers (MDIs), dry powder inhalers (DPIs), atomizers, nebulizers or liquid dose instillation (LDI) techniques to provide for effective drug delivery. With respect to inhalation therapies, those skilled in the art will appreciate that the hollow and porous microparticles of the present invention are particularly useful in DPIs. Conventional DPIs comprise powdered formulations and devices where a predetermined dose of medicament, either alone or in a blend with lactose carrier particles, is delivered as an aerosol of dry powder for inhalation.

The medicament is formulated in a way such that it readily disperses into discrete particles with a mass median aerodynamic diameter (MMAD) of the powders will characteristically range from about 0.5-100 microns D. In some embodiments, the medicament is formulated in a way such that it readily disperses into discrete particles with a MMAD of the powders will characteristically range from about 0.5-90, 0.5-80, 0.5-70, 0.5-60, 0.5-50, 0.5-40, 0.5-30, 0.5-20, 0.5-10, or 0.5-5 microns. In some embodiments, the medicament is formulated in a way such that it readily disperses into discrete particles with a MMAD of the powders will characteristically range from about 5-100, 10-100, 20-100, 30-100, 40-100, 50-100, 60-100, 70-100, 80-100, or 90-100 microns. In some embodiments where the formulation is designed for nasal administration, the medicament is formulated in a way such that it readily disperses into discrete particles with a MMAD of the powders will preferably range from about 10-40 microns.

As discussed above, the stabilized dispersions disclosed herein may also be administered to the nasal or pulmonary air passages of a patient via aerosolization, such as with a metered dose inhaler. MDIs are well known in the art and could easily be employed for administration of the claimed dispersions without undue experimentation. Breath activated MDIs, as well as those comprising other types of improvements which have been, or will be, developed are also compatible with the stabilized dispersions and present invention and, as such, are contemplated as being within the scope thereof. However, it should be emphasized that, in preferred embodiments, the stabilized dispersions may be administered with an MDI using a number of different routes including, but not limited to, topical, nasal, pulmonary or oral. Those skilled in the art will appreciate that, such routes are well known and that the dosing and administration procedures may be easily derived for the stabilized dispersions of the present invention.

Along with the aforementioned embodiments, the stabilized dispersions of the present invention may also be used in conjunction with nebulizers as disclosed in WO 99/16420, the disclosure of which is hereby incorporated in its entirety by reference, in order to provide an aerosolized medicament that may be administered to the pulmonary air passages of a patient in need thereof. Nebulizers are well known in the art and could easily be employed for administration of the claimed dispersions without undue experimentation. Breath activated nebulizers, as well as those comprising other types of improvements which have been, or will be, developed are also compatible with the stabilized dispersions and present invention and are contemplated as being within the scope thereof.

Along with DPIs, MDIs and nebulizers, it will be appreciated that the stabilized dispersions of the present invention may be used in conjunction with liquid dose instillation or LDI techniques as disclosed in, for example, WO 99/16421 hereby incorporated in its entirety by reference. Liquid dose instillation involves the direct administration of a stabilized dispersion to the lung. In this regard, direct pulmonary administration of bioactive polypeptides or peptide conjugates is particularly effective in the treatment of disorders especially where poor vascular circulation of diseased portions of a lung reduces the effectiveness of intravenous drug delivery. With respect to LDI the stabilized dispersions are preferably used in conjunction with partial liquid ventilation or total liquid ventilation. Moreover, the present invention may further comprise introducing a therapeutically beneficial amount of a physiologically acceptable gas (such as nitric oxide or oxygen) into the pharmaceutical microdispersion prior to, during or following administration.

### Method of Use

The invention also includes methods of using the composition of the invention for treating or preventing a coronaviral infection in a subject in need thereof. The coronavirus is any coronavirus as described herein. The method comprises the step of administering an effective amount of the composition to the subject. The infection can be an infection of the gastrointestinal tract or upper or lower respiratory tract, including for example the common cold, influenza, respiratory syncytial virus infection, Severe Acute Respiratory Syndrome, COVID-19, and Middle East respiratory syndrome. In specific aspects, the methods of the invention treat a viral respiratory infection, such as a SARS-CoV-2 (COVID-19), MERS-CoV, or HCoV-OC43 respiratory infection.

The invention also includes methods of using the composition of the invention for treating or preventing a paramyxovirus infection in a subject in need thereof. The method comprises the step of administering an effective amount of the composition to the subject. The invention also includes a novel method designed to effectively prevent or reduce the transmission of a coronavirus or variant thereof (preferably a SARS-CoV-2 variant) from an infected subject to other otherwise uninfected subjects, even when close contact occurs between the infected and uninfected groups. Consequently, it offers individuals an opportunity to maintain their normal daily activities in the event of a virus infection or contact with an infected person.

This invention presents a method designed to effectively prevent or reduce the transmission of paramyxovirus from an infected subject to other otherwise uninfected subjects, even when close contact occurs between the infected and uninfected groups. Consequently, it offers individuals an opportunity to maintain their normal daily activities in the event of a virus infection or contact with an infected person.

The term “preventing or reducing transmission” as used herein refers to a variety of situations where, in contact with the infected subject, the uninfected subject remains negative for covid or paramyxovirus; the uninfected subject does not show symptoms of infection or only shows minor symptoms; the uninfected subject remains negative for covid or paramyxovirus and does not show symptoms of infection or only shows minor symptoms; and the uninfected subject does not show symptoms of infection or only shows minor symptoms not withstanding a positive test result.

The term “infected” as used herein refers to the incident wherein a subject or organism that has been exposed to the virus and has had the virus enter their body, where it can potentially multiply and cause illness. Infection with, for example SARS-CoV-2 can lead to a wide range of symptoms, from mild or asymptomatic cases to severe respiratory distress and other complications. The infection can be an infection of the gastrointestinal tract or upper or lower respiratory tract, including the common cold, influenza, respiratory syncytial virus infection, Severe Acute Respiratory Syndrome, Middle East Respiratory Syndrome, COVID-19 or a disease caused by another emerging zoonotic virus, such as a zoonotic coronavirus. In specific aspects, the methods of the invention treat a viral respiratory infection, such as a SARS-CoV-2 (COVID-19) respiratory infection.

Paramyxovirus infection can also lead to a wide range of symptoms, from mild cases to severe respiratory distress and other complications. For example, infection with measles can lead to a wide range of symptoms, such as a high fever (may spike to more than 104° F.), cough, runny nose (coryza), red, watery eyes (conjunctivitis), and a rash. Measles can cause serious health complications, especially in children younger than 5 years of age. Common complications are ear infections and diarrhea. Serious complications include pneumonia and encephalitis. In some embodiments, the methods of the invention treat, prevent, or ameliorate a measles infection.

The term “transmission” used as herein refers to the process by which the virus is passed from an infected subject to an otherwise uninfected subject, resulting in the viral infection of the otherwise uninfected subject.

The SARS-Cov-2 variant can comprise one or more mutations in the viral fusion protein with reference to the wild type. The SARS-Cov-2 variant can comprise at least 1, at least 2, at least 3, at least 4, at least 5 mutations. In some embodiments, the variant comprises at least 10 mutations. In some embodiments, the variant comprises at least 15 mutations. In some embodiments, the variant comprises at least 20 mutations.

In some embodiments, the SARS-Cov-2 variant comprises at least 5 mutations wherein the at least 5 mutations are independently in the spike protein S1 subunit or the S2 subunit or combinations thereof.

In some embodiments, the at least 5 mutations are independently in N-Terminal domain (NTD), the receptor binding domain (RBD), the fusion peptide (FP) domain, the heptad repeat 1 (HR1) domain, or combinations thereof.

In some embodiments, the at least 5 mutations are independently selected from at least 5 mutations from the SAR-Cov-2 Alpha variant; at least 5 mutations from the SAR-Cov-2 Beta variant; at least 5 mutations from the SAR-Cov-2 Delta variant; or at least 5 mutations from the SAR-Cov-2 Omicron variant. In some embodiments, the at least 5 mutations are independently selected from at least 5 mutations from the SAR-Cov-2 Alpha variant. In some embodiments, the at least 5 mutations are independently selected from at least 5 mutations from the SAR-Cov-2 Beta variant. In some embodiments, the at least 5 mutations are independently selected from at least 5 mutations from the SAR-Cov-2 Delta variant. In some embodiments, the at least 5 mutations are independently selected from at least 5 mutations from the SAR-Cov-2 Omicron variant.

In some embodiments, the SARS-CoV-2 variant comprises at least one variant selected from B.1.1.7 (Alpha), B.1.351 (Beta), P.1 (Gamma), B.1.617.2 (Delta), B.1.429/B.1.427 (Epsilon), B.1.617.1 (Kappa), B.1.525 (Eta), B.1.526 (Iota), P.3 (Theta), P.2 (Zeta), and B.1.1.529 (Omicron).

In some embodiments, the SARS-CoV-2 variant comprises at least one variant selected from A.1-A.6, B.3-B.7, B.9, B.10, B.13-B.16, B.2, B.1 lineage, P.1, P.2, P.3, and R.1.

In some embodiments, the B.1 lineage comprises at least one of (including, but not limited to, B.1, B.1.1, B.1.1.7, B.1.1.7 with E484K, B.1.2, B.1.5-B.1.72, B.1.9, B.1.13, B.1.22, B.1.26, B.1.37, B.1.3-B.1.66, B.1.177, B.1.243, B.1.313, B.1.351, B.1.427, B.1.429, B.1.525, B.1.526, B.1.526.1, B.1.526.2, B.1.617, B.1.617.1, B.1.617.2, B.1.617.3, B.1.619, B.1.620, and B.1.621.

In some embodiments, the coronavirus is a MERS-CoV variant.

In some embodiments, the coronavirus is a HCoV-OC43 variant.

In some embodiments, the administration is achieved via nasal administration such as nasal spray, nasal drops, nasal gels, nasal powders, nasal aerosols, nasal pumps, nasal nebulizers, nasal inhalers. Preferably the nasal administration is achieved using an intranasal spray, an inhaler, or a nebulizer.

In some embodiments, the peptide conjugate is administered in combination with at least one other antiviral active agent or therapy.

The infected subject, preferably a human, can be an individual diagnosed with the infection and is either symptomatic, pre-symptomatic, or asymptomatic, or at risk for developing infection. For example, the subject can be at risk for developing the viral respiratory infection due to direct or indirect exposure or possible exposure to the virus (such as SARS-CoV-2 or a mutant thereof or a paramyxovirus), such as via exposure to an infected individual or a virus-contaminated fomite. The subject can be a resident of, or a visitor to, a community in which the viral respiratory infection has been identified, for example, the subject can be a family member of an infected individual or the subject can work in a health care setting caring for infected individuals. In some embodiments, the subject at risk for infection is asymptomatic and has tested negative for presence of the virus prior to the commencement of therapy. In specific examples, the subject can be at risk for developing COVID-19 due to exposure to the SARS-CoV-2 virus, for example, from the respiratory droplets or aerosols of an infected individual and/or contact with a contaminated fomite. In yet further aspects, the subject is suffering from COVID-19 including subjects suffering from mild, moderate, or severe COVID-19. In yet further aspects, the subject is suffering from paramyxovirus, for example, measles.

In some embodiments of the method of the invention, the infected subject suffers from another disease or condition, such as chronic obstructive pulmonary disease (COPD) or ulcerative colitis, which can be exacerbated by an infection.

The peptide conjugate is administered to the infected subject upon the discovery of the infection, before infection as a prophylactic measure, or within 72 hours of the discovery of the infection. The peptide conjugate is preferably administered to the infected subject before the infected subject comes into contact with other uninfected subjects. The peptide conjugate is preferably administered to the infected subject within 48 hours, 36 hours, 24 hours, 12 hours, or 8 hours of the discovery of the infection, or 48 hours, 36 hours, 24 hours, 12 hours, or 8 hours before the infected subject comes into contact with other uninfected subjects. Preferably the administration continues until the infected subject test negative for the coronavirus or a paramyxovirus, such as measles. Contact may involve sharing a residence, workplace, classroom, car or any enclosed area. The term “enclosed area” as used herein refers to any space that is enclosed or substantially enclosed by physical barriers, such as walls, fences, doors, or other structures. Enclosed areas can vary widely in size and purpose, ranging from small rooms or compartments to large buildings and structures.

In additional embodiments, the method further comprises an optional step of administering an effective amount of peptide conjugate to the other uninfected subject(s) before the uninfected subject(s) come into contact with the infected subject. In some cases, the method further comprises an optional step of administering an effective amount of peptide conjugate to the other uninfected subject(s) 48 hours, 36 hours, 24 hours, 12 hours, 8 hours before the uninfected subject(s) come into contact with the infected subject. In some cases, the administration to the uninfected subject(s) continues throughout the duration of physical contact with the infected subject or until the infected subject tests negative, whichever occurs first.

In alternative embodiments, provided is also a method for preventing or reducing the transmission of a coronavirus or variant thereof (preferably a SARS-CoV-2 variant) from an infected subject to other otherwise uninfected subjects when in contact with the infected subject, comprising administrating an effective amount of the peptide conjugate as described herein to the uninfected subject. The peptide conjugate is preferably administered to the uninfected subject 48 hours, 36 hours, 24 hours, 12 hours, 8 hours before the uninfected subject(s) come into contact with the infected subject. Preferably the administration continues throughout the duration of physical contact with the infected subject or until the infected subject tests negative, whichever occurs first.

In alternative embodiments, provided is also a method for preventing or reducing the transmission of paramyxovirus from an infected subject to other otherwise uninfected subjects when in contact with the infected subject, comprising administrating an effective amount of the peptide conjugate as described herein to the uninfected subject. The peptide conjugate is preferably administered to the uninfected subject 48 hours, 36 hours, 24 hours, 12 hours, 8 hours before the uninfected subject(s) come into contact with the infected subject. Preferably the administration continues throughout the duration of physical contact with the infected subject or until the infected subject tests negative, whichever occurs first.

The peptide conjugate is preferably administered in a pharmaceutical composition to the infected subject before the infected subject is symptomatic (e.g., pre-symptomatic), at the onset of symptoms, or within 24 hours of the onset of symptoms. The pharmaceutical composition can be administered at a variety of dosing schedules. For example, the pharmaceutical composition can be administered one or more times and over a course of one or more days. In some embodiments, the pharmaceutical composition is administered one or more times per day for one to 10 days. In some embodiments, the pharmaceutical composition is administered one or more times per day until the subject is asymptomatic and/or testing for the virus is negative.

The pharmaceutical composition can be administered to the nasal passages using routine methods and devices (see D. Marx et al., IntechOpen, DOI: 10.5772/59468. Available on the world wide web at intechopen.com/books/drug-discovery-and-development-from-molecules-to-medicine/intranasal-drug-administration-an-attractive-delivery-route-for-some-drugs). For example, the pharmaceutical composition can be administered to the nasal passages as drops or as an aerosol spray, for example, using an aerosol bottle or a multi-dose spray pump, which can provide a uniform metered dose. The volume per dose can be varied, but is typically from about 50 to about 150 μl. The desired volume will depend on the desired dose of the active agent and the concentration of the active agent in the composition.

Where delivery to the pulmonary system, or lungs, is desired it can be efficacious to aerosolize a low concentration solution of the active agent for an extended period, such as overnight.

In addition to the prevention or reduction of viral transmission from the infected subject to other uninfected subject(s), the method as described above also has a therapeutic effect on the treatment of the infected subject.

Provided is also a method of treating infection associated with a SARS-Cov-2 variant in a subject in need, wherein the method comprises administering an effective amount of the peptide conjugate as described herein.

Provided is also a method of treating infection associated with paramyxovirus in a subject in need, wherein the method comprises administering an effective amount of the peptide conjugate as described herein.

In any of the methods described herein, the paramyxovirus includes but is not limited to the measles, the mumps, the human parainfluenza virus (HPIV) or Nipah. Preferably, the paramyxovirus is measles. Preferably, the paramyxovirus is mumps. Preferably, the paramyxovirus is the human parainfluenza virus (HPIV). Preferably, the paramyxovirus is Nipah.

### Combination Therapies

The peptide conjugate or composition described herein can be co-administered with other active agents and therapies.

In some embodiments, the other active agent includes, but is not limited to, antibodies against SARS-CoV-2. Suitable antibodies are described in, for example, US 2022/0017604, US 2022/0017614; US 2021/0403550, US 2021/0395345, US 2021/0403537; US 2021/0388066, US 2021/0388065, US 2021/0347859, or US 2021/0309733, which are incorporated herein by reference. In some embodiments, the antibody is a monoclonal antibody such as casirivimab, imdevimab, bamlanivimab, or etesevimab. In some embodiments, the antibody is a monoclonal antibody therapy such as casirivimab plus imdevimab, bamlanivimab, or bamlanivimab plus etesevimab.

In some embodiments, the other active agent includes, but is not limited to, the measles, mumps, and rubella vaccine (MMR vaccine).

The active agents and compositions of the present invention are also intended for use with general care provided patients with viral infections, including parenteral fluids (including dextrose saline and Ringer's lactate) and nutrition, antibiotic (including metronidazole and cephalosporin antibiotics, such as ceftriaxone and cefuroxime) and/or antiviral prophylaxis, fever (e.g., acetaminophen) and pain medication, antiemetic (such as metoclopramide) and/or antidiarrheal agents, vitamin and mineral supplements (including Vitamin K and zinc sulfate), anti-inflammatory agents (such as ibuprofen), pain medications, and medications for other common diseases in the patient population, such as artemether, artesunate-lumefantrine combination therapy), quinolone antibiotics, such as ciprofloxacin, macrolide antibiotics, such as azithromycin, cephalosporin antibiotics, such as ceftriaxone, or aminopenicillins, such as ampicillin), or shigellosis.

The combination therapy may be administered as a simultaneous or sequential regimen. When administered sequentially, the combination may be administered in two or more administrations.

Co-administration of a peptide conjugate of the invention with one or more other active therapeutic agents generally refers to simultaneous or sequential administration of a peptide conjugate of the invention and one or more other active therapeutic agents, such that therapeutically effective amounts of the peptide conjugate of the invention and one or more other active therapeutic agents are both present in the body of the patient.

Co-administration includes administration of unit dosages of the peptide conjugates of the invention before or after administration of unit dosages of one or more other active therapeutic agents, for example, administration of the peptide conjugates of the invention within seconds, minutes, or hours of the administration of one or more other active therapeutic agents and/or as part of the same treatment regimen. For example, a unit dose of a peptide conjugate of the invention can be administered first, followed within seconds or minutes or days by administration of a unit dose of one or more other active therapeutic agents. Alternatively, a unit dose of one or more other therapeutic agents can be administered first, followed by administration of a unit dose of a peptide conjugate of the invention within seconds or minutes or days. In some cases, it may be desirable to administer a unit dose of a peptide conjugate of the invention first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a peptide conjugate of the invention.

The combination therapy may provide “synergy” and “synergistic,” i.e., the effect achieved when the active ingredients used together is greater than the sum of the effects that results from using the peptide conjugates separately.

As used herein, the words “a” and “an” are meant to include one or more unless otherwise specified. For example, the term “an agent” encompasses both a single agent and a combination of two or more agents.

The term “treating” or “treatment” as used herein covers the treatment of the disease or condition of interest (e.g., a respiratory infection) in a mammal, preferably a human, having the disease or condition of interest, and includes, for example: preventing or delaying the onset of the disease or condition from occurring in a mammal, in particular, when such mammal is at risk of developing the disease but has not yet become symptomatic and/or been diagnosed as having it; inhibiting the disease or condition, i.e., arresting its development; relieving the disease or condition, i.e., causing regression of the disease or condition; and/or stabilizing the disease or condition. Treatment includes ameliorating or lessening the severity of symptoms of the disease or condition, and/or inhibition of further progression or worsening of those symptoms. Treatment also includes shortening the time course and/or severity of a disease or condition compared to the expected or historical time course and/or severity of the disease.

As used herein the terms “preventing,” means causing the clinical symptoms of a disease or condition not to develop and includes inhibiting the onset of a viral infection in a subject that may be exposed to or predisposed to the viral infection but does not yet experience or display symptoms of the infection.

An “effective amount” or a “therapeutically effective amount” of a peptide conjugate or composition described herein refers to an amount of the peptide conjugate that is sufficient to achieve a specific effect or result, and/or prevents or treats the disease or condition and/or the symptoms therefore, for example, alleviating, in whole or in part, symptoms associated with the disorder or condition, or halts or slows further progression or worsening of those symptoms, or prevents or provides prophylaxis for the disorder or condition. The “effective amount” and “therapeutically effective amount” includes specifically an anti-viral amount of a peptide conjugate of the invention (alone or in combination with another active agent) or the composition described herein.

The term “polypeptide” as used herein refers to a polypeptide having no more than about 100 amino acids, or preferably, no more than about 80 amino acids. As the polypeptide of the invention is a coronaviral inhibitor that targets the fusion process, in some embodiments, “the polypeptide of the invention,” “peptide inhibitor,” “peptide fusion inhibitor,” “fusion peptide inhibitor,” or “fusion inhibitor” are used interchangeably.

The term “conjugate” or “peptide conjugate” are used interchangeably herein. Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

### EXAMPLES

Neutralizing activity against SARS-CoV-2 pseudovirus was measured using a single-round infection assay in 293T/ACE2 target cells. For each variant tested, pseudotyped virus particles were produced in 293T/17 cells (ATCC) by co-transfection of plasmids encoding codon-optimized SARS-CoV-2 full-length Spike, packaging plasmid, and luciferase reporter plasmid. For neutralization assays, serial dilutions of peptides were performed in duplicate followed by addition of pseudovirus. Pooled serum samples from convalescent COVID-19 patients or PBS/10% DMSO diluent buffer were used as positive and negative controls, respectively. Plates were incubated for 1 hour at 37° C. followed by addition of 293/ACE2 target cells (1×10⁴/well). Wells containing cells+pseudovirus (without sample) or cells alone acted as positive and negative infection controls, respectively. Assays were harvested on day 3 using Promega BrightGlo luciferase reagent and luminescence detected with a Promega GloMax luminometer. Titers are reported as the concentration of peptide that inhibited 50% or 80% virus infection (IC₅₀ and IC₈₀ titers, respectively). All neutralization experiments were repeated twice with similar results.

(SEQ ID NO. 1042)
DISGINASVVNIQKEIDRLNEVAKNLNESLIDLQELGSGSGC

TABLE 10
Neutralization Assay, WT D614G (IC50/IC80, μM),
JN.1 (IC50/IC80, μM) for Polypeptides

| polypeptide | MONOMER Neutralization | MONOMER Neutralization |
|---|---|---|
| sequence/ | Assay, WT D614G (IC50/ | Assay, JN.1 (IC50/ |
| SEQ ID NO. | IC80, μM) | IC80, μM) |
| 1042 | >10/>10 | >10/>10 |
| 602 | >10/>10 | >10/>10 |
| 604 | >10/>10 | 5.1/>10 |
| 605 | >10/>10 | >10/>10 |
| 607 | >10/>10 | >10/>10 |
| 608 | >10/>10 | >10/>10 |
| 612 | >10/>10 | >10/>10 |
| 613 | >10/>10 | >10/>10 |
| 617 | >10/>10 | >10/>10 |
| 621 | >10/>10 | >10/>10 |
| 625 | >10/>10 | >10/>10 |
| 630 | >10/>10 | >10/>10 |
| 632 | >10/>10 | >10/>10 |
| 633 | >10/>10 | >10/>10 |
| 634 | >10/>10 | >10/>10 |
| 636 | >10/>10 | >10/>10 |
| 637 | >10/>10 | >10/>10 |
| 638 | >10/>10 | >10/>10 |
| 639 | >10/>10 | >10/>10 |
| 640 | >10/>10 | >10/>10 |
| 641 | >10/>10 | >10/>10 |
| 642 | >10/>10 | >10/>10 |
| 644 | 2.4/9.9 | 2.7/8.9 |

TABLE 11
Neutralization Assay, WT D614G (IC50/IC80, μM), JN.1
(IC50/IC80, μM), KP.3 (IC50/IC80, μM) for Peptide
Conjugate (the Peptide Conjugate is represented by the
structure of Peptide Conjugate 10 in Table 9, comprising
the polypeptide sequences, respectively, as listed in Table 11)

| | CONJUGATE | CONJUGATE | CONJUGATE |
|---|---|---|---|
| polypeptide | Neutralization | Neutralization | Neutralization |
| sequence/ | Assay, WT D614G | Assay, JN.1 | Assay, KP.3 |
| SEQ ID NO. | (IC50/IC80, μM) | (IC50/IC80, μM) | (IC50/IC80, μM) |
| 949 | 0.276/2.116 | 0.156/0.808 | 0.169/0.614 |
| 952 | 0.604/4.588 | 0.192/1.006 | 0.247/1.339 |
| 953 | 0.762/7.966 | 0.456/2.116 | 0.427/2.544 |

Table 12 shows the ligand binding (EC₅₀ (μM)) to certain viral proteins. The ligands were conjugated to cholesterol as disclosed in PCT/US24/31869, filed on May 31, 2024, represented by the structure of Peptide Conjugate 12.

TABLE 12

| | | Activity of one-shot product | | |
|---|---|---|---|---|
| | | EC₅₀ (μM) | | |
| HR2 Source | | RSV_{A2} | RSV_{B18537} | hPIV_{C243} |
| HPIV3 | 3001 | <0.04 | 0.04 | 6.5 |
| | (SEQ ID NO. 10) | | | |
| | 3002 | 0.17 | 0.87 | 0.5 |
| | (SEQ ID NO. 11) | | | |
| | 3003 | 0.2 | 1.55 | 0.05 |
| | (SEQ ID NO. 12) | | | |
| | 3004 Native | 0.05 | <0.04 | >5.29 |
| | (SEQ ID NO. 9) | | | |

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It will also be understood that none of the embodiments described herein are mutually exclusive and may be combined in various ways without departing from the scope of the invention encompassed by the appended claims.

1. A polypeptide having 20 to 80 amino acid residues in length that is modified from a C-terminus heptad repeat (HRC) region of a viral fusion protein (such as coronaviral spike fusion protein, or a paramyxovirus fusion protein), represented by: 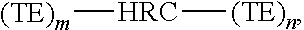
wherein m and n each are independently selected from 0 and 1;
HRC represents a HRC peptide sequence having 20 to 60 amino acid residues in length; and
TE represents a terminal extension sequence having 1-30 amino acid residues in length;
wherein the terminal extension (TE) sequence comprises a cysteine (Cys or C) or lysine (Lys or K) at the terminus.

2. The polypeptide of claim 1, wherein the TE sequence further comprises one or more amino acids selected from threonine (Thr or T), lysine (lys or K), glutamate (Glu or E), serine (Ser or S), glycine (Gly or G), valine (Val or V), glutamine (Gln or Q), leucine (Leu or L), alanine (Ala or A), phenylalanine (Phe or F), arginine (Arg or R), and aspartate (Asp or D).

3. The polypeptide of claim 1, wherein the TE sequence has 1 to 10 amino acid residues in length, and the amino acids in addition to C at the terminus are independently selected from G, S, E, L, A, V, and K, preferably G and S.

4. The polypeptide of claim 1, wherein the TE amino acid sequence is selected from:
C,
(GS)ₙ-GC,
(GSG)ₙ-C,
(G)ₙ-C,
(S)ₘ-(GGS)ₙ-C,
(S)ₘ-(GGGS)ₙ-C,
(S)ₘ-(GGGGS)ₙ-C,
(A)ₘ-(EAAAK)ₙ-(A)ₘ-C,
(EAAAK)ₙ-(GS)ₘ-GC,
(SA)ₙ-C,
(EL)ₙ-(GS)ₙ-GC,
(GSA)ₙ-(AG)ₘ-(SG)ₘ-(EF)ₘ-C,
and
an inverted sequence thereof,
wherein each n is independently selected from 1, 2, 3, 4, 5, and 6, and each m is independently selected from 0, 1, 2, 3, 4, 5, and 6.

5. The polypeptide of claim 1, wherein the TE sequence is located at the C-terminus of the polypeptide, and the TE sequence is an amino acid sequence that has at least 70%, 80%, 90%, 95%, 96%, 97%, 98%, or 99% identity to any one of the amino acid sequences with SEQ ID NO. 964-976 (Table 4).

6. The polypeptide of claim 1, wherein the TE sequence is located at the N-terminus of the polypeptide, and the TE sequence is an amino acid sequence that has at least 70%, 80%, 90%, 95%, 96%, 97%, 98%, or 99% identity to any one of the amino acid sequences with SEQ ID NO. 977-989 (Table 4a).

7. The polypeptide of claim 1, wherein the polypeptide is represented by HRC-TE, wherein TE is an amino acid sequence selected from amino acid sequences with SEQ ID NO. 964-976 (Table 4).

8. The polypeptide of claim 1, wherein the HRC peptide is represented by: 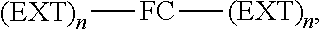
wherein each n is independently 0 or 1; each EXT independently represents an extension amino acid sequence; and FC represents a modified fusion core sequence.

9. The polypeptide of claim 8, wherein the HRC peptide is a SARS-Cov-2 HRC peptide, the FC is an amino acid sequence that has up to 15 amino acid mutations relative to IQKEIDRLNEVAKNLNESLID (SEQ ID NO. 13), an amino acid sequence that is a truncated sequence with respect to IQKEIDRLNEVAKNLNESLID (SEQ ID NO. 13), or an amino acid sequence that has up to 15 amino acid mutations relative to DILSENLNKAVENLRDIEKQI.

10. The polypeptide of claim 8, wherein the FC sequence is an amino acid sequence that has 1, 2, 3, 4, 5, 6, 7, 8, 9, or 10 amino acid mutations relative to IQKEIDRLNEVAKNLNESLID (SEQ ID NO. 13), and those amino acid mutations are independently located at the positions selected from Xaa1-Xaa21, wherein the positions of Xaa1-Xaa20 are represented by: Xaa1-Xaa2-Xaa3-Xaa4-Xaa5-Xaa6-Xaa7-Xaa8-Xaa9-Xaa10-Xaa11-Xaa12-Xaa13-Xaa14-Xaa15-Xaa16-Xaa17-Xaa18-Xaa19-Xaa20-Xaa21;
wherein, when Xaa1 is mutated, Xaa1 is A;
when Xaa2 is mutated, Xaa2 is K or E;
when Xaa3 is mutated, Xaa3 is Y, D, L, Orn, Dab, Dap, Mlz, Mly, or nme-K;
when Xaa4 is mutated, Xaa4 is P or K;
when Xaa5 is mutated, Xaa5 is A;
when Xaa6 is mutated, Xaa6 is selected from A, E, and K;
when Xaa7 is mutated, Xaa7 is selected from P, E, H, K, S, A, L, Orn, and Cit; preferably, P, E, S, H, K, Orn, and Cit;
when Xaa8 is mutated, Xaa8 is A;
when Xaa9 is mutated, Xaa9 is selected from E, D, or G;
when Xaa10 is mutated, Xaa10 is K;
when Xaa11 is mutated, Xaa11 is selected from K or I;
when Xaa12 is mutated, Xaa12 is I;
when Xaa13 is mutated, Xaa13 is D, E, Orn, Dab, Dap, Mlz, Mly, or nme-K;
when Xaa14 is mutated, Xaa14 is selected from P, A, E, S, and K;
when Xaa15 is mutated, Xaa15 is A;
when Xaa16 is mutated, Xaa16 is E;
when Xaa17 is mutated, Xaa17 is K;
when Xaa18 is mutated, Xaa18 is P or K;
when Xaa19 is mutated, Xaa19 is Y;
when Xaa20 is mutated, Xaa20 is A; and
when Xaa21 is mutated, Xaa21 is A, E, K, or G.

11. The polypeptide of claim 8, wherein the FC sequence is an amino acid sequence that has 1, 2, 3, 4, 5, 6, or 7 amino acid mutations relative to IQKEIDRLNEVAKNLNESLID (SEQ ID NO. 13), and the FC sequence is selected from:
Xaa1-QK-Xaa4-Xaa5-D-Xaa7-
LNEVAK-Xaa14-Xaa15-NESLID;
and
IQ-Xaa3-EI-Xaa6-Xaa7-LNEV-
Xaa12-Xaa13-NL-Xaa16-ESLI-
Xaa21.

12. The polypeptide of claim 8, wherein the FC sequence is an amino acid sequence that comprises at least one amino acid mutations relative to IQKEIDRLNEVAKNLNESLID (SEQ ID NO. 13), and the at least one amino acid mutation is A to I at Xaa12, N to G at Xaa16, or D to E at Xaa21; preferably the at least one amino acid mutation is A to I at Xaa12.

13. The polypeptide of claim 8, wherein the FC sequence is an amino acid sequence that comprises at least two amino acid mutations relative to IQKEIDRLNEVAKNLNESLID (SEQ ID NO. 13), and the at least two amino acid mutations are N to G at Xaa16 and D to E at Xaa21.

14. The polypeptide of claim 8, wherein the FC sequence is an amino acid sequence that comprises at least three amino acid mutations relative to IQKEIDRLNEVAKNLNESLID (SEQ ID NO. 13), and the at least three amino acid mutations are A to I at Xaa12, N to G at Xaa16, and D to E at Xaa21.

15. The polypeptide of claim 8, wherein the FC sequence is an amino acid represented by: Xaa1-Xaa2-Xaa3-Xaa4-Xaa5-Xaa6-Xaa7-Xaa8-Xaa9-Xaa10-Xaa11-Xaa12-Xaa13-Xaa14-Xaa15-Xaa16-Xaa17-Xaa18-Xaa19-Xaa20-Xaa21;
Xaa1 is I, A or L, preferably I.
Xaa2 is Q, T, K or E.
Xaa3 is K, Y, D, L, Orn, Dab, Dap, Mlz, Mly, or nme-K.
Xaa4 is E, P, A, K, T, or Y; preferably E, P or K.
Xaa5 is I, A, M, K, G, or E; preferably I, A or M.
Xaa6 is selected from D, A, E, K, L, M, and N; preferably D, A, E, K, L, and N.
Xaa7 is selected from R, P, E, H, K, S, A, L, Orn, and Cit; preferably, R, P, E, S, H, K, Orn, and Cit.
Xaa8 is L, A, or I.
Xaa9 is selected from N, E, D, G or Q; preferably N, E, D, or G.
Xaa10 is E, K, or Q; preferably E or K.
Xaa11 is selected from V, A, K, L, and I.
Xaa12 is A, I or V; preferably A or I.
Xaa13 is selected from K, D, E, Orn, Dab, Dap, Mlz, Mly, or nme-K.
Xaa14 is selected from N, P, A, E, V, S, and K; preferably Xaa14 is N.
Xaa15 is L or A; preferably L.
Xaa16 is N, E, G, P, or Q; preferably N or G.
Xaa17 is E, K, or Q; preferably E.
Xaa18 is S, P, or K; preferably S.
Xaa19 is L, I, or Y; preferably L.
Xaa20 is selected from I, A, and E; preferably, A.
Xaa21 is selected from D, A, E, K, N, and G; preferably D, A, E, K, and G; more preferably, D and E.

16. The polypeptide of claim 8, wherein the FC is an amino acid sequence that has at least 70%, 80%, 90%, 95%, 96%, 97%, 98%, or 99% identity to any one of the amino acid sequences with SEQ ID NO. 13-253.

17. The polypeptide of claim 8, wherein the EXT sequence at the N-terminus is absent or is an amino acid sequence having at least 50%, at least 60%, at least 70%, at least 80%, at least 90%, 95%, 96%, 97%, 98%, or 99% identity to any one of the amino acid sequences with SEQ ID NO. 990-1030; and wherein the EXT sequence at the C-terminus is absent or is an amino acid sequence having at least 50%, at least 60%, at least 70%, at least 80%, at least 90%, 95%, 96%, 97%, 98%, or 99% identity to any one of the amino acid sequences with SEQ ID NO. 1030-1040.

18. The polypeptide of claim 1, wherein the HRC peptide comprises an amino acid sequence that has up to 25 amino acid mutations (preferably up to 20, 10, 5, 4, 3, 2, amino acid mutations) relative to
(SEQ ID NO. 1)
DISGINASVVNIQKEIDRLNEVAKNLNESLIDLQEL.

19-24. (canceled)

25. The polypeptide of claim 1, wherein the HRC peptide is an amino acid sequence that has at least 70%, 80%, 90%, 95%, 96%, 97%, 98%, or 99% identity to any one of the amino acid sequences with SEQ ID NO. 1-3, 7-12, and 254-598 (Tables 2, 2a, and 2b).

26. The polypeptide of claim 1, wherein the polypeptide is an amino acid sequence that has at least 70%, 80%, 90%, 95%, 96%, 97%, 98%, or 99% identity to any one of the amino acid sequences with SEQ ID NO. 599-963 (Tables 3, 3a, and 3b).

27-28. (canceled)

29. A peptide conjugate that comprises a polypeptide of claim 1.

30. The peptide conjugate of claim 29, wherein the peptide conjugate comprises an animo acid sequence that has at least 90%, 95%, 97%, 98%, or 99% identity to any one of SEQ ID NO. 599-963, SEQ ID NO. 254-598, or SEQ ID NO. 15-253 (preferably, any one of SEQ ID NO. 599-963).

31. The peptide conjugate of claim 29, wherein the peptide conjugate comprises a moiety that is selected from a small molecule, a polymer, a protein, a peptide, and a lipid; and wherein the peptide conjugate optionally comprises a targeting peptide in addition to the polypeptide and the moiety, such as a receptor binding domain (RBD) binding peptide or an ACE2 targeting peptide.

32. (canceled)

33. The peptide conjugate of claim 31, wherein the peptide conjugate comprises a structure selected from Peptide Conjugates 1-12 as shown in Table 9.

34-35. (canceled)

36. A method for treating a disease or condition in a subject in need thereof, wherein the method comprises administering to the subject an effective amount of the peptide conjugate of claim 29.

37. The method of claim 36, wherein the disease or condition is associated with a coronavirus infection.

38. The method of claim 36, wherein the coronavirus is SAR-Cov-2 variant.

39-44. (canceled)


SEQUENCE LISTING (1040 sequences)

SEQ ID NO: 1 - protein, 36 aa, Coronavirus
DISGINASVVNIQKEIDRLNEVAKNLNESLIDLQEL

SEQ ID NO: 2 - protein, 36 aa, Coronavirus
SLTQINTTLLDLTYEMLSLQQVVKALNESYIDLKEL

SEQ ID NO: 3 - protein, 36 aa, Coronavirus
SLDYINVTFLDLQDEMNRLQEAIKVLNQSYINLDEI

SEQ ID NO: 4 - protein, 60 aa, Measles morbillivirus
MLNSQAIDNLRASLETTNQAIEAIRQAGQGMILAVQGVQDYINNELIPSMNQLSCDLIGQ

SEQ ID NO: 5 - protein, 60 aa, Nipah henipavirus
MKNADNINKLKSSIESTNEAVVKLQETAEKTVYVLTALQDYINTNLVPTIDKISCKQTEL

SEQ ID NO: 6 - protein, 60 aa, Human parainfluenza
KQARSDIEKLKEAIRDTNKAVQSVQSSIGNLIVAIKSVQDYVNKEIVPSIARLGCEAAGL

SEQ ID NO: 7 - protein, 36 aa, Measles morbillivirus
ISLERLDVGTNLGNAIAKLEDAKELLESSDQILRSM

SEQ ID NO: 8 - protein, 36 aa, Nipah henipavirus
VFTDKVDISSQISSMNQSLQQSKDYIKEAQRLLDTV

SEQ ID NO: 9 - protein, 36 aa, Human parainfluenza
VALDPIDISIELNKAKSDLEESKEWIRRSNQKLDSI

SEQ ID NO: 10 - protein, 36 aa, Human parainfluenza
VALDPIDISIVLNKIKSDLEESKEWIRRSNKILDSI

SEQ ID NO: 11 - protein, 36 aa, Human parainfluenza
VALDPIDISIVLNKIKSQLEESKWEIRRSNKILDSI

SEQ ID NO: 12 - protein, 36 aa, Human parainfluenza
VALDPIDFSIVLNKIKSQLEESKWEIRRSNKILDSI

SEQ ID NO: 13 - protein, 21 aa, synthetic construct
IQKEIDRLNEVAKNLNESLID

SEQ ID NO: 14 - protein, 21 aa, synthetic construct
DILSENLNKAVENLRDIEKQI

SEQ ID NO: 15 - protein, 21 aa, synthetic construct
IQKEIDRLNEVAKNLNESLID

SEQ ID NO: 16 - protein, 21 aa, synthetic construct
IQKPIDRLNEVAKNLNESLID

SEQ ID NO: 17 - protein, 21 aa, synthetic construct
IQKPIDPLNEVAKPLNEPLID

SEQ ID NO: 18 - protein, 21 aa, synthetic construct
AQKEADRANEAAKNANESLID

SEQ ID NO: 19 - protein, 21 aa, synthetic construct
IQKEIDRLNEVIKNLNESLID

SEQ ID NO: 20 - protein, 21 aa, synthetic construct
IQKEIARLNEVAKALNESLID

SEQ ID NO: 21 - protein, 21 aa, synthetic construct
IQKEIARLNEVAKALNESLAA

SEQ ID NO: 22 - protein, 21 aa, synthetic construct
IQKEIARLNEVIKALNESLID

SEQ ID NO: 23 - protein, 21 aa, synthetic construct
IQKEIERLNKVAKNLNESLID

SEQ ID NO: 24 - protein, 21 aa, synthetic construct
IQKKIEELNKKAEELNKKLID

SEQ ID NO: 25 - protein, 21 aa, synthetic construct
LQDEMNRLQEAIKVLNQSYIN

SEQ ID NO: 26 - protein, 21 aa, synthetic construct
LTYEMLSLQQVVKALNESYID

SEQ ID NO: 27 - protein, 21 aa, synthetic construct
IKKEIEHLNEIAKSLNESLID

SEQ ID NO: 28 - protein, 21 aa, synthetic construct
IKKEIEHLNEIAKNLNESLID

SEQ ID NO: 29 - protein, 21 aa, synthetic construct
IEYEIKKLEEVAKKLEESLID

SEQ ID NO: 30 - protein, 21 aa, synthetic construct
IEYEIKKLEEVAKKLEESYID

SEQ ID NO: 31 - protein, 21 aa, synthetic construct
DILSENLNKAVENLRDIEKQI

SEQ ID NO: 32 - protein, 21 aa, synthetic construct
EAAAKEAAAKEAAAKEAAAKE

SEQ ID NO: 33 - protein, 21 aa, synthetic construct
IQKEIDRLNEVAKNLNESLIE

SEQ ID NO: 34 - protein, 21 aa, synthetic construct
IQKEIDRLNEVAKNLNESLIK

SEQ ID NO: 35 - protein, 21 aa, synthetic construct
IQKEIDRLNEVAKNLNESLIG

SEQ ID NO: 36 - protein, 21 aa, synthetic construct
IQKEIDRLNEVAKNLGESLIG

SEQ ID NO: 37 - protein, 21 aa, synthetic construct
IQKEIDRLNEVAKNLPESLIG

SEQ ID NO: 38 - protein, 21 aa, synthetic construct
IQKEIDRLDEVAKNLNESLID

SEQ ID NO: 39 - protein, 21 aa, synthetic construct
IQKEIDRLGEVAKNLNESLID

SEQ ID NO: 40 - protein, 21 aa, synthetic construct
IQKEIDRLNEVAKNLGESLIE

SEQ ID NO: 41 - protein, 21 aa, synthetic construct
IQKEIDRLDEVAKNLPESLIE

SEQ ID NO: 42 - protein, 21 aa, synthetic construct
IQDEIEHLNELIDNLNESLID

SEQ ID NO: 43 - protein, 21 aa, synthetic construct
IQLEIEHLNELIENLGESLIE

SEQ ID NO: 44 - protein, 21 aa, synthetic construct
IQLEIEHLNEVIENLGESLIE

SEQ ID NO: 45 - protein, 21 aa, synthetic construct
IQLEIEHLNELIENLGESLIE

SEQ ID NO: 46 - protein, 21 aa, synthetic construct
IQLEIDHLNEVIENLGESLIE

SEQ ID NO: 47 - protein, 21 aa, synthetic construct
IQKEIDRLNEVIKNLGESLIE

SEQ ID NO: 48 - protein, 21 aa, synthetic construct
  MOD_RES 3: note = ornithine
  MOD_RES 7: note = ornithine
  MOD_RES 13: note = ornithine
IQXEIDXLNEVAXNLNESLID

SEQ ID NO: 49 - protein, 21 aa, synthetic construct
  MOD_RES 3: note = 2,4-diaminobutyric acid
  MOD_RES 13: note = 2,4-diaminobutyric acid
  MOD_RES 7: note = ornithine
IQXEIDXLNEVAXNLNESLID

SEQ ID NO: 50 - protein, 21 aa, synthetic construct
  MOD_RES 3: note = 2,3- diaminopropionic acid
  MOD_RES 13: note = 2,3- diaminopropionic acid
  MOD_RES 7: note = ornithine
IQXEIDXLNEVAXNLNESLID

SEQ ID NO: 51 - protein, 21 aa, synthetic construct
  MOD_RES 3: note = N-Methyl-Lysine
  MOD_RES 13: note = N-Methyl-Lysine
  MOD_RES 7: note = ornithine
IQXEIDXLNEVAXNLNESLID

SEQ ID NO: 52 - protein, 21 aa, synthetic construct
  MOD_RES 3: note = N-Dimethyl-Lysine
  MOD_RES 13: note = N-Dimethyl-Lysine
  MOD_RES 7: note = ornithine
IQXEIDXLNEVAXNLNESLID

SEQ ID NO: 53 - protein, 21 aa, synthetic construct
  MOD_RES 3: note = N alpha-methyl-L-lysine
  MOD_RES 7: note = ornithine
  MOD_RES 13: note = ornithine
IQXEIDXLNEVAXNLNESLID

SEQ ID NO: 54 - protein, 21 aa, synthetic construct
  MOD_RES 3: note = N alpha-methyl-L-lysine
  MOD_RES 7: note = ornithine
  MOD_RES 13: note = 2,4-diaminobutyric acid
IQXEIDXLNEVAXNLNESLID

SEQ ID NO: 55 - protein, 21 aa, synthetic construct
  MOD_RES 3: note = N alpha-methyl-L-lysine
  MOD_RES 7: note = ornithine
  MOD_RES 13: note = 2,3-diaminopropionic acid
IQXEIDXLNEVAXNLNESLID

SEQ ID NO: 56 - protein, 21 aa, synthetic construct
  MOD_RES 3: note = N alpha-methyl-L-lysine
  MOD_RES 7: note = ornithine
  MOD_RES 13: note = N-Methyl-Lysine
IQXEIDXLNEVAXNLNESLID

SEQ ID NO: 57 - protein, 21 aa, synthetic construct
  MOD_RES 3: note = N alpha-methyl-L-lysine
  MOD_RES 7: note = ornithine
  MOD_RES 13: note = N-Dimethyl-Lysine
IQXEIDXLNEVAXNLNESLID

SEQ ID NO: 58 - protein, 21 aa, synthetic construct
  MOD_RES 3: note = ornithine
  MOD_RES 13: note = ornithine
  MOD_RES 7: note = Citrulline
IQXEIDXLNEVAXNLNESLID

SEQ ID NO: 59 - protein, 21 aa, synthetic construct
  MOD_RES 3: note = 2,4-diaminobutyric acid
  MOD_RES 13: note = 2,4-diaminobutyric acid
  MOD_RES 7: note = Citrulline
IQXEIDXLNEVAXNLNESLID

SEQ ID NO: 60 - protein, 21 aa, synthetic construct
  MOD_RES 3: note = 2,3-diaminopropionic acid
  MOD_RES 13: note = 2,3-diaminopropionic acid
  MOD_RES 7: note = Citrulline
IQXEIDXLNEVAXNLNESLID

SEQ ID NO: 61 - protein, 21 aa, synthetic construct
  MOD_RES 3: note = N-Methyl-Lysine
  MOD_RES 13: note = N-Methyl-Lysine
  MOD_RES 7: note = Citrulline
IQXEIDXLNEVAXNLNESLID

SEQ ID NO: 62 - protein, 21 aa, synthetic construct
  MOD_RES 3: note = N-Dimethyl-Lysine
  MOD_RES 13: note = N-Dimethyl-Lysine
  MOD_RES 7: note = Citrulline
IQXEIDXLNEVAXNLNESLID

SEQ ID NO: 63 - protein, 21 aa, synthetic construct
  MOD_RES 3: note = N alpha-methyl-L-lysine
  MOD_RES 7: note = Citrulline
  MOD_RES 13: note = ornithine
IQXEIDXLNEVAXNLNESLID

SEQ ID NO: 64 - protein, 21 aa, synthetic construct
  MOD_RES 3: note = N alpha-methyl-L-lysine
  MOD_RES 7: note = Citrulline
  MOD_RES 13: note = 2,4-diaminobutyric acid
IQXEIDXLNEVAXNLNESLID

SEQ ID NO: 65 - protein, 21 aa, synthetic construct
  MOD_RES 3: note = N alpha-methyl-L-lysine
  MOD_RES 7: note = Citrulline
  MOD_RES 13: note = 2,3-diaminopropionic acid
IQXEIDXLNEVAXNLNESLID

SEQ ID NO: 66 - protein, 21 aa, synthetic construct
  MOD_RES 3: note = N alpha-methyl-L-lysine
  MOD_RES 7: note = Citrulline
  MOD_RES 13: note = N-Methyl-Lysine
IQXEIDXLNEVAXNLNESLID

SEQ ID NO: 67 - protein, 21 aa, synthetic construct
  MOD_RES 3: note = N alpha-methyl-L-lysine
  MOD_RES 7: note = Citrulline
  MOD_RES 13: note = N-Dimethyl-Lysine
IQXEIDXLNEVAXNLNESLID

SEQ ID NO: 68 - protein, 21 aa, synthetic construct
  MOD_RES 3: note = ornithine
  MOD_RES 13: note = ornithine
IQXEIDHLNEVIXNLNESLID

SEQ ID NO: 69 - protein, 21 aa, synthetic construct
  MOD_RES 3: note = 2,4-diaminobutyric acid
  MOD_RES 13: note = 2,4-diaminobutyric acid
IQXEIDHLNEVIXNLNESLID

SEQ ID NO: 70 - protein, 21 aa, synthetic construct
  MOD_RES 3: note = 2,3-diaminopropionic acid
  MOD_RES 13: note = 2,3-diaminopropionic acid
IQXEIDHLNEVIXNLNESLID

SEQ ID NO: 71 - protein, 21 aa, synthetic construct
  MOD_RES 3: note = ornithine
  MOD_RES 13: note = ornithine
IQXEIEHLNEVIXNLNESLID

SEQ ID NO: 72 - protein, 21 aa, synthetic construct
  MOD_RES 3: note = 2,4-diaminobutyric acid
  MOD_RES 13: note = 2,4-diaminobutyric acid
IQXEIEHLNEVIXNLNESLID

SEQ ID NO: 73 - protein, 21 aa, synthetic construct
  MOD_RES 3: note = 2,3-diaminopropionic acid
  MOD_RES 13: note = 2,3-diaminopropionic acid
IQXEIEHLNEVIXNLNESLID

SEQ ID NO: 74 - protein, 21 aa, synthetic construct
  MOD_RES 3: note = N-Methyl-Lysine
  MOD_RES 13: note = N-Methyl-Lysine
IQXEIEHLNEVIXNLNESLID

SEQ ID NO: 75 - protein, 21 aa, synthetic construct
  MOD_RES 3: note = N-Dimethyl-Lysine
  MOD_RES 13: note = N-Dimethyl-Lysine
IQXEIEHLNEVIXNLNESLID

SEQ ID NO: 76 - protein, 21 aa, synthetic construct
  MOD_RES 3: note = N alpha-methyl-L-lysine
  MOD_RES 13: note = ornithine
IQXEIEHLNEVIXNLNESLID

SEQ ID NO: 77 - protein, 21 aa, synthetic construct
  MOD_RES 3: note = N alpha-methyl-L-lysine
  MOD_RES 13: note = 2,4-diaminobutyric acid
IQXEIEHLNEVIXNLNESLID

SEQ ID NO: 78 - protein, 21 aa, synthetic construct
  MOD_RES 3: note = N alpha-methyl-L-lysine
  MOD_RES 13: note = 2,3-diaminopropionic acid
IQXEIEHLNEVIXNLNESLID

SEQ ID NO: 79 - protein, 21 aa, synthetic construct
  MOD_RES 3: note = N alpha-methyl-L-lysine
  MOD_RES 13: note = N-Methyl-Lysine
IQXEIEHLNEVIXNLNESLID

SEQ ID NO: 80 - protein, 21 aa, synthetic construct
  MOD_RES 3: note = N alpha-methyl-L-lysine
  MOD_RES 13: note = N-Dimethyl-Lysine
IQXEIEHLNEVIXNLNESLID

SEQ ID NO: 81 - protein, 21 aa, synthetic construct
  MOD_RES 3: note = ornithine
  MOD_RES 7: note = ornithine
  MOD_RES 13: note = ornithine
IQXEIDXLNEVIXNLNESLID

SEQ ID NO: 82 - protein, 21 aa, synthetic construct
  MOD_RES 3: note = 2,4-diaminobutyric acid
  MOD_RES 13: note = 2,4-diaminobutyric acid
  MOD_RES 7: note = ornithine
IQXEIDXLNEVIXNLNESLID

SEQ ID NO: 83 - protein, 21 aa, synthetic construct
  MOD_RES 3: note = 2,3-diaminopropionic acid
  MOD_RES 13: note = 2,3-diaminopropionic acid
  MOD_RES 7: note = ornithine
IQXEIDXLNEVIXNLNESLID

SEQ ID NO: 84 - protein, 21 aa, synthetic construct
  MOD_RES 3: note = N-Methyl-Lysine
  MOD_RES 13: note = N-Methyl-Lysine
  MOD_RES 7: note = ornithine
IQXEIDXLNEVIXNLNESLID

SEQ ID NO: 85 - protein, 21 aa, synthetic construct
  MOD_RES 3: note = N-Dimethyl-Lysine
  MOD_RES 13: note = N-Dimethyl-Lysine
  MOD_RES 7: note = ornithine
IQXEIDXLNEVIXNLNESLID

SEQ ID NO: 86 - protein, 21 aa, synthetic construct
  MOD_RES 3: note = N alpha-methyl-L-lysine
  MOD_RES 7: note = ornithine
  MOD_RES 13: note = ornithine
IQXEIDXLNEVIXNLNESLID

SEQ ID NO: 87 - protein, 21 aa, synthetic construct
  MOD_RES 3: note = N alpha-methyl-L-lysine
  MOD_RES 7: note = ornithine
  MOD_RES 13: note = 2,4-diaminobutyric acid
IQXEIDXLNEVIXNLNESLID

SEQ ID NO: 88 - protein, 21 aa, synthetic construct
  MOD_RES 3: note = N alpha-methyl-L-lysine
  MOD_RES 7: note = ornithine
  MOD_RES 13: note = 2,3-diaminopropionic acid
IQXEIDXLNEVIXNLNESLID

SEQ ID NO: 89 - protein, 21 aa, synthetic construct
  MOD_RES 3: note = N alpha-methyl-L-lysine
  MOD_RES 7: note = ornithine
  MOD_RES 13: note = N-Methyl-Lysine
IQXEIDXLNEVIXNLNESLID

SEQ ID NO: 90 - protein, 21 aa, synthetic construct
  MOD_RES 3: note = N alpha-methyl-L-lysine
  MOD_RES 7: note = ornithine
  MOD_RES 13: note = N-Dimethyl-Lysine
IQXEIDXLNEVIXNLNESLID

SEQ ID NO: 91 - protein, 21 aa, synthetic construct
  MOD_RES 3: note = ornithine
  MOD_RES 13: note = ornithine
  MOD_RES 7: note = Citrulline
IQXEIDXLNEVIXNLNESLID

SEQ ID NO: 92 - protein, 21 aa, synthetic construct
  MOD_RES 3: note = 2,4-diaminobutyric acid
  MOD_RES 13: note = 2,4-diaminobutyric acid
  MOD_RES 7: note = Citrulline
IQXEIDXLNEVIXNLNESLID

SEQ ID NO: 93 - protein, 21 aa, synthetic construct
  MOD_RES 3: note = 2,3-diaminopropionic acid
  MOD_RES 13: note = 2,3-diaminopropionic acid
  MOD_RES 7: note = Citrulline
IQXEIDXLNEVIXNLNESLID

SEQ ID NO: 94 - protein, 21 aa, synthetic construct
  MOD_RES 3: note = N-Methyl-Lysine
  MOD_RES 13: note = N-Methyl-Lysine
  MOD_RES 7: note = Citrulline
IQXEIDXLNEVIXNLNESLID

SEQ ID NO: 95 - protein, 21 aa, synthetic construct
  MOD_RES 3: note = N-Dimethyl-Lysine
  MOD_RES 13: note = N-Dimethyl-Lysine
  MOD_RES 7: note = Citrulline
IQXEIDXLNEVIXNLNESLID

SEQ ID NO: 96 - protein, 21 aa, synthetic construct
  MOD_RES 3: note = N alpha-methyl-L-lysine
  MOD_RES 7: note = Citrulline
  MOD_RES 13: note = ornithine
IQXEIDXLNEVIXNLNESLID

SEQ ID NO: 97 - protein, 21 aa, synthetic construct
  MOD_RES 3: note = N alpha-methyl-L-lysine
  MOD_RES 7: note = Citrulline
  MOD_RES 13: note = 2,4-diaminobutyric acid
IQXEIDXLNEVIXNLNESLID

SEQ ID NO: 98 - protein, 21 aa, synthetic construct
  MOD_RES 3: note = N alpha-methyl-L-lysine
  MOD_RES 7: note = Citrulline
  MOD_RES 13: note = 2,3-diaminopropionic acid
IQXEIDXLNEVIXNLNESLID

SEQ ID NO: 99 - protein, 21 aa, synthetic construct
  MOD_RES 3: note = N alpha-methyl-L-lysine
  MOD_RES 7: note = Citrulline
  MOD_RES 13: note = N-Methyl-Lysine
IQXEIDXLNEVIXNLNESLID

SEQ ID NO: 100 - protein, 21 aa, synthetic construct
  MOD_RES 3: note = N alpha-methyl-L-lysine
  MOD_RES 7: note = Citrulline
  MOD_RES 13: note = N-Dimethyl-Lysine
IQXEIDXLNEVIXNLNESLID

SEQ ID NO: 101 - protein, 21 aa, synthetic construct
  MOD_RES 3: note = ornithine
  MOD_RES 13: note = ornithine
IQXEIDHLNEVAXNLGESLIE

SEQ ID NO: 102 - protein, 21 aa, synthetic construct
  MOD_RES 3: note = 2,4-diaminobutyric acid
  MOD_RES 13: note = 2,4-diaminobutyric acid
IQXEIDHLNEVAXNLGESLIE

SEQ ID NO: 103 - protein, 21 aa, synthetic construct
  MOD_RES 3: note = 2,3-diaminopropionic acid
  MOD_RES 13: note = 2,3-diaminopropionic acid
IQXEIDHLNEVAXNLGESLIE

SEQ ID NO: 104 - protein, 21 aa, synthetic construct
  MOD_RES 3: note = ornithine
  MOD_RES 13: note = ornithine
IQXEIEHLNEVAXNLGESLIE

SEQ ID NO: 105 - protein, 21 aa, synthetic construct
  MOD_RES 3: note = 2,4-diaminobutyric acid
  MOD_RES 13: note = 2,4-diaminobutyric acid
IQXEIEHLNEVAXNLGESLIE

SEQ ID NO: 106 - protein, 21 aa, synthetic construct
  MOD_RES 3: note = 2,3-diaminopropionic acid
  MOD_RES 13: note = 2,3-diaminopropionic acid
IQXEIEHLNEVAXNLGESLIE

SEQ ID NO: 107 - protein, 21 aa, synthetic construct
  MOD_RES 3: note = N-Methyl-Lysine
  MOD_RES 13: note = N-Methyl-Lysine
IQXEIEHLNEVAXNLGESLIE

SEQ ID NO: 108 - protein, 21 aa, synthetic construct
  MOD_RES 3: note = N-Dimethyl-Lysine
  MOD_RES 13: note = N-Dimethyl-Lysine
IQXEIEHLNEVAXNLGESLIE

SEQ ID NO: 109 - protein, 21 aa, synthetic construct
  MOD_RES 3: note = N alpha-methyl-L-lysine
  MOD_RES 13: note = ornithine
IQXEIEHLNEVAXNLGESLIE

SEQ ID NO: 110 - protein, 21 aa, synthetic construct
  MOD_RES 3: note = N alpha-methyl-L-lysine
  MOD_RES 13: note = 2,4-diaminobutyric acid
IQXEIEHLNEVAXNLGESLIE

SEQ ID NO: 111 - protein, 21 aa, synthetic construct
  MOD_RES 3: note = N alpha-methyl-L-lysine
  MOD_RES 13: note = 2,3-diaminopropionic acid
IQXEIEHLNEVAXNLGESLIE

SEQ ID NO: 112 - protein, 21 aa, synthetic construct
  MOD_RES 3: note = N alpha-methyl-L-lysine
  MOD_RES 13: note = N-Methyl-Lysine
IQXEIEHLNEVAXNLGESLIE

SEQ ID NO: 113 - protein, 21 aa, synthetic construct
  MOD_RES 3: note = N alpha-methyl-L-lysine
  MOD_RES 13: note = N-Dimethyl-Lysine
IQXEIEHLNEVAXNLGESLIE

SEQ ID NO: 114 - protein, 21 aa, synthetic construct
  MOD_RES 3: note = ornithine
  MOD_RES 7: note = ornithine
  MOD_RES 13: note = ornithine
IQXEIDXLNEVAXNLGESLIE

SEQ ID NO: 115 - protein, 21 aa, synthetic construct
  MOD_RES 3: note = 2,4-diaminobutyric acid
  MOD_RES 13: note = 2,4-diaminobutyric acid
  MOD_RES 7: note = ornithine
IQXEIDXLNEVAXNLGESLIE

SEQ ID NO: 116 - protein, 21 aa, synthetic construct
  MOD_RES 3: note = 2,3-diaminopropionic acid
  MOD_RES 13: note = 2,3-diaminopropionic acid
  MOD_RES 7: note = ornithine
IQXEIDXLNEVAXNLGESLIE

SEQ ID NO: 117 - protein, 21 aa, synthetic construct
  MOD_RES 3: note = N-Methyl-Lysine
  MOD_RES 13: note = N-Methyl-Lysine
  MOD_RES 7: note = ornithine
IQXEIDXLNEVAXNLGESLIE

SEQ ID NO: 118 - protein, 21 aa, synthetic construct
  MOD_RES 3: note = N-Dimethyl-Lysine
  MOD_RES 13: note = N-Dimethyl-Lysine
  MOD_RES 7: note = ornithine
IQXEIDXLNEVAXNLGESLIE

SEQ ID NO: 119 - protein, 21 aa, synthetic construct
  MOD_RES 3: note = N alpha-methyl-L-lysine
  MOD_RES 7: note = ornithine
  MOD_RES 13: note = ornithine
IQXEIDXLNEVAXNLGESLIE

SEQ ID NO: 120 - protein, 21 aa, synthetic construct
  MOD_RES 3: note = N alpha-methyl-L-lysine
  MOD_RES 7: note = ornithine
  MOD_RES 13: note = 2,4-diaminobutyric acid
IQXEIDXLNEVAXNLGESLIE

SEQ ID NO: 121 - protein, 21 aa, synthetic construct
  MOD_RES 3: note = N alpha-methyl-L-lysine
  MOD_RES 7: note = ornithine
  MOD_RES 13: note = 2,3-diaminopropionic acid
IQXEIDXLNEVAXNLGESLIE

SEQ ID NO: 122 - protein, 21 aa, synthetic construct
  MOD_RES 3: note = N alpha-methyl-L-lysine
  MOD_RES 7: note = ornithine
  MOD_RES 13: note = N-Methyl-Lysine
IQXEIDXLNEVAXNLGESLIE

SEQ ID NO: 123 - protein, 21 aa, synthetic construct
  MOD_RES 3: note = N alpha-methyl-L-lysine
  MOD_RES 7: note = ornithine
  MOD_RES 13: note = N-Dimethyl-Lysine
IQXEIDXLNEVAXNLGESLIE

SEQ ID NO: 124 - protein, 21 aa, synthetic construct
  MOD_RES 3: note = ornithine
  MOD_RES 13: note = ornithine
  MOD_RES 7: note = Citrulline
IQXEIDXLNEVAXNLGESLIE

SEQ ID NO: 125 - protein, 21 aa, synthetic construct
  MOD_RES 3: note = 2,4-diaminobutyric acid
  MOD_RES 13: note = 2,4-diaminobutyric acid
  MOD_RES 7: note = Citrulline
IQXEIDXLNEVAXNLGESLIE

SEQ ID NO: 126 - protein, 21 aa, synthetic construct
  MOD_RES 3: note = 2,3-diaminopropionic acid
  MOD_RES 13: note = 2,3-diaminopropionic acid
  MOD_RES 7: note = Citrulline
IQXEIDXLNEVAXNLGESLIE

SEQ ID NO: 127 - protein, 21 aa, synthetic construct
  MOD_RES 3: note = N-Methyl-Lysine
  MOD_RES 13: note = N-Methyl-Lysine
  MOD_RES 7: note = Citrulline
IQXEIDXLNEVAXNLGESLIE

SEQ ID NO: 128 - protein, 21 aa, synthetic construct
  MOD_RES 3: note = N-Dimethyl-Lysine
  MOD_RES 13: note = N-Dimethyl-Lysine
  MOD_RES 7: note = Citrulline
IQXEIDXLNEVAXNLGESLIE

SEQ ID NO: 129 - protein, 21 aa, synthetic construct
  MOD_RES 3: note = N alpha-methyl-L-lysine
  MOD_RES 7: note = Citrulline
  MOD_RES 13: note = ornithine
IQXEIDXLNEVAXNLGESLIE

SEQ ID NO: 130 - protein, 21 aa, synthetic construct
  MOD_RES 3: note = N alpha-methyl-L-lysine
  MOD_RES 7: note = Citrulline
  MOD_RES 13: note = 2,4-diaminobutyric acid
IQXEIDXLNEVAXNLGESLIE

SEQ ID NO: 131 - protein, 21 aa, synthetic construct
  MOD_RES 3: note = N alpha-methyl-L-lysine
  MOD_RES 7: note = Citrulline
  MOD_RES 13: note = 2,3-diaminopropionic acid
IQXEIDXLNEVAXNLGESLIE

SEQ ID NO: 132 - protein, 21 aa, synthetic construct
  MOD_RES 3: note = N alpha-methyl-L-lysine
  MOD_RES 7: note = Citrulline
  MOD_RES 13: note = N-Methyl-Lysine
IQXEIDXLNEVAXNLGESLIE

SEQ ID NO: 133 - protein, 21 aa, synthetic construct
  MOD_RES 3: note = N alpha-methyl-L-lysine
  MOD_RES 7: note = Citrulline
  MOD_RES 13: note = N-Dimethyl-Lysine
IQXEIDXLNEVAXNLGESLIE

SEQ ID NO: 134 - protein, 21 aa, synthetic construct
  MOD_RES 3: note = ornithine
  MOD_RES 13: note = ornithine
IQXEIDHLNEVIXNLGESLIE

SEQ ID NO: 135 - protein, 21 aa, synthetic construct
  MOD_RES 3: note = 2,4-diaminobutyric acid
  MOD_RES 13: note = 2,4-diaminobutyric acid
IQXEIDHLNEVIXNLGESLIE

SEQ ID NO: 136 - protein, 21 aa, synthetic construct
  MOD_RES 3: note = 2,3-diaminopropionic acid
  MOD_RES 13: note = 2,3-diaminopropionic acid
IQXEIDHLNEVIXNLGESLIE

SEQ ID NO: 137 - protein, 21 aa, synthetic construct
  MOD_RES 3: note = ornithine
  MOD_RES 13: note = ornithine
IQXEIEHLNEVIXNLGESLIE

SEQ ID NO: 138 - protein, 21 aa, synthetic construct
  MOD_RES 3: note = 2,4-diaminobutyric acid
  MOD_RES 13: note = 2,4-diaminobutyric acid
IQXEIEHLNEVIXNLGESLIE

SEQ ID NO: 139 - protein, 21 aa, synthetic construct
  MOD_RES 3: note = 2,3-diaminopropionic acid
  MOD_RES 13: note = 2,3-diaminopropionic acid
IQXEIEHLNEVIXNLGESLIE

SEQ ID NO: 140 - protein, 21 aa, synthetic construct
  MOD_RES 3: note = N-Methyl-Lysine
  MOD_RES 13: note = N-Methyl-Lysine
IQXEIEHLNEVIXNLGESLIE

SEQ ID NO: 141 - protein, 21 aa, synthetic construct
  MOD_RES 3: note = N-Dimethyl-Lysine
  MOD_RES 13: note = N-Dimethyl-Lysine
IQXEIEHLNEVIXNLGESLIE

SEQ ID NO: 142 - protein, 21 aa, synthetic construct
  MOD_RES 3: note = N alpha-methyl-L-lysine
  MOD_RES 13: note = ornithine
IQXEIEHLNEVIXNLGESLIE

SEQ ID NO: 143 - protein, 21 aa, synthetic construct
  MOD_RES 3: note = N alpha-methyl-L-lysine
  MOD_RES 13: note = 2,4-diaminobutyric acid
IQXEIEHLNEVIXNLGESLIE

SEQ ID NO: 144 - protein, 21 aa, synthetic construct
  MOD_RES 3: note = N alpha-methyl-L-lysine
  MOD_RES 13: note = 2,3-diaminopropionic acid
IQXEIEHLNEVIXNLGESLIE

SEQ ID NO: 145 - protein, 21 aa, synthetic construct
  MOD_RES 3: note = N alpha-methyl-L-lysine
  MOD_RES 13: note = N-Methyl-Lysine
IQXEIEHLNEVIXNLGESLIE

SEQ ID NO: 146 - protein, 21 aa, synthetic construct
  MOD_RES 3: note = N alpha-methyl-L-lysine
  MOD_RES 13: note = N-Dimethyl-Lysine
IQXEIEHLNEVIXNLGESLIE

SEQ ID NO: 147 - protein, 21 aa, synthetic construct
  MOD_RES 3: note = ornithine
  MOD_RES 7: note = ornithine
  MOD_RES 13: note = ornithine
IQXEIDXLNEVIXNLGESLIE

SEQ ID NO: 148 - protein, 21 aa, synthetic construct
  MOD_RES 3: note = 2,4-diaminobutyric acid
  MOD_RES 13: note = 2,4-diaminobutyric acid
  MOD_RES 7: note = ornithine
IQXEIDXLNEVIXNLGESLIE

SEQ ID NO: 149 - protein, 21 aa, synthetic construct
  MOD_RES 3: note = 2,3-diaminopropionic acid
  MOD_RES 13: note = 2,3-diaminopropionic acid
  MOD_RES 7: note = ornithine
IQXEIDXLNEVIXNLGESLIE

SEQ ID NO: 150 - protein, 21 aa, synthetic construct
  MOD_RES 3: note = N-Methyl-Lysine
  MOD_RES 13: note = N-Methyl-Lysine
  MOD_RES 7: note = ornithine
IQXEIDXLNEVIXNLGESLIE

SEQ ID NO: 151 - protein, 21 aa, synthetic construct
  MOD_RES 3: note = N-Dimethyl-Lysine
  MOD_RES 13: note = N-Dimethyl-Lysine
  MOD_RES 7: note = ornithine
IQXEIDXLNEVIXNLGESLIE

SEQ ID NO: 152 - protein, 21 aa, synthetic construct
  MOD_RES 3: note = N alpha-methyl-L-lysine
  MOD_RES 7: note = ornithine
  MOD_RES 13: note = ornithine
IQXEIDXLNEVIXNLGESLIE

SEQ ID NO: 153 - protein, 21 aa, synthetic construct
  MOD_RES 3: note = N alpha-methyl-L-lysine
  MOD_RES 7: note = ornithine
  MOD_RES 13: note = 2,4-diaminobutyric acid
IQXEIDXLNEVIXNLGESLIE

SEQ ID NO: 154 - protein, 21 aa, synthetic construct
  MOD_RES 3: note = N alpha-methyl-L-lysine
  MOD_RES 7: note = ornithine
  MOD_RES 13: note = 2,3-diaminopropionic acid
IQXEIDXLNEVIXNLGESLIE

SEQ ID NO: 155 - protein, 21 aa, synthetic construct
  MOD_RES 3: note = N alpha-methyl-L-lysine
  MOD_RES 7: note = ornithine
  MOD_RES 13: note = N-Methyl-Lysine
IQXEIDXLNEVIXNLGESLIE

SEQ ID NO: 156 - protein, 21 aa, synthetic construct
  MOD_RES 3: note = N alpha-methyl-L-lysine
  MOD_RES 7: note = ornithine
  MOD_RES 13: note = N-Dimethyl-Lysine
IQXEIDXLNEVIXNLGESLIE

SEQ ID NO: 157 - protein, 21 aa, synthetic construct
  MOD_RES 3: note = ornithine
  MOD_RES 13: note = ornithine
  MOD_RES 7: note = Citrulline
IQXEIDXLNEVIXNLGESLIE

SEQ ID NO: 158 - protein, 21 aa, synthetic construct
  MOD_RES 3: note = 2,4-diaminobutyric acid
  MOD_RES 13: note = 2,4-diaminobutyric acid
  MOD_RES 7: note = Citrulline
IQXEIDXLNEVIXNLGESLIE

SEQ ID NO: 159 - protein, 21 aa, synthetic construct
  MOD_RES 3: note = 2,3-diaminopropionic acid
  MOD_RES 13: note = 2,3-diaminopropionic acid
  MOD_RES 7: note = Citrulline
IQXEIDXLNEVIXNLGESLIE

SEQ ID NO: 160 - protein, 21 aa, synthetic construct
  MOD_RES 3: note = N-Methyl-Lysine
  MOD_RES 13: note = N-Methyl-Lysine
  MOD_RES 7: note = Citrulline
IQXEIDXLNEVIXNLGESLIE

SEQ ID NO: 161 - protein, 21 aa, synthetic construct
  MOD_RES 3: note = N-Dimethyl-Lysine
  MOD_RES 13: note = N-Dimethyl-Lysine
  MOD_RES 7: note = Citrulline
IQXEIDXLNEVIXNLGESLIE

SEQ ID NO: 162 - protein, 21 aa, synthetic construct
  MOD_RES 3: note = N alpha-methyl-L-lysine
  MOD_RES 7: note = Citrulline
  MOD_RES 13: note = ornithine
IQXEIDXLNEVIXNLGESLIE

SEQ ID NO: 163 - protein, 21 aa, synthetic construct
  MOD_RES 3: note = N alpha-methyl-L-lysine
  MOD_RES 7: note = Citrulline
  MOD_RES 13: note = 2,4-diaminobutyric acid
IQXEIDXLNEVIXNLGESLIE

SEQ ID NO: 164 - protein, 21 aa, synthetic construct
  MOD_RES 3: note = N alpha-methyl-L-lysine
  MOD_RES 7: note = Citrulline
  MOD_RES 13: note = 2,3-diaminopropionic acid
IQXEIDXLNEVIXNLGESLIE

SEQ ID NO: 165 - protein, 21 aa, synthetic construct
  MOD_RES 3: note = N alpha-methyl-L-lysine
  MOD_RES 7: note = Citrulline
  MOD_RES 13: note = N-Methyl-Lysine
IQXEIDXLNEVIXNLGESLIE

SEQ ID NO: 166 - protein, 21 aa, synthetic construct
  MOD_RES 3: note = N alpha-methyl-L-lysine
  MOD_RES 7: note = Citrulline
  MOD_RES 13: note = N-Dimethyl-Lysine
IQXEIDXLNEVIXNLGESLIE

SEQ ID NO: 167 - protein, 25 aa, synthetic construct
IDRLNEVAKNLNESLIDLQELGKYE

SEQ ID NO: 168 - protein, 15 aa, synthetic construct
IQKEIDRLNEVAKNL

SEQ ID NO: 169 - protein, 25 aa, synthetic construct
IQKEIDRLNEVAKNLNESLIDLQEL

SEQ ID NO: 170 - protein, 18 aa, synthetic construct
IQKEIDRLNEVAKNLNES

SEQ ID NO: 171 - protein, 18 aa, synthetic construct
IQKEIDRLNEVAKNLNES

SEQ ID NO: 172 - protein, 22 aa, synthetic construct
IQKEIDRLNEVAKNLNESLIDL

SEQ ID NO: 173 - protein, 11 aa, synthetic construct
IQKEIDRLNEV

SEQ ID NO: 174 - protein, 21 aa, synthetic construct
IEYEIDRLNEVAKNLNESLID

SEQ ID NO: 175 - protein, 21 aa, synthetic construct
IQKEIDHLNEVAKNLNESLID

SEQ ID NO: 176 - protein, 21 aa, synthetic construct
IQSEIDSLNELIDNLNESLID

SEQ ID NO: 177 - protein, 21 aa, synthetic construct
IQDEIDHLNELIDNLNESLID

SEQ ID NO: 178 - protein, 21 aa, synthetic construct
IQLEIEHLNEVIENLGESLIE

SEQ ID NO: 179 - protein, 21 aa, synthetic construct
IQKEIDRLNEVIKNLGESLIE

SEQ ID NO: 180 - protein, 21 aa, synthetic construct
IQKEIDHLNEVIKNLGESLIE

SEQ ID NO: 181 - protein, 21 aa, synthetic construct
IQKEIEHLNEVIKNLGESLIE

SEQ ID NO: 182 - protein, 21 aa, synthetic construct
IQLEIEHLNEVAENLNESLID

SEQ ID NO: 183 - protein, 21 aa, synthetic construct
IQKEIEHLNEVAKNLNESLID

SEQ ID NO: 184 - protein, 21 aa, synthetic construct
IQLEIDRLNEVAKNLNESLID

SEQ ID NO: 185 - protein, 21 aa, synthetic construct
IQKEIDRLNEVAENLNESLID

SEQ ID NO: 186 - protein, 21 aa, synthetic construct
IQKEIDRLNEVAKNLNESLID

SEQ ID NO: 187 - protein, 23 aa, synthetic construct
IQLEIEHLNEVIENLGESLIELQ

SEQ ID NO: 188 - protein, 21 aa, synthetic construct
IQLEIEHLNELIENLGESLIE

SEQ ID NO: 189 - protein, 21 aa, synthetic construct
IQLEIDHLNELIENLGESLIE

SEQ ID NO: 190 - protein, 21 aa, synthetic construct
IQLEINHLNELIENLGESLIE

SEQ ID NO: 191 - protein, 21 aa, synthetic construct
IQLEILHLNELIENLGESLIE

SEQ ID NO: 192 - protein, 21 aa, synthetic construct
IQLEISHLNELIENLGESLIE

SEQ ID NO: 193 - protein, 21 aa, synthetic construct
IQLEIEHLNELIENLGESLID

SEQ ID NO: 194 - protein, 21 aa, synthetic construct
IQLEIEHLNELIENLNESLID

SEQ ID NO: 195 - protein, 21 aa, synthetic construct
IQQEIDRLNEVAKNLNESLID

SEQ ID NO: 196 - protein, 21 aa, synthetic construct
IQEEIDRLNEVAKNLNESLID

SEQ ID NO: 197 - protein, 21 aa, synthetic construct
IQSEIDRLNEVAKNLNESLID

SEQ ID NO: 198 - protein, 21 aa, synthetic construct
IQNEIDRLNEVAKNLNESLID

SEQ ID NO: 199 - protein, 21 aa, synthetic construct
IQKEIDNLNEVAKNLNESLID

SEQ ID NO: 200 - protein, 21 aa, synthetic construct
IQKEIDQLNEVAKNLNESLID

SEQ ID NO: 201 - protein, 21 aa, synthetic construct
IQKEIDELNEVAKNLNESLID

SEQ ID NO: 202 - protein, 21 aa, synthetic construct
IQKEIDRLNEIAKNLNESLID

SEQ ID NO: 203 - protein, 21 aa, synthetic construct
IQKEIDRLNEMAKNLNESLID

SEQ ID NO: 204 - protein, 21 aa, synthetic construct
IQKEIDRLNELAKNLNESLID

SEQ ID NO: 205 - protein, 21 aa, synthetic construct
IQKEIDRLNEAAKNLNESLID

SEQ ID NO: 206 - protein, 21 aa, synthetic construct
IQKEIDRLNETAKNLNESLID

SEQ ID NO: 207 - protein, 21 aa, synthetic construct
IQKEIDRLNEVSKNLNESLID

SEQ ID NO: 208 - protein, 21 aa, synthetic construct
IQKEIDRLNEVVKNLNESLID

SEQ ID NO: 209 - protein, 21 aa, synthetic construct
IQKEIDRLNEVCKNLNESLID

SEQ ID NO: 210 - protein, 21 aa, synthetic construct
IQKEIDRLNEVTKNLNESLID

SEQ ID NO: 211 - protein, 21 aa, synthetic construct
IQKEIDRLNEVGKNLNESLID

SEQ ID NO: 212 - protein, 21 aa, synthetic construct
IQKEIDRLNELIKNLNESLID

SEQ ID NO: 213 - protein, 21 aa, synthetic construct
IQKEIDRLNEIIKNLNESLID

SEQ ID NO: 214 - protein, 21 aa, synthetic construct
IQKEIDRLNEISKNLNESLID

SEQ ID NO: 215 - protein, 21 aa, synthetic construct
IQKEIDRLNEAIKNLNESLID

SEQ ID NO: 216 - protein, 21 aa, synthetic construct
IQKEIDRLNETIKNLNESLID

SEQ ID NO: 217 - protein, 21 aa, synthetic construct
IQKEIDRLNEVAQNLNESLID

SEQ ID NO: 218 - protein, 21 aa, synthetic construct
IQKEIDRLNEVASNLNESLID

SEQ ID NO: 219 - protein, 21 aa, synthetic construct
IQKEIDRLNEVANNLNESLID

SEQ ID NO: 220 - protein, 21 aa, synthetic construct
IQKEIDRLNEVAKNLGESLID

SEQ ID NO: 221 - protein, 21 aa, synthetic construct
IQKEIDRLNEVIKNLNESLID

SEQ ID NO: 222 - protein, 21 aa, synthetic construct
IQEEIEIILTVAIELNSTIIY

SEQ ID NO: 223 - protein, 21 aa, synthetic construct
ISEEISILLTVAEELESALIY

SEQ ID NO: 224 - protein, 21 aa, synthetic construct
IETEIQIILTVAIELNSTIIY

SEQ ID NO: 225 - protein, 21 aa, synthetic construct
IEEEIQIILQVAIELNSTIIY

SEQ ID NO: 226 - protein, 21 aa, synthetic construct
IQEYIETLLTVATELNSALIF

SEQ ID NO: 227 - protein, 21 aa, synthetic construct
IQEEIEIILQVAIELNSTIIY

SEQ ID NO: 228 - protein, 21 aa, synthetic construct
IQEYIETLLQVATELNSALIF

SEQ ID NO: 229 - protein, 21 aa, synthetic construct
IQTEIEIILTVAIELNSTIIY

SEQ ID NO: 230 - protein, 21 aa, synthetic construct
IQTEIDIILQVAIELNSTIIY

SEQ ID NO: 231 - protein, 21 aa, synthetic construct
IQTEIDIILTVAIELNSTIIY

SEQ ID NO: 232 - protein, 21 aa, synthetic construct
IQTEIEIILQVAIELNSTIIY

SEQ ID NO: 233 - protein, 21 aa, synthetic construct
IQEYIDQLLIVATELNSALIY

SEQ ID NO: 234 - protein, 21 aa, synthetic construct
IQEYIDTLLQVATELNSALIF

SEQ ID NO: 235 - protein, 21 aa, synthetic construct
IEEHIQTLLTVATELNSALIF

SEQ ID NO: 236 - protein, 21 aa, synthetic construct
IEDEIQTLLQVAIELNSALIF

SEQ ID NO: 237 - protein, 21 aa, synthetic construct
IEEHIQQLLQVAIELNSALIF

SEQ ID NO: 238 - protein, 21 aa, synthetic construct
IEEYIQTLLTVATELNSALIF

SEQ ID NO: 239 - protein, 21 aa, synthetic construct
IQEYIDQLLIVATELNSALIF

SEQ ID NO: 240 - protein, 21 aa, synthetic construct
IQEYIDQLLQVAIELNSALIF

SEQ ID NO: 241 - protein, 21 aa, synthetic construct
IQLEIEHLNEVIENLGESLIE

SEQ ID NO: 242 - protein, 21 aa, synthetic construct
IQLEIEHLNELIENLGESLIE

SEQ ID NO: 243 - protein, 21 aa, synthetic construct
IQEYIDHLLTVASELESALIY

SEQ ID NO: 244 - protein, 21 aa, synthetic construct
  MOD_RES 3: note = ornithine
  MOD_RES 13: note = ornithine
IQXEIEHLNEVIXNLNESLID

SEQ ID NO: 245 - protein, 21 aa, synthetic construct
  MOD_RES 3: note = ornithine
  MOD_RES 7: note = ornithine
  MOD_RES 13: note = ornithine
IQXEIDXLNEVIXNLNESLID

SEQ ID NO: 246 - protein, 21 aa, synthetic construct
  MOD_RES 3: note = ornithine
  MOD_RES 13: note = ornithine
IQXEIEHLNEVAXNLGESLIE

SEQ ID NO: 247 - protein, 21 aa, synthetic construct
  MOD_RES 3: note = ornithine
  MOD_RES 7: note = ornithine
  MOD_RES 13: note = ornithine
IQXEIDXLNEVAXNLGESLIE

SEQ ID NO: 248 - protein, 21 aa, synthetic construct
  MOD_RES 3: note = ornithine
  MOD_RES 13: note = ornithine
IQXEIEHLNEVIXNLGESLIE

SEQ ID NO: 249 - protein, 21 aa, synthetic construct
  MOD_RES 3: note = ornithine
  MOD_RES 7: note = ornithine
  MOD_RES 13: note = ornithine
IQXEIDXLNEVIXNLGESLIE

SEQ ID NO: 250 - protein, 21 aa, synthetic construct
IQKEIDRLNEVAKNLNESLID

SEQ ID NO: 251 - protein, 25 aa, synthetic construct
IQKEIDRLNEVAKNLNESLIDLQEL

SEQ ID NO: 252 - protein, 21 aa, synthetic construct
  MOD_RES 3: note = ornithine
  MOD_RES 7: note = Homoarginine
  MOD_RES 13: note = ornithine
IQXEIDXLNEVIXNLNESLID

SEQ ID NO: 253 - protein, 21 aa, synthetic construct
  MOD_RES 3: note = ornithine
  MOD_RES 7: note = ornithine
  MOD_RES 13: note = ornithine
IQXEIDXLNEIIXNLNESLID

SEQ ID NO: 254 - protein, 36 aa, synthetic construct; identical to SEQ ID NO: 1

SEQ ID NO: 255 - protein, 42 aa, synthetic construct
PDVDLGDISGINASVVNIQKEIDRLNEVAKNLNESLIDLQEL

SEQ ID NO: 256 - protein, 25 aa, synthetic construct
IQKEIDRLNEVAKNLNESLIDLQEL

SEQ ID NO: 257 - protein, 36 aa, synthetic construct
DISGINASVVNIQKPIDRLNEVAKNLNESLIDLQEL

SEQ ID NO: 258 - protein, 36 aa, synthetic construct
DISGINASVVNIQKPIDPLNEVAKPLNEPLIDLQEL

SEQ ID NO: 259 - protein, 36 aa, synthetic construct
DISGINASVVNAQKEADRANEAAKNANESLIDLQEL

SEQ ID NO: 260 - protein, 36 aa, synthetic construct
DISGINASVVNIQKEIDRLNEVIKNLNESLIDLQEL

SEQ ID NO: 261 - protein, 36 aa, synthetic construct
DISGINASVVNIQKEIARLNEVAKALNESLIDLQEL

SEQ ID NO: 262 - protein, 36 aa, synthetic construct
DIAAINASVANIQKEIARLNEVAKALNESLAALQAL

SEQ ID NO: 263 - protein, 36 aa, synthetic construct
DISGINASVVNIQKEIARLNEVIKALNESLIDLQEL

SEQ ID NO: 264 - protein, 36 aa, synthetic construct
DISGINASVVNIQKEIERLNKVAKNLNESLIDLQEL

SEQ ID NO: 265 - protein, 36 aa, synthetic construct
DISGINASVVEIQKKIEELNKKAEELNKKLIDLQEL

SEQ ID NO: 266 - protein, 36 aa, synthetic construct
DISGINASVVDIKKEIEHLNEIAKSLNESLIDLQEL

SEQ ID NO: 267 - protein, 36 aa, synthetic construct
DISGINASVVDIKKEIEHLNEIAKNLNESLIDLQEL

SEQ ID NO: 268 - protein, 36 aa, synthetic construct
SLDQINVTFLDLEYEMKKLEEAIKKLEESYIDLKEL

SEQ ID NO: 269 - protein, 36 aa, synthetic construct
DISQINASVVNIEYEIKKLEEVAKKLEESLIDLQEL

SEQ ID NO: 270 - protein, 36 aa, synthetic construct
SIDQINATFVDIEYEIKKLEEVAKKLEESYIDLQEL

SEQ ID NO: 271 - protein, 47 aa, synthetic construct
KNHTSPDVDLGDISGINASVVNIQKEIDRLNEVAKNLNESLIDLQEL

SEQ ID NO: 272 - protein, 36 aa, synthetic construct
LEQLDILSENLNKAVENLRDIEKQINVVSANIGSID

SEQ ID NO: 273 - protein, 40 aa, synthetic construct
EAAAKEAAAKEAAAKEAAAKEAAAKEAAAKEAAAKEAAAK

SEQ ID NO: 274 - protein, 43 aa, synthetic construct
PDVDLGGDISGINASVVNIQKEIDRLNEVAKNLNESLIDLQEL

SEQ ID NO: 275 - protein, 48 aa, synthetic construct
KNHTSPDVDLGGDISGINASVVNIQKEIDRLNEVAKNLNESLIDLQEL

SEQ ID NO: 276 - protein, 40 aa, synthetic construct
DLGDISGINASVVNIQKEIDRLNEVAKNLNESLIDLQELG

SEQ ID NO: 277 - protein, 24 aa, synthetic construct
VVNIQKEIDRLNEVAKNLNESLID

SEQ ID NO: 278 - protein, 36 aa, synthetic construct
DISGINASVVNIQKEIDRLNEVAKNLNESLIELQEL

SEQ ID NO: 279 - protein, 36 aa, synthetic construct
DISGINASVVNIQKEIDRLNEVAKNLNESLIKLQEL

SEQ ID NO: 280 - protein, 36 aa, synthetic construct
DISGINASVVNIQKEIDRLNEVAKNLNESLIGLQEL

SEQ ID NO: 281 - protein, 36 aa, synthetic construct
DISGINASVVNIQKEIDRLNEVAKNLGESLIGLQEL

SEQ ID NO: 282 - protein, 36 aa, synthetic construct
DISGINASVVNIQKEIDRLNEVAKNLPESLIGLQEL

SEQ ID NO: 283 - protein, 36 aa, synthetic construct
DISGINASVVNIQKEIDRLDEVAKNLNESLIDLQEL

SEQ ID NO: 284 - protein, 36 aa, synthetic construct
DISGINASVVNIQKEIDRLGEVAKNLNESLIDLQEL

SEQ ID NO: 285 - protein, 36 aa, synthetic construct
DISGINASVENIQKEIDRLNEVAKNLNESLIDLQEL

SEQ ID NO: 286 - protein, 36 aa, synthetic construct
DISGINASVVNIQKEIDRLNEVAKNLGESLIELQEL

SEQ ID NO: 287 - protein, 36 aa, synthetic construct
DISGINASVVNIQKEIDRLDEVAKNLPESLIELQEL

SEQ ID NO: 288 - protein, 28 aa, synthetic construct
IEEIQKKIEEIQKKIEEIQKKIEEIQKK

SEQ ID NO: 289 - protein, 39 aa, synthetic construct
EKLDIYSENLNKAVENLTGLEQKLLQQVDLTAYSYILDE

SEQ ID NO: 290 - protein, 40 aa, synthetic construct
IPNFGSLTQINTTLLDLTYEMLSLQQVVKALNESYIDLKE

SEQ ID NO: 291 - protein, 39 aa, synthetic construct
DLGDISGINLTILNIQDEIEHLNELIDNLNESLIDLQEL

SEQ ID NO: 292 - protein, 39 aa, synthetic construct
DLGDISGINLTILNIQLEIEHLNELIENLGESLIELQEL

SEQ ID NO: 293 - protein, 39 aa, synthetic construct
DLGDISGINLSVVNIQLEIEHLNEVIENLGESLIELQEL

SEQ ID NO: 294 - protein, 39 aa, synthetic construct
DLGDISGINLSILNIQLEIEHLNEVIENLGESLIELQEL

SEQ ID NO: 295 - protein, 38 aa, synthetic construct
LGDISGINLSILNIQLEIEHLNEVIENLGESLIELQEL

SEQ ID NO: 296 - protein, 38 aa, synthetic construct
LGDISGINLTILNIQLEIEHLNELIENLGESLIELQEL

SEQ ID NO: 297 - protein, 38 aa, synthetic construct
LGDISGINLSVVNIQLEIEHLNEVIENLGESLIELQEL

SEQ ID NO: 298 - protein, 38 aa, synthetic construct
LGDISGINLSVVNIQLEIDHLNEVIENLGESLIELQEL

SEQ ID NO: 299 - protein, 39 aa, synthetic construct
DLGDISGINLSVVNIQLEIDHLNEVIENLGESLIELQEL

SEQ ID NO: 300 - protein, 39 aa, synthetic construct
DLGDISGINLSVVNIQLEIEHLNELIENLGESLIELQEL

SEQ ID NO: 301 - protein, 36 aa, synthetic construct
DISGINASVVNIQKEIDRLNEVIKNLGESLIELQEL

SEQ ID NO: 302 - protein, 38 aa, synthetic construct
LGDISGINASVVNIQKEIDRLNEVIKNLGESLIELQEL

SEQ ID NO: 303 - protein, 38 aa, synthetic construct
  MOD_RES 9: note = 2-aminoisobutyric acid
  MOD_RES 16: note = ornithine
  MOD_RES 26: note = ornithine
LGDISGINXSVVNIQXEIDHLNEVAXNLNESLIDLQEL

SEQ ID NO: 304 - protein, 38 aa, synthetic construct; identical to SEQ ID NO: 303
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 305 - protein, 38 aa, synthetic construct; identical to SEQ ID NO: 303
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 306 - protein, 38 aa, synthetic construct
  MOD_RES 9: note = 2-aminoisobutyric acid
  MOD_RES 16: note = ornithine
  MOD_RES 26: note = ornithine
LGDISGINXSVVNIQXEIEHLNEVAXNLNESLIDLQEL

SEQ ID NO: 307 - protein, 38 aa, synthetic construct; identical to SEQ ID NO: 306
  MOD_RES 9: note = 2-aminoisobutyric acid
  MOD_RES 16: note = 2,4-diaminobutyric acid
  MOD_RES 16: note = 2,4-diaminobutyric acid

SEQ ID NO: 308 - protein, 38 aa, synthetic construct; identical to SEQ ID NO: 306
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 309 - protein, 38 aa, synthetic construct; identical to SEQ ID NO: 306
  MOD_RES 9: note = 2-aminoisobutyric acid
  MOD_RES 16: note = N-Methyl-Lysine
  MOD_RES 26: note = N-Methyl-Lysine

SEQ ID NO: 310 - protein, 38 aa, synthetic construct; identical to SEQ ID NO: 306
  MOD_RES 9: note = 2-aminoisobutyric acid
  MOD_RES 16: note = N-Dimethyl-Lysine
  MOD_RES 26: note = N-Dimethyl-Lysine

SEQ ID NO: 311 - protein, 38 aa, synthetic construct; identical to SEQ ID NO: 306
  MOD_RES 9: note = 2-aminoisobutyric acid
  MOD_RES 16: note = N alpha-methyl-L-lysine
  MOD_RES 26: note = ornithine

SEQ ID NO: 312 - protein, 38 aa, synthetic construct; identical to SEQ ID NO: 306
  MOD_RES 9: note = 2-aminoisobutyric acid
  MOD_RES 16: note = N alpha-methyl-L-lysine
  MOD_RES 26: note = 2,4-diaminobutyric acid

SEQ ID NO: 313 - protein, 38 aa, synthetic construct; identical to SEQ ID NO: 306
  MOD_RES 9: note = 2-aminoisobutyric acid
  MOD_RES 16: note = N alpha-methyl-L-lysine
  MOD_RES 26: note = 2,3-diaminopropionic acid

SEQ ID NO: 314 - protein, 38 aa, synthetic construct; identical to SEQ ID NO: 306
  MOD_RES 9: note = 2-aminoisobutyric acid
  MOD_RES 16: note = N alpha-methyl-L-lysine
  MOD_RES 26: note = N-Methyl-Lysine

SEQ ID NO: 315 - protein, 38 aa, synthetic construct; identical to SEQ ID NO: 306
  MOD_RES 9: note = 2-aminoisobutyric acid
  MOD_RES 16: note = N alpha-methyl-L-lysine
  MOD_RES 26: note = N-Dimethyl-Lysine

SEQ ID NO: 316 - protein, 38 aa, synthetic construct
  MOD_RES 9: note = 2-aminoisobutyric acid
  MOD_RES 16: note = ornithine
  MOD_RES 20: note = ornithine
  MOD_RES 26: note = ornithine
LGDISGINXSVVNIQXEIDXLNEVAXNLNESLIDLQEL

SEQ ID NO: 317 - protein, 38 aa, synthetic construct; identical to SEQ ID NO: 316
  MOD_RES 9: note = 2-aminoisobutyric acid
  MOD_RES 16: note = 2,4-diaminobutyric acid
  MOD_RES 26: note = 2,4-diaminobutyric acid
  MOD_RES 20: note = ornithine

SEQ ID NO: 318 - protein, 38 aa, synthetic construct; identical to SEQ ID NO: 316
  MOD_RES 9: note = 2-aminoisobutyric acid
  MOD_RES 16: note = 2,3-diaminopropionic acid
  MOD_RES 26: note = 2,3-diaminopropionic acid
  MOD_RES 20: note = ornithine

SEQ ID NO: 319 - protein, 38 aa, synthetic construct; identical to SEQ ID NO: 316
  MOD_RES 9: note = 2-aminoisobutyric acid
  MOD_RES 16: note = N-Methyl-Lysine
  MOD_RES 26: note = N-Methyl-Lysine
  MOD_RES 20: note = ornithine

SEQ ID NO: 320 - protein, 38 aa, synthetic construct; identical to SEQ ID NO: 316
  MOD_RES 9: note = 2-aminoisobutyric acid
  MOD_RES 16: note = N-Dimethyl-Lysine
  MOD_RES 26: note = N-Dimethyl-Lysine
  MOD_RES 20: note = ornithine

SEQ ID NO: 321 - protein, 38 aa, synthetic construct; identical to SEQ ID NO: 316
  MOD_RES 9: note = 2-aminoisobutyric acid
  MOD_RES 16: note = N alpha-methyl-L-lysine
  MOD_RES 20: note = ornithine
  MOD_RES 26: note = ornithine

SEQ ID NO: 322 - protein, 38 aa, synthetic construct; identical to SEQ ID NO: 316
  MOD_RES 9: note = 2-aminoisobutyric acid
  MOD_RES 16: note = N alpha-methyl-L-lysine
  MOD_RES 20: note = ornithine
  MOD_RES 26: note = 2,4-diaminobutyric acid

SEQ ID NO: 323 - protein, 38 aa, synthetic construct; identical to SEQ ID NO: 316
  MOD_RES 9: note = 2-aminoisobutyric acid
  MOD_RES 16: note = N alpha-methyl-L-lysine
  MOD_RES 20: note = ornithine
  MOD_RES 26: note = 2,3-diaminopropionic acid

SEQ ID NO: 324 - protein, 38 aa, synthetic construct; identical to SEQ ID NO: 316
  MOD_RES 9: note = 2-aminoisobutyric acid
  MOD_RES 16: note = N alpha-methyl-L-lysine
  MOD_RES 20: note = ornithine
  MOD_RES 26: note = N-Methyl-Lysine

SEQ ID NO: 325 - protein, 38 aa, synthetic construct; identical to SEQ ID NO: 316
  MOD_RES 9: note = 2-aminoisobutyric acid
  MOD_RES 16: note = N alpha-methyl-L-lysine
  MOD_RES 20: note = ornithine
  MOD_RES 26: note = N-Dimethyl-Lysine

SEQ ID NO: 326 - protein, 38 aa, synthetic construct; identical to SEQ ID NO: 316
  MOD_RES 9: note = 2-aminoisobutyric acid
  MOD_RES 16: note = ornithine
  MOD_RES 26: note = ornithine
  MOD_RES 20: note = Citrulline

SEQ ID NO: 327 - protein, 38 aa, synthetic construct; identical to SEQ ID NO: 316
  MOD_RES 9: note = 2-aminoisobutyric acid
  MOD_RES 16: note = 2,4-diaminobutyric acid
  MOD_RES 26: note = 2,4-diaminobutyric acid
  MOD_RES 20: note = Citrulline

SEQ ID NO: 328 - protein, 38 aa, synthetic construct; identical to SEQ ID NO: 316
  MOD_RES 9: note = 2-aminoisobutyric acid
  MOD_RES 16: note = 2,3-diaminopropionic acid
  MOD_RES 26: note = 2,3-diaminopropionic acid
  MOD_RES 20: note = Citrulline

SEQ ID NO: 329 - protein, 38 aa, synthetic construct; identical to SEQ ID NO: 316
  MOD_RES 9: note = 2-aminoisobutyric acid
  MOD_RES 16: note = N-Methyl-Lysine
  MOD_RES 26: note = N-Methyl-Lysine
  MOD_RES 20: note = Citrulline

SEQ ID NO: 330 - protein, 38 aa, synthetic construct; identical to SEQ ID NO: 316
  MOD_RES 9: note = 2-aminoisobutyric acid
  MOD_RES 16: note = N-Dimethyl-Lysine
  MOD_RES 26: note = N-Dimethyl-Lysine
  MOD_RES 20: note = Citrulline

SEQ ID NO: 331 - protein, 38 aa, synthetic construct; identical to SEQ ID NO: 316
  MOD_RES 9: note = 2-aminoisobutyric acid
  MOD_RES 16: note = N alpha-methyl-L-lysine
  MOD_RES 20: note = Citrulline
  MOD_RES 26: note = ornithine

SEQ ID NO: 332 - protein, 38 aa, synthetic construct; identical to SEQ ID NO: 316
  MOD_RES 9: note = 2-aminoisobutyric acid
  MOD_RES 16: note = N alpha-methyl-L-lysine
  MOD_RES 20: note = Citrulline
  MOD_RES 26: note = 2,4-diaminobutyric acid

SEQ ID NO: 333 - protein, 38 aa, synthetic construct; identical to SEQ ID NO: 316
  MOD_RES 9: note = 2-aminoisobutyric acid
  MOD_RES 16: note = N alpha-methyl-L-lysine
  MOD_RES 20: note = Citrulline
  MOD_RES 26: note = 2,3-diaminopropionic acid

SEQ ID NO: 334 - protein, 38 aa, synthetic construct; identical to SEQ ID NO: 316
  MOD_RES 9: note = 2-aminoisobutyric acid
  MOD_RES 16: note = N alpha-methyl-L-lysine
  MOD_RES 20: note = Citrulline
  MOD_RES 26: note = N-Methyl-Lysine

SEQ ID NO: 335 - protein, 38 aa, synthetic construct; identical to SEQ ID NO: 316
  MOD_RES 9: note = 2-aminoisobutyric acid
  MOD_RES 16: note = N alpha-methyl-L-lysine
  MOD_RES 20: note = Citrulline
  MOD_RES 26: note = N-Dimethyl-Lysine

SEQ ID NO: 336 - protein, 38 aa, synthetic construct
  MOD_RES 9: note = 2-aminoisobutyric acid
  MOD_RES 16: note = ornithine
  MOD_RES 26: note = ornithine
LGDISGINXSVVNIQXEIDHLNEVIXNLNESLIDLQEL

SEQ ID NO: 337 - protein, 38 aa, synthetic construct; identical to SEQ ID NO: 336
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 338 - protein, 38 aa, synthetic construct; identical to SEQ ID NO: 336
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 339 - protein, 38 aa, synthetic construct
  MOD_RES 9: note = 2-aminoisobutyric acid
  MOD_RES 16: note = ornithine
  MOD_RES 26: note = ornithine
LGDISGINXSVVNIQXEIEHLNEVIXNLNESLIDLQEL

SEQ ID NO: 340 - protein, 38 aa, synthetic construct; identical to SEQ ID NO: 339
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 341 - protein, 38 aa, synthetic construct; identical to SEQ ID NO: 339
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 342 - protein, 38 aa, synthetic construct; identical to SEQ ID NO: 339
  MOD_RES 9: note = 2-aminoisobutyric acid
  MOD_RES 16: note = N-Methyl-Lysine
  MOD_RES 26: note = N-Methyl-Lysine

SEQ ID NO: 343 - protein, 38 aa, synthetic construct; identical to SEQ ID NO: 339
  MOD_RES 9: note = 2-aminoisobutyric acid
  MOD_RES 16: note = N-Dimethyl-Lysine
  MOD_RES 26: note = N-Dimethyl-Lysine

SEQ ID NO: 344 - protein, 38 aa, synthetic construct; identical to SEQ ID NO: 339
  MOD_RES 9: note = 2-aminoisobutyric acid
  MOD_RES 16: note = N alpha-methyl-L-lysine
  MOD_RES 26: note = ornithine

SEQ ID NO: 345 - protein, 38 aa, synthetic construct; identical to SEQ ID NO: 339
  MOD_RES 9: note = 2-aminoisobutyric acid
  MOD_RES 16: note = N alpha-methyl-L-lysine
  MOD_RES 26: note = 2,4-diaminobutyric acid

SEQ ID NO: 346 - protein, 38 aa, synthetic construct; identical to SEQ ID NO: 339
  MOD_RES 9: note = 2-aminoisobutyric acid
  MOD_RES 16: note = N alpha-methyl-L-lysine
  MOD_RES 26: note = 2,3-diaminopropionic acid

SEQ ID NO: 347 - protein, 38 aa, synthetic construct; identical to SEQ ID NO: 339
  MOD_RES 9: note = 2-aminoisobutyric acid
  MOD_RES 16: note = N alpha-methyl-L-lysine
  MOD_RES 26: note = N-Methyl-Lysine

SEQ ID NO: 348 - protein, 38 aa, synthetic construct; identical to SEQ ID NO: 339
  MOD_RES 9: note = 2-aminoisobutyric acid
  MOD_RES 16: note = N alpha-methyl-L-lysine
  MOD_RES 26: note = N-Dimethyl-Lysine

SEQ ID NO: 349 - protein, 38 aa, synthetic construct
  MOD_RES 9: note = 2-aminoisobutyric acid
  MOD_RES 16: note = ornithine
  MOD_RES 20: note = ornithine
  MOD_RES 26: note = ornithine
LGDISGINXSVVNIQXEIDXLNEVIXNLNESLIDLQEL

SEQ ID NO: 350 - protein, 38 aa, synthetic construct; identical to SEQ ID NO: 349
  MOD_RES 9: note = 2-aminoisobutyric acid
  MOD_RES 16: note = 2,4-diaminobutyric acid
  MOD_RES 26: note = 2,4-diaminobutyric acid
  MOD_RES 20: note = ornithine

SEQ ID NO: 351 - protein, 38 aa, synthetic construct; identical to SEQ ID NO: 349
  MOD_RES 9: note = 2-aminoisobutyric acid
  MOD_RES 16: note = 2,3-diaminopropionic acid
  MOD_RES 26: note = 2,3-diaminopropionic acid
  MOD_RES 20: note = ornithine

SEQ ID NO: 352 - protein, 38 aa, synthetic construct; identical to SEQ ID NO: 349
  MOD_RES 9: note = 2-aminoisobutyric acid
  MOD_RES 16: note = N-Methyl-Lysine
  MOD_RES 26: note = N-Methyl-Lysine
  MOD_RES 20: note = ornithine

SEQ ID NO: 353 - protein, 38 aa, synthetic construct; identical to SEQ ID NO: 349
  MOD_RES 9: note = 2-aminoisobutyric acid
  MOD_RES 16: note = N-Dimethyl-Lysine
  MOD_RES 26: note = N-Dimethyl-Lysine
  MOD_RES 20: note = ornithine

SEQ ID NO: 354 - protein, 38 aa, synthetic construct; identical to SEQ ID NO: 349
  MOD_RES 9: note = 2-aminoisobutyric acid
  MOD_RES 16: note = N alpha-methyl-L-lysine
  MOD_RES 26: note = ornithine
  MOD_RES 20: note = ornithine

SEQ ID NO: 355 - protein, 38 aa, synthetic construct; identical to SEQ ID NO: 349
  MOD_RES 9: note = 2-aminoisobutyric acid
  MOD_RES 16: note = N alpha-methyl-L-lysine
  MOD_RES 20: note = ornithine
  MOD_RES 26: note = 2,4-diaminobutyric acid

SEQ ID NO: 356 - protein, 38 aa, synthetic construct; identical to SEQ ID NO: 349
  MOD_RES 9: note = 2-aminoisobutyric acid
  MOD_RES 16: note = N alpha-methyl-L-lysine
  MOD_RES 20: note = ornithine
  MOD_RES 26: note = 2,3-diaminopropionic acid

SEQ ID NO: 357 - protein, 38 aa, synthetic construct; identical to SEQ ID NO: 349
  MOD_RES 9: note = 2-aminoisobutyric acid
  MOD_RES 16: note = N alpha-methyl-L-lysine
  MOD_RES 20: note = ornithine
  MOD_RES 26: note = N-Methyl-Lysine

SEQ ID NO: 358 - protein, 38 aa, synthetic construct; identical to SEQ ID NO: 349
  MOD_RES 9: note = 2-aminoisobutyric acid
  MOD_RES 16: note = N alpha-methyl-L-lysine
  MOD_RES 20: note = ornithine
  MOD_RES 26: note = N-Dimethyl-Lysine

SEQ ID NO: 359 - protein, 38 aa, synthetic construct; identical to SEQ ID NO: 349
  MOD_RES 9: note = 2-aminoisobutyric acid
  MOD_RES 16: note = ornithine
  MOD_RES 20: note = Citrulline
  MOD_RES 26: note = ornithine

SEQ ID NO: 360 - protein, 38 aa, synthetic construct; identical to SEQ ID NO: 349
  MOD_RES 9: note = 2-aminoisobutyric acid
  MOD_RES 16: note = 2,4-diaminobutyric acid
  MOD_RES 26: note = 2,4-diaminobutyric acid
  MOD_RES 20: note = Citrulline

SEQ ID NO: 361 - protein, 38 aa, synthetic construct; identical to SEQ ID NO: 349
  MOD_RES 9: note = 2-aminoisobutyric acid
  MOD_RES 16: note = 2,3-diaminopropionic acid
  MOD_RES 26: note = 2,3-diaminopropionic acid
  MOD_RES 20: note = Citrulline

SEQ ID NO: 362 - protein, 38 aa, synthetic construct; identical to SEQ ID NO: 349
  MOD_RES 9: note = 2-aminoisobutyric acid
  MOD_RES 16: note = N-Methyl-Lysine
  MOD_RES 26: note = N-Methyl-Lysine
  MOD_RES 20: note = Citrulline

SEQ ID NO: 363 - protein, 38 aa, synthetic construct; identical to SEQ ID NO: 349
  MOD_RES 9: note = 2-aminoisobutyric acid
  MOD_RES 16: note = N-Dimethyl-Lysine
  MOD_RES 26: note = N-Dimethyl-Lysine
  NON_STD 20: note = Citrulline

SEQ ID NO: 364 - protein, 38 aa, synthetic construct; identical to SEQ ID NO: 349
  MOD_RES 9: note = 2-aminoisobutyric acid
  MOD_RES 16: note = N alpha-methyl-L-lysine
  MOD_RES 26: note = ornithine
  MOD_RES 20: note = Citrulline

SEQ ID NO: 365 - protein, 38 aa, synthetic construct; identical to SEQ ID NO: 349
  MOD_RES 9: note = 2-aminoisobutyric acid
  MOD_RES 16: note = N alpha-methyl-L-lysine
  MOD_RES 26: note = 2,4-diaminobutyric acid
  MOD_RES 20: note = Citrulline

SEQ ID NO: 366 - protein, 38 aa, synthetic construct; identical to SEQ ID NO: 349
  MOD_RES 9: note = 2-aminoisobutyric acid
  MOD_RES 16: note = N alpha-methyl-L-lysine
  MOD_RES 26: note = 2,3-diaminopropionic acid
  MOD_RES 20: note = Citrulline

SEQ ID NO: 367 - protein, 38 aa, synthetic construct; identical to SEQ ID NO: 349
  MOD_RES 9: note = 2-aminoisobutyric acid
  MOD_RES 16: note = N alpha-methyl-L-lysine
  MOD_RES 26: note = N-Methyl-Lysine
  MOD_RES 20: note = Citrulline

SEQ ID NO: 368 - protein, 38 aa, synthetic construct; identical to SEQ ID NO: 349
  MOD_RES 9: note = 2-aminoisobutyric acid
  MOD_RES 16: note = N alpha-methyl-L-lysine
  MOD_RES 26: note = N-Dimethyl-Lysine
  MOD_RES 20: note = Citrulline

SEQ ID NO: 369 - protein, 38 aa, synthetic construct
  MOD_RES 9: note = 2-aminoisobutyric acid
  MOD_RES 16: note = ornithine
  MOD_RES 26: note = ornithine
LGDISGINXSVVNIQXEIDHLNEVAXNLGESLIELQEL

SEQ ID NO: 370 - protein, 38 aa, synthetic construct; identical to SEQ ID NO: 369
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 371 - protein, 38 aa, synthetic construct; identical to SEQ ID NO: 369
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 372 - protein, 38 aa, synthetic construct
  MOD_RES 9: note = 2-aminoisobutyric acid
  MOD_RES 16: note = ornithine
  MOD_RES 26: note = ornithine
LGDISGINXSVVNIQXEIEHLNEVAXNLGESLIELQEL

SEQ ID NO: 373 - protein, 38 aa, synthetic construct; identical to SEQ ID NO: 372
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 374 - protein, 38 aa, synthetic construct; identical to SEQ ID NO: 372
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 375 - protein, 38 aa, synthetic construct; identical to SEQ ID NO: 372
  MOD_RES 9: note = 2-aminoisobutyric acid
  MOD_RES 16: note = N-Methyl-Lysine
  MOD_RES 26: note = N-Methyl-Lysine

SEQ ID NO: 376 - protein, 38 aa, synthetic construct; identical to SEQ ID NO: 372
  MOD_RES 9: note = 2-aminoisobutyric acid
  MOD_RES 16: note = N-Dimethyl-Lysine
  MOD_RES 26: note = N-Dimethyl-Lysine

SEQ ID NO: 377 - protein, 38 aa, synthetic construct; identical to SEQ ID NO: 372
  MOD_RES 9: note = 2-aminoisobutyric acid
  MOD_RES 16: note = N alpha-methyl-L-lysine
  MOD_RES 26: note = ornithine

SEQ ID NO: 378 - protein, 38 aa, synthetic construct; identical to SEQ ID NO: 372
  MOD_RES 9: note = 2-aminoisobutyric acid
  MOD_RES 16: note = N alpha-methyl-L-lysine
  MOD_RES 26: note = 2,4-diaminobutyric acid

SEQ ID NO: 379 - protein, 38 aa, synthetic construct; identical to SEQ ID NO: 372
  MOD_RES 9: note = 2-aminoisobutyric acid
  MOD_RES 16: note = N alpha-methyl-L-lysine
  MOD_RES 26: note = 2,3-diaminopropionic acid

SEQ ID NO: 380 - protein, 38 aa, synthetic construct; identical to SEQ ID NO: 372
  MOD_RES 9: note = 2-aminoisobutyric acid
  MOD_RES 16: note = N alpha-methyl-L-lysine
  MOD_RES 26: note = N-Methyl-Lysine

SEQ ID NO: 381 - protein, 38 aa, synthetic construct; identical to SEQ ID NO: 372
  MOD_RES 9: note = 2-aminoisobutyric acid
  MOD_RES 16: note = N alpha-methyl-L-lysine
  MOD_RES 26: note = N-Dimethyl-Lysine

SEQ ID NO: 382 - protein, 38 aa, synthetic construct
  MOD_RES 9: note = 2-aminoisobutyric acid
  MOD_RES 16: note = ornithine
  MOD_RES 26: note = ornithine
  MOD_RES 20: note = ornithine
LGDISGINXSVVNIQXEIDXLNEVAXNLGESLIELQEL

SEQ ID NO: 383 - protein, 38 aa, synthetic construct; identical to SEQ ID NO: 382
  MOD_RES 9: note = 2-aminoisobutyric acid
  MOD_RES 16: note = 2,4-diaminobutyric acid
  MOD_RES 26: note = 2,4-diaminobutyric acid
  MOD_RES 20: note = ornithine

SEQ ID NO: 384 - protein, 38 aa, synthetic construct; identical to SEQ ID NO: 382
  MOD_RES 9: note = 2-aminoisobutyric acid
  MOD_RES 16: note = 2,3-diaminopropionic acid
  MOD_RES 26: note = 2,3-diaminopropionic acid
  MOD_RES 20: note = ornithine

SEQ ID NO: 385 - protein, 38 aa, synthetic construct; identical to SEQ ID NO: 382
  MOD_RES 9: note = 2-aminoisobutyric acid
  MOD_RES 16: note = N-Methyl-Lysine
  MOD_RES 26: note = N-Methyl-Lysine
  MOD_RES 20: note = ornithine

SEQ ID NO: 386 - protein, 38 aa, synthetic construct; identical to SEQ ID NO: 382
  MOD_RES 9: note = 2-aminoisobutyric acid
  MOD_RES 16: note = N-Dimethyl-Lysine
  MOD_RES 26: note = N-Dimethyl-Lysine
  MOD_RES 20: note = ornithine

SEQ ID NO: 387 - protein, 38 aa, synthetic construct; identical to SEQ ID NO: 382
  MOD_RES 9: note = 2-aminoisobutyric acid
  MOD_RES 16: note = N alpha-methyl-L-lysine
  MOD_RES 26: note = ornithine
  MOD_RES 20: note = ornithine

SEQ ID NO: 388 - protein, 38 aa, synthetic construct; identical to SEQ ID NO: 382
  MOD_RES 9: note = 2-aminoisobutyric acid
  MOD_RES 16: note = N alpha-methyl-L-lysine
  MOD_RES 26: note = 2,4-diaminobutyric acid
  MOD_RES 20: note = ornithine

SEQ ID NO: 389 - protein, 38 aa, synthetic construct; identical to SEQ ID NO: 382
  MOD_RES 9: note = 2-aminoisobutyric acid
  MOD_RES 16: note = N alpha-methyl-L-lysine
  MOD_RES 26: note = 2,3-diaminopropionic acid
  MOD_RES 20: note = ornithine

SEQ ID NO: 390 - protein, 38 aa, synthetic construct; identical to SEQ ID NO: 382
  MOD_RES 9: note = 2-aminoisobutyric acid
  MOD_RES 16: note = N alpha-methyl-L-lysine
  MOD_RES 20: note = ornithine
  MOD_RES 26: note = N-Methyl-Lysine

SEQ ID NO: 391 - protein, 38 aa, synthetic construct; identical to SEQ ID NO: 382
  MOD_RES 9: note = 2-aminoisobutyric acid
  MOD_RES 16: note = N alpha-methyl-L-lysine
  MOD_RES 20: note = ornithine
  MOD_RES 26: note = N-Dimethyl-Lysine

SEQ ID NO: 392 - protein, 38 aa, synthetic construct; identical to SEQ ID NO: 382
  MOD_RES 9: note = 2-aminoisobutyric acid
  MOD_RES 16: note = ornithine
  MOD_RES 20: note = Citrulline
  MOD_RES 26: note = ornithine

SEQ ID NO: 393 - protein, 38 aa, synthetic construct; identical to SEQ ID NO: 382
  MOD_RES 9: note = 2-aminoisobutyric acid
  MOD_RES 16: note = 2,4-diaminobutyric acid
  MOD_RES 20: note = Citrulline
  MOD_RES 26: note = 2,4-diaminobutyric acid

SEQ ID NO: 394 - protein, 38 aa, synthetic construct; identical to SEQ ID NO: 382
  MOD_RES 9: note = 2-aminoisobutyric acid
  MOD_RES 16: note = 2,3-diaminopropionic acid
  MOD_RES 20: note = Citrulline
  MOD_RES 26: note = 2,3-diaminopropionic acid

SEQ ID NO: 395 - protein, 38 aa, synthetic construct; identical to SEQ ID NO: 382
  MOD_RES 9: note = 2-aminoisobutyric acid
  MOD_RES 16: note = N-Methyl-Lysine
  MOD_RES 20: note = Citrulline
  MOD_RES 26: note = N-Methyl-Lysine

SEQ ID NO: 396 - protein, 38 aa, synthetic construct; identical to SEQ ID NO: 382
  MOD_RES 9: note = 2-aminoisobutyric acid
  MOD_RES 16: note = N-Dimethyl-Lysine
  MOD_RES 20: note = Citrulline
  MOD_RES 26: note = N-Dimethyl-Lysine

SEQ ID NO: 397 - protein, 38 aa, synthetic construct; identical to SEQ ID NO: 382
  MOD_RES 9: note = 2-aminoisobutyric acid
  MOD_RES 16: note = N alpha-methyl-L-lysine
  MOD_RES 20: note = Citrulline
  MOD_RES 26: note = ornithine

SEQ ID NO: 398 - protein, 38 aa, synthetic construct; identical to SEQ ID NO: 382
  MOD_RES 9: note = 2-aminoisobutyric acid
  MOD_RES 16: note = N alpha-methyl-L-lysine
  MOD_RES 20: note = Citrulline
  MOD_RES 26: note = 2,4-diaminobutyric acid

SEQ ID NO: 399 - protein, 38 aa, synthetic construct; identical to SEQ ID NO: 382
  MOD_RES 9: note = 2-aminoisobutyric acid
  MOD_RES 16: note = N alpha-methyl-L-lysine
  MOD_RES 20: note = Citrulline
  MOD_RES 26: note = 2,3-diaminopropionic acid

SEQ ID NO: 400 - protein, 38 aa, synthetic construct; identical to SEQ ID NO: 382
  MOD_RES 9: note = 2-aminoisobutyric acid
  MOD_RES 16: note = N alpha-methyl-L-lysine
  MOD_RES 20: note = Citrulline
  MOD_RES 26: note = N-Methyl-Lysine

SEQ ID NO: 401 - protein, 38 aa, synthetic construct; identical to SEQ ID NO: 382
  MOD_RES 9: note = 2-aminoisobutyric acid
  MOD_RES 16: note = N alpha-methyl-L-lysine
  MOD_RES 20: note = Citrulline
  MOD_RES 26: note = N-Dimethyl-Lysine

SEQ ID NO: 402 - protein, 38 aa, synthetic construct
  MOD_RES 9: note = 2-aminoisobutyric acid
  MOD_RES 16: note = ornithine
  MOD_RES 26: note = ornithine
LGDISGINXSVVNIQXEIDHLNEVIXNLGESLIELQEL

SEQ ID NO: 403 - protein, 38 aa, synthetic construct; identical to SEQ ID NO: 402
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 404 - protein, 38 aa, synthetic construct; identical to SEQ ID NO: 402
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 405 - protein, 38 aa, synthetic construct
  MOD_RES 9: note = 2-aminoisobutyric acid
  MOD_RES 16: note = ornithine
  MOD_RES 26: note = ornithine
LGDISGINXSVVNIQXEIEHLNEVIXNLGESLIELQEL

SEQ ID NO: 406 - protein, 38 aa, synthetic construct; identical to SEQ ID NO: 405
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 407 - protein, 38 aa, synthetic construct; identical to SEQ ID NO: 405
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 408 - protein, 38 aa, synthetic construct; identical to SEQ ID NO: 405
  MOD_RES 9: note = 2-aminoisobutyric acid
  MOD_RES 16: note = N-Methyl-Lysine
  MOD_RES 26: note = N-Methyl-Lysine

SEQ ID NO: 409 - protein, 38 aa, synthetic construct; identical to SEQ ID NO: 405
  MOD_RES 9: note = 2-aminoisobutyric acid
  MOD_RES 16: note = N-Dimethyl-Lysine
  MOD_RES 26: note = N-Dimethyl-Lysine

SEQ ID NO: 410 - protein, 38 aa, synthetic construct; identical to SEQ ID NO: 405
  MOD_RES 9: note = 2-aminoisobutyric acid
  MOD_RES 16: note = N alpha-methyl-L-lysine
  MOD_RES 26: note = ornithine

SEQ ID NO: 411 - protein, 38 aa, synthetic construct; identical to SEQ ID NO: 405
  MOD_RES 9: note = 2-aminoisobutyric acid
  MOD_RES 16: note = N alpha-methyl-L-lysine
  MOD_RES 26: note = 2,4-diaminobutyric acid

SEQ ID NO: 412 - protein, 38 aa, synthetic construct; identical to SEQ ID NO: 405
  MOD_RES 9: note = 2-aminoisobutyric acid
  MOD_RES 16: note = N alpha-methyl-L-lysine
  MOD_RES 26: note = 2,3-diaminopropionic acid

SEQ ID NO: 413 - protein, 38 aa, synthetic construct; identical to SEQ ID NO: 405
  MOD_RES 9: note = 2-aminoisobutyric acid
  MOD_RES 16: note = N alpha-methyl-L-lysine
  MOD_RES 26: note = N-Methyl-Lysine

SEQ ID NO: 414 - protein, 38 aa, synthetic construct; identical to SEQ ID NO: 405
  MOD_RES 9: note = 2-aminoisobutyric acid
  MOD_RES 16: note = N alpha-methyl-L-lysine
  MOD_RES 26: note = N-Dimethyl-Lysine

SEQ ID NO: 415 - protein, 38 aa, synthetic construct
  MOD_RES 9: note = 2-aminoisobutyric acid
  MOD_RES 16: note = ornithine
  MOD_RES 20: note = ornithine
  MOD_RES 26: note = ornithine
LGDISGINXSVVNIQXEIDXLNEVIXNLGESLIELQEL

SEQ ID NO: 416 - protein, 38 aa, synthetic construct; identical to SEQ ID NO: 415
  MOD_RES 9: note = 2-aminoisobutyric acid
  MOD_RES 16: note = 2,4-diaminobutyric acid
  MOD_RES 20: note = ornithine
  MOD_RES 26: note = 2,4-diaminobutyric acid

SEQ ID NO: 417 - protein, 38 aa, synthetic construct; identical to SEQ ID NO: 415
  MOD_RES 9: note = 2-aminoisobutyric acid
  MOD_RES 16: note = 2,3-diaminopropionic acid
  MOD_RES 20: note = ornithine
  MOD_RES 26: note = 2,3-diaminopropionic acid

SEQ ID NO: 418 - protein, 38 aa, synthetic construct; identical to SEQ ID NO: 415
  MOD_RES 9: note = 2-aminoisobutyric acid
  MOD_RES 16: note = N-Methyl-Lysine
  MOD_RES 20: note = ornithine
  MOD_RES 26: note = N-Methyl-Lysine

SEQ ID NO: 419 - protein, 38 aa, synthetic construct; identical to SEQ ID NO: 415
  MOD_RES 9: note = 2-aminoisobutyric acid
  MOD_RES 16: note = N-Dimethyl-Lysine
  MOD_RES 20: note = ornithine
  MOD_RES 26: note = N-Dimethyl-Lysine

SEQ ID NO: 420 - protein, 38 aa, synthetic construct; identical to SEQ ID NO: 415
  MOD_RES 9: note = 2-aminoisobutyric acid
  MOD_RES 16: note = N alpha-methyl-L-lysine
  MOD_RES 20: note = ornithine
  MOD_RES 26: note = ornithine

SEQ ID NO: 421 - protein, 38 aa, synthetic construct; identical to SEQ ID NO: 415
  MOD_RES 9: note = 2-aminoisobutyric acid
  MOD_RES 16: note = N alpha-methyl-L-lysine
  MOD_RES 20: note = ornithine
  MOD_RES 26: note = 2,4-diaminobutyric acid

SEQ ID NO: 422 - protein, 38 aa, synthetic construct; identical to SEQ ID NO: 415
  MOD_RES 9: note = 2-aminoisobutyric acid
  MOD_RES 16: note = N alpha-methyl-L-lysine
  MOD_RES 20: note = ornithine
  MOD_RES 26: note = 2,3-diaminopropionic acid

SEQ ID NO: 423 - protein, 38 aa, synthetic construct; identical to SEQ ID NO: 415
  MOD_RES 9: note = 2-aminoisobutyric acid
  MOD_RES 16: note = N alpha-methyl-L-lysine
  MOD_RES 20: note = ornithine
  MOD_RES 26: note = N-Methyl-Lysine

SEQ ID NO: 424 - protein, 38 aa, synthetic construct; identical to SEQ ID NO: 415
  MOD_RES 9: note = 2-aminoisobutyric acid
  MOD_RES 16: note = N alpha-methyl-L-lysine
  MOD_RES 20: note = ornithine
  MOD_RES 26: note = N-Dimethyl-Lysine

SEQ ID NO: 425 - protein, 38 aa, synthetic construct; identical to SEQ ID NO: 415
  MOD_RES 9: note = 2-aminoisobutyric acid
  MOD_RES 16: note = ornithine
  MOD_RES 20: note = Citrulline
  MOD_RES 26: note = ornithine

SEQ ID NO: 426 - protein, 38 aa, synthetic construct; identical to SEQ ID NO: 415
  MOD_RES 9: note = 2-aminoisobutyric acid
  MOD_RES 16: note = 2,4-diaminobutyric acid
  MOD_RES 20: note = Citrulline
  MOD_RES 26: note = 2,4-diaminobutyric acid

SEQ ID NO: 427 - protein, 38 aa, synthetic construct; identical to SEQ ID NO: 415
  MOD_RES 9: note = 2-aminoisobutyric acid
  MOD_RES 16: note = 2,3-diaminopropionic acid
  MOD_RES 20: note = Citrulline
  MOD_RES 26: note = 2,3-diaminopropionic acid

SEQ ID NO: 428 - protein, 38 aa, synthetic construct; identical to SEQ ID NO: 415
  MOD_RES 9: note = 2-aminoisobutyric acid
  MOD_RES 16: note = N-Methyl-Lysine
  MOD_RES 20: note = Citrulline
  MOD_RES 26: note = N-Methyl-Lysine

SEQ ID NO: 429 - protein, 38 aa, synthetic construct; identical to SEQ ID NO: 415
  MOD_RES 9: note = 2-aminoisobutyric acid
  MOD_RES 16: note = N-Dimethyl-Lysine
  MOD_RES 20: note = Citrulline
  MOD_RES 26: note = N-Dimethyl-Lysine

SEQ ID NO: 430 - protein, 38 aa, synthetic construct; identical to SEQ ID NO: 415
  MOD_RES 9: note = 2-aminoisobutyric acid
  MOD_RES 16: note = N alpha-methyl-L-lysine
  MOD_RES 20: note = Citrulline
  MOD_RES 26: note = ornithine

SEQ ID NO: 431 - protein, 38 aa, synthetic construct; identical to SEQ ID NO: 415
  MOD_RES 9: note = 2-aminoisobutyric acid
  MOD_RES 16: note = N alpha-methyl-L-lysine
  MOD_RES 20: note = Citrulline
  MOD_RES 26: note = 2,4-diaminobutyric acid

SEQ ID NO: 432 - protein, 38 aa, synthetic construct; identical to SEQ ID NO: 415
  MOD_RES 9: note = 2-aminoisobutyric acid
  MOD_RES 16: note = N alpha-methyl-L-lysine
  MOD_RES 20: note = Citrulline
  MOD_RES 26: note = 2,3-diaminopropionic acid

SEQ ID NO: 433 - protein, 38 aa, synthetic construct; identical to SEQ ID NO: 415
  MOD_RES 9: note = 2-aminoisobutyric acid
  MOD_RES 16: note = N alpha-methyl-L-lysine
  MOD_RES 20: note = Citrulline
  MOD_RES 26: note = N-Methyl-Lysine

SEQ ID NO: 434 - protein, 38 aa, synthetic construct; identical to SEQ ID NO: 415
  MOD_RES 9: note = 2-aminoisobutyric acid
  MOD_RES 16: note = N alpha-methyl-L-lysine
  MOD_RES 20: note = Citrulline
  MOD_RES 26: note = N-Dimethyl-Lysine

SEQ ID NO: 435 - protein, 40 aa, synthetic construct
VDLGDISGINASVVNIQKEIDRLNEVAKNLNESLIDLQEL

SEQ ID NO: 436 - protein, 37 aa, synthetic construct
SVVNIQKEIDRLNEVAKNLNESLIDLQELGKYEQYIK

SEQ ID NO: 437 - protein, 23 aa, synthetic construct
GINASVVNIQKEIDRLNEVAKNL

SEQ ID NO: 438 - protein, 30 aa, synthetic construct
GINASVVNIQKEIDRLNEVAKNLNESLIDL

SEQ ID NO: 439 - protein, 37 aa, synthetic construct
GINASVVNIQKEIDRLNEVAKNLNESLIDLQELGKYE

SEQ ID NO: 440 - protein, 28 aa, synthetic construct
ISYVEITVPNIQKEIDRLNEVAKNLNES

SEQ ID NO: 441 - protein, 27 aa, synthetic construct
DISYVEITVPNIQKEIDRLNEVAKNLN

SEQ ID NO: 442 - protein, 33 aa, synthetic construct
ISYVEITVPNIQKEIDRLNEVAKNLNESLIDLQ

SEQ ID NO: 443 - protein, 34 aa, synthetic construct
DISYVEITVPNIQKEIDRLNEVAKNLNESLIDLQ

SEQ ID NO: 444 - protein, 40 aa, synthetic construct
PDVDLGDISYVEITVPNIQKEIDRLNEVAKNLNESLIDLQ

SEQ ID NO: 445 - protein, 30 aa, synthetic construct
PDVDLGDISYVEITVPNIQKEIDRLNEVAK

SEQ ID NO: 446 - protein, 32 aa, synthetic construct
PDVDLGDISGINASVVNIQKEIDRLNEVAKNL

SEQ ID NO: 447 - protein, 36 aa, synthetic construct
DISGINASVVNIQKEIDRLNEVAENLNESLIDLQEL

SEQ ID NO: 448 - protein, 36 aa, synthetic construct
DISGINASVVNIEYEIDRLNEVAKNLNESLIDLQEL

SEQ ID NO: 449 - protein, 36 aa, synthetic construct
DISGINASVVNIQKEIDHLNEVAKNLNESLIDLQEL

SEQ ID NO: 450 - protein, 40 aa, synthetic construct
PDLGDISGINESVVNIQSEIDSLNELIDNLNESLIDLQEL

SEQ ID NO: 451 - protein, 40 aa, synthetic construct; identical to SEQ ID NO: 450

SEQ ID NO: 452 - protein, 39 aa, synthetic construct
DLGDISGINESVVNIQSEIDSLNELIDNLNESLIDLQEL

SEQ ID NO: 453 - protein, 39 aa, synthetic construct; identical to SEQ ID NO: 452

SEQ ID NO: 454 - protein, 39 aa, synthetic construct
DLGDISGINQSVVNIQSEIDSLNELIDNLNESLIDLQEL

SEQ ID NO: 455 - protein, 39 aa, synthetic construct
DLGDISGINLSVVNIQSEIDSLNELIDNLNESLIDLQEL

SEQ ID NO: 456 - protein, 39 aa, synthetic construct
DLGDISGINTSVVNIQSEIDSLNELIDNLNESLIDLQEL

SEQ ID NO: 457 - protein, 40 aa, synthetic construct
PDLGDISGINESLLNIQSEIDSLNELIDNLNESLIDLQEL

SEQ ID NO: 458 - protein, 40 aa, synthetic construct
PDLGDISGINESILNIQSEIDSLNELIDNLNESLIDLQEL

SEQ ID NO: 459 - protein, 40 aa, synthetic construct
PDLGDISGINATILNIQSEIDSLNELIDNLNESLIDLQEL

SEQ ID NO: 460 - protein, 39 aa, synthetic construct
DLGDISGINATILNIQDEIDHLNELIDNLNESLIDLQEL

SEQ ID NO: 461 - protein, 39 aa, synthetic construct
DLGDISGINLTILNIQDEIDHLNELIDNLNESLIDLQEL

SEQ ID NO: 462 - protein, 39 aa, synthetic construct
DLGDISGINLTILNIQLEIEHLNEVIENLGESLIELQEL

SEQ ID NO: 463 - protein, 36 aa, synthetic construct; identical to SEQ ID NO: 301

SEQ ID NO: 464 - protein, 36 aa, synthetic construct
DISGINASVVNIQKEIDHLNEVIKNLGESLIELQEL

SEQ ID NO: 465 - protein, 36 aa, synthetic construct
DISGINASVVNIQKEIEHLNEVIKNLGESLIELQEL

SEQ ID NO: 466 - protein, 39 aa, synthetic construct
DLGDISGINASVVNIQKEIEHLNEVIKNLGESLIELQEL

SEQ ID NO: 467 - protein, 39 aa, synthetic construct; identical to SEQ ID NO: 466

SEQ ID NO: 468 - protein, 39 aa, synthetic construct
DLGDISGINLSVVNIQLEIEHLNEVAENLNESLIDLQEL

SEQ ID NO: 469 - protein, 39 aa, synthetic construct; identical to SEQ ID NO: 468

SEQ ID NO: 470 - protein, 38 aa, synthetic construct
LGDISGINASVVNIQKEIEHLNEVAKNLNESLIDLQEL

SEQ ID NO: 471 - protein, 38 aa, synthetic construct
LGDISGINASVVNIQLEIDRLNEVAKNLNESLIDLQEL

SEQ ID NO: 472 - protein, 38 aa, synthetic construct
LGDISGINLSVVNIQKEIDRLNEVAKNLNESLIDLQEL

SEQ ID NO: 473 - protein, 38 aa, synthetic construct
LGDISGINASVLNIQKEIDRLNEVAKNLNESLIDLQEL

SEQ ID NO: 474 - protein, 38 aa, synthetic construct
LGDISGINASIVNIQKEIDRLNEVAKNLNESLIDLQEL

SEQ ID NO: 475 - protein, 38 aa, synthetic construct
LGDISGINASLVNIQKEIDRLNEVAKNLNESLIDLQEL

SEQ ID NO: 476 - protein, 38 aa, synthetic construct
LGDISGINASILNIQKEIDRLNEVAKNLNESLIDLQEL

SEQ ID NO: 477 - protein, 38 aa, synthetic construct
LGDISGINASVVNIQKEIDRLNEVAENLNESLIDLQEL

SEQ ID NO: 478 - protein, 38 aa, synthetic construct
LGDISGINASVVNIQKEIDRLNEVAKNLNESLIDLQEL

SEQ ID NO: 479 - protein, 41 aa, synthetic construct
DLGDISGINLSVVNIQLEIEHLNEVIENLGESLIELQELGS

SEQ ID NO: 480 - protein, 39 aa, synthetic construct
DLGDISGINLTILDIQLEIEHLNELIENLGESLIELQEL

SEQ ID NO: 481 - protein, 39 aa, synthetic construct
DLGDISGINLTILNIQLEIDHLNELIENLGESLIELQEL

SEQ ID NO: 482 - protein, 39 aa, synthetic construct
DLGDISGINLTILNIQLEINHLNELIENLGESLIELQEL

SEQ ID NO: 483 - protein, 39 aa, synthetic construct
DLGDISGINLTILNIQLEILHLNELIENLGESLIELQEL

SEQ ID NO: 484 - protein, 39 aa, synthetic construct
DLGDISGINLTILNIQLEISHLNELIENLGESLIELQEL

SEQ ID NO: 485 - protein, 39 aa, synthetic construct
DLGDISGINLTILNIQLEIEHLNELIENLGESLIDLQEL

SEQ ID NO: 486 - protein, 39 aa, synthetic construct
DLGDISGINLTILNIQLEIEHLNELIENLNESLIDLQEL

SEQ ID NO: 487 - protein, 39 aa, synthetic construct
DLGDISGINSSVVNIQKEIDRLNEVAKNLNESLIDLQEL

SEQ ID NO: 488 - protein, 39 aa, synthetic construct
DLGDISGINGSVVNIQKEIDRLNEVAKNLNESLIDLQEL

SEQ ID NO: 489 - protein, 39 aa, synthetic construct
DLGDISGINTSVVNIQKEIDRLNEVAKNLNESLIDLQEL

SEQ ID NO: 490 - protein, 39 aa, synthetic construct
DLGDISGINASIVNIQKEIDRLNEVAKNLNESLIDLQEL

SEQ ID NO: 491 - protein, 39 aa, synthetic construct
DLGDISGINASLVNIQKEIDRLNEVAKNLNESLIDLQEL

SEQ ID NO: 492 - protein, 39 aa, synthetic construct
DLGDISGINASMVNIQKEIDRLNEVAKNLNESLIDLQEL

SEQ ID NO: 493 - protein, 39 aa, synthetic construct
DLGDISGINASVINIQKEIDRLNEVAKNLNESLIDLQEL

SEQ ID NO: 494 - protein, 39 aa, synthetic construct
DLGDISGINASVLNIQKEIDRLNEVAKNLNESLIDLQEL

SEQ ID NO: 495 - protein, 39 aa, synthetic construct
DLGDISGINASVMNIQKEIDRLNEVAKNLNESLIDLQEL

SEQ ID NO: 496 - protein, 39 aa, synthetic construct
DLGDISGINASIINIQKEIDRLNEVAKNLNESLIDLQEL

SEQ ID NO: 497 - protein, 39 aa, synthetic construct
DLGDISGINASLLNIQKEIDRLNEVAKNLNESLIDLQEL

SEQ ID NO: 498 - protein, 39 aa, synthetic construct
DLGDISGINASMMNIQKEIDRLNEVAKNLNESLIDLQEL

SEQ ID NO: 499 - protein, 39 aa, synthetic construct
DLGDISGINASVVNIQQEIDRLNEVAKNLNESLIDLQEL

SEQ ID NO: 500 - protein, 39 aa, synthetic construct
DLGDISGINASVVNIQEEIDRLNEVAKNLNESLIDLQEL

SEQ ID NO: 501 - protein, 39 aa, synthetic construct
DLGDISGINASVVNIQSEIDRLNEVAKNLNESLIDLQEL

SEQ ID NO: 502 - protein, 39 aa, synthetic construct
DLGDISGINASVVNIQNEIDRLNEVAKNLNESLIDLQEL

SEQ ID NO: 503 - protein, 39 aa, synthetic construct
DLGDISGINASVVNIQKEIDNLNEVAKNLNESLIDLQEL

SEQ ID NO: 504 - protein, 39 aa, synthetic construct
DLGDISGINASVVNIQKEIDQLNEVAKNLNESLIDLQEL

SEQ ID NO: 505 - protein, 39 aa, synthetic construct
DLGDISGINASVVNIQKEIDELNEVAKNLNESLIDLQEL

SEQ ID NO: 506 - protein, 39 aa, synthetic construct
DLGDISGINASVVNIQKEIDRLNEIAKNLNESLIDLQEL

SEQ ID NO: 507 - protein, 39 aa, synthetic construct
DLGDISGINASVVNIQKEIDRLNEMAKNLNESLIDLQEL

SEQ ID NO: 508 - protein, 39 aa, synthetic construct
DLGDISGINASVVNIQKEIDRLNELAKNLNESLIDLQEL

SEQ ID NO: 509 - protein, 39 aa, synthetic construct
DLGDISGINASVVNIQKEIDRLNEAAKNLNESLIDLQEL

SEQ ID NO: 510 - protein, 39 aa, synthetic construct
DLGDISGINASVVNIQKEIDRLNETAKNLNESLIDLQEL

SEQ ID NO: 511 - protein, 39 aa, synthetic construct
DLGDISGINASVVNIQKEIDRLNEVSKNLNESLIDLQEL

SEQ ID NO: 512 - protein, 39 aa, synthetic construct
DLGDISGINASVVNIQKEIDRLNEVVKNLNESLIDLQEL

SEQ ID NO: 513 - protein, 39 aa, synthetic construct
DLGDISGINASVVNIQKEIDRLNEVCKNLNESLIDLQEL

SEQ ID NO: 514 - protein, 39 aa, synthetic construct
DLGDISGINASVVNIQKEIDRLNEVTKNLNESLIDLQEL

SEQ ID NO: 515 - protein, 39 aa, synthetic construct
DLGDISGINASVVNIQKEIDRLNEVGKNLNESLIDLQEL

SEQ ID NO: 516 - protein, 39 aa, synthetic construct
DLGDISGINASVVNIQKEIDRLNELIKNLNESLIDLQEL

SEQ ID NO: 517 - protein, 39 aa, synthetic construct
DLGDISGINASVVNIQKEIDRLNEIIKNLNESLIDLQEL

SEQ ID NO: 518 - protein, 39 aa, synthetic construct
DLGDISGINASVVNIQKEIDRLNEISKNLNESLIDLQEL

SEQ ID NO: 519 - protein, 39 aa, synthetic construct
DLGDISGINASVVNIQKEIDRLNEAIKNLNESLIDLQEL

SEQ ID NO: 520 - protein, 39 aa, synthetic construct
DLGDISGINASVVNIQKEIDRLNETIKNLNESLIDLQEL

SEQ ID NO: 521 - protein, 39 aa, synthetic construct
DLGDISGINASVVNIQKEIDRLNEVAQNLNESLIDLQEL

SEQ ID NO: 522 - protein, 39 aa, synthetic construct
DLGDISGINASVVNIQKEIDRLNEVAENLNESLIDLQEL

SEQ ID NO: 523 - protein, 39 aa, synthetic construct
DLGDISGINASVVNIQKEIDRLNEVASNLNESLIDLQEL

SEQ ID NO: 524 - protein, 39 aa, synthetic construct
DLGDISGINASVVNIQKEIDRLNEVANNLNESLIDLQEL

SEQ ID NO: 525 - protein, 39 aa, synthetic construct
DLGDISGINASVVNIQKEIDRLNEVAKNLGESLIDLQEL

SEQ ID NO: 526 - protein, 39 aa, synthetic construct
DLGDISGINASIVNIQKEIDRLNEVIKNLNESLIDLQEL

SEQ ID NO: 527 - protein, 40 aa, synthetic construct
CGSGSGLEQLDILSENLNKAVENLRDIEKQINVVSANIGS

SEQ ID NO: 528 - protein, 34 aa, synthetic construct
EEEELTTLNEELSHLATLLEETVAELQALIDNIA

SEQ ID NO: 529 - protein, 36 aa, synthetic construct
QVEELLQQLQEQIASTQAELGKLQDVRAALEAQLAA

SEQ ID NO: 530 - protein, 36 aa, synthetic construct
DISSIAAAFVDIQEYIDHLLTVASELESALIYLIPL

SEQ ID NO: 531 - protein, 36 aa, synthetic construct
DISSIAAAFVDISEYIEHLLTVASELNSALIFLIPI

SEQ ID NO: 532 - protein, 36 aa, synthetic construct
DISSIAAAFVDISEYIEHLLTVASELESALIFLIPI

SEQ ID NO: 533 - protein, 36 aa, synthetic construct
DYSWIAAATVDIETEIQILLTVASELNSALIYLIPL

SEQ ID NO: 534 - protein, 36 aa, synthetic construct
DYSWIAAAFVDIETEIQILLQVATELNSALIFLIPI

SEQ ID NO: 535 - protein, 36 aa, synthetic construct
DYSWIAAAFVDIETEIQILLQVATELESALIFLIPI

SEQ ID NO: 536 - protein, 36 aa, synthetic construct
DYSWIAAAFVDIEEEIQILLQVATELNSALIFLIPL

SEQ ID NO: 537 - protein, 36 aa, synthetic construct
DYSWIAAAFVDIEEEIQILLQVATELESALIFLIPI

SEQ ID NO: 538 - protein, 36 aa, synthetic construct
DISSIAAAFVDIQTEIDIILTVANELNSALINLIPL

SEQ ID NO: 539 - protein, 36 aa, synthetic construct
DYSWIAAAFVDIETEIQILLQVATELNSALIFLIPL

SEQ ID NO: 540 - protein, 36 aa, synthetic construct
SLLAEIQQLQQEEAAALGKLQEAVGKLNELVKELLE

SEQ ID NO: 541 - protein, 36 aa, synthetic construct
QALQQLLQQAQEQTAQLLSKIQEALGKLEEAIKELL

SEQ ID NO: 542 - protein, 36 aa, synthetic construct
DALQQIQQLQAQQAEALGKLAAAVGELAANLAELAA

SEQ ID NO: 543 - protein, 36 aa, synthetic construct
AALAEIEKLKAEEAAALAKLQEAVGKLAEALAELAA

SEQ ID NO: 544 - protein, 36 aa, synthetic construct
GLLAQIKELQAQEAAALAKLAESVGKLAELLKELKE

SEQ ID NO: 545 - protein, 36 aa, synthetic construct
LAEIAALQAQAAEALSKLAASLGKLEEYLKKLQEIL

SEQ ID NO: 546 - protein, 36 aa, synthetic construct
AAAEQIAALQQQIAKLQAELGKLQDERAKLEAELAA

SEQ ID NO: 547 - protein, 36 aa, synthetic construct
SAATLAQIEALQAEIGKLQAEVNQLQAQLAQLQELL

SEQ ID NO: 548 - protein, 36 aa, synthetic construct
LLAEIEALKAEEASALAKLASSLGKLAEYLAKLKEL

SEQ ID NO: 549 - protein, 36 aa, synthetic construct
AAAVAAQVAQLQAETAAALGKLQEAVAALQAALAAL

SEQ ID NO: 550 - protein, 36 aa, synthetic construct
AAAAAELAAAASKLQDSLSSLASALSTAQELAKLAE

SEQ ID NO: 551 - protein, 36 aa, synthetic construct
SAAAAEAAAWQAKLQDALAKTASAKAEAEKLKKELE

SEQ ID NO: 552 - protein, 36 aa, synthetic construct
SAAAAEAAAYQAKLQASLSETASASSEASKLASLLS

SEQ ID NO: 553 - protein, 36 aa, synthetic construct
DALLAAAQAQIDAALGKLQELLSEAEALAKKAAELE

SEQ ID NO: 554 - protein, 36 aa, synthetic construct
AAAAAAAKAEELAAEQASVLGQLQDIVAATAALLAA

SEQ ID NO: 555 - protein, 36 aa, synthetic construct
AALAAIAASQAEAAALLAKLQEAVGKTAALQAEAEA

SEQ ID NO: 556 - protein, 36 aa, synthetic construct
GLEELQKLQEERAKAESELGKLQDVQAQLQAQLQAL

SEQ ID NO: 557 - protein, 36 aa, synthetic construct
SLLQQLLEQAAQLQATAAALGELAAAVAALQEALAA

SEQ ID NO: 558 - protein, 36 aa, synthetic construct
AAATVAAAQAAFAAALAKAQAAAAAAQAALAAQLAQ

SEQ ID NO: 559 - protein, 36 aa, synthetic construct
ADVAATLAALAAQQASLAAQLAKLQKEAAELAALLA

SEQ ID NO: 560 - protein, 36 aa, synthetic construct
SISWIPAAFVDIQEEIEIILTVAIELNSTIIYLIPL

SEQ ID NO: 561 - protein, 36 aa, synthetic construct
DISSIAAAFVDISEEISILLTVAEELESALIYLIPL

SEQ ID NO: 562 - protein, 36 aa, synthetic construct
SISWIPAAFVDIETEIQIILTVAIELNSTIIYLIPL

SEQ ID NO: 563 - protein, 36 aa, synthetic construct
SISWIPAAFVDIEEEIQIILQVAIELNSTIIYLIPL

SEQ ID NO: 564 - protein, 36 aa, synthetic construct
DISWIAAAYVDIQEYIETLLTVATELNSALIFLIPI

SEQ ID NO: 565 - protein, 36 aa, synthetic construct
SISWIPAAFVDIQEEIEIILQVAIELNSTIIYLIPL

SEQ ID NO: 566 - protein, 36 aa, synthetic construct
DISSIAAATVDIQEYIETLLQVATELNSALIFLIPI

SEQ ID NO: 567 - protein, 36 aa, synthetic construct
SISWIPAAFVDIQTEIEIILTVAIELNSTIIYLIPL

SEQ ID NO: 568 - protein, 36 aa, synthetic construct
SISWIPAAFVDIQTEIDIILQVAIELNSTIIYLIPL

SEQ ID NO: 569 - protein, 36 aa, synthetic construct
SISWIPAAFVDIQTEIDIILTVAIELNSTIIYLIPL

SEQ ID NO: 570 - protein, 36 aa, synthetic construct
SISWIPAAFVDIQTEIEIILQVAIELNSTIIYLIPL

SEQ ID NO: 571 - protein, 36 aa, synthetic construct
DISSIIAATVDIQEYIDQLLIVATELNSALIYLIPI

SEQ ID NO: 572 - protein, 36 aa, synthetic construct
DISSIIAAFVDIQEYIDTLLQVATELNSALIFLIPI

SEQ ID NO: 573 - protein, 36 aa, synthetic construct
DISSIIAAFVDIEEHIQTLLTVATELNSALIFLIPI

SEQ ID NO: 574 - protein, 36 aa, synthetic construct
SISWISASFVDIQTEIEIILQVAIELNSTIIYLIPL

SEQ ID NO: 575 - protein, 36 aa, synthetic construct
DISSIAAAFVDISEEISILLTVAEELESALIYLIPI

SEQ ID NO: 576 - protein, 36 aa, synthetic construct
DISWIAAAYVDIEEHIQTLLTVATELNSALIFLIPI

SEQ ID NO: 577 - protein, 36 aa, synthetic construct
DVSSIEASYVDIEDEIQTLLQVAIELNSALIFLIPI

SEQ ID NO: 578 - protein, 36 aa, synthetic construct
DISSIEASYVDIEEHIQQLLQVAIELNSALIFLIPI

SEQ ID NO: 579 - protein, 36 aa, synthetic construct
DISWIAAAFVDIEEYIQTLLTVATELNSALIFLIPI

SEQ ID NO: 580 - protein, 36 aa, synthetic construct
DISSIIAAFVDIQEYIDQLLIVATELNSALIFLIPI

SEQ ID NO: 581 - protein, 36 aa, synthetic construct
DISSIEASFVDIQEYIDQLLQVAIELNSALIFLIPI

SEQ ID NO: 582 - protein, 36 aa, synthetic construct
DISGINLSILNIQLEIEHLNEVIENLGESLIELQEL

SEQ ID NO: 583 - protein, 36 aa, synthetic construct
DISGINLTILNIQLEIEHLNELIENLGESLIELQEL

SEQ ID NO: 584 - protein, 36 aa, synthetic construct
DISGINLSVVNIQLEIEHLNELIENLGESLIELQEL

SEQ ID NO: 585 - protein, 36 aa, synthetic construct; identical to SEQ ID NO: 530

SEQ ID NO: 586 - protein, 36 aa, synthetic construct
  MOD_RES 7: note = 2-aminoisobutyric acid
  MOD_RES 14: note = ornithine
  MOD_RES 24: note = ornithine
DISGINXSVVNIQXEIEHLNEVIXNLNESLIDLQEL

SEQ ID NO: 587 - protein, 36 aa, synthetic construct
  MOD_RES 7: note = 2-aminoisobutyric acid
  MOD_RES 14: note = ornithine
  MOD_RES 24: note = ornithine
  MOD_RES 18: note = ornithine
DISGINXSVVNIQXEIDXLNEVIXNLNESLIDLQEL

SEQ ID NO: 588 - protein, 36 aa, synthetic construct
  MOD_RES 7: note = 2-aminoisobutyric acid
  MOD_RES 14: note = ornithine
  MOD_RES 24: note = ornithine
DISGINXSVVNIQXEIEHLNEVAXNLGESLIELQEL

SEQ ID NO: 589 - protein, 36 aa, synthetic construct
  MOD_RES 7: note = 2-aminoisobutyric acid
  MOD_RES 14: note = ornithine
  MOD_RES 24: note = ornithine
  MOD_RES 18: note = ornithine
DISGINXSVVNIQXEIDXLNEVAXNLGESLIELQEL

SEQ ID NO: 590 - protein, 36 aa, synthetic construct
  MOD_RES 7: note = 2-aminoisobutyric acid
  MOD_RES 14: note = ornithine
  MOD_RES 24: note = ornithine
DISGINXSVVNIQXEIEHLNEVIXNLGESLIELQEL

SEQ ID NO: 591 - protein, 36 aa, synthetic construct
  MOD_RES 7: note = 2-aminoisobutyric acid
  MOD_RES 14: note = ornithine
  MOD_RES 24: note = ornithine
  MOD_RES 18: note = ornithine
DISGINXSVVNIQXEIDXLNEVIXNLGESLIELQEL

SEQ ID NO: 592 - protein, 38 aa, synthetic construct; identical to SEQ ID NO: 349
  MOD_RES 9: note = 2-aminoisobutyric acid
  MOD_RES 16: note = ornithine
  MOD_RES 20: note = ornithine
  MOD_RES 26: note = ornithine

SEQ ID NO: 593 - protein, 36 aa, synthetic construct; identical to SEQ ID NO: 587
  MOD_RES 7: note = 2-aminoisobutyric acid
  MOD_RES 14: note = ornithine
  MOD_RES 18: note = ornithine
  MOD_RES 24: note = ornithine

SEQ ID NO: 594 - protein, 36 aa, synthetic construct; identical to SEQ ID NO: 587
  MOD_RES 7: note = 2-aminoisobutyric acid
  MOD_RES 14: note = ornithine
  MOD_RES 18: note = ornithine
  MOD_RES 24: note = ornithine

SEQ ID NO: 595 - protein, 36 aa, synthetic construct
  MOD_RES 14: note = ornithine
  MOD_RES 18: note = ornithine
  MOD_RES 24: note = ornithine
DISGINASVVNIQXEIDXLNEVIXNLNESLIDLQEL

SEQ ID NO: 596 - protein, 36 aa, synthetic construct; identical to SEQ ID NO: 587
  MOD_RES 7: note = 2-aminoisobutyric acid
  MOD_RES 14: note = ornithine
  MOD_RES 24: note = ornithine
  MOD_RES 18: note = Homoarginine

SEQ ID NO: 597 - protein, 36 aa, synthetic construct; identical to SEQ ID NO: 595
  MOD_RES 14: note = ornithine
  MOD_RES 18: note = Homoarginine
  MOD_RES 24: note = ornithine

SEQ ID NO: 598 - protein, 36 aa, synthetic construct
  MOD_RES 7: note = 2-aminoisobutyric acid
  MOD_RES 14: note = ornithine
  MOD_RES 24: note = ornithine
  MOD_RES 18: note = ornithine
DISGINXSIVNIQXEIDXLNEIIXNLNESLIDLQEL

SEQ ID NO: 599 - protein, 48 aa, synthetic construct
PDVDLGDISGINASVVNIQKEIDRLNEVAKNLNESLIDLQELGSGSGC

SEQ ID NO: 600 - protein, 31 aa, synthetic construct
IQKEIDRLNEVAKNLNESLIDLQELGSGSGC

SEQ ID NO: 601 - protein, 42 aa, synthetic construct
DISGINASVVNIQKPIDRLNEVAKNLNESLIDLQELGSGSGC

SEQ ID NO: 602 - protein, 42 aa, synthetic construct
DISGINASVVNIQKPIDPLNEVAKPLNEPLIDLQELGSGSGC

SEQ ID NO: 603 - protein, 42 aa, synthetic construct
DISGINASVVNAQKEADRANEAAKNANESLIDLQELGSGSGC

SEQ ID NO: 604 - protein, 42 aa, synthetic construct
DISGINASVVNIQKEIDRLNEVIKNLNESLIDLQELGSGSGC

SEQ ID NO: 605 - protein, 42 aa, synthetic construct
DISGINASVVNIQKEIARLNEVAKALNESLIDLQELGSGSGC

SEQ ID NO: 606 - protein, 42 aa, synthetic construct
DIAAINASVANIQKEIARLNEVAKALNESLAALQALGSGSGC

SEQ ID NO: 607 - protein, 42 aa, synthetic construct
DISGINASVVNIQKEIARLNEVIKALNESLIDLQELGSGSGC

SEQ ID NO: 608 - protein, 42 aa, synthetic construct
DISGINASVVNIQKEIERLNKVAKNLNESLIDLQELGSGSGC

SEQ ID NO: 609 - protein, 42 aa, synthetic construct
DISGINASVVEIQKKIEELNKKAEELNKKLIDLQELGSGSGC

SEQ ID NO: 610 - protein, 42 aa, synthetic construct
SLDYINVTFLDLQDEMNRLQEAIKVLNQSYINLDEIGSGSGC

SEQ ID NO: 611 - protein, 42 aa, synthetic construct
SLTQINTTLLDLTYEMLSLQQVVKALNESYIDLKELGSGSGC

SEQ ID NO: 612 - protein, 42 aa, synthetic construct
DISGINASVVDIKKEIEHLNEIAKSLNESLIDLQELGSGSGC

SEQ ID NO: 613 - protein, 42 aa, synthetic construct
DISGINASVVDIKKEIEHLNEIAKNLNESLIDLQELGSGSGC

SEQ ID NO: 614 - protein, 42 aa, synthetic construct
SLDQINVTFLDLEYEMKKLEEAIKKLEESYIDLKELGSGSGC

SEQ ID NO: 615 - protein, 42 aa, synthetic construct
DISQINASVVNIEYEIKKLEEVAKKLEESLIDLQELGSGSGC

SEQ ID NO: 616 - protein, 42 aa, synthetic construct
SIDQINATFVDIEYEIKKLEEVAKKLEESYIDLQELGSGSGC

SEQ ID NO: 617 - protein, 53 aa, synthetic construct
KNHTSPDVDLGDISGINASVVNIQKEIDRLNEVAKNLNESLIDLQELGSGSGC

SEQ ID NO: 618 - protein, 42 aa, synthetic construct
DISGINASVVNIQKEIDRLNEVAKNLNESLIDLQELGGGGSC

SEQ ID NO: 619 - protein, 45 aa, synthetic construct
DISGINASVVNIQKEIDRLNEVAKNLNESLIDLQELGGGGGGGGC

SEQ ID NO: 620 - protein, 43 aa, synthetic construct
DISGINASVVNIQKEIDRLNEVAKNLNESLIDLQELGGGGGGC

SEQ ID NO: 621 - protein, 42 aa, synthetic construct
DISGINASVVNIQKEIDRLNEVAKNLNESLIDLQELEAAAKC

SEQ ID NO: 622 - protein, 47 aa, synthetic construct
DISGINASVVNIQKEIDRLNEVAKNLNESLIDLQELEAAAKEAAAKC

SEQ ID NO: 623 - protein, 44 aa, synthetic construct
DISGINASVVNIQKEIDRLNEVAKNLNESLIDLQELAEAAAKAC

SEQ ID NO: 624 - protein, 40 aa, synthetic construct
DISGINASVVNIQKEIDRLNEVAKNLNESLIDLQELGSGC

SEQ ID NO: 625 - protein, 37 aa, synthetic construct
DISGINASVVNIQKEIDRLNEVAKNLNESLIDLQELC

SEQ ID NO: 626 - protein, 49 aa, synthetic construct
DISGINASVVNIQKEIDRLNEVAKNLNESLIDLQELGSAGSAAGSGEFC

SEQ ID NO: 627 - protein, 55 aa, synthetic construct
DISGINASVVNIQKEIDRLNEVAKNLNESLIDLQELKESGSVSSEQLAQFRSLDC

SEQ ID NO: 628 - protein, 51 aa, synthetic construct
DISGINASVVNIQKEIDRLNEVAKNLNESLIDLQELEGKSSGSGSESKSTC

SEQ ID NO: 629 - protein, 42 aa, synthetic construct
LEQLDILSENLNKAVENLRDIEKQINVVSANIGSIDGSGSGC

SEQ ID NO: 630 - protein, 41 aa, synthetic construct
EAAAKEAAAKEAAAKEAAAKEAAAKEAAAKEAAAKEAAAKC

SEQ ID NO: 631 - protein, 46 aa, synthetic construct
EAAAKEAAAKEAAAKEAAAKEAAAKEAAAKEAAAKEAAAKGSGSGC

SEQ ID NO: 632 - protein, 49 aa, synthetic construct
PDVDLGGDISGINASVVNIQKEIDRLNEVAKNLNESLIDLQELGSGSGC

SEQ ID NO: 633 - protein, 54 aa, synthetic construct
KNHTSPDVDLGGDISGINASVVNIQKEIDRLNEVAKNLNESLIDLQELGSGSGC

SEQ ID NO: 634 - protein, 46 aa, synthetic construct
DLGDISGINASVVNIQKEIDRLNEVAKNLNESLIDLQELGGSGSGC

SEQ ID NO: 635 - protein, 30 aa, synthetic construct
VVNIQKEIDRLNEVAKNLNESLIDGSGSGC

SEQ ID NO: 636 - protein, 42 aa, synthetic construct
DISGINASVVNIQKEIDRLNEVAKNLNESLIELQELGSGSGC

SEQ ID NO: 637 - protein, 42 aa, synthetic construct
DISGINASVVNIQKEIDRLNEVAKNLNESLIKLQELGSGSGC

SEQ ID NO: 638 - protein, 42 aa, synthetic construct
DISGINASVVNIQKEIDRLNEVAKNLNESLIGLQELGSGSGC

SEQ ID NO: 639 - protein, 42 aa, synthetic construct
DISGINASVVNIQKEIDRLNEVAKNLGESLIGLQELGSGSGC

SEQ ID NO: 640 - protein, 42 aa, synthetic construct
DISGINASVVNIQKEIDRLNEVAKNLPESLIGLQELGSGSGC

SEQ ID NO: 641 - protein, 42 aa, synthetic construct
DISGINASVVNIQKEIDRLDEVAKNLNESLIDLQELGSGSGC

SEQ ID NO: 642 - protein, 42 aa, synthetic construct
DISGINASVVNIQKEIDRLGEVAKNLNESLIDLQELGSGSGC

SEQ ID NO: 643 - protein, 42 aa, synthetic construct
DISGINASVENIQKEIDRLNEVAKNLNESLIDLQELGSGSGC

SEQ ID NO: 644 - protein, 42 aa, synthetic construct
DISGINASVVNIQKEIDRLNEVAKNLGESLIELQELGSGSGC

SEQ ID NO: 645 - protein, 42 aa, synthetic construct
DISGINASVVNIQKEIDRLDEVAKNLPESLIELQELGSGSGC

SEQ ID NO: 646 - protein, 42 aa, synthetic construct
CGSGSGDISGINASVVNIQKEIDRLNEVAKNLNESLIDLQEL

SEQ ID NO: 647 - protein, 34 aa, synthetic construct
IEEIQKKIEEIQKKIEEIQKKIEEIQKKGSGSGC

SEQ ID NO: 648 - protein, 45 aa, synthetic construct
EKLDIYSENLNKAVENLTGLEQKLLQQVDLTAYSYILDEGSGSGC

SEQ ID NO: 649 - protein, 46 aa, synthetic construct
IPNFGSLTQINTTLLDLTYEMLSLQQVVKALNESYIDLKEGSGSGC

SEQ ID NO: 650 - protein, 43 aa, synthetic construct
DLGDISGINLTILNIQDEIEHLNELIDNLNESLIDLQELGSGC

SEQ ID NO: 651 - protein, 43 aa, synthetic construct
DLGDISGINLTILNIQLEIEHLNELIENLGESLIELQELGSGC

SEQ ID NO: 652 - protein, 43 aa, synthetic construct
DLGDISGINLSVVNIQLEIEHLNEVIENLGESLIELQELGSGC

SEQ ID NO: 653 - protein, 43 aa, synthetic construct
DLGDISGINLSILNIQLEIEHLNEVIENLGESLIELQELGSGC

SEQ ID NO: 654 - protein, 42 aa, synthetic construct
LGDISGINLSILNIQLEIEHLNEVIENLGESLIELQELGSGC

SEQ ID NO: 655 - protein, 42 aa, synthetic construct
LGDISGINLTILNIQLEIEHLNELIENLGESLIELQELGSGC

SEQ ID NO: 656 - protein, 45 aa, synthetic construct
DLGDISGINLSILNIQLEIEHLNEVIENLGESLIELQELGSGSGC

SEQ ID NO: 657 - protein, 44 aa, synthetic construct
LGDISGINLSILNIQLEIEHLNEVIENLGESLIELQELGSGSGC

SEQ ID NO: 658 - protein, 44 aa, synthetic construct
LGDISGINLSVVNIQLEIEHLNEVIENLGESLIELQELGSGSGC

SEQ ID NO: 659 - protein, 44 aa, synthetic construct
LGDISGINLSVVNIQLEIDHLNEVIENLGESLIELQELGSGSGC

SEQ ID NO: 660 - protein, 45 aa, synthetic construct
DLGDISGINLSVVNIQLEIDHLNEVIENLGESLIELQELGSGSGC

SEQ ID NO: 661 - protein, 45 aa, synthetic construct
DLGDISGINLSVVNIQLEIEHLNELIENLGESLIELQELGSGSGC

SEQ ID NO: 662 - protein, 38 aa, synthetic construct
DISGINASVVNIQKEIDRLNEVIKNLGESLIELQELGC

SEQ ID NO: 663 - protein, 40 aa, synthetic construct
LGDISGINASVVNIQKEIDRLNEVIKNLGESLIELQELGC

SEQ ID NO: 664 - protein, 40 aa, synthetic construct
  MOD_RES 9: note = 2-aminoisobutyric acid
  MOD_RES 16: note = ornithine
  MOD_RES 26: note = ornithine
LGDISGINXSVVNIQXEIDHLNEVAXNLNESLIDLQELGC

SEQ ID NO: 665 - protein, 40 aa, synthetic construct; identical to SEQ ID NO: 664
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 666 - protein, 40 aa, synthetic construct; identical to SEQ ID NO: 664
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 667 - protein, 40 aa, synthetic construct
  MOD_RES 9: note = 2-aminoisobutyric acid
  MOD_RES 16: note = ornithine
  MOD_RES 26: note = ornithine
LGDISGINXSVVNIQXEIEHLNEVAXNLNESLIDLQELGC

SEQ ID NO: 668 - protein, 40 aa, synthetic construct; identical to SEQ ID NO: 667
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 669 - protein, 40 aa, synthetic construct; identical to SEQ ID NO: 667
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 670 - protein, 40 aa, synthetic construct; identical to SEQ ID NO: 667
  MOD_RES 9: note = 2-aminoisobutyric acid
  MOD_RES 16: note = N-Methyl-Lysine
  MOD_RES 26: note = N-Methyl-Lysine

SEQ ID NO: 671 - protein, 40 aa, synthetic construct; identical to SEQ ID NO: 667
  MOD_RES 9: note = 2-aminoisobutyric acid
  MOD_RES 16: note = N-Dimethyl-Lysine
  MOD_RES 26: note = N-Dimethyl-Lysine

SEQ ID NO: 672 - protein, 40 aa, synthetic construct; identical to SEQ ID NO: 667
  MOD_RES 9: note = 2-aminoisobutyric acid
  MOD_RES 16: note = N alpha-methyl-L-lysine
  MOD_RES 26: note = ornithine

SEQ ID NO: 673 - protein, 40 aa, synthetic construct; identical to SEQ ID NO: 667
  MOD_RES 9: note = 2-aminoisobutyric acid
  MOD_RES 16: note = N alpha-methyl-L-lysine
  MOD_RES 26: note = 2,4-diaminobutyric acid

SEQ ID NO: 674 - protein, 40 aa, synthetic construct; identical to SEQ ID NO: 667
  MOD_RES 9: note = 2-aminoisobutyric acid
  MOD_RES 16: note = N alpha-methyl-L-lysine
  MOD_RES 26: note = 2,3-diaminopropionic acid

SEQ ID NO: 675 - protein, 40 aa, synthetic construct; identical to SEQ ID NO: 667
  MOD_RES 9: note = 2-aminoisobutyric acid
  MOD_RES 16: note = N alpha-methyl-L-lysine
  MOD_RES 26: note = N-Methyl-Lysine

SEQ ID NO: 676 - protein, 40 aa, synthetic construct; identical to SEQ ID NO: 667
  MOD_RES 9: note = 2-aminoisobutyric acid
  MOD_RES 16: note = N alpha-methyl-L-lysine
  MOD_RES 26: note = N-Dimethyl-Lysine

SEQ ID NO: 677 - protein, 40 aa, synthetic construct
  MOD_RES 9: note = 2-aminoisobutyric acid
  MOD_RES 16: note = ornithine
  MOD_RES 20: note = ornithine
  MOD_RES 26: note = ornithine
LGDISGINXSVVNIQXEIDXLNEVAXNLNESLIDLQELGC

SEQ ID NO: 678 - protein, 40 aa, synthetic construct; identical to SEQ ID NO: 677
  MOD_RES 9: note = 2-aminoisobutyric acid
  MOD_RES 16: note = 2,4-diaminobutyric acid
  MOD_RES 20: note = ornithine
  MOD_RES 26: note = 2,4-diaminobutyric acid

SEQ ID NO: 679 - protein, 40 aa, synthetic construct; identical to SEQ ID NO: 677
  MOD_RES 9: note = 2-aminoisobutyric acid
  MOD_RES 16: note = 2,3-diaminopropionic acid
  MOD_RES 20: note = ornithine
  MOD_RES 26: note = 2,3-diaminopropionic acid

SEQ ID NO: 680 - protein, 40 aa, synthetic construct; identical to SEQ ID NO: 677
  MOD_RES 9: note = 2-aminoisobutyric acid
  MOD_RES 16: note = N-Methyl-Lysine
  MOD_RES 20: note = ornithine
  MOD_RES 26: note = N-Methyl-Lysine

SEQ ID NO: 681 - protein, 40 aa, synthetic construct; identical to SEQ ID NO: 677
  MOD_RES 9: note = 2-aminoisobutyric acid
  MOD_RES 16: note = N-Dimethyl-Lysine
  MOD_RES 20: note = ornithine
  MOD_RES 26: note = N-Dimethyl-Lysine

SEQ ID NO: 682 - protein, 40 aa, synthetic construct; identical to SEQ ID NO: 677
  MOD_RES 9: note = 2-aminoisobutyric acid
  MOD_RES 16: note = N alpha-methyl-L-lysine
  MOD_RES 20: note = ornithine
  MOD_RES 26: note = ornithine

SEQ ID NO: 683 - protein, 40 aa, synthetic construct; identical to SEQ ID NO: 677
  MOD_RES 9: note = 2-aminoisobutyric acid
  MOD_RES 16: note = N alpha-methyl-L-lysine
  MOD_RES 20: note = ornithine
  MOD_RES 26: note = 2,4-diaminobutyric acid

SEQ ID NO: 684 - protein, 40 aa, synthetic construct; identical to SEQ ID NO: 677
  MOD_RES 9: note = 2-aminoisobutyric acid
  MOD_RES 16: note = N alpha-methyl-L-lysine
  MOD_RES 20: note = ornithine
  MOD_RES 26: note = 2,3-diaminopropionic acid

SEQ ID NO: 685 - protein, 40 aa, synthetic construct; identical to SEQ ID NO: 677
  MOD_RES 9: note = 2-aminoisobutyric acid
  MOD_RES 16: note = N alpha-methyl-L-lysine
  MOD_RES 20: note = ornithine
  MOD_RES 26: note = N-Methyl-Lysine

SEQ ID NO: 686 - protein, 40 aa, synthetic construct; identical to SEQ ID NO: 677
  MOD_RES 9: note = 2-aminoisobutyric acid
  MOD_RES 16: note = N alpha-methyl-L-lysine
  MOD_RES 20: note = ornithine
  MOD_RES 26: note = N-Dimethyl-Lysine

SEQ ID NO: 687 - protein, 40 aa, synthetic construct; identical to SEQ ID NO: 677
  MOD_RES 9: note = 2-aminoisobutyric acid
  MOD_RES 16: note = ornithine
  MOD_RES 20: note = Citrulline
  MOD_RES 26: note = ornithine

SEQ ID NO: 688 - protein, 40 aa, synthetic construct; identical to SEQ ID NO: 677
  MOD_RES 9: note = 2-aminoisobutyric acid
  MOD_RES 16: note = 2,4-diaminobutyric acid
  MOD_RES 20: note = Citrulline
  MOD_RES 26: note = 2,4-diaminobutyric acid

SEQ ID NO: 689 - protein, 40 aa, synthetic construct; identical to SEQ ID NO: 677
  MOD_RES 9: note = 2-aminoisobutyric acid
  MOD_RES 16: note = 2,3-diaminopropionic acid
  MOD_RES 20: note = Citrulline
  MOD_RES 26: note = 2,3-diaminopropionic acid

SEQ ID NO: 690 - protein, 40 aa, synthetic construct; identical to SEQ ID NO: 677
  MOD_RES 9: note = 2-aminoisobutyric acid
  MOD_RES 16: note = N-Methyl-Lysine
  MOD_RES 20: note = Citrulline
  MOD_RES 26: note = N-Methyl-Lysine

SEQ ID NO: 691 - protein, 40 aa, synthetic construct; identical to SEQ ID NO: 677
  MOD_RES 9: note = 2-aminoisobutyric acid
  MOD_RES 16: note = N-Dimethyl-Lysine
  MOD_RES 20: note = Citrulline
  MOD_RES 26: note = N-Dimethyl-Lysine

SEQ ID NO: 692 - protein, 40 aa, synthetic construct; identical to SEQ ID NO: 677
  MOD_RES 9: note = 2-aminoisobutyric acid
  MOD_RES 16: note = N alpha-methyl-L-lysine
  MOD_RES 20: note = Citrulline
  MOD_RES 26: note = ornithine

SEQ ID NO: 693 - protein, 40 aa, synthetic construct; identical to SEQ ID NO: 677
  MOD_RES 9: note = 2-aminoisobutyric acid
  MOD_RES 16: note = N alpha-methyl-L-lysine
  MOD_RES 20: note = Citrulline
  MOD_RES 26: note = 2,4-diaminobutyric acid

SEQ ID NO: 694 - protein, 40 aa, synthetic construct; identical to SEQ ID NO: 677
  MOD_RES 9: note = 2-aminoisobutyric acid
  MOD_RES 16: note = N alpha-methyl-L-lysine
  MOD_RES 20: note = Citrulline
  MOD_RES 26: note = 2,3-diaminopropionic acid

SEQ ID NO: 695 - protein, 40 aa, synthetic construct; identical to SEQ ID NO: 677
  MOD_RES 9: note = 2-aminoisobutyric acid
  MOD_RES 16: note = N alpha-methyl-L-lysine
  MOD_RES 20: note = Citrulline
  MOD_RES 26: note = N-Methyl-Lysine

SEQ ID NO: 696 - protein, 40 aa, synthetic construct; identical to SEQ ID NO: 677
  MOD_RES 9: note = 2-aminoisobutyric acid
  MOD_RES 16: note = N alpha-methyl-L-lysine
  MOD_RES 20: note = Citrulline
  MOD_RES 26: note = N-Dimethyl-Lysine

SEQ ID NO: 697 - protein, 40 aa, synthetic construct
  MOD_RES 9: note = 2-aminoisobutyric acid
  MOD_RES 16: note = ornithine
  MOD_RES 26: note = ornithine
LGDISGINXSVVNIQXEIDHLNEVIXNLNESLIDLQELGC

SEQ ID NO: 698 - protein, 40 aa, synthetic construct; identical to SEQ ID NO: 697
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 699 - protein, 40 aa, synthetic construct; identical to SEQ ID NO: 697
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 700 - protein, 40 aa, synthetic construct
  MOD_RES 9: note = 2-aminoisobutyric acid
  MOD_RES 16: note = ornithine
  MOD_RES 26: note = ornithine
LGDISGINXSVVNIQXEIEHLNEVIXNLNESLIDLQELGC

SEQ ID NO: 701 - protein, 40 aa, synthetic construct; identical to SEQ ID NO: 700
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 702 - protein, 40 aa, synthetic construct; identical to SEQ ID NO: 700
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 703 - protein, 40 aa, synthetic construct; identical to SEQ ID NO: 700
  MOD_RES 9: note = 2-aminoisobutyric acid
  MOD_RES 16: note = N-Methyl-Lysine
  MOD_RES 26: note = N-Methyl-Lysine

SEQ ID NO: 704 - protein, 40 aa, synthetic construct; identical to SEQ ID NO: 700
  MOD_RES 9: note = 2-aminoisobutyric acid
  MOD_RES 16: note = N-Dimethyl-Lysine
  MOD_RES 26: note = N-Dimethyl-Lysine

SEQ ID NO: 705 - protein, 40 aa, synthetic construct; identical to SEQ ID NO: 700
  MOD_RES 9: note = 2-aminoisobutyric acid
  MOD_RES 16: note = N alpha-methyl-L-lysine
  MOD_RES 26: note = ornithine

SEQ ID NO: 706 - protein, 40 aa, synthetic construct; identical to SEQ ID NO: 700
  MOD_RES 9: note = 2-aminoisobutyric acid
  MOD_RES 16: note = N alpha-methyl-L-lysine
  MOD_RES 26: note = 2,4-diaminobutyric acid

SEQ ID NO: 707 - protein, 40 aa, synthetic construct; identical to SEQ ID NO: 700
  MOD_RES 9: note = 2-aminoisobutyric acid
  MOD_RES 16: note = N alpha-methyl-L-lysine
  MOD_RES 26: note = 2,3-diaminopropionic acid

SEQ ID NO: 708 - protein, 40 aa, synthetic construct; identical to SEQ ID NO: 700
  MOD_RES 9: note = 2-aminoisobutyric acid
  MOD_RES 16: note = N alpha-methyl-L-lysine
  MOD_RES 26: note = N-Methyl-Lysine

SEQ ID NO: 709 - protein, 40 aa, synthetic construct; identical to SEQ ID NO: 700
  MOD_RES 9: note = 2-aminoisobutyric acid
  MOD_RES 16: note = N alpha-methyl-L-lysine
  MOD_RES 26: note = N-Dimethyl-Lysine

SEQ ID NO: 710 - protein, 40 aa, synthetic construct
  MOD_RES 9: note = 2-aminoisobutyric acid
  MOD_RES 16: note = ornithine
  MOD_RES 20: note = ornithine
  MOD_RES 26: note = ornithine
LGDISGINXSVVNIQXEIDXLNEVIXNLNESLIDLQELGC

SEQ ID NO: 711 - protein, 40 aa, synthetic construct; identical to SEQ ID NO: 710
  MOD_RES 9: note = 2-aminoisobutyric acid
  MOD_RES 16: note = 2,4-diaminobutyric acid
  MOD_RES 20: note = ornithine
  MOD_RES 26: note = 2,4-diaminobutyric acid

SEQ ID NO: 712 - protein, 40 aa, synthetic construct; identical to SEQ ID NO: 710
  MOD_RES 9: note = 2-aminoisobutyric acid
  MOD_RES 16: note = 2,3-diaminopropionic acid
  MOD_RES 20: note = ornithine
  MOD_RES 26: note = 2,3-diaminopropionic acid

SEQ ID NO: 713 - protein, 40 aa, synthetic construct; identical to SEQ ID NO: 710
  MOD_RES 9: note = 2-aminoisobutyric acid
  MOD_RES 16: note = N-Methyl-Lysine
  MOD_RES 20: note = ornithine
  MOD_RES 26: note = N-Methyl-Lysine

SEQ ID NO: 714 - protein, 40 aa, synthetic construct; identical to SEQ ID NO: 710
  MOD_RES 9: note = 2-aminoisobutyric acid
  MOD_RES 16: note = N-Dimethyl-Lysine
  MOD_RES 20: note = ornithine
  MOD_RES 26: note = N-Dimethyl-Lysine

SEQ ID NO: 715 - protein, 40 aa, synthetic construct; identical to SEQ ID NO: 710
  MOD_RES 9: note = 2-aminoisobutyric acid
  MOD_RES 16: note = N alpha-methyl-L-lysine
  MOD_RES 20: note = ornithine
  MOD_RES 26: note = ornithine

SEQ ID NO: 716 - protein, 40 aa, synthetic construct; identical to SEQ ID NO: 710
  MOD_RES 9: note = 2-aminoisobutyric acid
  MOD_RES 16: note = N alpha-methyl-L-lysine
  MOD_RES 20: note = ornithine
  MOD_RES 26: note = 2,4-diaminobutyric acid

SEQ ID NO: 717 - protein, 40 aa, synthetic construct; identical to SEQ ID NO: 710
  MOD_RES 9: note = 2-aminoisobutyric acid
  MOD_RES 16: note = N alpha-methyl-L-lysine
  MOD_RES 20: note = ornithine
  MOD_RES 26: note = 2,3-diaminopropionic acid

SEQ ID NO: 718 - protein, 40 aa, synthetic construct; identical to SEQ ID NO: 710
  MOD_RES 9: note = 2-aminoisobutyric acid
  MOD_RES 16: note = N alpha-methyl-L-lysine
  MOD_RES 20: note = ornithine
  MOD_RES 26: note = N-Methyl-Lysine

SEQ ID NO: 719 - protein, 40 aa, synthetic construct; identical to SEQ ID NO: 710
  MOD_RES 9: note = 2-aminoisobutyric acid
  MOD_RES 16: note = N alpha-methyl-L-lysine
  MOD_RES 20: note = ornithine
  MOD_RES 26: note = N-Dimethyl-Lysine

SEQ ID NO: 720 - protein, 40 aa, synthetic construct; identical to SEQ ID NO: 710
  MOD_RES 9: note = 2-aminoisobutyric acid
  MOD_RES 16: note = ornithine
  MOD_RES 20: note = Citrulline
  MOD_RES 26: note = ornithine

SEQ ID NO: 721 - protein, 40 aa, synthetic construct; identical to SEQ ID NO: 710
  MOD_RES 9: note = 2-aminoisobutyric acid
  MOD_RES 16: note = 2,4-diaminobutyric acid
  MOD_RES 20: note = Citrulline
  MOD_RES 26: note = 2,4-diaminobutyric acid

SEQ ID NO: 722 - protein, 40 aa, synthetic construct; identical to SEQ ID NO: 710
  MOD_RES 9: note = 2-aminoisobutyric acid
  MOD_RES 16: note = 2,3-diaminopropionic acid
  MOD_RES 20: note = Citrulline
  MOD_RES 26: note = 2,3-diaminopropionic acid

SEQ ID NO: 723 - protein, 40 aa, synthetic construct; identical to SEQ ID NO: 710
  MOD_RES 9: note = 2-aminoisobutyric acid
  MOD_RES 16: note = N-Methyl-Lysine
  MOD_RES 20: note = Citrulline
  MOD_RES 26: note = N-Methyl-Lysine

SEQ ID NO: 724 - protein, 40 aa, synthetic construct; identical to SEQ ID NO: 710
  MOD_RES 9: note = 2-aminoisobutyric acid
  MOD_RES 16: note = N-Dimethyl-Lysine
  MOD_RES 20: note = Citrulline
  MOD_RES 26: note = N-Dimethyl-Lysine

SEQ ID NO: 725 - protein, 40 aa, synthetic construct; identical to SEQ ID NO: 710
  MOD_RES 9: note = 2-aminoisobutyric acid
  MOD_RES 16: note = N alpha-methyl-L-lysine
  MOD_RES 20: note = Citrulline
  MOD_RES 26: note = ornithine

SEQ ID NO: 726 - protein, 40 aa, synthetic construct; identical to SEQ ID NO: 710
  MOD_RES 9: note = 2-aminoisobutyric acid
  MOD_RES 16: note = N alpha-methyl-L-lysine
  MOD_RES 20: note = Citrulline
  MOD_RES 26: note = 2,4-diaminobutyric acid

SEQ ID NO: 727 - protein, 40 aa, synthetic construct; identical to SEQ ID NO: 710
  MOD_RES 9: note = 2-aminoisobutyric acid
  MOD_RES 16: note = N alpha-methyl-L-lysine
  MOD_RES 20: note = Citrulline
  MOD_RES 26: note = 2,3-diaminopropionic acid

SEQ ID NO: 728 - protein, 40 aa, synthetic construct; identical to SEQ ID NO: 710
  MOD_RES 9: note = 2-aminoisobutyric acid
  MOD_RES 16: note = N alpha-methyl-L-lysine
  MOD_RES 20: note = Citrulline
  MOD_RES 26: note = N-Methyl-Lysine

SEQ ID NO: 729 - protein, 40 aa, synthetic construct; identical to SEQ ID NO: 710
  MOD_RES 9: note = 2-aminoisobutyric acid
  MOD_RES 16: note = N alpha-methyl-L-lysine
  MOD_RES 20: note = Citrulline
  MOD_RES 26: note = N-Dimethyl-Lysine

SEQ ID NO: 730 - protein, 40 aa, synthetic construct
  MOD_RES 9: note = 2-aminoisobutyric acid
  MOD_RES 16: note = ornithine
  MOD_RES 26: note = ornithine
LGDISGINXSVVNIQXEIDHLNEVAXNLGESLIELQELGC

SEQ ID NO: 731 - protein, 40 aa, synthetic construct; identical to SEQ ID NO: 730
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 732 - protein, 40 aa, synthetic construct; identical to SEQ ID NO: 730
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 733 - protein, 40 aa, synthetic construct
  MOD_RES 9: note = 2-aminoisobutyric acid
  MOD_RES 16: note = ornithine
  MOD_RES 26: note = ornithine
LGDISGINXSVVNIQXEIEHLNEVAXNLGESLIELQELGC

SEQ ID NO: 734 - protein, 40 aa, synthetic construct; identical to SEQ ID NO: 733
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 735 - protein, 40 aa, synthetic construct; identical to SEQ ID NO: 733
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 736 - protein, 40 aa, synthetic construct; identical to SEQ ID NO: 733
  MOD_RES 9: note = 2-aminoisobutyric acid
  MOD_RES 16: note = N-Methyl-Lysine
  MOD_RES 26: note = N-Methyl-Lysine

SEQ ID NO: 737 - protein, 40 aa, synthetic construct; identical to SEQ ID NO: 733
  MOD_RES 9: note = 2-aminoisobutyric acid
  MOD_RES 16: note = N-Dimethyl-Lysine
  MOD_RES 26: note = N-Dimethyl-Lysine

SEQ ID NO: 738 - protein, 40 aa, synthetic construct; identical to SEQ ID NO: 733
  MOD_RES 9: note = 2-aminoisobutyric acid
  MOD_RES 16: note = N alpha-methyl-L-lysine
  MOD_RES 26: note = ornithine

SEQ ID NO: 739 - protein, 40 aa, synthetic construct; identical to SEQ ID NO: 733
  MOD_RES 9: note = 2-aminoisobutyric acid
  MOD_RES 16: note = N alpha-methyl-L-lysine
  MOD_RES 26: note = 2,4-diaminobutyric acid

SEQ ID NO: 740 - protein, 40 aa, synthetic construct; identical to SEQ ID NO: 733
  MOD_RES 9: note = 2-aminoisobutyric acid
  MOD_RES 16: note = N alpha-methyl-L-lysine
  MOD_RES 26: note = 2,3-diaminopropionic acid

SEQ ID NO: 741 - protein, 40 aa, synthetic construct; identical to SEQ ID NO: 733
  MOD_RES 9: note = 2-aminoisobutyric acid
  MOD_RES 16: note = N alpha-methyl-L-lysine
  MOD_RES 26: note = N-Methyl-Lysine

SEQ ID NO: 742 - protein, 40 aa, synthetic construct; identical to SEQ ID NO: 733
  MOD_RES 9: note = 2-aminoisobutyric acid
  MOD_RES 16: note = N alpha-methyl-L-lysine
  MOD_RES 26: note = N-Dimethyl-Lysine

SEQ ID NO: 743 - protein, 40 aa, synthetic construct
  MOD_RES 9: note = 2-aminoisobutyric acid
  MOD_RES 16: note = ornithine
  MOD_RES 20: note = ornithine
  MOD_RES 26: note = ornithine
LGDISGINXSVVNIQXEIDXLNEVAXNLGESLIELQELGC

SEQ ID NO: 744 - protein, 40 aa, synthetic construct; identical to SEQ ID NO: 743
  MOD_RES 9: note = 2-aminoisobutyric acid
  MOD_RES 16: note = 2,4-diaminobutyric acid
  MOD_RES 20: note = ornithine
  MOD_RES 26: note = 2,4-diaminobutyric acid

SEQ ID NO: 745 - protein, 40 aa, synthetic construct; identical to SEQ ID NO: 743
  MOD_RES 9: note = 2-aminoisobutyric acid
  MOD_RES 16: note = 2,3-diaminopropionic acid
  MOD_RES 20: note = ornithine
  MOD_RES 26: note = 2,3-diaminopropionic acid

SEQ ID NO: 746 - protein, 40 aa, synthetic construct; identical to SEQ ID NO: 743
  MOD_RES 9: note = 2-aminoisobutyric acid
  MOD_RES 16: note = N-Methyl-Lysine
  MOD_RES 20: note = ornithine
  MOD_RES 26: note = N-Methyl-Lysine

SEQ ID NO: 747 - protein, 40 aa, synthetic construct; identical to SEQ ID NO: 743
  MOD_RES 9: note = 2-aminoisobutyric acid
  MOD_RES 16: note = N-Dimethyl-Lysine
  MOD_RES 20: note = ornithine
  MOD_RES 26: note = N-Dimethyl-Lysine

SEQ ID NO: 748 - protein, 40 aa, synthetic construct; identical to SEQ ID NO: 743
  MOD_RES 9: note = 2-aminoisobutyric acid
  MOD_RES 16: note = N alpha-methyl-L-lysine
  MOD_RES 20: note = ornithine
  MOD_RES 26: note = ornithine

SEQ ID NO: 749 - protein, 40 aa, synthetic construct; identical to SEQ ID NO: 743
  MOD_RES 9: note = 2-aminoisobutyric acid
  MOD_RES 16: note = N alpha-methyl-L-lysine
  MOD_RES 20: note = ornithine
  MOD_RES 26: note = 2,4-diaminobutyric acid

SEQ ID NO: 750 - protein, 40 aa, synthetic construct; identical to SEQ ID NO: 743
  MOD_RES 9: note = 2-aminoisobutyric acid
  MOD_RES 16: note = N alpha-methyl-L-lysine
  MOD_RES 20: note = ornithine
  MOD_RES 26: note = 2,3-diaminopropionic acid

SEQ ID NO: 751 - protein, 40 aa, synthetic construct; identical to SEQ ID NO: 743
  MOD_RES 9: note = 2-aminoisobutyric acid
  MOD_RES 16: note = N alpha-methyl-L-lysine
  MOD_RES 20: note = ornithine
  MOD_RES 26: note = N-Methyl-Lysine

SEQ ID NO: 752 - protein, 40 aa, synthetic construct; identical to SEQ ID NO: 743
  MOD_RES 9: note = 2-aminoisobutyric acid
  MOD_RES 16: note = N alpha-methyl-L-lysine
  MOD_RES 20: note = ornithine
  MOD_RES 26: note = N-Dimethyl-Lysine

SEQ ID NO: 753 - protein, 40 aa, synthetic construct; identical to SEQ ID NO: 743
  MOD_RES 9: note = 2-aminoisobutyric acid
  MOD_RES 16: note = ornithine
  MOD_RES 20: note = Citrulline
  MOD_RES 26: note = ornithine

SEQ ID NO: 754 - protein, 40 aa, synthetic construct; identical to SEQ ID NO: 743
  MOD_RES 9: note = 2-aminoisobutyric acid
  MOD_RES 16: note = 2,4-diaminobutyric acid
  MOD_RES 20: note = Citrulline
  MOD_RES 26: note = 2,4-diaminobutyric acid

SEQ ID NO: 755 - protein, 40 aa, synthetic construct; identical to SEQ ID NO: 743
  MOD_RES 9: note = 2-aminoisobutyric acid
  MOD_RES 16: note = 2,3-diaminopropionic acid2,3-diaminopropionic acid
  MOD_RES 20: note = Citrulline
  MOD_RES 26: note = 2,3-diaminopropionic acid

SEQ ID NO: 756 - protein, 40 aa, synthetic construct; identical to SEQ ID NO: 743
  MOD_RES 9: note = 2-aminoisobutyric acid
  MOD_RES 16: note = N-Methyl-Lysine
  MOD_RES 20: note = Citrulline
  MOD_RES 26: note = N-Methyl-Lysine

SEQ ID NO: 757 - protein, 40 aa, synthetic construct; identical to SEQ ID NO: 743
  MOD_RES 9: note = 2-aminoisobutyric acid
  MOD_RES 16: note = N-Dimethyl-Lysine
  MOD_RES 20: note = Citrulline
  MOD_RES 26: note = N-Dimethyl-Lysine

SEQ ID NO: 758 - protein, 40 aa, synthetic construct; identical to SEQ ID NO: 743
  MOD_RES 9: note = 2-aminoisobutyric acid
  MOD_RES 16: note = N alpha-methyl-L-lysine
  MOD_RES 20: note = Citrulline
  MOD_RES 26: note = ornithine

SEQ ID NO: 759 - protein, 40 aa, synthetic construct; identical to SEQ ID NO: 743
  MOD_RES 9: note = 2-aminoisobutyric acid
  MOD_RES 16: note = N alpha-methyl-L-lysine
  MOD_RES 20: note = Citrulline
  MOD_RES 26: note = 2,4-diaminobutyric acid

SEQ ID NO: 760 - protein, 40 aa, synthetic construct; identical to SEQ ID NO: 743
  MOD_RES 9: note = 2-aminoisobutyric acid
  MOD_RES 16: note = N alpha-methyl-L-lysine
  MOD_RES 20: note = Citrulline
  MOD_RES 26: note = 2,3-diaminopropionic acid

SEQ ID NO: 761 - protein, 40 aa, synthetic construct; identical to SEQ ID NO: 743
  MOD_RES 9: note = 2-aminoisobutyric acid
  MOD_RES 16: note = N alpha-methyl-L-lysine
  MOD_RES 20: note = Citrulline
  MOD_RES 26: note = N-Methyl-Lysine

SEQ ID NO: 762 - protein, 40 aa, synthetic construct; identical to SEQ ID NO: 743
  MOD_RES 9: note = 2-aminoisobutyric acid
  MOD_RES 16: note = N alpha-methyl-L-lysine
  MOD_RES 20: note = Citrulline
  MOD_RES 26: note = N-Dimethyl-Lysine

SEQ ID NO: 763 - protein, 40 aa, synthetic construct
  MOD_RES 9: note = 2-aminoisobutyric acid
  MOD_RES 16: note = ornithine
  MOD_RES 26: note = ornithine
LGDISGINXSVVNIQXEIDHLNEVIXNLGESLIELQELGC

SEQ ID NO: 764 - protein, 40 aa, synthetic construct; identical to SEQ ID NO: 763
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 765 - protein, 40 aa, synthetic construct; identical to SEQ ID NO: 763
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 766 - protein, 40 aa, synthetic construct
  MOD_RES 9: note = 2-aminoisobutyric acid
  MOD_RES 16: note = ornithine
  MOD_RES 26: note = ornithine
LGDISGINXSVVNIQXEIEHLNEVIXNLGESLIELQELGC

SEQ ID NO: 767 - protein, 40 aa, synthetic construct; identical to SEQ ID NO: 766
  MOD_RES 9: note = 2-aminoisobutyric acid
  MOD_RES 16: note = 2,4-diaminobutyric acid
  MOD_RES 26: note = 2,4-diaminobutyric acid

SEQ ID NO: 768 - protein, 40 aa, synthetic construct; identical to SEQ ID NO: 766
  MOD_RES 9: note = 2-aminoisobutyric acid
  MOD_RES 16: note = 2,3-diaminopropionic acid
  MOD_RES 26: note = 2,3-diaminopropionic acid

SEQ ID NO: 769 - protein, 40 aa, synthetic construct; identical to SEQ ID NO: 766
  MOD_RES 9: note = 2-aminoisobutyric acid
  MOD_RES 16: note = N-Methyl-Lysine
  MOD_RES 26: note = N-Methyl-Lysine

SEQ ID NO: 770 - protein, 40 aa, synthetic construct; identical to SEQ ID NO: 766
  MOD_RES 9: note = 2-aminoisobutyric acid
  MOD_RES 16: note = N-Dimethyl-Lysine
  MOD_RES 26: note = N-Dimethyl-Lysine

SEQ ID NO: 771 - protein, 40 aa, synthetic construct; identical to SEQ ID NO: 766
  MOD_RES 9: note = 2-aminoisobutyric acid
  MOD_RES 16: note = N alpha-methyl-L-lysine
  MOD_RES 26: note = ornithine

SEQ ID NO: 772 - protein, 40 aa, synthetic construct; identical to SEQ ID NO: 766
  MOD_RES 9: note = 2-aminoisobutyric acid
  MOD_RES 16: note = N alpha-methyl-L-lysine
  MOD_RES 26: note = 2,4-diaminobutyric acid

SEQ ID NO: 773 - protein, 40 aa, synthetic construct; identical to SEQ ID NO: 766
  MOD_RES 9: note = 2-aminoisobutyric acid
  MOD_RES 16: note = N alpha-methyl-L-lysine
  MOD_RES 26: note = 2,3-diaminopropionic acid

SEQ ID NO: 774 - protein, 40 aa, synthetic construct; identical to SEQ ID NO: 766
  MOD_RES 9: note = 2-aminoisobutyric acid
  MOD_RES 16: note = N alpha-methyl-L-lysine
  MOD_RES 26: note = N-Methyl-Lysine

SEQ ID NO: 775 - protein, 40 aa, synthetic construct; identical to SEQ ID NO: 766
  MOD_RES 9: note = 2-aminoisobutyric acid
  MOD_RES 16: note = N alpha-methyl-L-lysine
  MOD_RES 26: note = N-Dimethyl-Lysine

SEQ ID NO: 776 - protein, 40 aa, synthetic construct
  MOD_RES 9: note = 2-aminoisobutyric acid
  MOD_RES 16: note = ornithine
  MOD_RES 20: note = ornithine
  MOD_RES 26: note = ornithine
LGDISGINXSVVNIQXEIDXLNEVIXNLGESLIELQELGC

SEQ ID NO: 777 - protein, 40 aa, synthetic construct; identical to SEQ ID NO: 776
  MOD_RES 9: note = 2-aminoisobutyric acid
  MOD_RES 16: note = 2,4-diaminobutyric acid
  MOD_RES 20: note = ornithine
  MOD_RES 26: note = 2,4-diaminobutyric acid

SEQ ID NO: 778 - protein, 40 aa, synthetic construct; identical to SEQ ID NO: 776
  MOD_RES 9: note = 2-aminoisobutyric acid
  MOD_RES 16: note = 2,3-diaminopropionic acid
  MOD_RES 20: note = ornithine
  MOD_RES 26: note = 2,3-diaminopropionic acid

SEQ ID NO: 779 - protein, 40 aa, synthetic construct; identical to SEQ ID NO: 776
  MOD_RES 9: note = 2-aminoisobutyric acid
  MOD_RES 16: note = N-Methyl-Lysine
  MOD_RES 20: note = ornithine
  MOD_RES 26: note = N-Methyl-Lysine

SEQ ID NO: 780 - protein, 40 aa, synthetic construct; identical to SEQ ID NO: 776
  MOD_RES 9: note = 2-aminoisobutyric acid
  MOD_RES 16: note = N-Dimethyl-Lysine
  MOD_RES 20: note = ornithine
  MOD_RES 26: note = N-Dimethyl-Lysine

SEQ ID NO: 781 - protein, 40 aa, synthetic construct; identical to SEQ ID NO: 776
  MOD_RES 9: note = 2-aminoisobutyric acid
  MOD_RES 16: note = N alpha-methyl-L-lysine
  MOD_RES 20: note = ornithine
  MOD_RES 26: note = ornithine

SEQ ID NO: 782 - protein, 40 aa, synthetic construct; identical to SEQ ID NO: 776
  MOD_RES 9: note = 2-aminoisobutyric acid
  MOD_RES 16: note = N alpha-methyl-L-lysine
  MOD_RES 20: note = ornithine
  MOD_RES 26: note = 2,4-diaminobutyric acid

SEQ ID NO: 783 - protein, 40 aa, synthetic construct; identical to SEQ ID NO: 776
  MOD_RES 9: note = 2-aminoisobutyric acid
  MOD_RES 16: note = N alpha-methyl-L-lysine
  MOD_RES 20: note = ornithine
  MOD_RES 26: note = 2,3-diaminopropionic acid

SEQ ID NO: 784 - protein, 40 aa, synthetic construct; identical to SEQ ID NO: 776
  MOD_RES 9: note = 2-aminoisobutyric acid
  MOD_RES 16: note = N alpha-methyl-L-lysine
  MOD_RES 20: note = ornithine
  MOD_RES 26: note = N-Methyl-Lysine

SEQ ID NO: 785 - protein, 40 aa, synthetic construct; identical to SEQ ID NO: 776
  MOD_RES 9: note = 2-aminoisobutyric acid
  MOD_RES 16: note = N alpha-methyl-L-lysine
  MOD_RES 20: note = ornithine
  MOD_RES 26: note = N-Dimethyl-Lysine

SEQ ID NO: 786 - protein, 40 aa, synthetic construct; identical to SEQ ID NO: 776
  MOD_RES 9: note = 2-aminoisobutyric acid
  MOD_RES 16: note = ornithine
  MOD_RES 20: note = Citrulline
  MOD_RES 26: note = ornithine

SEQ ID NO: 787 - protein, 40 aa, synthetic construct; identical to SEQ ID NO: 776
  MOD_RES 9: note = 2-aminoisobutyric acid
  MOD_RES 16: note = 2,4-diaminobutyric acid
  MOD_RES 20: note = Citrulline
  MOD_RES 26: note = 2,4-diaminobutyric acid

SEQ ID NO: 788 - protein, 40 aa, synthetic construct; identical to SEQ ID NO: 776
  MOD_RES 9: note = 2-aminoisobutyric acid
  MOD_RES 16: note = 2,3-diaminopropionic acid
  MOD_RES 20: note = Citrulline
  MOD_RES 26: note = 2,3-diaminopropionic acid

SEQ ID NO: 789 - protein, 40 aa, synthetic construct; identical to SEQ ID NO: 776
  MOD_RES 9: note = 2-aminoisobutyric acid
  MOD_RES 16: note = N-Methyl-Lysine
  MOD_RES 20: note = Citrulline
  MOD_RES 26: note = N-Methyl-Lysine

SEQ ID NO: 790 - protein, 40 aa, synthetic construct; identical to SEQ ID NO: 776
  MOD_RES 9: note = 2-aminoisobutyric acid
  MOD_RES 16: note = N-Dimethyl-Lysine
  MOD_RES 20: note = Citrulline
  MOD_RES 26: note = N-Dimethyl-Lysine

SEQ ID NO: 791 - protein, 40 aa, synthetic construct; identical to SEQ ID NO: 776
  MOD_RES 9: note = 2-aminoisobutyric acid
  MOD_RES 16: note = N alpha-methyl-L-lysine
  MOD_RES 20: note = Citrulline
  MOD_RES 26: note = ornithine

SEQ ID NO: 792 - protein, 40 aa, synthetic construct; identical to SEQ ID NO: 776
  MOD_RES 9: note = 2-aminoisobutyric acid
  MOD_RES 16: note = N alpha-methyl-L-lysine
  MOD_RES 20: note = Citrulline
  MOD_RES 26: note = 2,4-diaminobutyric acid

SEQ ID NO: 793 - protein, 40 aa, synthetic construct; identical to SEQ ID NO: 776
  MOD_RES 9: note = 2-aminoisobutyric acid
  MOD_RES 16: note = N alpha-methyl-L-lysine
  MOD_RES 20: note = Citrulline
  MOD_RES 26: note = 2,3-diaminopropionic acid

SEQ ID NO: 794 - protein, 40 aa, synthetic construct; identical to SEQ ID NO: 776
  MOD_RES 9: note = 2-aminoisobutyric acid
  MOD_RES 16: note = N alpha-methyl-L-lysine
  MOD_RES 20: note = Citrulline
  MOD_RES 26: note = N-Dimethyl-Lysine

SEQ ID NO: 795 - protein, 40 aa, synthetic construct; identical to SEQ ID NO: 776
  MOD_RES 9: note = 2-aminoisobutyric acid
  MOD_RES 16: note = N alpha-methyl-L-lysine
  MOD_RES 20: note = Citrulline
  MOD_RES 26: note = N-Dimethyl-Lysine

SEQ ID NO: 796 - protein, 40 aa, synthetic construct; identical to SEQ ID NO: 435

SEQ ID NO: 797 - protein, 37 aa, synthetic construct; identical to SEQ ID NO: 436

SEQ ID NO: 798 - protein, 29 aa, synthetic construct
GINASVVNIQKEIDRLNEVAKNLGSGSGC

SEQ ID NO: 799 - protein, 36 aa, synthetic construct
GINASVVNIQKEIDRLNEVAKNLNESLIDLGSGSGC

SEQ ID NO: 800 - protein, 43 aa, synthetic construct
GINASVVNIQKEIDRLNEVAKNLNESLIDLQELGKYEGSGSGC

SEQ ID NO: 801 - protein, 28 aa, synthetic construct
ISYVEITVPNIQKEIDRLNEVAKNLNES

SEQ ID NO: 802 - protein, 29 aa, synthetic construct
DISYVEITVPNIQKEIDRLNEVAKNLNES

SEQ ID NO: 803 - protein, 35 aa, synthetic construct
ISYVEITVPNIQKEIDRLNEVAKNLNESLIDLQEL

SEQ ID NO: 804 - protein, 36 aa, synthetic construct
DISYVEITVPNIQKEIDRLNEVAKNLNESLIDLQEL

SEQ ID NO: 805 - protein, 42 aa, synthetic construct
PDVDLGDISYVEITVPNIQKEIDRLNEVAKNLNESLIDLQEL

SEQ ID NO: 806 - protein, 32 aa, synthetic construct
PDVDLGDISYVEITVPNIQKEIDRLNEVAKNL

SEQ ID NO: 807 - protein, 37 aa, synthetic construct
DISGINASVVNIQKEIDRLNEVAKNLNESLIDLQELG

SEQ ID NO: 808 - protein, 32 aa, synthetic construct; identical to SEQ ID NO: 446

SEQ ID NO: 809 - protein, 42 aa, synthetic construct
DISGINASVVNIQKEIDRLNEVAENLNESLIDLQELGSGSGC

SEQ ID NO: 810 - protein, 42 aa, synthetic construct
DISGINASVVNIEYEIDRLNEVAKNLNESLIDLQELGSGSGC

SEQ ID NO: 811 - protein, 42 aa, synthetic construct
DISGINASVVNIQKEIDHLNEVAKNLNESLIDLQELGSGSGC

SEQ ID NO: 812 - protein, 46 aa, synthetic construct
PDLGDISGINESVVNIQSEIDSLNELIDNLNESLIDLQELGSGSGC

SEQ ID NO: 813 - protein, 42 aa, synthetic construct
PDLGDISGINESVVNIQSEIDSLNELIDNLNESLIDLQELGC

SEQ ID NO: 814 - protein, 45 aa, synthetic construct
DLGDISGINESVVNIQSEIDSLNELIDNLNESLIDLQELGSGSGC

SEQ ID NO: 815 - protein, 41 aa, synthetic construct
DLGDISGINESVVNIQSEIDSLNELIDNLNESLIDLQELGC

SEQ ID NO: 816 - protein, 41 aa, synthetic construct
DLGDISGINQSVVNIQSEIDSLNELIDNLNESLIDLQELGC

SEQ ID NO: 817 - protein, 41 aa, synthetic construct
DLGDISGINLSVVNIQSEIDSLNELIDNLNESLIDLQELGC

SEQ ID NO: 818 - protein, 41 aa, synthetic construct
DLGDISGINTSVVNIQSEIDSLNELIDNLNESLIDLQELGC

SEQ ID NO: 819 - protein, 42 aa, synthetic construct
PDLGDISGINESLLNIQSEIDSLNELIDNLNESLIDLQELGC

SEQ ID NO: 820 - protein, 42 aa, synthetic construct
PDLGDISGINESILNIQSEIDSLNELIDNLNESLIDLQELGC

SEQ ID NO: 821 - protein, 42 aa, synthetic construct
PDLGDISGINATILNIQSEIDSLNELIDNLNESLIDLQELGC

SEQ ID NO: 822 - protein, 41 aa, synthetic construct
DLGDISGINATILNIQDEIDHLNELIDNLNESLIDLQELGC

SEQ ID NO: 823 - protein, 43 aa, synthetic construct
DLGDISGINLTILNIQDEIDHLNELIDNLNESLIDLQELGSGC

SEQ ID NO: 824 - protein, 43 aa, synthetic construct
DLGDISGINLTILNIQLEIEHLNEVIENLGESLIELQELGSGC

SEQ ID NO: 825 - protein, 42 aa, synthetic construct
DISGINASVVNIQKEIDRLNEVIKNLGESLIELQELGSGSGC

SEQ ID NO: 826 - protein, 42 aa, synthetic construct
DISGINASVVNIQKEIDHLNEVIKNLGESLIELQELGSGSGC

SEQ ID NO: 827 - protein, 42 aa, synthetic construct
DISGINASVVNIQKEIEHLNEVIKNLGESLIELQELGSGSGC

SEQ ID NO: 828 - protein, 45 aa, synthetic construct
DLGDISGINASVVNIQKEIEHLNEVIKNLGESLIELQELGSGSGC

SEQ ID NO: 829 - protein, 43 aa, synthetic construct
DLGDISGINASVVNIQKEIEHLNEVIKNLGESLIELQELGSGC

SEQ ID NO: 830 - protein, 43 aa, synthetic construct
DLGDISGINLSVVNIQLEIEHLNEVAENLNESLIDLQELGSGC

SEQ ID NO: 831 - protein, 41 aa, synthetic construct
DLGDISGINLSVVNIQLEIEHLNEVAENLNESLIDLQELGC

SEQ ID NO: 832 - protein, 44 aa, synthetic construct
LGDISGINASVVNIQKEIEHLNEVAKNLNESLIDLQELGSGSGC

SEQ ID NO: 833 - protein, 44 aa, synthetic construct
LGDISGINASVVNIQLEIDRLNEVAKNLNESLIDLQELGSGSGC

SEQ ID NO: 834 - protein, 44 aa, synthetic construct
LGDISGINLSVVNIQKEIDRLNEVAKNLNESLIDLQELGSGSGC

SEQ ID NO: 835 - protein, 44 aa, synthetic construct
LGDISGINASVLNIQKEIDRLNEVAKNLNESLIDLQELGSGSGC

SEQ ID NO: 836 - protein, 44 aa, synthetic construct
LGDISGINASIVNIQKEIDRLNEVAKNLNESLIDLQELGSGSGC

SEQ ID NO: 837 - protein, 44 aa, synthetic construct
LGDISGINASLVNIQKEIDRLNEVAKNLNESLIDLQELGSGSGC

SEQ ID NO: 838 - protein, 44 aa, synthetic construct
LGDISGINASILNIQKEIDRLNEVAKNLNESLIDLQELGSGSGC

SEQ ID NO: 839 - protein, 44 aa, synthetic construct
LGDISGINASVVNIQKEIDRLNEVAENLNESLIDLQELGSGSGC

SEQ ID NO: 840 - protein, 42 aa, synthetic construct
LGDISGINASVVNIQKEIDRLNEVAKNLNESLIDLQELGSGC

SEQ ID NO: 841 - protein, 45 aa, synthetic construct
DLGDISGINLSVVNIQLEIEHLNEVIENLGESLIELQELGSGSGC

SEQ ID NO: 842 - protein, 43 aa, synthetic construct
DLGDISGINLTILDIQLEIEHLNELIENLGESLIELQELGSGC

SEQ ID NO: 843 - protein, 43 aa, synthetic construct
DLGDISGINLTILNIQLEIDHLNELIENLGESLIELQELGSGC

SEQ ID NO: 844 - protein, 43 aa, synthetic construct
DLGDISGINLTILNIQLEINHLNELIENLGESLIELQELGSGC

SEQ ID NO: 845 - protein, 43 aa, synthetic construct
DLGDISGINLTILNIQLEILHLNELIENLGESLIELQELGSGC

SEQ ID NO: 846 - protein, 43 aa, synthetic construct
DLGDISGINLTILNIQLEISHLNELIENLGESLIELQELGSGC

SEQ ID NO: 847 - protein, 43 aa, synthetic construct
DLGDISGINLTILNIQLEIEHLNELIENLGESLIDLQELGSGC

SEQ ID NO: 848 - protein, 43 aa, synthetic construct
DLGDISGINLTILNIQLEIEHLNELIENLNESLIDLQELGSGC

SEQ ID NO: 849 - protein, 41 aa, synthetic construct
DLGDISGINSSVVNIQKEIDRLNEVAKNLNESLIDLQELGC

SEQ ID NO: 850 - protein, 41 aa, synthetic construct
DLGDISGINGSVVNIQKEIDRLNEVAKNLNESLIDLQELGC

SEQ ID NO: 851 - protein, 41 aa, synthetic construct
DLGDISGINTSVVNIQKEIDRLNEVAKNLNESLIDLQELGC

SEQ ID NO: 852 - protein, 41 aa, synthetic construct
DLGDISGINASIVNIQKEIDRLNEVAKNLNESLIDLQELGC

SEQ ID NO: 853 - protein, 41 aa, synthetic construct
DLGDISGINASLVNIQKEIDRLNEVAKNLNESLIDLQELGC

SEQ ID NO: 854 - protein, 41 aa, synthetic construct
DLGDISGINASMVNIQKEIDRLNEVAKNLNESLIDLQELGC

SEQ ID NO: 855 - protein, 41 aa, synthetic construct
DLGDISGINASVINIQKEIDRLNEVAKNLNESLIDLQELGC

SEQ ID NO: 856 - protein, 41 aa, synthetic construct
DLGDISGINASVLNIQKEIDRLNEVAKNLNESLIDLQELGC

SEQ ID NO: 857 - protein, 41 aa, synthetic construct
DLGDISGINASVMNIQKEIDRLNEVAKNLNESLIDLQELGC

SEQ ID NO: 858 - protein, 41 aa, synthetic construct
DLGDISGINASIINIQKEIDRLNEVAKNLNESLIDLQELGC

SEQ ID NO: 859 - protein, 41 aa, synthetic construct
DLGDISGINASLLNIQKEIDRLNEVAKNLNESLIDLQELGC

SEQ ID NO: 860 - protein, 41 aa, synthetic construct
DLGDISGINASMMNIQKEIDRLNEVAKNLNESLIDLQELGC

SEQ ID NO: 861 - protein, 41 aa, synthetic construct
DLGDISGINASVVNIQQEIDRLNEVAKNLNESLIDLQELGC

SEQ ID NO: 862 - protein, 41 aa, synthetic construct
DLGDISGINASVVNIQEEIDRLNEVAKNLNESLIDLQELGC

SEQ ID NO: 863 - protein, 41 aa, synthetic construct
DLGDISGINASVVNIQSEIDRLNEVAKNLNESLIDLQELGC

SEQ ID NO: 864 - protein, 41 aa, synthetic construct
DLGDISGINASVVNIQNEIDRLNEVAKNLNESLIDLQELGC

SEQ ID NO: 865 - protein, 41 aa, synthetic construct
DLGDISGINASVVNIQKEIDNLNEVAKNLNESLIDLQELGC

SEQ ID NO: 866 - protein, 41 aa, synthetic construct
DLGDISGINASVVNIQKEIDQLNEVAKNLNESLIDLQELGC

SEQ ID NO: 867 - protein, 41 aa, synthetic construct
DLGDISGINASVVNIQKEIDELNEVAKNLNESLIDLQELGC

SEQ ID NO: 868 - protein, 41 aa, synthetic construct
DLGDISGINASVVNIQKEIDRLNEIAKNLNESLIDLQELGC

SEQ ID NO: 869 - protein, 41 aa, synthetic construct
DLGDISGINASVVNIQKEIDRLNEMAKNLNESLIDLQELGC

SEQ ID NO: 870 - protein, 41 aa, synthetic construct
DLGDISGINASVVNIQKEIDRLNELAKNLNESLIDLQELGC

SEQ ID NO: 871 - protein, 41 aa, synthetic construct
DLGDISGINASVVNIQKEIDRLNEAAKNLNESLIDLQELGC

SEQ ID NO: 872 - protein, 41 aa, synthetic construct
DLGDISGINASVVNIQKEIDRLNETAKNLNESLIDLQELGC

SEQ ID NO: 873 - protein, 41 aa, synthetic construct
DLGDISGINASVVNIQKEIDRLNEVSKNLNESLIDLQELGC

SEQ ID NO: 874 - protein, 41 aa, synthetic construct
DLGDISGINASVVNIQKEIDRLNEVVKNLNESLIDLQELGC

SEQ ID NO: 875 - protein, 41 aa, synthetic construct
DLGDISGINASVVNIQKEIDRLNEVCKNLNESLIDLQELGC

SEQ ID NO: 876 - protein, 41 aa, synthetic construct
DLGDISGINASVVNIQKEIDRLNEVTKNLNESLIDLQELGC

SEQ ID NO: 877 - protein, 41 aa, synthetic construct
DLGDISGINASVVNIQKEIDRLNEVGKNLNESLIDLQELGC

SEQ ID NO: 878 - protein, 41 aa, synthetic construct
DLGDISGINASVVNIQKEIDRLNELIKNLNESLIDLQELGC

SEQ ID NO: 879 - protein, 41 aa, synthetic construct
DLGDISGINASVVNIQKEIDRLNEIIKNLNESLIDLQELGC

SEQ ID NO: 880 - protein, 41 aa, synthetic construct
DLGDISGINASVVNIQKEIDRLNEISKNLNESLIDLQELGC

SEQ ID NO: 881 - protein, 41 aa, synthetic construct
DLGDISGINASVVNIQKEIDRLNEAIKNLNESLIDLQELGC

SEQ ID NO: 882 - protein, 41 aa, synthetic construct
DLGDISGINASVVNIQKEIDRLNETIKNLNESLIDLQELGC

SEQ ID NO: 883 - protein, 41 aa, synthetic construct
DLGDISGINASVVNIQKEIDRLNEVAQNLNESLIDLQELGC

SEQ ID NO: 884 - protein, 41 aa, synthetic construct
DLGDISGINASVVNIQKEIDRLNEVAENLNESLIDLQELGC

SEQ ID NO: 885 - protein, 41 aa, synthetic construct
DLGDISGINASVVNIQKEIDRLNEVASNLNESLIDLQELGC

SEQ ID NO: 886 - protein, 41 aa, synthetic construct
DLGDISGINASVVNIQKEIDRLNEVANNLNESLIDLQELGC

SEQ ID NO: 887 - protein, 41 aa, synthetic construct
DLGDISGINASVVNIQKEIDRLNEVAKNLGESLIDLQELGC

SEQ ID NO: 888 - protein, 41 aa, synthetic construct
DLGDISGINASIVNIQKEIDRLNEVIKNLNESLIDLQELGC

SEQ ID NO: 889 - protein, 42 aa, synthetic construct
CGSGSGLEQLDILSENLNKAVENLRDIEKQINVVSANIGSID

SEQ ID NO: 890 - protein, 36 aa, synthetic construct
EEEELTTLNEELSHLATLLEETVAELQALIDNIAST

SEQ ID NO: 891 - protein, 38 aa, synthetic construct
QVEELLQQLQEQIASTQAELGKLQDVRAALEAQLAALC

SEQ ID NO: 892 - protein, 38 aa, synthetic construct
DISSIAAAFVDIQEYIDHLLTVASELESALIYLIPLGC

SEQ ID NO: 893 - protein, 38 aa, synthetic construct
DISSIAAAFVDISEYIEHLLTVASELNSALIFLIPIGC

SEQ ID NO: 894 - protein, 38 aa, synthetic construct
DISSIAAAFVDISEYIEHLLTVASELESALIFLIPIGC

SEQ ID NO: 895 - protein, 38 aa, synthetic construct
DYSWIAAATVDIETEIQILLTVASELNSALIYLIPLGC

SEQ ID NO: 896 - protein, 38 aa, synthetic construct
DYSWIAAAFVDIETEIQILLQVATELNSALIFLIPIGC

SEQ ID NO: 897 - protein, 38 aa, synthetic construct
DYSWIAAAFVDIETEIQILLQVATELESALIFLIPIGC

SEQ ID NO: 898 - protein, 38 aa, synthetic construct
DYSWIAAAFVDIEEEIQILLQVATELNSALIFLIPLGC

SEQ ID NO: 899 - protein, 38 aa, synthetic construct
DYSWIAAAFVDIEEEIQILLQVATELESALIFLIPIGC

SEQ ID NO: 900 - protein, 38 aa, synthetic construct
DISSIAAAFVDIQTEIDIILTVANELNSALINLIPLGC

SEQ ID NO: 901 - protein, 38 aa, synthetic construct
DYSWIAAAFVDIETEIQILLQVATELNSALIFLIPLGC

SEQ ID NO: 902 - protein, 38 aa, synthetic construct
SLLAEIQQLQQEEAAALGKLQEAVGKLNELVKELLELC

SEQ ID NO: 903 - protein, 38 aa, synthetic construct
QALQQLLQQAQEQTAQLLSKIQEALGKLEEAIKELLEC

SEQ ID NO: 904 - protein, 38 aa, synthetic construct
DALQQIQQLQAQQAEALGKLAAAVGELAANLAELAAIC

SEQ ID NO: 905 - protein, 38 aa, synthetic construct
AALAEIEKLKAEEAAALAKLQEAVGKLAEALAELAAIC

SEQ ID NO: 906 - protein, 38 aa, synthetic construct
GLLAQIKELQAQEAAALAKLAESVGKLAELLKELKEIC

SEQ ID NO: 907 - protein, 38 aa, synthetic construct
LAEIAALQAQAAEALSKLAASLGKLEEYLKKLQEILEC

SEQ ID NO: 908 - protein, 38 aa, synthetic construct
AAAEQIAALQQQIAKLQAELGKLQDERAKLEAELAALC

SEQ ID NO: 909 - protein, 38 aa, synthetic construct
SAATLAQIEALQAEIGKLQAEVNQLQAQLAQLQELLQC

SEQ ID NO: 910 - protein, 38 aa, synthetic construct
LLAEIEALKAEEASALAKLASSLGKLAEYLAKLKELLC

SEQ ID NO: 911 - protein, 38 aa, synthetic construct
AAAVAAQVAQLQAETAAALGKLQEAVAALQAALAALQC

SEQ ID NO: 912 - protein, 38 aa, synthetic construct
AAAAAELAAAASKLQDSLSSLASALSTAQELAKLAEEC

SEQ ID NO: 913 - protein, 38 aa, synthetic construct
SAAAAEAAAWQAKLQDALAKTASAKAEAEKLKKELEEC

SEQ ID NO: 914 - protein, 38 aa, synthetic construct
SAAAAEAAAYQAKLQASLSETASASSEASKLASLLSEC

SEQ ID NO: 915 - protein, 38 aa, synthetic construct
DALLAAAQAQIDAALGKLQELLSEAEALAKKAAELEAC

SEQ ID NO: 916 - protein, 38 aa, synthetic construct
AAAAAAAKAEELAAEQASVLGQLQDIVAATAALLAALC

SEQ ID NO: 917 - protein, 38 aa, synthetic construct
AALAAIAASQAEAAALLAKLQEAVGKTAALQAEAEAIC

SEQ ID NO: 918 - protein, 38 aa, synthetic construct
GLEELQKLQEERAKAESELGKLQDVQAQLQAQLQALQC

SEQ ID NO: 919 - protein, 38 aa, synthetic construct
SLLQQLLEQAAQLQATAAALGELAAAVAALQEALAALC

SEQ ID NO: 920 - protein, 38 aa, synthetic construct
AAATVAAAQAAFAAALAKAQAAAAAAQAALAAQLAQLC

SEQ ID NO: 921 - protein, 38 aa, synthetic construct
ADVAATLAALAAQQASLAAQLAKLQKEAAELAALLAAC

SEQ ID NO: 922 - protein, 36 aa, synthetic construct; identical to SEQ ID NO: 560

SEQ ID NO: 923 - protein, 36 aa, synthetic construct; identical to SEQ ID NO: 561

SEQ ID NO: 924 - protein, 36 aa, synthetic construct; identical to SEQ ID NO: 562

SEQ ID NO: 925 - protein, 36 aa, synthetic construct; identical to SEQ ID NO: 563

SEQ ID NO: 926 - protein, 36 aa, synthetic construct; identical to SEQ ID NO: 564

SEQ ID NO: 927 - protein, 36 aa, synthetic construct; identical to SEQ ID NO: 565

SEQ ID NO: 928 - protein, 36 aa, synthetic construct; identical to SEQ ID NO: 566

SEQ ID NO: 929 - protein, 36 aa, synthetic construct; identical to SEQ ID NO: 567

SEQ ID NO: 930 - protein, 36 aa, synthetic construct; identical to SEQ ID NO: 568

SEQ ID NO: 931 - protein, 36 aa, synthetic construct; identical to SEQ ID NO: 569

SEQ ID NO: 932 - protein, 36 aa, synthetic construct; identical to SEQ ID NO: 570

SEQ ID NO: 933 - protein, 36 aa, synthetic construct; identical to SEQ ID NO: 571

SEQ ID NO: 934 - protein, 36 aa, synthetic construct; identical to SEQ ID NO: 572

SEQ ID NO: 935 - protein, 36 aa, synthetic construct; identical to SEQ ID NO: 573

SEQ ID NO: 936 - protein, 36 aa, synthetic construct; identical to SEQ ID NO: 574

SEQ ID NO: 937 - protein, 36 aa, synthetic construct; identical to SEQ ID NO: 575

SEQ ID NO: 938 - protein, 36 aa, synthetic construct; identical to SEQ ID NO: 576

SEQ ID NO: 939 - protein, 36 aa, synthetic construct; identical to SEQ ID NO: 577

SEQ ID NO: 940 - protein, 36 aa, synthetic construct; identical to SEQ ID NO: 578

SEQ ID NO: 941 - protein, 36 aa, synthetic construct; identical to SEQ ID NO: 579

SEQ ID NO: 942 - protein, 36 aa, synthetic construct; identical to SEQ ID NO: 580

SEQ ID NO: 943 - protein, 36 aa, synthetic construct; identical to SEQ ID NO: 581

SEQ ID NO: 944 - protein, 37 aa, synthetic construct
DISGINLSILNIQLEIEHLNEVIENLGESLIELQELC

SEQ ID NO: 945 - protein, 37 aa, synthetic construct
DISGINLTILNIQLEIEHLNELIENLGESLIELQELC

SEQ ID NO: 946 - protein, 37 aa, synthetic construct
DISGINLSVVNIQLEIEHLNELIENLGESLIELQELC

SEQ ID NO: 947 - protein, 37 aa, synthetic construct
DISSIAAAFVDIQEYIDHLLTVASELESALIYLIPLC

SEQ ID NO: 948 - protein, 37 aa, synthetic construct
  MOD_RES 7: note = 2-aminoisobutyric acid
  MOD_RES 14: note = ornithine
  MOD_RES 24: note = ornithine
DISGINXSVVNIQXEIEHLNEVIXNLNESLIDLQELC

SEQ ID NO: 949 - protein, 37 aa, synthetic construct
  MOD_RES 7: note = 2-aminoisobutyric acid
  MOD_RES 14: note = ornithine
  MOD_RES 18: note = ornithine
  MOD_RES 24: note = ornithine
DISGINXSVVNIQXEIDXLNEVIXNLNESLIDLQELC

SEQ ID NO: 950 - protein, 37 aa, synthetic construct
  MOD_RES 7: note = 2-aminoisobutyric acid
  MOD_RES 14: note = ornithine
  MOD_RES 24: note = ornithine
DISGINXSVVNIQXEIEHLNEVAXNLGESLIELQELC

SEQ ID NO: 951 - protein, 37 aa, synthetic construct
  MOD_RES 7: note = 2-aminoisobutyric acid
  MOD_RES 14: note = ornithine
  MOD_RES 18: note = ornithine
  MOD_RES 24: note = ornithine
DISGINXSVVNIQXEIDXLNEVAXNLGESLIELQELC

SEQ ID NO: 952 - protein, 37 aa, synthetic construct
  MOD_RES 7: note = 2-aminoisobutyric acid
  MOD_RES 14: note = ornithine
  MOD_RES 24: note = ornithine
DISGINXSVVNIQXEIEHLNEVIXNLGESLIELQELC

SEQ ID NO: 953 - protein, 37 aa, synthetic construct
  MOD_RES 7: note = 2-aminoisobutyric acid
  MOD_RES 14: note = ornithine
  MOD_RES 18: note = ornithine
  MOD_RES 24: note = ornithine
DISGINXSVVNIQXEIDXLNEVIXNLGESLIELQELC

SEQ ID NO: 954 - protein, 38 aa, synthetic construct; identical to SEQ ID NO: 349

SEQ ID NO: 955 - protein, 41 aa, synthetic construct
DISGINASVVNIQKEIDRLNEVAKNLNESLIDLQELGSGSG

SEQ ID NO: 956 - protein, 41 aa, synthetic construct
DISGINASVVNIQKEIDRLNEVAKNLNESLIDLQELEAAAK

SEQ ID NO: 957 - protein, 46 aa, synthetic construct
DISGINASVVNIQKEIDRLNEVAKNLNESLIDLQELEAAAKEAAAK

SEQ ID NO: 958 - protein, 36 aa, synthetic construct; identical to SEQ ID NO: 587
  MOD_RES 7: note = 2-aminoisobutyric acid
  MOD_RES 14: note = ornithine
  MOD_RES 18: note = ornithine
  MOD_RES 24: note = ornithine

SEQ ID NO: 959 - protein, 41 aa, synthetic construct
  MOD_RES 7: note = 2-aminoisobutyric acid
  MOD_RES 14: note = ornithine
  MOD_RES 18: note = ornithine
  MOD_RES 24: note = ornithine
DISGINXSVVNIQXEIDXLNEVIXNLNESLIDLQELGSGSG

SEQ ID NO: 960 - protein, 36 aa, synthetic construct; identical to SEQ ID NO: 595
  MOD_RES 14: note = ornithine
  MOD_RES 18: note = ornithine
  MOD_RES 24: note = ornithine

SEQ ID NO: 961 - protein, 36 aa, synthetic construct; identical to SEQ ID NO: 587
  MOD_RES 7: note = 2-aminoisobutyric acid
  MOD_RES 14: note = ornithine
  MOD_RES 18: note = Homoarginine
  MOD_RES 24: note = ornithine

SEQ ID NO: 962 - protein, 36 aa, synthetic construct; identical to SEQ ID NO: 595
  MOD_RES 14: note = ornithine
  MOD_RES 24: note = ornithine
  MOD_RES 18: note = Homoarginine

SEQ ID NO: 963 - protein, 36 aa, synthetic construct; identical to SEQ ID NO: 598
  MOD_RES 7: note = 2-aminoisobutyric acid
  MOD_RES 14: note = ornithine
  MOD_RES 18: note = ornithine
  MOD_RES 24: note = ornithine

SEQ ID NO: 964 - protein, 6 aa, synthetic construct
GSGSGC

SEQ ID NO: 965 - protein, 6 aa, synthetic construct
GGGGSC

SEQ ID NO: 966 - protein, 9 aa, synthetic construct
GGGGGGGGC

SEQ ID NO: 967 - protein, 7 aa, synthetic construct
GGGGGGC

SEQ ID NO: 968 - protein, 5 aa, synthetic construct
EAAAK

SEQ ID NO: 969 - protein, 6 aa, synthetic construct
EAAAKC

SEQ ID NO: 970 - protein, 11 aa, synthetic construct
EAAAKEAAAKC

SEQ ID NO: 971 - protein, 8 aa, synthetic construct
AEAAAKAC

SEQ ID NO: 972 - protein, 4 aa, synthetic construct
GSGC

SEQ ID NO: 973 - protein, 13 aa, synthetic construct
GSAGSAAGSGEFC

SEQ ID NO: 974 - protein, 19 aa, synthetic construct
KESGSVSSEQLAQFRSLDC

SEQ ID NO: 975 - protein, 15 aa, synthetic construct
EGKSSGSGSESKSTC

SEQ ID NO: 976 - protein, 8 aa, synthetic construct
ELGSGSGC

SEQ ID NO: 977 - protein, 6 aa, synthetic construct
CGSGSG

SEQ ID NO: 978 - protein, 8 aa, synthetic construct
CGSGSGLE

SEQ ID NO: 979 - protein, 9 aa, synthetic construct
CGGGGGGGG

SEQ ID NO: 980 - protein, 7 aa, synthetic construct
CGGGGGG

SEQ ID NO: 981 - protein, 5 aa, synthetic construct
KAAAE

SEQ ID NO: 982 - protein, 6 aa, synthetic construct
CKAAAE

SEQ ID NO: 983 - protein, 11 aa, synthetic construct
CKAAAEKAAAE

SEQ ID NO: 984 - protein, 8 aa, synthetic construct
CAKAAAEA

SEQ ID NO: 985 - protein, 4 aa, synthetic construct
CGSG

SEQ ID NO: 986 - protein, 13 aa, synthetic construct
CFEGSGAASGASG

SEQ ID NO: 987 - protein, 19 aa, synthetic construct
CDLSRFQALQESSVSGSEK

SEQ ID NO: 988 - protein, 15 aa, synthetic construct
CTSKSESGSGSSKGE

SEQ ID NO: 989 - protein, 6 aa, synthetic construct
CSGGGG

SEQ ID NO: 990 - protein, 11 aa, synthetic construct
DISGINASVVN

SEQ ID NO: 991 - protein, 17 aa, synthetic construct
PDVDLGDISGINASVVN

SEQ ID NO: 992 - protein, 11 aa, synthetic construct
DIAAINASVAN

SEQ ID NO: 993 - protein, 11 aa, synthetic construct
DISGINASVVE

SEQ ID NO: 994 - protein, 11 aa, synthetic construct
SLDYINVTFLD

SEQ ID NO: 995 - protein, 11 aa, synthetic construct
SLTQINTTLLD

SEQ ID NO: 996 - protein, 11 aa, synthetic construct
DISGINASVVD

SEQ ID NO: 997 - protein, 11 aa, synthetic construct
SLDQINVTFLD

SEQ ID NO: 998 - protein, 11 aa, synthetic construct
DISQINASVVN

SEQ ID NO: 999 - protein, 11 aa, synthetic construct
SIDQINATFVD

SEQ ID NO: 1000 - protein, 22 aa, synthetic construct
KNHTSPDVDLGDISGINASVVN

SEQ ID NO: 1001 - protein, 15 aa, synthetic construct
EAAAKEAAAKEAAAK

SEQ ID NO: 1002 - protein, 18 aa, synthetic construct
PDVDLGGDISGINASVVN

SEQ ID NO: 1003 - protein, 23 aa, synthetic construct
KNHTSPDVDLGGDISGINASVVN

SEQ ID NO: 1004 - protein, 14 aa, synthetic construct
DLGDISGINASVVN

SEQ ID NO: 1005 - skipped

SEQ ID NO: 1006 - protein, 11 aa, synthetic construct
DISGINASVEN

SEQ ID NO: 1007 - protein, 14 aa, synthetic construct
DLGDISGINLTILN

SEQ ID NO: 1008 - protein, 14 aa, synthetic construct
DLGDISGINLSVVN

SEQ ID NO: 1009 - protein, 13 aa, synthetic construct
LGDISGINLTILN

SEQ ID NO: 1010 - protein, 14 aa, synthetic construct
DLGDISGINLSILN

SEQ ID NO: 1011 - protein, 13 aa, synthetic construct
LGDISGINLSILN

SEQ ID NO: 1012 - protein, 13 aa, synthetic construct
LGDISGINLSVVN

SEQ ID NO: 1013 - protein, 11 aa, synthetic construct
DISSIAAATVD

SEQ ID NO: 1014 - protein, 11 aa, synthetic construct
SISWIPAAFVD

SEQ ID NO: 1015 - protein, 11 aa, synthetic construct
DISSIIAATVD

SEQ ID NO: 1016 - protein, 11 aa, synthetic construct
SISWISASFVD

SEQ ID NO: 1017 - protein, 11 aa, synthetic construct
DISWIAAAYVD

SEQ ID NO: 1018 - protein, 11 aa, synthetic construct
DVSSIEASYVD

SEQ ID NO: 1019 - protein, 11 aa, synthetic construct
DISSIEASYVD

SEQ ID NO: 1020 - protein, 11 aa, synthetic construct
DISWIAAAFVD

SEQ ID NO: 1021 - protein, 11 aa, synthetic construct
DISSIIAAFVD

SEQ ID NO: 1022 - protein, 11 aa, synthetic construct
DISSIEASFVD

SEQ ID NO: 1023 - protein, 11 aa, synthetic construct
DISGINLSILN

SEQ ID NO: 1024 - protein, 11 aa, synthetic construct
DISGINLTILN

SEQ ID NO: 1025 - protein, 11 aa, synthetic construct
DISGINLSVVN

SEQ ID NO: 1026 - protein, 11 aa, synthetic construct
DISSIAAAFVD

SEQ ID NO: 1027 - protein, 13 aa, synthetic construct
  MOD_RES 7: note = 2-aminoisobutyric acid
DISGINXSVVNIQ

SEQ ID NO: 1028 - protein, 13 aa, synthetic construct
  MOD_RES 9: note = 2-aminoisobutyric acid
LGDISGINXSVVN

SEQ ID NO: 1029 - protein, 11 aa, synthetic construct
  MOD_RES 7: note = 2-aminoisobutyric acid
DISGINXSVVN

SEQ ID NO: 1030 - protein, 11 aa, synthetic construct
  MOD_RES 7: note = 2-aminoisobutyric acid
DISGINXSIVN

SEQ ID NO: 1031 - protein, 4 aa, synthetic construct
LQEL

SEQ ID NO: 1032 - protein, 4 aa, synthetic construct
LQAL

SEQ ID NO: 1033 - protein, 4 aa, synthetic construct
LDEI

SEQ ID NO: 1034 - protein, 4 aa, synthetic construct
LKEL

SEQ ID NO: 1035 - protein, 4 aa, synthetic construct
AAAK

SEQ ID NO: 1036 - protein, 5 aa, synthetic construct
LQELG

SEQ ID NO: 1037 - protein, 4 aa, synthetic construct
IQKK

SEQ ID NO: 1038 - protein, 4 aa, synthetic construct
ILDE

SEQ ID NO: 1039 - protein, 4 aa, synthetic construct
DLKE

SEQ ID NO: 1040 - protein, 4 aa, synthetic construct
LIPL